(19) United States
(12) Patent Application Publication
(10) Pub. No.: US 2024/0100192 A1
(43) Pub. Date: Mar. 28, 2024

(54) PROGRAMMABLE RNA WRITING USING CRISPR EFFECTORS AND TRANS-SPLICING TEMPLATES

(71) Applicant: Massachusetts Institute of Technology, Cambridge, MA (US)

(72) Inventors: Omar Osama Abudayyeh, Cambridge, MA (US); Jonathan S. Gootenberg, Cambridge, MA (US); Wenyuan Zhou, Cambridge, MA (US); Alisan Kayabolen, Brookline, MA (US); Cian Franz Schmitt-Ulms, Cambridge, MA (US)

(21) Appl. No.: 18/455,380

(22) Filed: Aug. 24, 2023

Related U.S. Application Data
(60) Provisional application No. 63/373,519, filed on Aug. 25, 2022.

(51) Int. Cl. A61K 48/00; A61P 43/00; C12N 9/22; C12N 15/63

(52) U.S. Cl. CPC A61K 48/0066; A61P 43/00; C12N 9/22; C12N 15/63

(57) ABSTRACT

This disclosure provides systems, methods, and compositions for site specific genetic engineering comprising the use of CRISPR effectors and trans-splicing. The disclosure also relates to methods of using the systems and compositions for treating diseases as well as diagnostics.

### CROSS REFERENCE TO RELATED APPLICATION

This application claims the benefit of U.S. Provisional Patent Application Ser. No. 63/373,519, filed Aug. 25, 2022. The entire content of the above-referenced patent application is herein incorporated by reference in its entirety.

### STATEMENT REGARDING FEDERALLY SPONSORED RESEARCH OR DEVELOPMENT

This invention was made with government support under Grant Nos. 1-R56-HG011857-01 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 30, 2023, is named 744063_083474-035_SL.xlm and is 1,201,176 bytes in size.

### FIELD

This disclosure relates to non-naturally occurring systems and compositions for site specific genetic engineering comprising the use of CRISPR effectors and trans-splicing templates. The disclosure also relates to methods of using the systems and compositions for the prevention and treatment of diseases.

### BACKGROUND

While gene editing technologies have revolutionized the ability to program DNA editing with high efficiency in diverse tissues, there remain several challenges with DNA editing, including permanent off-targets, concern for permanent correction of certain diseases, and some diseases being better targeted by other modalities than gene editing. For example, treatment of triplet repeat disorders with gene editing remains difficult, due to the difficulty of targeting repeat regions in the genome and the need to make large and precise deletions, without causing off-target genome rearrangements and other undesired effects on the genome.

RNA modifications, however, may offer a better approach with notable features: 1) temporal and reversible modification of genetic diseases, 2) minimal off-targets which are reversible and less harmful, and 3) more versatile editing beyond genome editing. For example, with triplet repeat disorders, an RNA writing strategy could allow for collapse of the repeats to the exact desired number, an approach that would be more successful than gene editing or RNA knockdown strategies that have failed. To accomplish RNA writing, which involves all possible base edits (transitions and transversions), small or large insertions, and small or large replacements (e.g., exon swapping), some approaches have been developed, such as trans-splicing, but with limited success.

Therefore, there is a need for more effective tools for gene editing and delivery.

### SUMMARY

In one aspect, the disclosure provides a composition for nucleic acid editing comprising a trans-splicing template polynucleotide comprising a cargo guide sequence complementary to a portion of an intron or exon sequence of a target RNA sequence. The trans-splicing template polynucleotide optionally further comprises an integration sequence, a 3′ splicing site sequence, a branch point sequence, and/or a polypyrimidine tract sequence, wherein each sequence is operably connected in any order. The composition further comprises a Cas7-11 enzyme sequence coupled to a guide RNA sequence that is complementary to a portion of the intron or exon sequence of the target RNA sequence that is upstream, downstream, or overlapping of the portion of the intron or exon sequence that is complementary to the cargo guide sequence.

In some embodiments, the trans-splicing template comprises a nucleic acid sequence about 80% identical, about 90% identical, about 95% identical, about 99% identical, or identical to any one of the nucleic acid sequences of SEQ ID NOS: 106-171 and 179-184.

In some embodiments, the Cas7-11 enzyme sequence comprises a nucleic acid sequence about 80% identical, about 90% identical, about 95% identical, about 99% identical, or identical to any one of the nucleic acid sequences of SEQ ID NOS: 1-18.

In some embodiments, the guide RNA comprises a nucleic acid sequence about 80% identical, about 90% identical, about 95% identical, about 99% identical, or identical to any one of the nucleic acid sequences of SEQ ID NOS: 19-105 and 205-261.

In another aspect, the disclosure provides a method of editing the target RNA sequence in a cell. The method comprises providing to the cell the trans-splicing template polynucleotide, a vector comprising the trans-splicing template polynucleotide, or a particle comprising the trans-splicing template polynucleotide. The method further comprises providing to the cell a polynucleotide translating the Cas7-11 enzyme sequence, a vector comprising a polynucleotide translating the Cas7-11 enzyme sequence, or a particle comprising a polynucleotide translating the Cas7-11 enzyme sequence. The method further comprises providing to the cell a polynucleotide expressing the guide RNA sequence, a vector comprising a polynucleotide expressing the guide RNA sequence, or a particle comprising a polynucleotide expressing the guide RNA sequence. The method further comprises editing the target RNA sequence via 3′ trans-splicing.

In another aspect, the disclosure provides a method of treating or preventing a genetically inherited disease in a subject in need thereof, the method comprises administering to the subject an effective amount of the trans-splicing template polynucleotide, a vector comprising the trans-splicing template polynucleotide, or a particle comprising the trans-splicing template polynucleotide. The method further comprises administering to the subject an effective amount of a polynucleotide translating the Cas7-11 enzyme sequence, a vector comprising a polynucleotide translating the Cas7-11 enzyme sequence, or a particle comprising a polynucleotide translating the Cas7-11 enzyme sequence. The method further comprises administering to the subject an effective amount of a polynucleotide expressing the guide RNA sequence, a vector comprising a polynucleotide expressing the guide RNA sequence, or a particle comprising a polynucleotide expressing the guide RNA sequence.

In some embodiments, the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

In another aspect, the disclosure provides a composition for nucleic acid editing comprising a trans-splicing template polynucleotide comprising a cargo guide sequence complementary to a portion of an intron or exon sequence of a target RNA sequence. The trans-splicing template polynucleotide optionally further comprises an integration sequence, a 5′ splicing site sequence, an intronic signal enhancer (ISE) sequence, and/or an exonic signal enhancer (ESE) sequence, wherein each sequence is operably connected in any order. The composition further comprises a Cas7-11 enzyme sequence coupled to a guide RNA sequence that is complementary to a portion of the intron or exon sequence of the target RNA sequence that is upstream, downstream, or overlapping of the portion of the intron or exon sequence that is complementary to the cargo guide sequence.

In some embodiments, the trans-splicing template comprises a nucleic acid sequence about 80% identical, about 90% identical, about 95% identical, about 99% identical, or identical to any one of the nucleic acid sequences of SEQ ID NOS: 106-171 and 179-184.

In some embodiments, the Cas7-11 enzyme sequence comprises a nucleic acid sequence about 80% identical, about 90% identical, about 95% identical, about 99% identical, or identical to any one of the nucleic acid sequences of SEQ ID NOS: 1-18.

In some embodiments, the guide RNA comprises a nucleic acid sequence about 80% identical, about 90% identical, about 95% identical, about 99% identical, or identical to any one of the nucleic acid sequences of SEQ ID NOS: 19-105 and 205-261.

In another aspect, the disclosure provides a method of editing the target RNA sequence in a cell, the method comprising providing to the cell the trans-splicing template polynucleotide, a vector comprising the trans-splicing template polynucleotide, or a particle comprising the trans-splicing template polynucleotide. The method further comprises providing to the cell a polynucleotide translating the Cas7-11 enzyme sequence, a vector comprising a polynucleotide translating the Cas7-11 enzyme sequence, or a particle comprising a polynucleotide translating the Cas7-11 enzyme sequence. The method further comprises providing to the cell a polynucleotide expressing the guide RNA sequence, a vector comprising a polynucleotide expressing the guide RNA sequence, or a particle comprising a polynucleotide expressing the guide RNA sequence. The method further comprises editing the target RNA sequence via 5′ trans-splicing.

In another aspect, the disclosure provides a method of treating or preventing a genetically inherited disease in a subject in need thereof, the method comprising administering to the subject an effective amount of the trans-splicing template polynucleotide, a vector comprising the trans-splicing template polynucleotide, or a particle comprising the trans-splicing template polynucleotide. The method further comprises administering to the subject an effective amount of a polynucleotide translating the Cas7-11 enzyme sequence, a vector comprising a polynucleotide translating the Cas7-11 enzyme sequence, or a particle comprising a polynucleotide translating the Cas7-11 enzyme sequence. The method further comprises administering to the subject an effective amount of a polynucleotide expressing the guide RNA sequence, a vector comprising a polynucleotide expressing the guide RNA sequence, or a particle comprising a polynucleotide expressing the guide RNA sequence.

In some embodiments, the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

In another aspect, the disclosure provides a composition for nucleic acid editing comprising a trans-splicing template polynucleotide comprising a first cargo guide sequence complementary to a portion of the first intron or exon sequence of a target RNA sequence and a second cargo guide sequence complementary to a portion of the second intron or exon sequence of the target RNA sequence. The trans-splicing template polynucleotide optionally further comprises an integration sequence, a 3′ splicing site sequence, a 5′ splicing site sequence, a branch point sequence, and/or a polypyrimidine tract sequence, wherein each sequence is operably connected in any order. The composition further comprises a first Cas7-11 enzyme sequence coupled to a first guide RNA sequence that is complementary to a portion of the first intron or exon sequence of the target RNA sequence that is upstream, downstream, or overlapping of the portion of the intron sequence that is complementary to the first cargo guide sequence. The composition optionally further comprises a second Cas7-11 enzyme sequence coupled to a second guide RNA sequence that is complementary to a portion of the second intron or exon sequence of the target RNA sequence that is upstream, downstream, or overlapping of the portion of the intron sequence that is complementary to the second cargo guide sequence.

In some embodiments, the trans-splicing template comprises a nucleic acid sequence about 80% identical, about 90% identical, about 95% identical, about 99% identical, or identical to any one of the nucleic acid sequences of SEQ ID NOS: 106-171 and 179-184.

In some embodiments, the Cas7-11 enzyme sequence comprises a nucleic acid sequence about 80% identical, about 90% identical, about 95% identical, about 99% identical, or identical to any one of the nucleic acid sequences of SEQ ID NOS: 1-18.

In some embodiments, the guide RNA comprises a nucleic acid sequence about 80% identical, about 90% identical, about 95% identical, about 99% identical, or identical to any one of the nucleic acid sequences of SEQ ID NOS: 19-105 and 205-261.

In another aspect, the disclosure provides a method of editing the target RNA sequence in a cell, the method comprising providing to the cell the trans-splicing template polynucleotide, a vector comprising the trans-splicing template polynucleotide, or a particle comprising the trans-splicing template polynucleotide. The method further comprises providing to the cell a polynucleotide translating the first Cas7-11 enzyme sequence, a vector comprising a polynucleotide translating the first Cas7-11 enzyme sequence, or a particle comprising a polynucleotide translating the first Cas7-11 enzyme sequence. The method further comprises providing to the cell a polynucleotide translating the second Cas7-11 enzyme sequence, a vector comprising a polynucleotide translating the second Cas7-11 enzyme sequence, or a particle comprising a polynucleotide translating the second Cas7-11 enzyme sequence. The method further comprises providing to the cell a polynucleotide expressing the guide RNA sequence, a vector comprising a polynucleotide expressing the guide RNA sequence, or a particle comprising a polynucleotide expressing the guide RNA sequence. The method further comprises editing the target RNA sequence via internal trans-splicing.

In another aspect, the disclosure provides a method of treating or preventing a genetically inherited disease in a subject in need thereof, the method comprising administering to the subject an effective amount of the trans-splicing template polynucleotide, a vector comprising the trans-splicing template polynucleotide, or a particle comprising the trans-splicing template polynucleotide. The method further comprises administering to the subject an effective amount of a polynucleotide translating the first Cas7-11 enzyme sequence, a vector comprising a polynucleotide translating the first Cas7-11 enzyme sequence, or a particle comprising a polynucleotide translating the first Cas7-11 enzyme sequence. The method further comprises administering to the subject an effective amount of a polynucleotide translating the second Cas7-11 enzyme sequence, a vector comprising a polynucleotide translating the second Cas7-11 enzyme sequence, or a particle comprising a polynucleotide translating the second Cas7-11 enzyme sequence. The method further comprises administering to the subject an effective amount of a polynucleotide expressing the guide RNA sequence, a vector comprising a polynucleotide expressing the guide RNA sequence, or a particle comprising a polynucleotide expressing the guide RNA sequence.

In some embodiments, the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

These and other aspects and embodiments of the applicants' teaching are set forth herein.

### BRIEF DESCRIPTION OF THE DRAWINGS

Aspects, features, benefits and advantages of the embodiments described herein will be apparent with regard to the following description, appended claims, and accompanying drawings where:

FIG. 1 is a schematic showing a DiCas7-11-assisted 3′ trans-splicing through target transcript cleavage of a luciferase reporter;

FIG. 2 is a schematic showing a DiCas7-11-assisted 5′ trans-splicing through target transcript cleavage;

FIG. 3 is a schematic showing a DiCas7-11-assisted internal trans-splicing through target transcript cleavage;

FIG. 4A is a schematic showing a DiCas7-11-assisted 3′ trans-splicing through target transcript cleavage;

FIG. 4B is a heat chart showing a DiCas7-11-assisted 3′ trans-splicing activity (fold change of normalized Gluc luminescence) on a 5′-fragment of Gluc pre-mRNA target (1-76 aa);

FIG. 5A is a heat chart showing a DiCas7-11-assisted 3′ trans-splicing activity (fold change of normalized Gluc luminescence) on a 5′-fragment of Gluc pre-mRNA target (1-76 aa) with midi prepped plasmids and a smaller panel of Cas7-11 guides;

FIG. 5B is a heat chart showing a DiCas7-11-assisted 3′ trans-splicing activity (fold change o trans-splicing efficiency by GNS) on a 5′-fragment of Gluc pre-mRNA target (1-76 aa) with midi prepped plasmids and a smaller panel of Cas7-11 guides;

FIG. 6A is a schematic showing a DiCas7-11-assisted internal trans-splicing through target transcript cleavage;

FIG. 6B is a heat chart showing a DiCas7-11-assisted internal trans-splicing activity (Gluc/Cluc fold change) on a Gluc pre-mRNA target, with 3×STOP codon inserted to the 37-76 aa coding sequenced flanked by intron 46 and intron 48 of COL7A1 in the targeted Gluc pre-mRNA;

FIG. 6C is a heat chart showing a DiCas7-11-assisted internal trans-splicing activity (measured by NGS) on a Gluc pre-mRNA target, with 3×STOP codon inserted to the 37-76 aa coding sequenced flanked by intron 46 and intron 48 of COL7A1 in the targeted Gluc pre-mRNA;

FIG. 7A is a heat chart showing a DiCas7-11-assisted 3′ trans-splicing activity (trans-splicing efficiency % by NGS) targeting intron 2 of MALAT pre-mRNA;

FIG. 7B is a heat chart showing a DiCas7-11-assisted 3′ trans-splicing activity (trans-splicing efficiency % by NGS) targeting intron 5 of STAT3 pre-mRNA;

FIG. 8 is a heat chart showing a DiCas7-11-assisted 3′ trans-splicing activity (trans-splicing efficiency % by NGS) targeting STAT3 pre-mRNA;

FIG. 9 is an image of a gel showing the measurement of DiCas7-11-assisted 3′ trans-splicing of STAT3 via a protein-based readout according to embodiments of the present teachings;

FIG. 10A is a schematic showing a DiCas7-11-assisted 3′ trans-splicing on a 5′-fragment of Gluc pre-mRNA target with a fusion of cargo guide;

FIG. 10B is a bar graph showing a 3′ trans-splicing activity (fold change of normalized Gluc luminescence) on the 5′-fragment of Gluc pre-mRNA target with a cargo template that contains a Cas7-11 guide as well as a target binding domain (i.e., cargo guide);

FIG. 11A is a schematic showing a Cas7-11-MCP fusion protein assisted 3′ trans-splicing on a 5′-fragment of Gluc pre-mRNA target with a fusion of cargo guide and MS2 hairpin (MS2-hyb-cargo);

FIG. 11B is a schematic showing a Cas7-11-MCP fusion protein assisted 3′ trans-splicing on a 5′-fragment of Gluc pre-mRNA target with a fusion of cargo guide and MS2 hairpin (hyb-MS2-cargo);

FIG. 11C is a heat chart showing the 3′ trans-splicing activity (fold change of normalized Gluc luminescence) on the 5′-fragment of Gluc pre-mRNA target with a fusion of cargo guide and MS2 hairpin as well as Cas7-11-MCP fusion proteins;

FIG. 12A is a schematic showing a DiCas7-11-assisted internal trans-splicing through target transcript cleavage;

FIG. 12B is heat graph showing the internal trans-splicing activity (fold change of normalized Gluc luminescence) on a Gluc pre-mRNA target, with 3×STOP codon inserted to the 37-76 aa coding sequenced flanked by intron 46 and intron 48 of COL7A1 in the targeted Gluc pre-mRNA;

FIG. 12C is heat graph showing the internal trans-splicing activity (trans-splicing efficiency % by NGS) on a Gluc pre-mRNA target, with 3×STOP codon inserted to the 37-76 aa coding sequenced flanked by intron 46 and intron 48 of COL7A1 in the targeted Gluc pre-mRNA;

FIG. 13 is bar graph showing 3′ endogenous trans-splicing rates (%) for the gene STAT3 using one common cargo replacing STAT3 exon 6 and either a Cas7-11 guide RNA targeting the STAT3 intron 5 or a guide scrambled sequence;

FIG. 14 is bar graph showing 3′ endogenous trans-splicing rates (%) for the gene STAT3 using one common cargo replacing STAT3 exon 6 and a set of Cas7-11 or Cas13 guides targeting intron 5 or a scrambled sequence, arrayed around a position that induced efficient splicing for Cas7-11 constructs;

FIG. 15 is a bar graph showing 3′ endogenous trans-splicing rates (%) for the gene STAT3, using one common cargo replacing STAT3 exon 6 and a set of Cas7-11 or Cas13 guides targeting intron 5 or a scrambled sequence, arrayed around a position that induced efficient splicing for Cas7-11 constructs;

FIG. 16 is a bar graph showing 3′ endogenous trans-splicing rates (%) for the gene PABPC1 using one common cargo replacing the PABPC1 terminal exon 14 and either a PABPC1 intron 13 or scrambled guide;

FIG. 17 is a bar graph showing 3′ endogenous trans-splicing rates (%) for the gene STAT3 using one common cargo replacing the STAT3 exon 6 and either a STAT3 intron 5 or scrambled guide;

FIG. 18 is a bar graph showing 3′ endogenous trans-splicing rates (%) for the gene PPIB using one common cargo replacing the PPIB terminal exon and either a PPIB intron 4 or scrambled guide;

FIG. 19 is a bar graph showing 3′ endogenous trans-splicing rates (%) for the gene TOP2A using one common cargo replacing the TOP2A exon 21 and either a TOP2A intron 20 or scrambled guide;

FIG. 20 is a bar graph showing 3′ endogenous trans-splicing rates (%) for the gene PPIB using one common cargo replacing the PPIB terminal exon and either a PPIB intron 4 or scrambled guide;

FIG. 21 is a bar graph showing 3′ endogenous trans-splicing rates (%) for the gene STAT3 using one common cargo replacing the STAT3 exon 6 and either a STAT3 intron 5 or scrambled guide;

FIG. 22 is a bar graph showing 3′ endogenous trans-splicing rates (%) for the gene TOP2A using one common cargo replacing the TOP2A exon 21 and either a TOP2A intron 20 or scrambled guide;

FIG. 23 is a bar graph showing 3′ endogenous trans-splicing rates (%) for the gene SHANK3 using one common cargo replacing the SHANK3 exon 21 and either a SHANK3 intron 20 or scrambled guide;

FIG. 24 is a bar graph showing 3′ endogenous trans-splicing rates (%) for the gene SHANK3 using one common cargo replacing the SHANK3 exon 21 and either a SHANK3 intron 20 or scrambled guide;

FIG. 25 is a bar graph showing 5′ endogenous trans-splicing rates (%) for the gene HTT using one common cargo replacing HTT exon 1 and either a HTT intron 1 or scrambled guide;

FIG. 26 is graph showing 3′ endogenous trans-splicing rates (%) for the gene STAT3 using one common cargo replacing STAT3 exon 6 and a targeting or nontargeting guide for STAT3 intron 5;

FIG. 27 is a bar graph showing 3′ endogenous trans-splicing rates (%) for the gene STAT3 using one common cargo structure replacing STAT3 exon 6 and a targeting or nontargeting guide for STAT3 intron 5;

FIG. 28 is a bar graph showing 3′ endogenous trans-splicing rates (%) for the gene STAT3 using one common cargo structure replacing STAT3 exon 6 and a targeting or nontargeting guide for STAT3 intron 5;

FIG. 29 is a bar graph showing 3′ endogenous trans-splicing rate (%) for the gene STAT3 using one common cargo structure replacing STAT3 exon 6 and a targeting or nontargeting guide for STAT3 intron 5;

FIG. 30 is a bar graph showing 3′ endogenous trans-splicing rates (%) for the gene PPIB using one common cargo replacing the PPIB terminal exon and either a PPIB intron 4 or scrambled guide;

FIG. 31 is a bar graph showing 3′ endogenous trans-splicing rates (%) for the gene PPIB using one common cargo replacing the PPIB terminal exon and either a PPIB intron 4 or scrambled guide;

FIG. 32 is a bar graph showing 3′ endogenous trans-splicing rates (%) for the gene PPIB using one common cargo replacing the PPIB terminal exon and either a PPIB intron 4 or scrambled guide;

FIG. 33A is a heat map chart showing 3′ endogenous trans-splicing rate (%) for the genes PPIB (PP), USF1 (U), STAT3 (S), PABPC1 (PA), and TOP2A(T) edited simultaneously within the same conditions using a target guide (FIG. 33A). FIG. 33B is a non-target (NT) guide (FIG. 33B);

FIG. 34 is a bar graph showing 3′ endogenous trans-splicing rates (%) for the gene STAT3 using one common cargo structure replacing STAT3 exon 6 and a targeting or nontargeting guide for STAT3 intron 5;

FIG. 35 is a bar graph showing 3′ endogenous trans-splicing rates (%) for the gene PPIB using one common cargo replacing the PPIB terminal exon and either a PPIB intron 4 or scrambled guide;

FIG. 36 is a bar graph showing 3′ endogenous trans-splicing rates (%) for the gene PPIB using one common cargo replacing the PPIB terminal exon and either a PPIB intron 4 or scrambled guide;

FIG. 37 is a bar graph showing 3′ endogenous trans-splicing rates (%) for the gene PPIB using one common structure cargo replacing the PPIB terminal exon and either a PPIB intron 4 or scrambled guide;

FIG. 38 is a bar graph showing 3′ endogenous trans-splicing rates (%) for the gene STAT3, using one common cargo structure replacing STAT3 exon 6 and a targeting or nontargeting guide for STAT3 intron 5;

FIG. 39 is a bar graph showing 3′ endogenous trans-splicing rates (%) for the gene PPIB using one common structure cargo replacing the PPIB terminal exon and either a PPIB intron 4 or scrambled guide;

FIG. 40 is a bar graph showing 3′ endogenous trans-splicing rates (%) for the gene SHANK3, exon 21;

FIG. 41 is a bar graph showing 3′ endogenous trans-splicing rates (%) for the gene SHANK3, using one common cargo replacing the SHANK3 exon 21 and either a SHANK3 intron 20 or scrambled guide;

FIG. 42 is a bar graph showing 3′ endogenous trans-splicing rates (%) for the gene STAT3 using one common cargo structure replacing STAT3 exon 6 and a targeting or nontargeting guide for STAT3 intron 5;

FIG. 43 is a bar graph showing 3′ endogenous trans-splicing rates (%) for the genes PPIB and STAT3 either alone or edited simultaneously;

FIG. 44 is a bar graph showing 3′ endogenous trans-splicing rates (%) for the gene STAT3 using one common cargo structure replacing STAT3 exon 6 and a targeting or nontargeting guide for STAT3 intron 5;

FIG. 45 is a bar graph showing 3′ endogenous trans-splicing rates (%) for the gene SHANK3 using one common cargo structure replacing SHANK3 exon 21 and a targeting or nontargeting guide for SHANK3 intron 20;

FIG. 46 is a bar graph showing 3′ endogenous trans-splicing rates (%) for the gene STAT3 using one common cargo structure replacing STAT3 exon 6 and a targeting or nontargeting guide for STAT3 intron 5 combined on a single plasmid;

FIG. 47 is a bar graph showing 3′ endogenous trans-splicing rates (%) for the gene SHANK3 using one common cargo structure replacing SHANK3 exon 21 and a targeting or nontargeting guide for SHANK3 intron 20 combined on a single plasmid;

FIG. 48A is bar graph showing 3′ endogenous trans-splicing rate (%) for the gene STAT3 using one common cargo replacing STAT3 exon 6 and either a Cas7-11 guide RNA targeting the STAT3 intron 5 or a guide scrambled sequence;

FIG. 48B is a bar graph showing 3′ endogenous trans-splicing rates (%) for the gene PPIB using one common structure cargo replacing the PPIB terminal exon and either a PPIB intron 4 or scrambled guide with the same set of truncated spliceosome fusions;

FIG. 49 is a bar graph showing 3′ endogenous trans-splicing rates (%) for the gene SHANK3 using conventional or lentiviral vectors;

FIG. 50 is a bar graph showing 3′ endogenous trans-splicing rates (%) for the gene SHANK3 using different volumes of 2 lentiviruses either alone or in combination;

FIG. 51A is a is a Western blot image showing protein readouts of 3′ endogenous trans-splicing of PPIB gene using a cargo replacing the PPIB terminal exon and containing 1× or 3×Flag or 1×HA tags, and either a PPIB intron 4 targeting or scrambled guide RNA. Bands around 15 kDa shows background expression of the cargo, while the faint band around 25 kDa represents the product of targeted trans-splicing;

FIG. 51B is a bar graph showing 3′ endogenous trans-splicing rates (%) for the gene PPIB as a confirmation of the Western blot;

FIG. 52A is a is a Western blot image showing protein readouts of 3′ trans-splicing of USF1 gene using 4 different components. Green bands around 28 kDa shows background expression of the cargo, while red bands around 33 and 35 kDa show spliced and un-spliced reporter, respectively. The band around 55 kDa which is brighter with targeting guide represents the product of targeted trans-splicing;

FIG. 52B is a bar graph showing 3′ trans-splicing rates (%) for the gene USF1 as a confirmation of the Western blot;

FIG. 53 is a bar graph showing 3′ trans-splicing rates (%) for the gLuc gene in a reporter plasmid;

FIG. 54 is a bar graph showing 5′ splicing rates for HTT exon 1, using cargos that bind to intron 1 and a guide targeting the terminal end of the cargo hybridization or a scrambled NT sequence;

FIG. 55 is a bar graph showing 5′ splicing rate (%) for HTT exon 1, using cargo constructs with hybridization regions that bind intron 1 of the HTT premRNA and either a scrambled guide or a guide that binds and cleaves the end of the hybridization;

FIG. 56 is a bar graph showing 5′ splicing rates for HTT exon 1 using cargos that bind to intron 1 and a guide targeting the terminal end of the cargo hybridization or a scrambled NT sequence combined on a single plasmid;

FIG. 57 is a bar graph showing 5′ splicing rate for HTT exon 1, using cargos that bind to intron 1 and a guide targeting the terminal end of the cargo hybridization or a scrambled NT sequence;

FIG. 58 is a bar graph showing 5′ splicing rates for HTT exon 1 using cargos that bind to intron 1 and a guide targeting the terminal end of the cargo hybridization or a scrambled NT sequence;

FIG. 59A is a bar graph showing 5′ splicing rates for HTT exon 1 using cargos that bind to intron 1 and a guide targeting the terminal end of the cargo hybridization or a scrambled NT sequence combined on a single plasmid;

FIG. 59B is a bar graph showing 5′ splicing rates (%) for HTT gene.

FIG. 60 is a bar graph showing 5′ splicing rates (%) for USF1 exon 9 using cargo constructs with hybridization regions that bind intron 9 of the USF1 premRNA and either a scrambled guide or a guide that binds and cleaves upstream of the hybridization region;

FIG. 61A is a bar graph showing 5′ trans-splicing rates (%) for HTT exon 1 using cargo constructs with hybridization regions that bind intron 1 of the HTT premRNA and either a scrambled guide or a guide that binds and cleaves the end of the hybridization;

FIG. 61B is a bar graph showing 3′ trans-splicing of SHANK3 exon 21 with a cargo and guide binding within intron 20;

FIG. 62 is a bar graph showing 5′ splicing rates (%) for PABPC1 exon 1 using cargo constructs with hybridization regions that bind intron 1 of the PABPC1 premRNA and either a scrambled guide or a guide that binds and cleaves the end of the hybridization;

FIG. 63 is a bar graph showing 5′ trans-splicing rates (%) for RPL41 exon 1 using cargo constructs with hybridization regions that bind intron 1 of the RPL41 premRNA and either a scrambled guide or a guide that binds and cleaves the end of the hybridization;

FIG. 64 is a bar graph showing 5′ trans-splicing rates (%) for HTT exon 1 using the original cargo construct and either a scrambled guide or a guide targeting the cargo RNA or intron 1 of the HTT premRNA;

FIG. 65 is a bar graph showing 5′ splicing rates (%) for HTT exon 1 using either the original cargo construct and either a scrambled guide or a guide targeting the cargo RNA or intron 1 of the HTT premRNA;

FIG. 66 is a bar graph showing 5′ splicing rate for HTT exon 1 using cargos that bind to intron 1 and a guide targeting the terminal end of the cargo hybridization or a scrambled NT sequence combined on a single plasmid; and

FIG. 67 is a bar graph showing 5′ splicing rates (%) for HTT exon 1 using a cargo construct with a hybridization region that binds intron 1 of the HTT premRNA.

These and other aspects of the applicants' teaching are set forth herein.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The section headings used herein are for organizational purposes only and are not to be construed as limiting the subject matter described.

### Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural referents unless the context clearly dictates otherwise.

The term “optional” or “optionally” means that the subsequent described event, circumstance, or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosed disclosure. It is to be understood that the value to which the modifier “about” or “approximately” refers is itself also specifically, and preferably, disclosed.

As used herein, the terms “operably connected” and “operably linked” are used interchangeably and refer to a linkage of polynucleotide sequence elements or polypeptide sequence elements in a functional relationship. For example, a polynucleotide sequence is operably connected when it is placed into a functional relationship with another polynucleotide sequence. In some embodiments, a transcription regulatory polynucleotide sequence e.g., a promoter, enhancer, or other expression control element is operably-linked to a polynucleotide sequence that encodes a protein if it affects the transcription of the polynucleotide sequence that encodes the protein.

The determination of “percent identity” between two sequences (e.g., polypeptide or polynucleotides) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (See, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

The terms “polynucleotide,” “nucleic acid,” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

The terms “protein” and “polypeptide” are used interchangeably herein and refer to a polymer of at least two amino acids linked by a peptide bond.

As used herein, the term “RNA” or “RNA polynucleotide” refers to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

A “therapeutically effective amount” of a therapeutic agent (e.g., a composition or system described herein) refers to any amount of the therapeutic agent that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

As used herein, the term “functional fragment” in reference to a protein refers to a fragment of a reference protein that retains at least one particular function. For example, a functional fragment of an aptamer binding protein can refer to a fragment of the protein that retains the ability to bind the cognate aptamer. Not all functions of the reference protein need be retained by a functional fragment of the protein. In some instances, one or more functions are selectively reduced or eliminated.

Compositions and Systems

The present disclosure provides (non-naturally occurring or engineered) systems for editing a nucleic acid such as a gene or a product thereof (e.g., the encoded RNA or protein). In some embodiments, the systems may be an engineered, non-naturally occurring system suitable for modifying post-translational modification sites on proteins encoded by a target nucleic acid sequence. In certain cases, the target nucleic acid sequence is RNA, e.g., mRNA or a fragment thereof. In certain cases, the target nucleic acid sequence is DNA, e.g., a gene or a fragment thereof. In general, the system may comprise, for example and without limitation, one or more Cas protein (e.g., Cas7-11) or/and catalytic inactive (dead) Cas protein (e.g., dead Cas7-11), one or more guide molecules (e.g., guide RNA), and one or more template (e.g., trans-splicing template). The guide sequence may be designed to have a degree of complementarity with a target sequence.

CRISPR-Cas

Some embodiments disclosed herein are directed to CRISPR-Cas (clustered regularly interspaced short palindromic repeats associated proteins) systems. In the conflict between bacterial hosts and their associated viruses, CRISPR-Cas systems provide an adaptive defense mechanism that utilizes programmed immune memory. CRISPR-Cas systems provide their defense through three stages: adaptation, the integration of short nucleic acid sequences into the CRISPR array that serves as memory of past infections; expression, the transcription of the CRISPR array into a pre-crRNA (CRISPR RNA) transcript and processing of the pre-crRNA into functional crRNA species targeting foreign nucleic acids; and interference, the programming of CRISPR effectors by crRNA to cleave nucleic acid of foreign threats. Across all CRISPR-Cas systems, these fundamental stages display enormous variation, including the identity of the target nucleic acid (either RNA, DNA, or both) and the diverse domains and proteins involved in the effector ribonucleoprotein complex of the system.

CRISPR-Cas systems can be broadly split into two classes based on the architecture of the effector modules involved in pre-crRNA processing and interference. Class 1 systems have multi-subunit effector complexes composed of many proteins, whereas Class 2 systems rely on single-effector proteins with multi-domain capabilities for crRNA binding and interference; Class 2 effectors often provide pre-crRNA processing activity as well. Class 1 systems contain 3 types (type I, III, and IV) and 33 subtypes, including the RNA and DNA targeting type III-systems. Class 2 CRISPR families encompass 3 types (type II, V, and VI) and 17 subtypes of systems, including the RNA-guided DNases Cas9 and Cas12 and the RNA-guided RNase Cas13. Continual sequencing of novel bacterial genomes and metagenomes uncovers new diversity of CRISPR-Cas systems and their evolutionary relationships, necessitating experimental work that reveals the function of these systems and develops them into new tools.

Among the currently known CRISPR-Cas systems, only the type III and type VI systems have been demonstrated to bind and target RNA, and these two systems have substantially different properties, the most distinguishing being their membership in Class 1 and Class 2, respectively. Characterized subtypes of type III, which span type III-A, B, and C systems, target both RNA and DNA species through an effector complex containing multiple Cas7 (Csm3/5 or Cmr1/4/6) RNA nuclease units in association with a single Cas10 (Csm1 or Cmr2) DNA nuclease. The RNA nuclease activity of Cas7 is mediated through acidic residues in the repeat-associated mysterious proteins (RAMP) domains, which cut at stereotyped intervals in the guide:target duplex. Type III systems also have a target restriction and cannot efficiently target protospacers in vivo if there is extended homology between the 5′ “tag” of the crRNA and the “anti-tag” 3′ of the protospacer in the target, although this binding does not block RNA cleavage in vitro. In type III systems, pre-crRNA processing is carried out by either host factors or the associated Cas6 family protein, which can physically complex with the effector machinery.

In contrast to type III systems, type VI systems contain a single CRISPR effector Cas13 that can only affect RNA interference, mediated through basic catalytic residues of dual HEPN domains. This interference requires a protospacer flanking sequence (PFS), although the influence of the PFS varies between orthologs and families. Importantly, the RNA cleavage activity of Cas13, once triggered by crRNA:target duplex formation, is indiscriminate, and activated Cas13 enzymes will cleave other RNA species in vitro, in bacterial hosts, and mammalian cells. This activity, termed the collateral effect, has been applied to CRISPR-based nucleic acid detection technologies. In addition to the RNA interference activity, the Cas13 family members contain pre-crRNA processing activity. Just as single-effector DNA targeting systems have given rise to numerous genome editing applications, Cas13 family members have been applied to a suite of RNA-targeting technologies in both bacterial and eukaryotic cells, including RNA knockdown, RNA editing, RNA tracking, epitranscriptome editing, translational upregulation, epi-transcriptomic reading and writing via N6-Methyladenosine, and isoform modulation.

The novel type III-E system was recently identified from genomes of 8 bacterial species and is characterized as a fusion of several Cas7 proteins and a putative Cas11 (Csm2)-like small subunit. The domain composition suggests the fusion of multiple type III effector module domains involved in crRNA binding into a single protein effector that is predicted to process pre-crRNA given its homology with Cas5 (Csm4) and conserved aspartates. The lack of other putative effector nucleases in these CRISPR loci raise the additional possibility that this fusion protein is capable of crRNA-directed RNA cleavage. If so, this system would blur the distinction of Class 1 and Class 2 systems, as it would have domains homologous to other Class 1 systems and possess a single effector module characteristic of Class 2 systems. Beyond the single effector module present in all subtype III-E loci, a majority of type III-E family members contain a putative ancillary gene with a CHAT domain, which is a caspase family protease associated with programmed cell death (PCD), suggesting involvement of PCD-mediated antiviral strategies, as has been observed with type III and VI systems.

Type III-E system associated effector is a programmable RNase. This system can provide defense against RNA phage and be programmed to target exogenous mRNA species when expressed heterologously in bacteria. Orthologs of Cas7-11 are capable of both processing of pre-crRNA and crRNA-directed cleavage of RNA targets and determine catalytic residues underlying programmed RNA cleavage. A direct evolutionary path of Cas7-11 can be traced from individual Cas7 and Cas11 effector proteins of subtype III-D1 variant, through an intermediate, a partially fused effector Cas7×3 of the subtype III-D2 variant, to the singe-effector architecture of subtype III-E that is so far unique among the Class 1 CRISPR-Cas systems. Cas7-11 most likely originated from two type III-D variants. Three Cas7 domains (domains 3, 4 and 5) are derived from subtype III-D2 that contains the Cas7×3 effector protein along with Cas10 and another Cas7-like domain fused to a Cas5-like domain. The origin of the N-terminal Cas7 and putative Cas11 domain of Cas7-11 is most likely derived from a III-D1 variant, where both genes are stand-alone.

Cas7-11 differs from Cas13, in terms of both domain organization and activity. Cas13 RNA cleavage is enacted by dual HEPN domains with basic catalytic residues, and this cleavage, once triggered, is indiscriminate. In contrast, Cas7-11 utilizes at least two of four Cas7-like domains with acidic catalytic residues to generate stereotyped cleavage at the target binding site in cis. Furthermore, Cas13 targeting is restricted by the requirement for a PFS, which Cas7-11 does not require, and the DR of Cas7-11-associated crRNA is substantially shorter. Because of these unique features, Cas7-11 may have distinct advantages for RNA targeting and transcriptome engineering biotechnology applications.

Regulation of interference by accessory proteins has been observed in both type III and type VI systems, and other proteins in the D. ishimotonii type III-E locus can regulate activity of DisCas7-11a. Notably, TPR-CHAT had a strong inhibitory effect on DisCas7-11a phage interference, raising the possibility that unrestricted DisCas7-11a activity could be detrimental for the host. Alternatively, as TPR-CHAT is a caspase family protease associated with programmed cell death (PCD), it is possible that TPR-CHAT is activated by DisCas7-11a and leads to host death, which could mimic death due to phage in these assays. TPR-CHAT caspase activity could be activated by DisCas7-11a and cause PCD through general proteolysis, analogous to PCD triggered by Cas13 collateral activity.

Similar to Class 2 CRISPR effectors such as Cas9, Cas12, and Cas13, Cas7-11 is highly active in mammalian cells, with substantial knockdown activity on both reporter and endogenous transcripts. Moreover, via inactivation of active sites through mutagenesis, the catalytically inactive dCas7-11 enzyme can be used to recruit ADAR2DD for efficient site-specific A-to-I editing on transcripts. These applications establish Cas7-11 as the basis for an RNA-targeting toolbox that has several benefits compared to Cas13, including the lack of sequence preferences and collateral activity, the latter of which has been shown to induce toxicity in certain cell types. A Cas7-11 toolbox may serve as the basis for multiple RNA technologies, including RNA knockdown, RNA editing, translation modulation, RNA recruitment, RNA tracking, splicing control, RNA stabilization, and potentially even diagnostics.

CRISPR-Cas Proteins and Guides

In some embodiments, the system comprises one or more components of a CRISPR-Cas system. For example, the system may comprise a Cas protein, a guide molecule, or a combination thereof.

In the methods and systems of the present disclosure use is made of a CRISPR-Cas protein and corresponding guide molecule. More particularly, the CRISPR-Cas protein is a class 2 CRISPR-Cas protein. In certain embodiments, said CRISPR-Cas protein is a Cas7-11. The Cas7-11 may be Cas7-11a, Cas7-11b, Cas7-11c, or Cas7-11d. The CRISPR-Cas system does not require the generation of customized proteins to target specific sequences but rather a single Cas protein can be programmed by guide molecule to recognize a specific nucleic acid target, in other words the Cas enzyme protein can be recruited to a specific nucleic acid target locus of interest using said guide molecule.

CRISPR-Cas Proteins

In some embodiments, the systems may comprise a CRISPR-Cas protein. In certain examples, the CRISPR-Cas protein may be a catalytically inactive (dead) Cas protein. The catalytically inactive (dead) Cas protein may have impaired (e.g., reduced or no) nuclease activity. In some cases, the dead Cas protein may have nickase activity. In some cases, the dead Cas protein may be dead Cas 15 protein. For example, the dead Cas 15 may be dead Cas7-11a, dead Cas7-11b, dead Cas7-11c, or dead Cas7-11d. In some embodiments, the system may comprise a nucleotide sequence encoding the dead Cas protein.

In its unmodified form, a CRISPR-Cas protein is a catalytically active protein. This implies that upon formation of a nucleic acid-targeting complex (comprising a guide RNA hybridized to a target sequence) one or both DNA strands in or near (e.g., within 1, 2, 3, 4, 5, 6, 7, 8, 9, 10, 20, 50, or more base pairs from) the target sequence is modified (e.g., cleaved). As used herein the term “sequence(s) associated with a target locus of interest” refers to sequences near the vicinity of the target sequence (e.g., within 1, 2, 3, 4, 5, 6, 7, 8, 9, 10, 20, 50, or more base pairs from the target sequence, wherein the target sequence is comprised within a target locus of interest). The unmodified catalytically active Cas7-11 protein generates a staggered cut, whereby the cut sites are typically within the target sequence. More particularly, the staggered cut is typically 13-23 nucleotides distal to the PAM. In particular embodiments, the cut on the non-target strand is 17 nucleotides downstream of the PAM (i.e. between nucleotide 17 and 18 downstream of the PAM), while the cut on the target strand (i.e. strand hybridizing with the guide sequence) occurs a further 4 nucleotides further from the sequence complementary to the PAM (this is 21 nucleotides upstream of the complement of the PAM on the 3′ strand or between nucleotide 21 and 22 upstream of the complement of the PAM).

In the methods according to the present disclosure, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the Cas7-11 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e., a nickase. More particularly, in the context of the present disclosure, the nickase ensures cleavage within the non-target sequence, i.e., the sequence which is on the opposite DNA strand of the target sequence and 3′ of the PAM sequence.

In some embodiments, a CRISPR-Cas protein is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. In these embodiments, the CRISPR-Cas protein is used as a generic DNA binding protein. The mutations may be artificially introduced mutations or gain- or loss-of-function mutations.

In addition to the mutations described above, the CRISPR-Cas protein may be additionally modified. As used herein, the term “modified” with regard to a CRISPR-Cas protein generally refers to a CRISPR-Cas protein having one or more modifications or mutations (including point mutations, truncations, insertions, deletions, chimeras, fusion proteins, etc.) compared to the wild type Cas protein from which it is derived. A modification by truncation can refer to an engineered truncation that is based on structure function analysis and not naturally occurring. By derived is meant that the derived enzyme is largely based, in the sense of having a high degree of sequence homology with, a wildtype enzyme, but that it has been mutated (modified) in some way as known in the art or as described herein. The modification can be fusions of effectors like fluorophore, proteins involved in translation modulation (e.g., eIF4E, eIF4A, and eIF4G) and proteins involved with epitranscriptomic modulation (e.g., pseudouridine synthase and m6a writer/readers), and splicing factors involved with changing splicing. Cas7-11 could also be used for sensing RNA for diagnostic purposes.

In some embodiments, the C-terminus of the Cas7-11 effector can be truncated. For example, at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, at least 350 amino acids, or any ranges that are made of any two or more points in the above list may be truncated at the C-terminus of the Cas7-11 effector. For example, up to 120 amino acids, up to 140 amino acids, up to 160 amino acids, up to 180 amino acids, up to 200 amino acids, up to 250 amino acids, up to 300 amino acids, up to 350 amino acids, up to 400 amino acids, or any ranges that are made of any two or more points in the above list may be truncated at the C-terminus of the Cas7-11 effector.

In some embodiments, the N-terminus of the Cas7-11 effector protein may be truncated. For example, at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, at least 350 amino acids, or any ranges that are made of any two or more points in the above list may be truncated at the N-terminus of the Cas7-11 effector. For examples, up to 120 amino acids, up to 140 amino acids, up to 160 amino acids, up to 180 amino acids, up to 200 amino acids, up to 250 amino acids, up to 300 amino acids, up to 350 amino acids, up to 400 amino acids, or any ranges that are made of any two or more points in the above list may be truncated at the N-terminus of the Cas7-11 effector.

In some embodiments, both the N- and the C-termini of the Cas7-11 effector protein may be truncated. For example, at least 20 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 40 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 60 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 80 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 100 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 120 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 140 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 160 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 180 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 200 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 220 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 240 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 260 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 280 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 300 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 350 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 20 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 40 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 60 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 80 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 100 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 120 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 140 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 160 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 180 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 200 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 220 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 240 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 260 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 280 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 300 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 350 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector.

In some embodiments, the Cas7-11 effector comprises a deletion of the INS domain. For example, at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, at least 350 amino acids, or any ranges that are made of any two or more points in the above list of the INS domain may be deleted.

In some embodiments, the INS domain of the Cas7-11 effector is replaced by a linker. See, e.g., Reddy Chichili, V. P., Kumar, V., & Sivaraman, J., “Linkers in the structural biology of protein-protein interactions,” Protein science: a publication of the Protein Society, 22(2), 153-167 (2013); https://doi.org/10.1002/pro.2206, incorporated herewith in its entirety by reference. For example, the INS domain of the Cas7-11 effector may be replaced by a GG, GGG, GS, GGS, GGGS (SEQ ID NO: 172), and/or GGGGS linker (SEQ ID NO: 173). For example, the INS domain of the Cas7-11 effector may be replaced by a (GG)x (SEQ ID NO: 174), (GGG)x (SEQ ID NO: 175), (GGS)x (SEQ ID NO: 176), (GGGS)x (SEQ ID NO: 177), and/or a (GGGGS)x linker (SEQ ID NO: 178), wherein x is independently 1, 2, 3, 4, 5, 6, 7, 8, 9, 10, 11, or 12. For example, the INS domain of the Cas7-11 effector may be replaced by a linker with at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or any ranges that are made of any two or more points in the above list.

The additional modifications of the CRISPR-Cas protein may or may not cause an altered functionality. By means of example, and in particular with reference to CRISPR-Cas protein, modifications which do not result in an altered functionality include for instance codon optimization for expression into a particular host, or providing the nuclease with a particular marker (e.g., for visualization). Modifications with may result in altered functionality may also include mutations, including point mutations, insertions, deletions, truncations (including split nucleases), etc. Fusion proteins may without limitation include for instance fusions with heterologous domains or functional domains (e.g., localization signals, catalytic domains, etc.). In certain embodiments, various modifications may be combined (e.g., a mutated nuclease which is catalytically inactive, and which further is fused to a functional domain, such as for instance to induce DNA methylation or another nucleic acid modification, such as including without limitation a break (e.g., by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” Cas proteins) or decreased specificity, or altered PAM recognition), altered activity (e.g., increased or decreased catalytic activity, including catalytically inactive nucleases or nickases), and/or altered stability (e.g., fusions with destabilization domains). Suitable heterologous domains include without limitation a nuclease, a ligase, a repair protein, a methyltransferase, (viral) integrase, a recombinase, a transposase, an argonaute, a cytidine deaminase, a retron, a group II intron, a phosphatase, a phosphorylase, a sulpfurylase, a kinase, a polymerase, an exonuclease, etc. Examples of all these modifications are known in the art. It will be understood that a “modified” nuclease as referred to herein, and in particular a “modified” Cas or “modified” CRISPR-Cas system or complex preferably still has the capacity to interact with or bind to the poly-nucleic acid (e.g., in complex with the guide molecule). Such modified Cas protein can be combined with the deaminase protein or active domain thereof as described herein.

In certain embodiments, CRISPR-Cas protein may comprise one or more modifications resulting in enhanced activity and/or specificity, such as including mutating residues that stabilize the targeted or non-targeted strand (e.g., eCas9; “Rationally engineered Cas9 nucleases with improved specificity”, Slaymaker et al. (2016), Science, 351(6268):84-88, incorporated herewith in its entirety by reference). In certain embodiments, the altered or modified activity of the engineered CRISPR protein comprises increased targeting efficiency or decreased off-target binding. In certain embodiments, the altered activity of the engineered CRISPR protein comprises modified cleavage activity. In certain embodiments, the altered activity comprises increased cleavage activity as to the target polynucleotide loci. In certain embodiments, the altered activity comprises decreased cleavage activity as to the target polynucleotide loci. In certain embodiments, the altered activity comprises decreased cleavage activity as to off-target polynucleotide loci. In certain embodiments, the altered or modified activity of the modified nuclease comprises altered helicase kinetics. In certain embodiments, the modified nuclease comprises a modification that alters association of the protein with the nucleic acid molecule comprising RNA (in the case of a Cas protein), or a strand of the target polynucleotide loci, or a strand of off-target polynucleotide loci. In an aspect of the disclosure, the engineered CRISPR protein comprises a modification that alters formation of the CRISPR complex. In certain embodiments, the altered activity comprises increased cleavage activity as to off-target polynucleotide loci. Accordingly, in certain embodiments, there is increased specificity for target polynucleotide loci as compared to off-target polynucleotide loci. In other embodiments, there is reduced specificity for target polynucleotide loci as compared to off-target polynucleotide loci. In certain embodiments, the mutations result in decreased off-target effects (e.g., cleavage or binding properties, activity, or kinetics), such as in case for Cas proteins for instance resulting in a lower tolerance for mismatches between target and guide RNA. Other mutations may lead to increased off-target effects (e.g., cleavage or binding properties, activity, or kinetics). Other mutations may lead to increased or decreased on-target effects (e.g., cleavage or binding properties, activity, or kinetics). In certain embodiments, the mutations result in altered (e.g., increased or decreased) helicase activity, association, or formation of the functional nuclease complex (e.g., CRISPR-Cas complex). In certain embodiments, as described above, the mutations result in an altered PAM recognition, i.e., a different PAM may be (in addition or in the alternative) be recognized, compared to the unmodified Cas protein. Particularly preferred mutations include positively charged residues and/or (evolutionary) conserved residues, such as conserved positively charged residues, in order to enhance specificity. In certain embodiments, such residues may be mutated to uncharged residues, such as alanine.

Type-III CRISPR-Cas Proteins

The application describes methods using Type-III CRISPR-Cas proteins. This is exemplified herein with Cas7-11, whereby a number of orthologs or homologs have been identified. It will be apparent to the skilled person that further orthologs or homologs can be identified and that any of the functionalities described herein may be engineered into other orthologs, including chimeric enzymes comprising fragments from multiple orthologs.

Computational methods of identifying novel CRISPR-Cas loci are described in EP3009511 or US2016208243 and may comprise the following steps: detecting all contigs encoding the Cas1 protein; identifying all predicted protein coding genes within 20 kB of the cas1 gene; comparing the identified genes with Cas protein-specific profiles and predicting CRISPR arrays; selecting unclassified candidate CRISPR-Cas loci containing proteins larger than 500 amino acids (>500 aa); analyzing selected candidates using methods such as PSI-BLAST and HH11Pred to screen for known protein domains, thereby identifying novel Class 2 CRISPR-Cas loci (see also Schmakov et al. 2015, Mol Cell. 60(3):385-97). In addition to the above-mentioned steps, additional analysis of the candidates may be conducted by searching metagenomics databases for additional homologs. Additionally, or alternatively, to expand the search to non-autonomous CRISPR-Cas systems, the same procedure can be performed with the CRISPR array used as the seed.

In one aspect the detecting all contigs encoding the Cas1 protein is performed by GenemarkS, a gene prediction program as further described in “GeneMarkS: a self-training method for prediction of gene starts in microbial genomes. Implications for finding sequence motifs in regulatory regions.” John Besemer, Alexandre Lomsadze and Mark Borodovsky, Nucleic Acids Research (2001) 29, pp 2607-2618, herein incorporated by reference.

In one aspect the identifying all predicted protein coding genes is carried out by comparing the identified genes with Cas protein-specific profiles and annotating them according to NCBI Conserved Domain Database (CDD) which is a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM). In a further aspect, CRISPR arrays were predicted using a PILER-CR program which is a public domain software for finding CRISPR repeats as described in “PILER-CR: fast and accurate identification of CRISPR repeats,” Edgar, R. C., BMC Bioinformatics, January 20; 8:18(2007), herein incorporated by reference.

In a further aspect, the case-by-case analysis is performed using PSI-BLAST (Position-Specific Iterative Basic Local Alignment Search Tool). PSI-BLAST derives a position-specific scoring matrix (PSSM) or profile from the multiple sequence alignment of sequences detected above a given score threshold using protein-protein BLAST. This PSSM is used to further search the database for new matches and updated for subsequent iterations with these newly detected sequences. Thus, PSI-BLAST provides a means of detecting distant relationships between proteins.

In another aspect, the case-by-case analysis is performed using HHpred, a method for sequence database searching and structure prediction that is as easy to use as BLAST or PSI-BLAST and that is at the same time much more sensitive in finding remote homologs. In fact, HHpred's sensitivity is competitive with the most powerful servers for structure prediction currently available. HHpred is the first server that is based on the pairwise comparison of profile hidden Markov models (HMMs). Whereas most conventional sequence search methods search sequence databases such as UniProt or the NR, HHpred searches alignment databases, like Pfam or SMART. This greatly simplifies the list of hits to a number of sequence families instead of a clutter of single sequences. All major publicly available profile and alignment databases are available through HHpred. HHpred accepts a single query sequence or a multiple alignment as input. Within only a few minutes it returns the search results in an easy-to-read format similar to that of PSI-BLAST. Search options include local or global alignment and scoring secondary structure similarity. HHpred can produce pairwise query-template sequence alignments, merged query-template multiple alignments (e.g., for transitive searches), as well as 3D structural models calculated by the MODELLER software from HHpred alignments.

Deactivated/Inactivated Cas7-11 Proteins

Where the Cas7-11 protein has nuclease activity, the Cas7-11 protein may be modified to have diminished nuclease activity e.g., nuclease inactivation of at least 70%, at least 80%, at least 90%, at least 95%, at least 97%, or 100% as compared with the wild type enzyme; or to put in another way, a Cas7-11 enzyme having advantageously about 0% of the nuclease activity of the non-mutated or wild type Cas7-11 enzyme or CRISPR-Cas protein, or no more than about 3% or about 5% or about 10% of the nuclease activity of the non-mutated or wild type Cas7-11 enzyme.

Modified Cas7-11 Enzymes

In particular embodiments, it is of interest to make use of an engineered Cas7-11 protein as defined herein, such as Cas7-11, wherein the protein complexes with a nucleic acid molecule comprising RNA to form a CRISPR complex, wherein when in the CRISPR complex, the nucleic acid molecule targets one or more target polynucleotide loci, the protein comprises at least one modification compared to unmodified Cas7-11 protein, and wherein the CRISPR complex comprising the modified protein has altered activity as compared to the complex comprising the unmodified Cas7-11 protein. It is to be understood that when referring herein to CRISPR “protein,” the Cas7-11 protein is an unmodified or modified CRISPR-Cas protein (e.g., having increased or decreased or the same (or no) enzymatic activity, such as without limitation including Cas7-11. The term “CRISPR protein” may be used interchangeably with “CRISPR-Cas protein”, irrespective of whether the CRISPR protein has altered, such as increased or decreased (or no) enzymatic activity, compared to the wild type CRISPR protein.

Computational analysis of the primary structure of Cas7-11 nucleases reveals 5 distinct domain regions.

Based on the above information, mutants can be generated which lead to inactivation of the enzyme or which modify the double strand nuclease to nickase activity. In alternative embodiments, this information is used to develop enzymes with reduced off-target effects.

In certain of the above-described Cas7-11 enzymes, the enzyme is modified by mutation of one or more residues (in the Cas7-like domains as well as the small subunit).

Orthologs of Cas7-11

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related or are only partially structurally related. An “orthologue” of a protein as used herein is a protein of a different species which performs the same or a similar function as the protein it is an orthologue of Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci.

The present disclosure encompasses the use of a Cas7-11 effector protein, derived from a Cas7-11 locus denoted as subtype III-E. Herein such effector proteins are also referred to as “Cas7-1 ip”, e.g., a Cas7-11 protein (and such effector protein or Cas7-11 protein or protein derived from a Cas7-11 locus is also called “CRISPR-Cas protein”).

In particular embodiments, the effector protein is a Cas7-11 effector protein from an organism from a genus comprising Candidatus Jettenia caeni, Candidatus Scalindua brodae, Desulfobacteraceae, Candidatus Magnetomorum, Desulfonema Ishimotonii, Candidatus Brocadia, Deltaproteobacteria, Syntrophorhabdaceae, or Nitrospirae.

Delivery Cas7-11 Effector

In some embodiments, the Cas7-11 effector and/or peptide sequence are introduced into a cell as a nucleic acid encoding each protein. The nucleic acid introduced into the eukaryotic cell is a plasmid DNA or viral vector. In some embodiments, the Cas7-11 effector and/or peptide sequence are introduced into a cell via a ribonucleoprotein (RNP).

Preferably, delivery is in the form of a vector which may be a viral vector, such as a lenti- or baculo- or adeno-viral/adeno-associated viral vectors, but other means of delivery are known (such as yeast systems, microvesicles, gene guns/means of attaching vectors to gold nanoparticles) and are provided. The viral vector may be selected from a variety of families/genera of viruses, including, but not limited to Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Birnaviridae, Totiviridae, Partitiviridae, Filoviridae, Orthomyxoviridae, Deltavirusa, Leviviridae, Picornaviridae, Marnaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, Idaeovirusa, and Herpesviridae.

A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. For example, baculoviruses may be used for expression in insect cells. These insect cells may, in turn be useful for producing large quantities of further vectors, such as AAV or lentivirus adapted for delivery of the present disclosure. Also envisaged is a method of delivering the Cas7-11 effector and/or peptide sequence comprising delivering to a cell mRNAs encoding each.

In some embodiments, expression of a nucleic acid sequence encoding the Cas7-11 effector and/or peptide sequence may be driven by a promoter. In some embodiments, a single promoter drives expression of a nucleic acid sequence encoding the Cas7-11 effector. In some embodiments, the Cas7-11 effector and guide sequence(s) are operably linked to and expressed from the same promoter. In some embodiments, the Cas7-11 and guide sequence(s) are expressed from different promoters. For example, the promoter(s) can be, but are not limited to, a UBC promoter, a PGK promoter, an EF1A promoter, a CMV promoter, an EFS promoter, a SV40 promoter, and a TRE promoter. The promoter may be a weak or a strong promoter. The promoter may be a constitutive promoter or an inducible promoter. In some embodiments, the promoter can also be an AAV ITR, and can be advantageous for eliminating the need for an additional promoter element, which can take up space in the vector. The additional space freed up by use of an AAV ITR can be used to drive the expression of additional elements, such as guide sequences. In some embodiments, the promoter may be a tissue specific promoter.

In some embodiments, an enzyme coding sequence encoding Cas7-11 effector and/or peptide sequence is codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas7-11 effector correspond to the most frequently used codon for a particular amino acid.

In some embodiments, a vector encodes a Cas7-11 effector and/or peptide sequence comprising one or more nuclear localization sequences (NLSs), such as about or more than about 1, 2, 3, 4, 5, 6, 7, 8, 9, 10, or more NLSs. In some embodiments, the Cas7-11 protein comprises about or more than 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, bur other types of NLS are known. In some embodiments, the NLS is between two domains, for example between the Cas7-11 effector protein and the viral protein. The NLS may also be between two functional domains separated or flanked by a glycine-serine linker.

In general, the one or more NLSs are of sufficient strength to drive accumulation of the Cas7-11 effector and/or peptide sequence in a detectable amount in the nucleus of a eukaryotic cell. In general, strength of nuclear localization activity may derive from the number of NLSs in the Cas7-11 effector and/or other peptide sequences, the particular NLS used, or a combination of these factors. Detection of accumulation in the nucleus may be performed by any suitable technique. For example, a detectable marker may be fused to the Cas7-11 effector and/or peptide sequence, such that location within a cell may be visualized, such as in combination with a means for detecting the location of the nucleus (e.g., a stain specific for the nucleus such as DAPI). Examples of detectable markers include fluorescent proteins (such as green fluorescent proteins, or GFP; RFP; CFP), and epitope tags (HA tag, FLAG tag, SNAP tag). Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some aspects, the disclosure provides methods comprising delivering one or more polynucleotides, such as one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a Cas protein in combination with (and optionally complexed) with a guide sequence is delivered to a cell. Conventional viral and non-viral based gene transfer methods can be used to introduce nucleic acids in mammalian cells or target tissues. Such methods can be used to administer nucleic acids encoding a Cas7-11 effector and/or a polypeptide to cells in culture, or in a host organism. Non-viral vector delivery systems include DNA plasmids, RNA (e.g., a transcript of a vector described herein), naked nucleic acid, nucleic acid complexed with a delivery vehicle, such as a liposome, and ribonucleoprotein. Viral vector delivery systems include DNA and RNA viruses, which have either episomal or integrated genomes after delivery to the cell. For a review of gene therapy procedures, see Anderson, Science 256:808-8313 (1992); Navel and Felgner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994), which are incorporated herein by reference in their entirety.

The Cas7-11 effector and/or peptide sequence can be delivered using adeno-associated virus (AAV), lentivirus, adenovirus, or other viral vector types, or combinations thereof. In some embodiments, one or more Cas7-11 effectors and/or one or more guide RNAs can be packaged into one or more viral vectors. In some embodiments, the Cas7-11 effector and/or peptide sequence can be delivered via AAV as a trans-splicing system, similar to Lai et al. (Nature Biotechnology, 2005, DOI: 10.1038/nbt1153). In some embodiments, the viral vector is delivered to the tissue of interest by, for example, an intramuscular injection, while other times the viral delivery is via intravenous, transdermal, intranasal, oral, mucosal, intrathecal, intracranial or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (e.g., vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In certain embodiments, delivery of the Cas7-11 and/or peptide sequence to a cell is non-viral. In certain embodiments, the non-viral delivery system is selected from a ribonucleoprotein, cationic lipid vehicle, electroporation, nucleofection, calcium phosphate transfection, transfection through membrane disruption using mechanical shear forces, mechanical transfection, and nanoparticle delivery.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. Cell lines are available from a variety of sources known to those with skill in the art (see, e.g., the American Type Culture Collection (ATCC) (Manassas, VA). In some embodiments, a cell transfected with one or more vectors described herein is used to establish a new cell line comprising one or more vector-derived sequences.

Guide Molecules

The system may comprise a guide molecule. The guide molecule may comprise a guide sequence. In certain cases, the guide sequence may be linked to a direct repeat sequence. In some cases, the system may comprise a nucleotide sequence encoding the guide molecule. The guide molecule may form a complex with the dead Cas7-11 protein and directs the complex to bind the target RNA sequence at one or more codons encoding an amino acid that is post-translationally modified. The guide sequence may be capable of hybridizing with a target RNA sequence comprising an Adenine or Cytidine encoding said amino acid to form an RNA duplex, wherein said guide sequence comprises a non-pairing nucleotide at a position corresponding to said Adenine or Cytidine resulting in a mismatch in the RNA duplex formed. The guide sequence may comprise one or more mismatch corresponding to different adenosine sites in the target sequence. In certain cases, guide sequence may comprise multiple mismatches corresponding to different adenosine sites in the target sequence. In cases where two guide molecules are used, the guide sequence of each of the guide molecules may comprise a mismatch corresponding to a different adenosine site in the target sequence.

In general, a CRISPR system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. The precise sequence and length requirements for the PAM differ depending on the Cas7-11 protein used, but PAMs are typically 2-8 base pair sequences adjacent the protospacer (that is, the target sequence). Examples of the natural PAM sequences for different Cas7-11 orthologues are provided herein below and the skilled person will be able to identify further PAM sequences for use with a given Cas7-11 protein. In certain embodiments, the Cas7-11 protein has been modified to recognize a non-natural PAM, such as recognizing a PAM having a sequence or comprising a sequence YCN, YCV, AYV, TYV, RYN, RCN, TGYV, NTTN, TTN, TRTN, TYTV, TYCT, TYCN, TRTN, NTTN, TACT, TYCC, TRTC, TATV, NTTV, TTV, TSTG, TVTS, TYYS, TCYS, TBYS, TCYS, TNYS, TYYS, TNTN, TSTG, TTCC, TCCC, TATC, TGTG, TCTG, TYCV, or TCTC.

The terms “guide molecule” and “guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In the context of the present disclosure the target nucleic acid sequence or target sequence is the sequence comprising the target adenosine to be deaminated also referred to herein as the “target adenosine”. In some embodiments, except for the intended dA-C mismatch, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In some embodiments, the guide molecule comprises a guide sequence that is designed to have at least one mismatch with the target sequence, such that an RNA duplex formed between the guide sequence and the target sequence comprises a non-pairing C in the guide sequence opposite to the target A for deamination on the target sequence. In some embodiments, aside from this A-C mismatch, the degree of complementarity, when optimally aligned using a suitable alignment algorithm, is about or more than about 50%, 60%, 75%, 80%, 85%, 90%, 95%, 97.5%, 99%, or more. In some cases, the distance between the non-pairing C and the 5′ end of the guide sequence is from about 10 to about 50, e.g., from about 10 to about 20, from about 15 to about 25, from about 20 to about 30, from about 25 to about 35, from about 30 to about 40, from about 35 to about 45, or from about 40 to about 50 nucleotides (nt) in length. In certain example. In some cases, the distance between the non-pairing C and the 3′ end of the guide sequence is from about 10 to about 50, e.g., from about 10 to about 20, from about 15 to about 25, from about 20 to about 30, from about 25 to about 35, from about 30 to about 40, from about 35 to about 45, or from about 40 to about 50 nucleotides (nt) in length. In one example, the distance between the non-pairing C and the 5′ end of said guide sequence is from about 20 to about 30 nucleotides.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence has a length from about 10 to about 100, e.g., from about 20 to about 60, from about 20 to about 55, from about 20 to about 53, from about 25 to about 53, from about 29 to about 53, from about 20 to about 30, from about 25 to about 35, from about 30 to about 40, from about 35 to about 45, from about 40 to about 50, from about 45 to about 55, from about 50 to about 60, from about 55 to about 65, from about 60 to about 70, from about 70 to about 80, from about 80 to about 90, or from about 90 to about 100 nucleotides (nt) long that is capable of forming an RNA duplex with a target sequence. In certain example, the guide sequence has a length from about 20 to about 53 nt capable of forming said RNA duplex with said target sequence. In certain example, the guide sequence has a length from about 25 to about 53 nt capable of forming said RNA duplex with said target sequence. In certain example, the guide sequence has a length from about 29 to about 53 nt capable of forming said RNA duplex with said target sequence. In certain example, the guide sequence has a length from about 40 to about 50 nt capable of forming said RNA duplex with said target sequence. In some examples, the guide sequence comprises a non-pairing Cytosine at a position corresponding to said Adenine resulting in an A-C mismatch in the RNA duplex formed. The guide sequence is selected so as to ensure that it hybridizes to the target sequence comprising the adenosine to be deaminated.

In some embodiments, the guide sequence is about 10 nt to about 100 nt long and hybridizes to the target DNA strand to form an almost perfectly matched duplex, except for having a dA-C mismatch at the target adenosine site. Particularly, in some embodiments, the dA-C mismatch is located close to the center of the target sequence (and thus the center of the duplex upon hybridization of the guide sequence to the target sequence), thereby restricting the nucleotide deaminase to a narrow editing window (e.g., about 4 bp wide). In some embodiments, the target sequence may comprise more than one target adenosine to be deaminated. In further embodiments, the target sequence may further comprise one or more dA-C mismatch 3′ to the target adenosine site. In some embodiments, to avoid off-target editing at an unintended Adenine site in the target sequence, the guide sequence can be designed to comprise a non-pairing Guanine at a position corresponding to said unintended Adenine to introduce a dA-G mismatch, which is catalytically unfavorable for certain nucleotide deaminases such as ADAR1 and ADAR2. See Wong et al., RNA 7:846-858 (2001), which is incorporated herein by reference in its entirety.

In some embodiments, the sequence of the guide molecule (direct repeat and/or spacer) is selected to reduce the degree of secondary structure within the guide molecule. In some embodiments, about or less than about 75%, 50%, 40%, 30%, 25%, 20%, 15%, 10%, 5%), 1%), or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas7-11. Accordingly, in particular embodiments, the guide molecule is adjusted to avoid cleavage by Cas7-11 or other RNA-cleaving enzymes.

In some embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The disclosure accordingly comprehends a guide molecule that responds to normal or pathological physiological conditions, including without limitation pH, hypoxia, O₂ concentration, temperature, protein concentration, enzymatic concentration, lipid structure, light exposure, mechanical disruption (e.g., ultrasound waves), magnetic fields, electric fields, or electromagnetic radiation.

Trans-Splicing and Trans-Splicing Template

Generally, trans-splicing relies on the recruitment of an RNA template to a pre-mRNA without any active targeting domains and involves competition with the cis target. Combining trans-splicing with programmable RNA guided CRISPR systems can help boost the efficiency of the trans-splicing mechanism, enabling any potential type of RNA edit, insertion (e.g., correction of a mutation, a transgene), deletion, or replacement to be incorporated into endogenous transcripts. This combination can be used, for example and without limitation, to edit a polynucleotide in a cell, treat or prevent a genetically inherited diseases, and engineering cells (e.g., CAR-T cells) via editing of a transgene.

The system disclosed herein may comprise a splicing protein selected from the group consisting of RMB17, SF3B6, U2AF1, and U2AF2.

The systems disclosed herein may comprise a trans-splicing template polynucleotide. The trans-splicing template polynucleotide can comprise one or more cargo guide sequences, one or more an integration sequences, one or more a 3′ and/or 5′ splicing site sequences, one or more branch point sequences, and/or one or more polypyrimidine tract sequences. The cargo guide sequence can be complementary to a portion of one or more intron and/or exon sequences of a target RNA sequence. Each of the sequences from the trans-splicing template polynucleotide can be operably connected in any order.

The systems disclosed herein may comprise a Cas7-11 enzyme sequence coupled to one or more guide RNA sequences that is complementary to one or more portions of an intron and/or exon sequences of the target RNA sequence that is upstream, downstream, or overlapping of the portion of the intron and/or exon sequences that is complementary to a cargo guide sequence. The Cas7-11 enzyme may also be directly (no intervening linker) or indirectly (XTEN linker intervening) fused to a splicing protein at their N- or C-terminals.

The systems disclosed herein may comprise a target RNA sequence comprising one or more intron and/or exon sequences, one or more 3′ and/or 5′ splicing site sequences, and/or one or more a 5′-terminal and/or 3′-terminal fragment sequences. The one or more intron and/or exon sequences can comprise one or more branch point sequences and one or more polypyrimidine tract sequences. Each of the sequences from the target RNA sequence is operably connected in any order.

In some embodiments, the trans-splicing is a 5′ trans splicing, a 3′ trans splicing, or an internal trans splicing.

Pharmaceutical Compositions

Pharmaceutical compositions described herein comprise at least one component of an editing system described herein (e.g., an editing polypeptide) and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing at least one component of an editing system described herein (e.g., an editing polypeptide) and formulating it into a pharmaceutically acceptable composition by the addition of one or more pharmaceutically acceptable excipient. In some embodiments, the pharmaceutical composition comprises a single component described herein (e.g., an editing polypeptide). In some embodiments, the pharmaceutical composition comprises a plurality of the components described herein (e.g., an editing polypeptide, a ttRNA, a targeting gRNA, etc.).

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein in order to deliver the editing system or composition to a target cell. Non-limiting embodiments include parenteral administration, such as intramuscular, intradermal, subcutaneous, transcutaneous, or mucosal administration. In one embodiment, the pharmaceutical composition is formulated for intravenous administration. In one embodiment, the pharmaceutical composition is formulated for administration by intramuscular, intradermal, or subcutaneous injection. Injectables can be prepared in conventional forms, either as liquid solutions or suspensions. The injectables can contain one or more excipients. Exemplary excipients include, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients (e.g., carriers) used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

Kits

Also provided herein are kits comprising at least one pharmaceutical composition described herein. In addition, the kit may comprise a liquid vehicle for solubilizing or diluting, and/or technical instructions. The technical instructions of the kit may contain information about administration and dosage and subject groups. In some embodiments, the kit contains a single container comprising a single pharmaceutical composition described herein. In some embodiments, the kit at least two separate containers, each comprising a different pharmaceutical composition described herein (e.g., a first container comprising a pharmaceutical composition comprising one component of an editing system described herein, e.g., an editing polypeptide described herein, and a second container comprising a second pharmaceutical composition comprising a second component of an editing system described herein, e.g., a ttRNA).

Methods of Use

Provided herein are various methods of using the editing systems, compositions, pharmaceutical compositions described herein and any one or more of the components thereof (e.g., an editing polypeptide).

In one aspect, provided herein are methods of editing a target polynucleotide, the method comprising contacting the target polynucleotide with an editing system, composition, pharmaceutical composition, or any component thereof (e.g., an editing polypeptide). In some embodiments, the target polynucleotide is or is within a gene. In some embodiments, the target polynucleotide is or is within a genome.

In one aspect, provided herein are methods of editing a target polynucleotide within a cell, the method comprising introducing into the cell an editing system, composition, pharmaceutical composition, or any component thereof (e.g., an editing polypeptide). In some embodiments, the target polynucleotide is or is within a gene. In some embodiments, the target polynucleotide is or is within a genome.

In one aspect, provided herein are methods of editing a target polynucleotide within a cell in a subject, the method comprising administering to the subject an editing system, composition, pharmaceutical composition, or any component thereof (e.g., an editing polypeptide), in an amount sufficient to deliver the editing system, composition, pharmaceutical composition, or component to a cell in the subject. In some embodiments, the target polynucleotide is or is within a gene. In some embodiments, the target polynucleotide is or is within a genome.

In one aspect, provided herein are methods of delivering an editing system, composition, pharmaceutical composition, or any component thereof (e.g., an editing polypeptide) to a cell comprising contacting the cell with the editing system, composition, pharmaceutical composition, or component thereof, in an amount sufficient to deliver the editing system, composition, pharmaceutical composition, or any component thereof to the cell.

In one aspect, provided herein are methods of delivering an editing system, composition, pharmaceutical composition, or any component thereof (e.g., an editing polypeptide) to a subject, the method comprising administering the editing system, composition, pharmaceutical composition, or component thereof to the subject.

In one aspect, provided herein are methods of delivering an editing system, composition, pharmaceutical composition, or any component thereof (e.g., an editing polypeptide) to a cell in a subject, the method comprising administering the editing system, composition, pharmaceutical composition, or component thereof to the subject, in an amount sufficient to deliver the editing system, composition, pharmaceutical composition, or component to a cell in the subject.

In one aspect, provided herein are methods of treating a subject diagnosed with or suspected of having a disease associated with a genetic mutation comprising administering a composition or system described herein to the subject in an amount sufficient to correct the genetic mutation. Exemplary diseases associated with a genetic mutation, include, but are not limited to cystic fibrosis, muscular dystrophy, hemochromatosis, Tay-Sachs, Huntington disease, Congenital Deafness, Sickle cell anemia, Familial hypercholesterolemia, adenosine deaminase (ADA) deficiency, X-linked SCID (X-SCID), and Wiskott-Aldrich syndrome (WAS).

In some embodiments, the genetic mutation is in one of the following genes: GBA, BTK, ADA, CNGB3, CNGA3, ATF6, GNAT2, ABCA1, ABCA7, APOE, CETP, LIPC, MMP9, PLTP, VTN, ABCA4, MFSD8, TLR3, TLR4, ERCC6, HMCN1, HTRA1, MCDR4, MCDR5, ARMS2, C2, C3, CFB, CFH, JAG1, NOTCH2, CACNAlF, SERPINA1, TTR, GSN, B2M, APOA2, APOA1, OSMR, ELP4, PAX6, ARG, ASL, PITX2, FOXC1, BBS1, BBS10, BBS2, BBS9, MKKS, MKS1, BBS4, BBS7, TTC8, ARL6, BBS5, BBS12, TRIM32, CEP290, ADIPOR1, BBIP1, CEP19, IFT27, LZTFL1, DMD, BEST1, HBB, CYP4V2, AMACR, CYP7B1, HSD3B7, AKR1D1, OPN1SW, NR2F1, RLBP1, RGS9, RGS9BP, PROM1, PRPH2, GUCY2D, CACD, CHM, ALAD, ASS1, SLC25A13, OTC, ACADVL, ETFDH, TMEM67, CC2D2A, RPGRIP1L, KCNV2, CRX, GUCA1A, CERKL, CDHR1, PDE6C, TTLL5, RPGR, CEP78, C21orf2, C80RF37, RPGRIP1, ADAM9, POC1B, PITPNM3, RAB28, CACNA2D4, AIPL1, UNC119, PDE6H, OPN1LW, RIMS1, CNNM4, IFT81, RAX2, RDH5, SEMA4A, CORD17, PDE6B, GRK1, SAG, RHO, CABP4, GNB3, SLC24A1, GNAT1, GRM6, TRPM1, LRIT3, TGFBI, TACSTD2, KRT12, OVOL2, CPS1, UGT1A1, UGT1A9, UGT1A8, UGT1A7, UGT1A6, UGT1A5, UGT1A4, CFTR, DLD, EFEMP1, ABCC2, ZNF408, LRP5, FZD4, TSPAN12, EVR3, APOB, SLC2A2, LOC106627981, GBA1, NR2E3, OAT, SLC40A1, F8, F9, UROD, CPOX, HFE, JH, LDLR, EPHX1, TJP2, BAAT, NBAS, LARS1, HAMP, HJV, RS1, ADAMTS18, LRAT, RPE65, LCA5, MERTK, GDF6, RD3, CCT2, CLUAP1, DTHD1, NMNAT1, SPATA7, IFT140, IMPDH1, OTX2, RDH12, TULP1, CRB1, MT-ND4, MT-ND1, MT-ND6, BCKDHA, BCKDHB, DBT, MMAB, ARSB, GUSB, NAGS, NPC1, NPC2, NDP, OPA1, OPA3, OPA4, OPA5, RTN4IP1, TMEM126A, OPA6, OPA8, ACO2, PAH, PRKCSH, SEC63, GAA, UROS, PPOX, HPX, HMOX1, HMBS, MIR223, CYP1B1, LTBP2, AGXT, ATP8B1, ABCB11, ABCB4, FECH, ALAS2, PRPF31, RP1, EYS, TOPORS, USH2A, CNGA1, C2ORF71, RP2, KLHL7, ORF1, RP6, RP24, RP34, ROM1, ADGRA3, AGBL5, AHR, ARHGEF18, CA4, CLCC1, DHDDS, EMC1, FAM161A, HGSNAT, HK1, IDH3B, KIAA1549, KIZ, MAK, NEUROD1, NRL, PDE6A, PDE6G, PRCD, PRPF3, PRPF4, PRPF6, PRPF8, RBP3, REEP6, SAMD11, SLC7A14, SNRNP200, SPP2, ZNF513, NEK2, NEK4, NXNL1, OFD1, RP1L1, RP22, RP29, RP32, RP63, RP9, RGR, POMGNT1, DHX38, ARL3, COL2A1, SLCO1B1, SLCO1B3, KCNJ13, TIMP3, ELOVL4, TFR2, FAH, HPD, MYO7A, CDH23, PCDH15, DFNB31, GPR98, USH1C, USH1G, CIB2, CLRN1, HARS, ABHD12, ADGRV1, ARSG, CEP250, IMPG1, IMPG2, VCAN, G6PC1, ATP7B, HTT, STAT3, PABPC1, PPIB, TOP2A, SHANK3, USF1, gLuc, and RPL41.

In some embodiments, the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

Sequences

Table 1 below shows Cas7-11 sequences for trans-splicing.

TABLE 1

| SEQ ID | | |
|---|---|---|
| NO | ID | Sequence |
| 1 | huDiCas7- | MTTTMKISIEFLEPFRMTKWQESTRRNKNNKEFVRGQAFARWH |
| | 11 | RNKKDNTKGRPYITGTLLRSAVIRSAENLLTLSDGKISEKTCCPG |
| | | KFDTEDKDRLLQLRQRSTLRWTDKNPCPDNAETYCPFCELLGRS |
| | | GNDGKKAEKKDWRFRIHFGNLSLPGKPDFDGPKAIGSQRVLNR |
| | | VDFKSGKAHDFFKAYEVDHTRFPRFEGEITIDNKVSAEARKLLC |
| | | DSLKFTDRLCGALCVIRFDEYTPAADSGKQTENVQAEPNANLAE |
| | | KTAEQIISILDDNKKTEYTRLLADAIRSLRRSSKLVAGLPKDHDG |
| | | KDDHYLWDIGKKKKDENSVTIRQILTTSADTKELKNAGKWREF |
| | | CEKLGEALYLKSKDMSGGLKITRRILGDAEFHGKPDRLEKSRSV |
| | | SIGSVLKETVVCGELVAKTPFFFGAIDEDAKQTDLQVLLTPDNKY |
| | | RLPRSAVRGILRRDLQTYFDSPCNAELGGRPCMCKTCRIMRGITV |
| | | MDARSEYNAPPEIRHRTRINPFTGTVAEGALFNMEVAPEGIVFPF |
| | | QLRYRGSEDGLPDALKTVLKWWAEGQAFMSGAASTGKGRFRM |
| | | ENAKYETLDLSDENQRNDYLKNWGWRDEKGLEELKKRLNSGL |
| | | PEPGNYRDPKWHEINVSIEMASPFINGDPIRAAVDKRGTDVVTFV |
| | | KYKAEGEEAKPVCAYKAESFRGVIRSAVARIHMEDGVPLTELTH |
| | | SDCECLLCQIFGSEYEAGKIRFEDLVFESDPEPVTFDHVAIDRFTG |
| | | GAADKKKFDDSPLPGSPARPLMLKGSFWIRRDVLEDEEYCKALG |
| | | KALADVNNGLYPLGGKSAIGYGQVKSLGIKGDDKRISRLMNPAF |
| | | DETDVAVPEKPKTDAEVRIEAEKVYYPHYFVEPHKKVEREEKPC |
| | | GHQKFHEGRLTGKIRCKLITKTPLIVPDTSNDDFFRPADKEARKE |
| | | KDEYHKSYAFFRLHKQIMIPGSELRGMVSSVYETVTNSCFRIFDE |
| | | TKRLSWRMDADHQNVLQDFLPGRVTADGKHIQKFSETARVPFY |
| | | DKTQKHFDILDEQEIAGEKPVRMWVKRFIKRLSLVDPAKHPQKK |
| | | QDNKWKRRKEGIATFIEQKNGSYYFNVVTNNGCTSFHLWHKPD |
| | | NFDQEKLEGIQNGEKLDCWVRDSRYQKAFQEIPENDPDGWECK |
| | | EGYLHVVGPSKVEFSDKKGDVINNFQGTLPSVPNDWKTIRTNDF |
| | | KNRKRKNEPVFCCEDDKGNYYTMAKYCETFFFDLKENEEYEIPE |
| | | KARIKYKELLRVYNNNPQAVPESVFQSRVARENVEKLKSGDLV |
| | | YFKHNEKYVEDIVPVRISRTVDDRMIGKRMSADLRPCHGDWVE |
| | | DGDLSALNAYPEKRLLLRHPKGLCPACRLFGTGSYKGRVRFGFA |
| | | SLENDPEWLIPGKNPGDPFHGGPVMLSLLERPRPTWSIPGSDNKF |
| | | KVPGRKFYVHHHAWKTIKDGNHPTTGKAIEQSPNNRTVEALAG |
| | | GNSFSFEIAFENLKEWELGLLIHSLQLEKGLAHKLGMAKSMGFG |
| | | SVEIDVESVRLRKDWKQWRNGNSEIPNWLGKGFAKLKEWFRDE |
| | | LDFIENLKKLLWFPEGDQAPRVCYPMLRKKDDPNGNSGYEELK |
| | | DGEFKKEDRQKKLTTPWTPWA |
| 2 | NLS- | MKRTADGSEFESPKKKRKVTTTMKISIEFLEPFRMTKWQESTRR |
| | huDisC | NKNNKEFVRGQAFARWHRNKKDNTKGRPYITGTLLRSAVIRSA |
| | as7-11- | ENLLTLSDGKISEKTCCPGKFDTEDKDRLLQLRQRSTLRWTDKN |
| | NLS | PCPDNAETYCPFCELLGRSGNDGKKAEKKDWRFRIHFGNLSLPG |
| | | KPDFDGPKAIGSQRVLNRVDFKSGKAHDFFKAYEVDHTRFPRFE |
| | | GEITIDNKVSAEARKLLCDSLKFTDRLCGALCVIRFDEYTPAADS |
| | | GKQTENVQAEPNANLAEKTAEQIISILDDNKKTEYTRLLADAIRS |
| | | LRRSSKLVAGLPKDHDGKDDHYLWDIGKKKKDENSVTIRQILTT |
| | | SADTKELKNAGKWREFCEKLGEALYLKSKDMSGGLKITRRILGD |
| | | AEFHGKPDRLEKSRSVSIGSVLKETVVCGELVAKTPFFFGAIDED |
| | | AKQTDLQVLLTPDNKYRLPRSAVRGILRRDLQTYFDSPCNAELG |
| | | GRPCMCKTCRIMRGITVMDARSEYNAPPEIRHRTRINPFTGTVAE |
| | | GALFNMEVAPEGIVFPFQLRYRGSEDGLPDALKTVLKWWAEGQ |
| | | AFMSGAASTGKGRFRMENAKYETLDLSDENQRNDYLKNWGWR |
| | | DEKGLEELKKRLNSGLPEPGNYRDPKWHEINVSIEMASPFINGDP |
| | | IRAAVDKRGTDVVTFVKYKAEGEEAKPVCAYKAESFRGVIRSA |
| | | VARIHMEDGVPLTELTHSDCECLLCQIFGSEYEAGKIRFEDLVFE |
| | | SDPEPVTFDHVAIDRFTGGAADKKKFDDSPLPGSPARPLMLKGSF |
| | | WIRRDVLEDEEYCKALGKALADVNNGLYPLGGKSAIGYGQVKS |
| | | LGIKGDDKRISRLMNPAFDETDVAVPEKPKTDAEVRIEAEKVYY |
| | | PHYFVEPHKKVEREEKPCGHQKFHEGRLTGKIRCKLITKTPLIVP |
| | | DTSNDDFFRPADKEARKEKDEYHKSYAFFRLHKQIMIPGSELRG |
| | | MVSSVYETVTNSCFRIFDETKRLSWRMDADHQNVLQDFLPGRV |
| | | TADGKHIQKFSETARVPFYDKTQKHFDILDEQEIAGEKPVRMWV |
| | | KRFIKRLSLVDPAKHPQKKQDNKWKRRKEGIATFIEQKNGSYYF |
| | | NVVTNNGCTSFHLWHKPDNFDQEKLEGIQNGEKLDCWVRDSRY |
| | | QKAFQEIPENDPDGWECKEGYLHVVGPSKVEFSDKKGDVINNFQ |
| | | GTLPSVPNDWKTIRTNDFKNRKRKNEPVFCCEDDKGNYYTMAK |
| | | YCETFFFDLKENEEYEIPEKARIKYKELLRVYNNNPQAVPESVFQ |
| | | SRVARENVEKLKSGDLVYFKHNEKYVEDIVPVRISRTVDDRMIG |
| | | KRMSADLRPCHGDWVEDGDLSALNAYPEKRLLLRHPKGLCPAC |
| | | RLFGTGSYKGRVRFGFASLENDPEWLIPGKNPGDPFHGGPVMLS |
| | | LLERPRPTWSIPGSDNKFKVPGRKFYVHHHAWKTIKDGNHPTTG |
| | | KAIEQSPNNRTVEALAGGNSFSFEIAFENLKEWELGLLIHSLQLE |
| | | KGLAHKLGMAKSMGFGSVEIDVESVRLRKDWKQWRNGNSEIP |
| | | NWLGKGFAKLKEWFRDELDFIENLKKLLWFPEGDQAPRVCYPM |
| | | LRKKDDPNGNSGYEELKDGEFKKEDRQKKLTTPWTPWAKRTA |
| | | DGSEFESPKKKRKV |
| 3 | huCjcCas7- | MHTILPIHLTFLEPYRLAEWHAKADRKKNKRYLRGMSFAQWHK |
| | 11 | DKDGIGKPYITGTLLRSAVLNAAEELISLNQGMWAKEPCCNGKF |
| | | ETEKDKPAVLRKRPTIQWKTGRPAICDPEKQEKKDACPLCMLLG |
| | | RFDKAGKRHRDNKYDKHDYDIHFDNLNLITDKKFSHPDDIASER |
| | | ILNRVDYTTGKAHDYFKVWEVDDDQWWQFTGTITMHDDCSKA |
| | | KGLLLASLCFVDKLCGALCRIEVTGNNSQDENKEYAHPDTGIITS |
| | | LNLKYQNNSTIHQDAVPLSGSAHDNDEPPVHDNDSSLDNDTITL |
| | | LSMKAKEIVGAFRESGKIEKARTLADVIRAMRLQKPDIWEKLPK |
| | | GINDKHHLWDREVNGKKLRNILEELWRLMNKRNAWRTFCEVL |
| | | GNELYRCYKEKTGGIVLRFRTLGETEYYPEPEKTEPCLISDNSIPIT |
| | | PLGGVKEWIIIGRLKAETPFYFGVQSSFDSTQDDLDLVPDIVNTD |
| | | EKLEANEQTSFRILMDKKGRYRIPRSLIRGVLRRDLRTAFGGSGC |
| | | IVELGRMIPCDCKVCAIMRKITVMDSRSENIELPDIRYRIRLNPYT |
| | | ATVDEGALFDMEIGPEGITFPFVFRYRGEDALPRELWSVIRYWM |
| | | DGMAWLGGSGSTGKGRFALIDIKVFEWDLCNEEGLKAYICSRGL |
| | | RGIEKEVLLENKTIAEITNLFKTEEVKFFESYSKHIKQLCHECIINQ |
| | | ISFLWGLRSYYEYLGPLWTEVKYEIKIASPLLSSDTISALLNKDNI |
| | | DCIAYEKRKWENGGIKFVPTIKGETIRGIVRMAVGKRSGDLGMD |
| | | DHEDCSCTLCTIFGNEHEAGKLRFEDLEVVEEKLPSEQNSDSNKI |
| | | PFGPVQDGDGNREKECVTAVKSYKKKLIDHVAIDRFHGGAEDK |
| | | MKFNTLPLAGSFEKPIILKGRFWIKKDIVKDYKKKIEDAMVDIRD |
| | | GLYPIGGKTGIGYGWVTDLTILNPQSGFQIPVKKDISPEPGTYSTY |
| | | PSHSTPSLNKGHIYYPHYFLAPANTVHREQEMIGHEQFHKEQKG |
| | | ELLVSGKIVCTLKTVTPLIIPDTENEDAFGLQNTYSGHKNYQFFHI |
| | | NDEIMVPGSEIRGMISSVYEAITNSCFRVYDETKYITRRLSPEKKD |
| | | ESNDKNKSQDDASQKIRKGLVKKTDEGFSIIEVERYSMKTKGGT |
| | | KLVDKVYRLPLYDSEAVIASIQFEQYGEKNEKRNAKIRAAIKRNE |
| | | VIAEVARKNLIFLRSLTPEELKKVLQGEILVKFSLKSGKNPNDYL |
| | | AELHENGTERGLIKFTGLNMVNIKNVNEEDKDENDTWDWEKLN |
| | | IFHNAHEKRNSLKQGYPRPVLKFIKDRVEYTIPKRCERIFCIPVKN |
| | | TIEYKVSSKVCKQYKDVLSDYEKNFGHINKIFTTKIQKRELTDGD |
| | | LVYFIPNEGADKTVQAIMPVPLSRITDSRTLGERLPHKNLLPCVH |
| | | EVNEGLLSGILDSLDKKLLSIHPEGLCPTCRLFGTTYYKGRVRFG |
| | | FANLMNKPKWLTERENGCGGYVTLPLLERPRLTWSVPSDKCDV |
| | | PGRKFYIHHNGWQEVLRNNDITPKTENNRTVEPLAADNRFTFDV |
| | | YFENLREWELGLLCYCLELEPGMGHKLGMGKPMGFGSVKIAIE |
| | | RLQTFTVHQDGINWKPSENEIGVYVQKGREKLVEWFTPSAPHKN |
| | | MEWNGVKHIKDLRSLLSIPGDKPTVKYPTLNKDAEGAISDYTYE |
| | | RLSDTKLLPHDKRVEYLRTPWSPWNAFVKEAEYSPSEKSDEKGR |
| | | ETIRTKPKSLPSVKSIGKVKWFDEGKGFGILIMDDGKEVSISKNSI |
| | | RGNILLKKGQKVTFHIVQGLIPKAEDIEIAK |
| 4 | hsmCas7- | MTKIPISLTFLEPFRLVDWVSESERDKSEFLRGLSFARWHRIKNQ |
| | 11 | REDENQGRPYITGTLLRSAVIKAAEELIFLNGGKWQSEECCNGQF |
| | | KGSKAKYRKVECPRRRHRATLKWTDNTCSDYHNACPFCLLLGC |
| | | LKPNSKENSDIHFSNLSLPNKQIFKNPPEIGIRRILNRVDFTTGKAQ |
| | | DYFYVWEVEHSMCPKFQGTVKINEDMPKYNVVKDLLISSIQFVD |
| | | KLCGALCVIEIGKTKNYICQSFSSNIPEEEIKKLAQEIRDILKGEDA |
| | | LDKMRVLADTVLQMRTKGPEIVNELPRGIEKKGGHWLWDKLRL |
| | | RKKFKEIANNYKDSWQELCEKLGNELYISYKELTGGIAVKKRIIG |
| | | ETEYRKIPEQEISFLPSKAGYSYEWIILGKLISENPFFFGKETKTEE |
| | | QIDMQILLTKDGRYRLPRSVLRGALRRDLRLVIGSGCDVELGSK |
| | | RPCPCPVCRIMRRVTLKDARSDYCKPPEVRKRIRINPLTGTVQKG |
| | | ALFTMEVAPEGISFPFQLRFRGEDKFHDALQNVLVWWKEGKLFL |
| | | GGGASTGKGRFKLEIEHVLKWDLKNNFHSYLQYKGLRDKGDFN |
| | | SIKEIEGLKVETEEFKVKKPFPWSCVEYTIFIESPFVSGDPVEAVL |
| | | DSSNTDLVTFKKYKLEESKEVFAIKGESIRGVFRTAVGKNEGKLT |
| | | TENEHEDCTCILCRLFGNEHETGKVRFEDLELINDSAPKRLDHVA |
| | | IDRFTGGAKEQAKFDDSPLIGSPDSPLEFTGIVWVRDDIDEEEKK |
| | | ALKSAFLDIKSGYYPLGGKKGVGYGWVSNLKIESGPEWLRLEV |
| | | QEKSSQENVLSPVILSEVMDIEFNPPKIDENGVYFPYAFLRPLNEV |
| | | KRTREPIGHNEWKKSLISGYLTCRLELLTPLIIPDTSEEVIKEKVN |
| | | NGEHPVYKFFRLGGHLCIPAAEIRGMISSVYEALTNSCFRVEDEK |
| | | RLISWRMTAEEAKRPDPKKSEEQNRMRFRPGRIIKKDKKFYAQE |
| | | MLELRIPVYDNKDKRNEISQNDPTRPSEYNHPTEPERIFFSNAEKI |
| | | RNFLKRNSNYLHGSTPLLFRQWSISNRYDKIALIGNKSQGHLKFT |
| | | GPNKIEVSEGTKCPKYETIPGRDEWDKAVHNYVEPGKFVTVISR |
| | | KKGQKPKAVQRRRNVPAFCCYDYNTNRCFVMNKRCERVFKVS |
| | | RDKPKYEIPPDAIRRYEHVLRKYRENWERYDIPEVFRTRLPGDGE |
| | | TLNEGDLVYFRLDENNRVLDIIPVSISRISDTQYLGRRLPDHLRSC |
| | | VRECLYEGWGDCKPCKLSLFPEKMWIRINPEGLCPACHLFGTQV |
| | | YKGRVRFGFARAGSNWKFREEQLTLPRFETPRPTWVIPKRKDEY |
| | | QIPGRKFYLHHNGWEEIYKKNKKNEIKKEKNNATFEVLKQGTFY |
| | | FKVFFENLELWELGLLIFSAELGGEEFAHKLGHGKALGFGSVKIS |
| | | VDKIILRRDPGQFEQRGQKFKRDAVDKGFCVLENRFGKTNFKIY |
| | | LNNFLQLLYWPNNKKVKVRYPYLRQEDDPEKLPGYVELKKHQ |
| | | MLKDDNRYSLFARPRAVWLKWTEMVQRDKS |
| 5 | hvsCas7- | MKSIPITLTFLEPYRILPWAEKGKRDKKEYLRGANYVRLHKDKN |
| | 11 | GKFKPYITGTLIRSAVLSAIEMLLDITNGEWNGKECCLAKFHTEG |
| | | EKPSFLRKKPIYIRAEKDEICTSRETACPLCLILGREDKAEKKEKD |
| | | KEKFDVHFSNLNLYSSKEFSTIEELAPKRALNRIEQYTGKAQDYF |
| | | TVYEALNKEFWTFKGRIRIKEDIYDKVTDLLFSALRCVEKIAGAL |
| | | CRIEIDKEPSQQKGFVKRQLSKQAKEDIEKIFQVVKDAQKLRLLS |
| | | DCFRELTRMANKDELALPLGPEDDGHYLWDKIKVEGKTLRIFLR |
| | | NCFSQYKDNWLCFCDEASKKGYQKYREKRHKLTDRELPTATPK |
| | | HFAEKKDPQISPIYIDKDDKVYEWIIVGRLIAQTPFHFGDEEKAEG |
| | | AILLTPDNRFRLPRTALRGILRRDLKLAGASACEVEVGRSEPCPC |
| | | DVCKIMRRVTLLDTVSEDLRDFLPELRKRIRINPQSGTVAEGALF |
| | | DTEVGPEGLSFPFVLRYKCEKLPDSLTTVLCWWQEGLAFLSGES |
| | | ATGKGRFRLEINGAFVWDLQKGLFNYIKNHGFRGEERLFLEGNE |
| | | AELEKMGIQINTELLQPEMIKKEKNFTDFPYDLIKYQLNISSPLLL |
| | | NDPIRAIALYEGEGKAPDAVFFKKYVFENGKIEEKPCFKAESIRGI |
| | | FRTAVGRIKNVLTKNHEDCICVLCHLFGNVHETGRLKFEDLKIVS |
| | | GQEEKFFDHVAIDRFLGGAKEKYKFDDKPIIGAPDTPIVLEGKIW |
| | | VKKDINDEAKETLSQAFSDINTGIYYLGANGSIGYGWIEEVKALK |
| | | APSWLKIKEKPNFEKDTSLNISAIMNEFKKDIQTLNLDKTYLPYG |
| | | FLKLLEKVKRTSSPITHERFYENHLTGFIECSLKVLSPLIIPDTETPE |
| | | KEENGHKYYHFLKIDNKPIIPGAEIRGAVSSIYEALTNSCFRVFGE |
| | | KKVLSWRMEGKDAKEFMPGRVSKKKGKLYMVKMQALRLPVY |
| | | DNPALANEIRSGSIYEKYKNSKVEIIFFQTVEGIRKFLRGNFNNVE |
| | | WKKVLVTGIDPLAILPSQKIPGNDKWVKNLQSKISPVRGYFKFTG |
| | | PNKIETKRREEEKDEKLRTKANKVSCLQKDKWYEAMHNHVEY |
| | | KQDYTPPNSPKTEPLERPRNIPCFVCSDKEKIYRMTKRCERVFVS |
| | | LGENAPKYEIPISAIKRYEVILSAYRENWERNKTPELFRTRLPGDG |
| | | RTLNEDDLVYFRADENEKVKDIIPVCISRIVDEVPLIKRLSQELWP |
| | | CVLAECPLLGFECKKCELEGLPEKIWFRINKDGLCPACRLFGTQI |
| | | YKSRVRFSFAYAKNWKFYDGYITLPRLESPRATWLILKEKDKHY |
| | | IKYKVCGRKFYLHNSTYEDIINNSKKEKEKKTENNASFEVLKEGE |
| | | FTFKVYFENLENWELGLLLLSLTGLGEAIKIGHAKPLGFGSVKIE |
| | | AKKIYFREEAGKFHPCEKADEYLKKGLNKLTSWFGKNEINEHM |
| | | RNLLLFMTYYQNLPKVKYPDFDGYAKWRCSYVEQDKVEYFQN |
| | | RWIVAS |
| 6 | dpbaCas7- | MASEDDDTPTLRKVLKDEINGQEDMWRKFCEALGNSLYDLSKK |
| | 11 | AKERKRTEALPRLLGETEIYGLPMRENKEDEPLPSSLTYKFKWLI |
| | | AGELRAETPFFFGTEVQEGQTSATILLNRDGYFRLPRSVIRGALR |
| | | RDLRLVMGNDGCNMPIGGQMCECGVCRVMRHIVIEDGLSDCKI |
| | | PPEVRHRIRLNCHTGTVEEGALFDMETGYQGMTFPFRLYCETEN |
| | | SDLDSYLWEVLNNWQNGQSLFGGDTGTGFGRFELTEPKVFLWN |
| | | FSKKEKHEAYLLNRGFKGQMPVQDVKTKSFKTKTWFQIHRELDI |
| | | SPKKLPWYSTDYRFNVTSPLISRDPIGAMLDPRNTDAIMVRKTVF |
| | | CPDPNAKNRPAPATVYMIKGESIRGILRSIVVRNEELYDTDHEDC |
| | | DCILCRLFGSIHQQGSLRFEDAEVQNSVSDKKMDHVAIDRFTGG |
| | | GVDQMKFDDYPLPGCPAQPLILEGKFWVKDDIDDESKSALEKAF |
| | | ADFRDGLVSLGGLGAIGYGQIGDFELIGGSADWLNLPKPEENRT |
| | | DVPCGDRSAQGPEIKISLDADKIYHPHFFLKPSDKNVYRERELVS |
| | | HAKKKGPDGKSLFTGKITCRLSTEGPVFIPDTDLGEDYFEMQASH |
| | | KKHKNYGFFRINGNVAIPGSSIRGMISSVFEALTNSCFRVFDQER |
| | | YLSRSEKPDPTELTKYYPGKVKRDGNKFFILKMKDFFRLPLYDF |
| | | DFEGEAESLRPNYDEDRNEEENKGKNKNTQKVKNAVEFNIKMA |
| | | GFAKHNRDFLKKYKEQEIKDIFMGKKKVYFTAGKHKPNEAHDN |
| | | DKIALLTKGSNKKAEKGYFKFTGPGMVNVKAGVEGEECDFHID |
| | | ESDPDVYWNMSSILPHNQIKWRPSQKKEYPRPVLKCVKDGTEY |
| | | VMLKRSEHVFAEASSEDSYPVPGKVRKQFNSISRDNVQNTDHLS |
| | | SMFQSRRLHDELSHGDLVYFRHDEKRKVTDIAYVRVSRTVDDR |
| | | PMGKRFKNESLRPCNHVCVEGCDECPDRCKELEDYFSPHPEGLC |
| | | PACHLFGTTDYKGRVSFGLGWHESNTPKWYMPEDNSQKGSHLT |
| | | LPLLERPRPTWSMPNKKSEIPGRKFYVHHPWSVDKIRNRQFDPA |
| | | KEKQPDDVIKPNENNRTVEPLGKGNEFTFEVRFNNLREWELGLL |
| | | LYSLELEDNMAHKLGMGKALGMGSARIKAEAIELRCESAGQNA |
| | | ELKDKAAFVRKGFEFLEIDKPGENDPMNFDHIRQLRELLWFLPE |
| | | NVSANVRYPMLEKEDDGTPGYTDFIKQEEPSTGKRNPSYLSSEK |
| | | RRNILQTPWKHWYLIPPFQASAQSETVFEGTVKWFDDKKGFGFI |
| | | KINDGGKDVFVHHSSIVGTGFKSLNEGDSVAFKMGVGPKGPCAE |
| | | KVKKIGN |
| 7 | CsbCas7- | MNITVELTFFEPYRLVEWFDWDARKKSHSAMRGQAFAQWTWK |
| | 11 | GKGRTAGKSFITGTLVRSAVIKAEELLSLNNGKWEGVPCCNGS |
| | | FQTDESKGKKPSFLRKRHTLQWQANNKNICDKEEACPFCILLGR |
| | | FDNAGKVHERNKDYDIHFSNFDLDHKQEKNDLRLVDIASGRILN |
| | | RVDFDTGKAKDYFRTWEADYETYGTYTGRITLRNEHAKKLLLA |
| | | SLGFVDKLCGALCRIEVIKKSESPLPSDTKEQSYTKDDTVEVLSE |
| | | DHNDELRKQAEVIVEAFKQNDKLEKIRILADAIRTLRLHGEGVIE |
| | | KDELPDGKEERDKGHHLWDIKVQGTALRTKLKELWQSNKDIG |
| | | WRKFTEMLGSNLYLIYKKETGGVSTRFRILGDTEYYSKAHDSEG |
| | | SDLFIPVTPPEGIETKEWIIVGRLKAATPFYFGVQQPSDSIPGKEKK |
| | | SEDSLVINEHTSFNILLDKENRYRIPRSALRGALRRDLRTAFGSGC |
| | | NVSLGGQILCNCKVCIEMRRITLKDSVSDFSEPPEIRYRIAKNPGT |
| | | ATVEDGSLFDIEVGPEGLTFPFVLRYRGHKFPEQLSSVIRYWEEN |
| | | DGKNGMAWLGGLDSTGKGRFALKDIKIFEWDLNQKINEYIKER |
| | | GMRGKEKELLEMGESSLPDGLIPYKFFEERECLFPYKENLKPQW |
| | | SEVQYTIEVGSPLLTADTISALTEPGNRDAIAYKKRVYNDGNNAI |
| | | EPEPRFAVKSETHRGIFRTAVGRRTGDLGKEDHEDCTCDMCIIFG |
| | | NEHESSKIRFEDLELINGNEFEKLEKHIDHVAIDRFTGGALDKAK |
| | | FDTYPLAGSPKKPLKLKGRFWIKKGFSGDHKLLITTALSDIRDGL |
| | | YPLGSKGGVGYGWVAGISIDDNVPDDFKEMINKTEMPLPEEVEE |
| | | SNNGPINNDYVHPGHQSPKQDHKNKNIYYPHYFLDSGSKVYRE |
| | | KDIITHEEFTEELLSGKINCKLETLTPLIIPDTSDENGLKLQGNKPG |
| | | HKNYKFFNINGELMIPGSELRGMLRTHFEALTKSCFAIFGEDSTL |
| | | SWRMNADEKDYKIDSNSIRKMESQRNPKYRIPDELQKELRNSGN |
| | | GLFNRLYTSERRFWSDVSNKFENSIDYKREILRCAGRPKNYKGGI |
| | | IRQRKDSLMAEELKVHRLPLYDNFDIPDSAYKANDHCRKSATCS |
| | | TSRGCRERFTCGIKVRDKNRVFLNAANNNRQYLNNIKKSNHDL |
| | | YLQYLKGEKKIRFNSKVITGSERSPIDVIAELNERGRQTGFIKLSG |
| | | LNNSNKSQGNTGTTFNSGWDRFELNILLDDLETRPSKSDYPRPRL |
| | | LFTKDQYEYNITKRCERVFEIDKGNKTGYPVDDQIKKNYEDILDS |
| | | YDGIKDQEVAERFDTFTRGSKLKVGDLVYFHIDGDNKIDSLIPVR |
| | | ISRKCASKTLGGKLDKALHPCTGLSDGLCPGCHLFGTTDYKGRV |
| | | KFGFAKYENGPEWLITRGNNPERSLTLGVLESPRPAFSIPDDESEI |
| | | PGRKFYLHHNGWRIIRQKQLEIRETVQPERNVTTEVMDKGNVFS |
| | | FDVRFENLREWELGLLLQSLDPGKNIAHKLGKGKPYGFGSVKIKI |
| | | DSLHTFKINSNNDKIKRVPQSDIREYINKGYQKLIEWSGNNSIQK |
| | | GNVLPQWHVIPHIDKLYKLLWVPFLNDSKLEPDVRYPVLNEESK |
| | | GYIEGSDYTYKKLGDKDNLPYKTRVKGLTTPWSPWNPFQVIAE |
| | | HEEQEVNVTGSRPSVTDKIERDGKMV |
| 8 | DsbaCas7- | MKITLRFLEPFRMLDWIRPEERISGNKAFQRGLTFARWHKSKAD |
| | 11 | DKGKPFITGTLLRSAVIRAAEHLLVLSKGKVGEKACCPGKFLTET |
| | | DTETNKAPTMFLRKRPTLKWTDRKGCDPDFPCPLCELLGPGAVG |
| | | KKEGEAGINSYVNFGNLSFPGDTGYSNAREIAVRRVVNRVDYAS |
| | | GKAHDFFRIFEVDHIAFPCFHGEIAFGENVSSQARNLLQDSLRFT |
| | | DRLCGALCVIRYDGDIPKCGKTAPLPETESIQNAAEETARAIVRV |
| | | FHGGRKDPEQAQIDKAEQIQLLSAAVRELGRDKKKVSALPLNHE |
| | | GKEDHYLWDKKAGGETIRTILKAAAEKEAVANQWRQFCIELSE |
| | | ELYKEAKKAHGGLEPARRIMGDAEFSDKSVPDTVSHSIGISVEKE |
| | | TIIMGTLKAETPFFFGIESKEKKQTDLMLLLDGQNHYRIPRSALR |
| | | GILRRDIRSVLGTGCNAEVGGRPCLCPVCRIMKNITVMDTRSSTD |
| | | TLPEVRPRIRLNPFTGSVQEKALFNMEMGTEGIEFPFVLSYRGKK |
| | | TLPKELRNVLNWWTEGKAFLGGAASTGKSIFQLSDIHAFSSDLS |
| | | DETARESYLSNHGWRGIMENSIVHESPLEGGAGGCSFGLSDLPK |
| | | LGWHAEDLKLSDIEKYKPFHRQKISVKITLNSPFLNGDPVRALTE |
| | | DVADIVSFKKYTQGGEKIIYAYKSESFRGVVRTALGLRNQGNDD |
| | | ITGKKNVPLIALTHQDCECMLCRFFGSEYEAGRLYFEDLTFESEP |
| | | EPRRFDHVAIDRFTGGAVNQKKFDDRSLVPGKEGFMTLIGCFW |
| | | MRKDKELSRNEIEELGKAFADIRDGLYPLGAKGSMGYGQVAEL |
| | | SIVDDEDSDDENNPAKLLAESMKNASPSLGTPTSLKKKDAGLSL |
| | | RFDENADYYPYYFLEPEKSVHRDPVPPGHEEAFRGGLLTGRITCR |
| | | LTVRTPLIVPNTETDDAFNMKEKAGKKKDAYHKSYRFFTLNRVP |
| | | MIPGSEIRGMISSVFEALSNSCFRIFDEKYRLSWRMDADVKELEQ |
| | | FKPGRVADDGKRIEEMKEIRYPFYDRTYPERNAQNGYFRWDARI |
| | | SLTDNSMRKMEKDGVPRNVIYKLNTLKNKAYKSEKSFLFDLKN |
| | | KAGGVGRYKKLVLKHAEVRGGEIPYYSHPTPTDCKLLSLVGPNR |
| | | QLCRQDTLVQYRIIKHRRGAKPEEDFMFVGTPSENQKGHKENND |
| | | HGGGYLKISGPNKIEKENVLTSGVPSVPENMGAVVHNCPPRLVE |
| | | VTVRCGRKQEEECKRKRLVPEYVCADPEKKVTYTMTKRCERIFL |
| | | EKSRRIIPFTNDAVDKFEILVKEYRRNAEQQDTPEAFQTILPENGT |
| | | VNPGDLLYFREEKGKAAEIVPVRISRKVDDRHIGKRIDPELRPCH |
| | | GEWIEDGDLSKLDAYPAEKKLLTRHPKGLCPACRVFGTGSYKSR |
| | | VRFGFAALKGTPKWLKEDPAEPSQGKGITLPLLERPRPTWAVLH |
| | | NDKENSEIPGRKFYVHHNGWKGISEGIHPISGENIEPDENNRTVE |
| | | VLDKGNRFVFELSFENLEPRELGLLIHSLQLEKGLAHKLGMAKS |
| | | MGFGSVEIDVESVRVKHRSGEWDYKDGETVDGWIEEGKRGVA |
| | | AKGKANDLRKLLYLPGEKQNPHVHYPTLKKEKKGDPPGYEDLK |
| | | KSFREKKLNRRKMLTTLWEPWHK |
| 9 | CmaCas7- | MLKLKVKITYFQPFRVIPWIKEDDRNSDRNYLRGGTFARWHKD |
| | 11 | KKDDIHGKPYITGTLLRSALFTEIEKIKIHHSDFIHCCNAIDRTEGK |
| | | HQPSFLRKRPVYTENKNIQACNKCPLCLIMGRGDDRGEDLKKKK |
| | | HYNGKHYQNWTVHFSNFDTQATFYWKDIVQKRILNRVDQTCG |
| | | KAKDFFKVCEVDHIACPTLNGIIRINDEKLSQEEISKIKQLIAVGL |
| | | AQIESLAGGICRIDITNQNHDDLIKSFFETKPSKILQPNLKESGEER |
| | | FELAKLELLAEYLTQSFDANQKEQQLRRLADAIRDLRKYSPDYL |
| | | KDLPKGKKGGRTSIWNKKVADDFTLRDCLKNQKIPNELWRQFC |
| | | EGLGREVYKISKNISNRSDAKPRLLGETEYAGLPLRKEDEKEYSP |
| | | TYQNQESLPKTKWIISGELQAITPFYIGHVNKTSHTRSTIFLNMNG |
| | | QFCIPRSTLRGALRRDLRLVFGDSCNTPVGSRVCYCQVCQIMRCI |
| | | KFEDALSDVDSPPEVRHRIRLNCHTGVVEEGALFDMETGFQGMI |
| | | FPFRLYYESKNEIMSQHLYEVLNNWTNGQAFFGGEAGTGFGRFK |
| | | LLNNEVFLWEIDGEEEDYLQYLFSRGYKGIETDEIKKVADPIKW |
| | | KTLFTKLEIPPEKIPLTQLNYTLTIDSPLISRDPIAAMLDNRNPDAV |
| | | MVKKTILVYEQDSSTHKNVPKEVPKYFIKSETIRGLLRSIISRTEIK |
| | | LEDGKKERIFNLDHEDCDCLQCRLFGNVHQQGILRFEDAEITNK |
| | | NVSDCCIDHVAIDRFTGGGVEKMKFNDYPLSASPKNCLNLKGSI |
| | | WITSALKDSEKEALSKALSELKYGYASLGGLSAIGYGRVKELTL |
| | | EENDIIQLTEITESNLNSQSRLSLKPDVKKELSNNHFYYPHYFIKP |
| | | APKEVVRESRLISHVQGHDTEGEFLLTGKIKCRLQTLGPLFIANN |
| | | DKGDDYFELQHNNPGHLNYAFFRINDHIAIPGASIRGMISSVFETL |
| | | THSCFRVMDDKKYLTRRVIPESETTQKRKSGRYQVEESDPDLFP |
| | | GRVQKKGNKYKIEKMDEIVRLPIYDNFSLVERIREYHYSEECASY |
| | | VPSVKKAIDYNRMLAQAADSNREFLYNHPEAKSILQGKKEVYYI |
| | | LHKQESKNRGKTKEINPNARYACLTDENTPGSRKGFIKFTGPDM |
| | | VTVNKELKSKIAPIYDPEWEKDIPDWERSNQESNHKYSFILHNEI |
| | | EMRSSQKKKYPRPVFICKKNGVEYRMQKRCERIFDFTKEEEKDK |
| | | EIVIPQKVVSQYNAILKDNKENTETIPGLFNSKMVNKELEDGDLV |
| | | YFKYKEGKVTELTPVAISRKTDNKPMGKRFPKISINGKMKPNDS |
| | | LRSCSHTCTEDCDDCPNLCESVKDYFKPHPDGLCPACHLFGTTF |
| | | YKSRLSFGLAWLENNAKWYISNDFQQKDSKKEKGGKLTLPLLE |
| | | RPRPTWSMPNNNAEVPGRKFYVHHPWSVENIKNNQGNQKDISL |
| | | KPDSDAIKIKENNRTIEPLGKDNVFNFEISFNNLRDWELGLLLYAI |
| | | ELEDHLAHKLGMAKAFGMGSVKIEIKNLLIKGSINDISKAELIKK |
| | | GFKKLGIDSLEKDDLSEYLHIKQLREILWFSDKPVGTIEYPKLEN |
| | | KTNSRIPSYTDFVQEKDHETGFKNPKYQNLKSRLHILQNPWNAW |
| | | WKNEE |
| 10 | CbfCas7- | MSKTDDKIDIKLTFLEPYRMVNWLENGLRMTDPRYLRGLSFAR |
| | 11 | WHRNKNGKAGRPYITGTLLRSAVIRAAEELLSLNLGKWGKQLC |
| | | CPGQFETEREMRKNKTFLRRRPTPAWSAETKKEICTTHGSACAF |
| | | CLLLGRRLHGGKEDVNEDAPGSCRKPVGFGNLSLPFQPTKRQIQ |
| | | DVCKERVLNRVDFRTGKAQDYFRVFEIDHEDWGVYTGEITITEP |
| | | RVQEMLEASLKFVDTLCGALCRIEIVGSADETKRTTSSKEGCPAS |
| | | TTTRDCSSSENDDTSPEDPVREDLKKIAHVIANAFQNSGNREKVH |
| | | ALADAIRAMRLEESSIINTLPKGKSEKTTEQIEVNKHYLWDEIPV |
| | | NDTSVRHILIEQWRRWQSKKDDPEWWKFCDFLGECLYKEYKKL |
| | | TSGIQSRARVMGETEYYGALGMPDKVIPLLKSDKTKEWILVGSL |
| | | KAETPFFFGLETEQTEEVEHTSLRLVMDKKGRFRIPRSVLRGALR |
| | | RDMRIAFDSGCDVKLGSPLPCDCSVCQVMRSITIKDSRSEAGKLP |
| | | QIRHRIRLNPFSGTVDEGALFDIEVAPEGVIFPFVMRYRGEEFPPA |
| | | LLSVIRYWQDGKAWLGGEGATGKGRFALAKDLKMYEWKLED |
| | | KSLHAYIDTYGHRGNEHAIGTGQGIDGFRSGSLSDLLSDISKESFR |
| | | DPLASYHNYLDKRWIKVGYQITIGAPLLSADPIGALLDPNNVDAI |
| | | VFEKMKLDGDQVKYLPAIKGETIRGIVRTALGKRNNLLAKNDH |
| | | DDCTCSLCAIFGNENETGKIRFEDLEVYDKDIAKKIDHVAIDRFT |
| | | GGARDQMKFDTLPLIGSPERPLRLKGLFWMRRDVSPDEKARILL |
| | | AFLEIREGLYPIGGKTGSGYGWVSDLEFDGDAPEAFKEMNSKRG |
| | | KQASFKEKISFRYPSGAPKHIQNLKATSFYYPHYFLEPGSKVIREQ |
| | | KMIGHEQYYESYPSGASGEKLLSGRIICSMTTHTPLIVPDTGVIKD |
| | | PENKHATYDFFQMNNAIMIPGSEIRGMISAVYEAMTNSCFRIFHE |
| | | KQYLTRRISPEDKELREFIPGIVRIINGDVYIEKAEREYRLPLYDD |
| | | VHIITNYEELEYEKYIKKNPGREQKIKNAHRFNKNIARIAESNRN |
| | | YLCSLDRAVRREILSGRKKVNFRLVKVNDNKNPDKEAVELCKT |
| | | GPLEGLVKFSGLNAVNISNLRPGTAEEGFDAKWDMWSLNIILNR |
| | | MDVRNSQKKEYPRPALHFNHDGKEYTIPKRCERVFVRAEAGKR |
| | | AETEGSYKVPRKVQEQYQNILRDYESNIGHIDNTFRTLIENCGLN |
| | | NGSLVYFKPDNSRKEVVAITPVKISRKTDRLPQGDRFPHTSSDLR |
| | | PCVRDCLDTEGDIRMLENSPFKRLFHIHPEGLCPACQLFGTTNYR |
| | | GRVRFGFASLSDGPKWFRKDEGNETCHITLPLLERPRPTWSMPD |
| | | DTSTIPGRKFYVHHMGYETVKKNQRTLVKTENNRTVKALDKEN |
| | | EFTFEVFFENLREWELGLLLHCLELEPEMGHKLGMGKPLGFGSV |
| | | KIRIDKLQKCVVNVKDGCVLWEPEEDKIQHYIAKGLGKLTTWFG |
| | | KEWDRLEHIQGLRSLQRLLPL |
| 11 | sstCas7- | MIINITVKFLGPFRMLEWTDPDNRNRKNREFMRGQAFARWHNS |
| | 11 | NPQKGSQPYITGTLVRSAVIRSAENLLMLSEGKVGKEKCCPGEFR |
| | | TENRKKRDAMLHLRQRSTLQWKTDKPLCNGKSLCPICELLGRRI |
| | | GKTDEVKKKGDFRIHFGNLTPLNRYDDPSDIGTQRTLNRVDYAT |
| | | GKAHDFFKVWEIDHSLLSVFQGKISIADNIGDGATKLLEDSLRFT |
| | | DRLCGAICVISYDCIENSDGKENGKTGEAAHIMGESDAGKTDAE |
| | | NIANAIADMMGTAGEPEKLRILADAVRALRIGKNTVSQLPLDHE |
| | | GKENHHLWDIGEGKSIRELLLEKAESLPSDQWRKFCEDVGEILY |
| | | LKSKDPTGGLTVSQRILGDEAFWSKADRQLNPSAVSIPVTTETLI |
| | | CGKLISETPFFFGTEIEDAKHTNLKVLLDRQNRYRLPRSAIRGVLR |
| | | RDLRTAFGGKGCNVELGGRPCLCDVCRIMRGITIMDARSEYAEP |
| | | PEIRHRIRLNPYTGTVAEGALFDMELGPQGLSFDFILRYRGKGKSI |
| | | PKALRNVLKWWTKGQAFLSGAASTGKGIFRLDDLKYISFDLSDK |
| | | DKRKDYLDNYGWRNRIEALSLEKMPLDRMNDYAEPLWQKVSV |
| | | EIEIGSPFLNGDPIRALIEKDGSDIVSFRKYADDSGKEVYAYKAES |
| | | FRGVVRAALARQHFDKEGKPLDKEGKPLLTLIHQDCECLICRLF |
| | | GSEHETGRLRFEDLLFDPQPEPMIFDHVAIDRFTGGAVDKKKFD |
| | | DCSLPGTPGHPLTLKGCFWIRKELEKPDEDKSEREALSKALADIH |
| | | NGLYPLGGKGAIGYGQVMNLKIKGAGDVIKAALQSESSRMSAS |
| | | EPEHKKPDSGLKLSFDDKKAVYYPHYFLKPAAEEVNRKPIPTGH |
| | | ETLNSGLLTGKIRCRLTTRTPLIVPDTSNDDFFQTGVEGHESYAFF |
| | | SVNGDIMLPGSEIRGMLSSVYEALTNSCFRVFDEGYRLSWRMEA |
| | | DRNVLMQFKPGRVTDNGLRIEEMKEYRYPFYDRDCSDKKSQEA |
| | | YFDEWERSITLTDDSLEKMAERKGDISPKDLKVLKSLKGKNYKS |
| | | TEGLLAAFKDKGGDTGGNILGLIFKYAERIGDVPRYEHPTDTDR |
| | | MMLSLSEYNRNQKSDGKRAYKIIKPASKLGKGAYFMFAGTSVE |
| | | NKRICNPACTDKANKSVKGYLKISGPNKLEKYNISEPELDGVPED |
| | | RNCQIIHNRIYLRKIFVANAKKRKERDRLVGEFACYDPEKKVTYS |
| | | MTKRCERIFIKDRGRTLPITHEASELFEILVQEYRENAKRQDTPEV |
| | | FQTLLPDNGRLNPGDLVYFREEKGKTVEIIPVRISRKIDDSPIGKR |
| | | LREDLRPCHGEWIEGDDLSQLSEYPEKKLFTRNTEGLCPACRLFG |
| | | TGAYKGRLRFGFAKLENDPKWLMKNSDGPSHGGPLTLPLLERP |
| | | RPTWSMPDDTLNRLKKDGKQEPKKQKGKKGPQVPGRKFYVHH |
| | | DGWKEINCGCHPTTKENIVQNQNNRTVEPLDKGNTFSFEICFENL |
| | | EPYELGLLLYTLELEKGLAHKLGMAKPMGFGSIDIEVENVSLRT |
| | | DSGQWKDANEQISEWTDKGKKDAGKWFKTDWEAAEHIKNLKK |
| | | LLFLPGEEQNPRVIYPALKQKDIPNSRLPGYEELKKNLNMEKRKE |
| | | MLTTPWAPWHPIKK |
| 12 | hvmCas7- | MTQITIQVTFFHPFRVVPWNHRDHRKTDRKYLRGGTFAKWHCT |
| | 11 | ASEGKSGRPYITGTLLRSALFAEIEKLIAFHDPFKCCRGKDKTEN |
| | | GNAKPLFLRRRPRADCDPCGTCPLCLLMGRSDTVRRDAKKQKK |
| | | DWSVHFCNLREATERSFNWKETAIERIVNRVDPSSGKAKDYMRI |
| | | WEIDPLVCSQFNGIITINLDTDNAGKVKLLMAAGLAQINILAGSIC |
| | | RADIISEDHDALIKQFMAIDVREPEVSTSFPLQDDELNNAPAGCG |
| | | DDEISTDQPVGHNLVDRVRISKIAESIEDVESQEQKAQQLRRMAD |
| | | AIRDLRRSKPDETTLDALPKGKTDKDNSVWDKPLKKDILPSPRM |
| | | PASEDDDTPTLRKVLKDEINGQEDMWRKFCEALGNSLYDLSKK |
| | | AKERKRTEALPRLLGETEIYGLPMRENKEDEPLPSSLTYKFKWLI |
| | | AGELRAETPFFFGTEVQEGQTSATILLNRDGYFRLPRSVIRGALR |
| | | RDLRLVMGNDGCNMPIGGQMCECGVCRVMRHIVIEDGLSDCKI |
| | | PPEVRHRIRLNCHTGTVEEGALFDMETGYQGMTFPFRLYCETEN |
| | | SDLDSYLWEVLNNWQNGQSLFGGDTGTGFGRFELTEPKVFLWN |
| | | FSKKEKHEAYLLNRGFKGQMPVQDVKTKSFKTKTWFQIHRELDI |
| | | SPKKLPWYSTDYRFNVTSPLISRDPIGAMLDPRNTDAIMVRKTVF |
| | | CPDPNAKNRPAPATVYMIKGESIRGILRSIVVRNEELYDTDHEDC |
| | | DCILCRLFGSIHQQGSLRFEDAEVQNSVSDKKMDHVAIDRFTGG |
| | | GVDQMKFDDYPLPGCPAQPLILEGKFWVKDDIDDESKSALEKAF |
| | | ADFRDGLVSLGGLGAIGYGQIGDFELIGGSADWLNLPKPEENRT |
| | | DVPCGDRSAQGPEIKISLDADKIYHPHFFLKPSDKNVYRERELVS |
| | | HAKKKGPDGKSLFTGKITCRLSTEGPVFIPDTDLGEDYFEMQASH |
| | | KKHKNYGFFRINGNVAIPGSSIRGMISSVFEALTNSCFRVFDQER |
| | | YLSRSEKPDPTELTKYYPGKVKRDGNKFFILKMKDFFRLPLYDF |
| | | DFEGEAESLRPNYDEDRNEEENKGKNKNTQKVKNAVEFNIKMA |
| | | GFAKHNRDFLKKYKEQEIKDIFMGKKKVYFTAGKHKPNEAHDN |
| | | DKIALLTKGSNKKAEKGYFKFTGPGMVNVKAGVEGEECDFHID |
| | | ESDPDVYWNMSSILPHNQIKWRPSQKKEYPRPVLKCVKDGTEY |
| | | VMLKRSEHVFAEASSEDSYPVPGKVRKQFNSISRDNVQNTDHLS |
| | | SMFQSRRLHDELSHGDLVYFRHDEKRKVTDIAYVRVSRTVDDR |
| | | PMGKRFKNESLRPCNHVCVEGCDECPDRCKELEDYFSPHPEGLC |
| | | PACHLFGTTDYKGRVSFGLGWHESNTPKWYMPEDNSQKGSHLT |
| | | LPLLERPRPTWSMPNKKSEIPGRKFYVHHPWSVDKIRNRQFDPA |
| | | KEKQPDDVIKPNENNRTVEPLGKGNEFTFEVRENNLREWELGLL |
| | | LYSLELEDNMAHKLGMGKALGMGSARIKAEAIELRCESAGQNA |
| | | ELKDKAAFVRKGFEFLEIDKPGENDPMNFDHIRQLRELLWFLPE |
| | | NVSANVRYPMLEKEDDGTPGYTDFIKQEEPSTGKRNPSYLSSEK |
| | | RRNILQTPWKHWYLIPPFQASAQSETVFEGTVKWFDDKKGFGFI |
| | | KINDGGKDVFVHHSSIVGTGFKSLNEGDSVAFKMGVGPKGPCAE |
| | | KVKKIGN |
| 13 | hreCas7- | MSVEEFYVRLTFLEPFRVVPWVRNGDERKGDRIYQRGGTYARW |
| | 11 | HKINDSHGQPYITGTMLRSAVLREIENTLTLHNTYGCCPGGTRTT |
| | | EGKLEKPLYLRRRDGFEFENHAEKPCSEEDPCPLCLIQGRFDKLR |
| | | RDEKKQFVRQGNISFCSVNFSNLNISSGIKSFSWEEIAVSRVVNRV |
| | | DPNSGKAKDFFRVWEIDHKLCPNFLGKMSISLSEKLEDVKALLA |
| | | VGLAQVNVLSGALCRVDIIDPETQKDTVHQHLIQQFVTRIQDKE |
| | | KGDAADIPAFTLPPAGLSPSSNEWNDTIKSLAEKIRKIKELEQGQ |
| | | KLRQMADVIRELRRKTPAYLDQLPAGKPEGRESIWEKTPTGETL |
| | | TLRQLLKSANVPGESWRAFCEELGEQLYRLEKNLYSHARPLPRL |
| | | LGETEFYGQPARKSDDPPMIRASYRAFPSYVWVLDGILRAETPFY |
| | | FGTETSEGQTSQAIILCPDGSYRLPRSLLRGVIRRDLRAILGTGCN |
| | | VSLGKVRPCSCPVCEIMRRITVQQGVSSYREPAEVRQRIRSNPHT |
| | | GTVEEGALFDLETGPQGMTFPFRLYFRTRSPYIDRALWLTINHW |
| | | QEGKAIFGGDIGVGMGRFRLENLQIRSADLVSRRDFSLYLRARG |
| | | LKGLSREEVTRIGLNEEQWEAVMADDPGTHYNPFPWEKISYTLL |
| | | IHSPLISNDPIAAMLDHDNKDAVMVQKTVLFVDESGNYSQMPH |
| | | HFLKGSGIRGACRFLLGRKDAPNENGLTYFEADHEECDCLLCSL |
| | | FGSKHYQGKLRFEDAELQDEVEAIKCDHVAIDRFHGGTVHRMK |
| | | YDDYPLPGSPNRPLRIKGNIWVKRDLSDTEKEAVKDVLTELRDG |
| | | LIPLGANGGAGYGRIQRLMIDDGPGWLALPERKEDERPQPSFSPV |
| | | SLGPVHVNLKSGSDTADVYYYHPHYFLEPPSQTVSRELDIISHAR |
| | | TRDSGGEALLTGRILCRLITRGPIFIPDTNNDNAFGLEGGIGHKNY |
| | | RFFRINDELAIPGSELRGMVSSVYEALTNSCFRIMEEGRYLSRRM |
| | | GADEFKDFHPGIVVDGAKIREMKRYRLPLYDTPDKTSRTKEMTC |
| | | PELFTRKDGRPERAKKFNEEIAKVAVQNRAYLLSLDEKERREVL |
| | | LGNREVTFDECPDDEYSDDEYSELKYAQKYKDFIAVLKKNGQK |
| | | RGYIKFTGPNTANKKNEDAPDKNYRSDWDPFKLNILLESDPECR |
| | | VSNIHCYPRPLLVCIKDKAEYRIHKRCEAIFCSIGSPSDLYDIPQKV |
| | | SNQYRTILQDYNDNTGKIVEIFRTQIKHDQLTTGDLVYFKPAANG |
| | | QVNAVIPVSISRKTDENPLAKRFKNDSLRPCAGLCVEDCNECPAR |
| | | CKKVADYFNPHPRGLCPACHLFGTTFYKGRVRFGFAWLTGEDG |
| | | APRWYKGPDPCDSGKGRPMTIPLLERPRPTWSIPDNSFDIPGRKF |
| | | YVHHPYSVDGIDGETRTPNNRTIEPLAEGNEFVFDIDFENLRDWE |
| | | LGLLLYSLELEDSLAHKLGLGKPLGFGTVQINIRGISLKNGSKGW |
| | | DTKTGDDKNQWIKKGFAHLGIDIKEANERPYIKQLRELLWVPTG |
| | | DNLPHVRYPELESKTKDVPGYTSLLKEKDLADRVSLLKAPWKP |
| | | WKPWSGTAPHPDKGTNRLRASIVERDRIQRKTDTAKPEKKEETK |
| | | VGKSSSSDIEKRYVGTVKWFNDKKGYGFILYGTDEEIFVHRSGV |
| | | ADNSIPKEGQKVGFRIERGARGSHAVEVKAIE |
| 14 | fmCas7- | MPRFQLSLTFFDEPFRLIEWTDKSNRNSANTQWMRGQGFARWH |
| | 11 | KITLEKGFPFVTGTAVRSKIIREVEALLSRNKGTWNGIPCCSGFFD |
| | | TKGPSPTHLRYRPTLEWEYGKTVCTSEADVCPLCLLLGRFDQAG |
| | | KKSDTPCQSTDYHVHWENLSAGVAQYRLEDIAQKRTSNRVDFF |
| | | SKKAHDHYGVWEVTAVKNLLGYIYISDAITESHQKTVISLLKAA |
| | | LSFTDTLCGANCKLELSDEPVDSIHSNQSASNFNPHSGAAPSQCS |
| | | QSMPPFNMDQETKELANTLCKAFTGNMRHLRTLADAVREMRR |
| | | MSPGISSLPRGRLNKEGEITAHYLWDERIDEKTIRQVLEDTIELSP |
| | | ARSIIYKNWISFCNQLGQKLYERAKDNDPILERKRPLGEAAFSKV |
| | | PTSSHAPRHDMNSRVKGGFTREWIIVGTLRALTPFYMGTGSQAG |
| | | KQTSMPTLQDSNDHFRLPRTALRGALRRDINQASDGMGCVVEL |
| | | GPHNLCSCPVCQVLRQIRLLDTKSKFSMPPAIRQKICKNPVLSIVN |
| | | EGSLFDVELGIEGETFPFVMRYRGGAKIPDTIITVLSWWKNERLFI |
| | | GGESGTGRGRFVLECPRIFCWDVEKGQNDYIQYHGFRNKEDELL |
| | | SVYSTVSGLAEKNDVNLNNARDFSFDKICWEVQFDGPVLTGDPL |
| | | AALFHGNTDSVFYKKPILKSGEKEPSYQWAIKSDTVRGLIRSAFG |
| | | KRDALLIKSHEDCDCLLCEAFGSKHHEGKLRFEDLTPKSDEIKTY |
| | | RMDHVAIDRISGGAVDQCKYDDEPLVGTSKHPLVFKGMFWINR |
| | | DSSVEMQRALIAAFKEIRDGLYPLGSNGGTGYGWISHLAITNGPD |
| | | WLNLEEVPLPQPTADIPVEECTAEPYPKFQKPDLDQNAVYYPHY |
| | | FLQPGKPAERERHPVSHDHIDDKLLTGRLVCTLTTKTPLIIPDTQT |
| | | NTMLPPNDAPEGHKSFRFFRIDDEVLIPGSEIRGMVSTVFEALTGS |
| | | CFRVINQKAHLSWRINADMAKHYRPGRIIQNNEKMFIQPYKMFR |
| | | LPFYAGFDPRNCLSEKQLLGIEPVKLWVKDFVASLVKPQTDIDIE |
| | | WKEKIGFVRVTGPNKVEVDSSNTPDPSLPECESDWKDIHITEDGS |
| | | TPSKNDRVYRCQLKGVTYTVAKWCEAFWVKDEGKKPITVNAE |
| | | AINRYHLIMKSYQDNPQSPPIIFRSLPVLNYKQDQKIIGSMIFYRES |
| | | AKSDKIVNEIIPVKISRTADTELLAKHLPNNDFLPCAATCLNECDT |
| | | CNAKTCKFLPLYREGYPVNGLCPSCHLFGTTGYQGRVRFGFAK |
| | | MNGNAKFCQGGERPEDRAVTLPLQERPKLTWVMPNENSTIPGR |
| | | KFFLHHQGWKKIVDEGKNPINGDVIEPDANNRTVEPLAAGNDFS |
| | | FEVFFENLREWELGLLRYTLELESELAHKLGMGKAFGFGSVKIKI |
| | | KSVDLRKQGEWEKATNTLVSEDKKSSWYNIHTVNNLRTALYYV |
| | | EDDKIQVNYPKLKKDNESDNRPGYVEMKKTAFPVRDILTTPWW |
| | | PWWPPTPPPMNQSGNQSYARSEEPARITESQPEVYKTGTVKFYK |
| | | HDKKFGFITMDGRENIHFAGNQICRPETSLQSGDKVKFIEGENYK |
| | | GPTALKVERLKG |
| 15 | smCas7- | MRLKINIHFLEPFRLIEWHEQDRRNKGNSRWQRGQSFARWHRR |
| | 11 | KDNDQGRPYITGTLLRSVVIRAVEEELARPDTAWQSCGGLFITPD |
| | | GQTKPQHLRHRATVRARQTAKDKCADRQSACPFCLLLGRFDQV |
| | | GKDGDKKGEGLRFDVRFSNLDLPKDFSPRDFDGPQEIGSRRTINR |
| | | VDDETGKAHDFFSIWEVDAVREFQGEIVLAADLPSRDQVESLLH |
| | | HALGFVDRLCGARCVISIADQKPAEREERTVAAGDEKATIADYD |
| | | QVKGLPYTRLRPLADAVRNLRQLDLAELNKPDGKFLPPGRVNK |
| | | DGRRVPHYVWDIPLGKGDTLRKRLEFLAASCEGDQAKWRNICE |
| | | SEGQALYEKSKKLKDSPAAPGRHLGAAEQVRPPQPPVSYSEESIN |
| | | SDLPLAEWIITGTLRAETPFAIGMDAPIDDDQTSSRTLVDRDGRY |
| | | RLPRSTLRGILRRDLSLASGDQGCQVRLGPERPCTCPVCLILRQV |
| | | VIADTVSETTVPADIRQRIRRNPITGTAADGGLFDTERGPKGAGF |
| | | PFSLRYRGHAPMPKALRTVLQWWSAGKCFAGSDGGVGCGRFA |
| | | LDNLEVYRWDLGTFAFRQAYSENNGLRSPEEEFDLAVIHELAEG |
| | | LAKEDGQKILKGTEPFTCWQERSWQFSFTGPLLQGDPLAALNSD |
| | | TADIISFRRTVVDNGEVLREPVLRGEGLRGLLRTAVGRVAGDDL |
| | | LTRSHQDCKCEICQLFGSEHRAGILRFEDLPPVSPTTVADKRLDH |
| | | VAIDRFDQSVVEKYDDRPLVGSPKQPLVFKGCFWVQTSGMTHQ |
| | | LTELLAQAWRDIAAGHYPVGGKGGIGYGWINSLVVDGEKITCRP |
| | | DGDSISLTTVTGDIPPRPALTPPAGAIYYPHYFLPPNPEHKPKRSD |
| | | KIIGHHTFATDPDSFTGRITCKLEVVTPLIVPDTEGEQPKDQHKNF |
| | | PFFKINDEIMLPGAPLWAAVSQVYEALTNSCFRVMKQKRFLSWR |
| | | MEAEDYKDFYPGRVLDGGKQIKKMGDKAIRMPLYDDSTATGSI |
| | | KDDQLISDCCPKSDEKLQKALATNQKIALAAKHNQEYLAQLSPD |
| | | EREEALQGLKKVSFWTESLANNEAPPFLIAKLGEERGKPKRAGY |
| | | LKITGPNNANIANTNNPDDGGYIPSWKDQFDYSFRLLGPPRCLPN |
| | | TKGNREYPRPGFTCVIDGKEYSLTKRCERIFEDISGGENQVVRAV |
| | | TERVREQYREILASYRANAAGIAEGFRTRMYDTEELRENDLVYF |
| | | KTAKQADGKERVVAISPVCISREADDRPLGKRLPAGFQPCSHVC |
| | | LEDCNTCSAKNCPVPLYREGWPVNGLCPACRLFGAQMYKGRV |
| | | NFGFARLPDDKQPETKTLTLPLLERPRPTWVLPKSVKGSNTEDA |
| | | TIPGRKFYLRHDGWRIVMAGTNPITGESIEKTANNATVEAIMPGA |
| | | TFTFDIVCENLDQQELGLLLYSLELEEGMSHTLGRGKPLGFGNV |
| | | RIKVEKIEKRLSDGSRREMIPPKGAGLFMTDKVQDALRGLTEGG |
| | | DWHQRPHISGLRRLLTRYPEIKARYPKLSQGEDKEPGYIELKSQK |
| | | DENGVPIYNPNRELRVSENGPLPWFLLAKK |
| 16 | omCas7- | MIPDLRSLVVHISFLTPYRQAPWFPPEKRRNNNRDWLRMQSYAR |
| | 11 | WHKVAPEEGHPFITGTLLRSRVIRAVEEELCLANGIWRGVACCP |
| | | GEFNSQAKKKPKHLRRRTTLQWYPEGAKSCSKQDGRENACPFC |
| | | LLLDRFGGEKSEEGRKKNNDYDVHFSNLNPFYPGSSPKVWSGPE |
| | | EIGRLRTLNRIDRLTTKAQDFFRIYEVDQVRDFFGTITLAGDLPRK |
| | | VDVEFLLRRGLGFVSTLCGAQCEIKVVDLKKKQNNKEDSILPVS |
| | | EVPFFLEPEVLAKMCQDVFPSGKLRMLADVILRLREEGPDNLTLP |
| | | MGSQGLGGRLPHHLWDVPLVSKDRETQTLRSCLEKIAAQCKSE |
| | | QTQFRLFCQKLGSSLFRINKGVYLAPNSKISPEPCLDPSKTIRTKG |
| | | PVPGKQKHRFSLLPPFEWIITGTLKAQTPFFIPDEQGSHDHTSRKI |
| | | LLTRDFYYRLPRSLLRGIIRRDLHEATDKGGCRVELAPDVPCTCQ |
| | | VCRLLGRMLLADTTSTTKVAPDMRHRVGVDRSCGIVRDGALFD |
| | | TEYGIEGVCFPLEIRYRGNKDLEGPIRQLLSWWQQGLLFLGGDF |
| | | GIGKGRFRLENMKIHRWDLRDESARADYVQKCGLRRGVGDDT |
| | | AINLEKDLSLNLPESGYPWKKHAWKLSFQVPLLTADPIMAQTRH |
| | | EEDSVYFQKRIFTSDGRVVLVPALRGEGLRGLLRTAVSRAYGISL |
| | | INDEHEDCDCPLCKIFGNEHHAGMLRFDDMVPVGTWNDKKIDH |
| | | VSCSRFDASVVNKFDDRSLVGSPDSPLHFEGTFWLHRDFQNDVE |
| | | IKTALQDFADGLYSIGGKGGIGYGWLFDMEIPRSLRKLNSGFREA |
| | | SSIQDALLDSAKEIPLSAPLTFTPVKGAVYNPYYYLPFPAEKPERC |
| | | LVPPSHARLQSDRYTGCLTCELETVSPLLLPDTCREKDGNYKEYP |
| | | SFRLNNTPMIPGAGLRAAVSQVYEVLTNSCIRIMDQGQTLSWRM |
| | | STSEHKDYQPGKITDNGRKIQPMGKQAIRLPLYDEVIHHVSTPGD |
| | | TDDLEKLKAIVLELTRPWKELPEEQKKKRFEKCKNILDGRMLQQ |
| | | KELRALENSGFAYWRDKTSLTFDSFLKDAIEQEYPRYSGDYQRI |
| | | KALVVNITLPWKLLKKEERHKRFDKCRRILKGQQPLTKDERKAL |
| | | EESGFANWHGRELLFDRFLKDENSCLIKAETTDRVIASVAKNNR |
| | | DYLFEIKQQDFARYKRIIQGLERVPFSLRSLAKSKETSFQIACLGL |
| | | RRGRFLRKGYLKISGPNNANVEISGGSHSNSGYSDIWDDPLDFSF |
| | | RLSGKSELRPNTQKTREYPRPSFTCTVDGKQYTVNKRCERVFED |
| | | SAAPAIELPRMVREGYKGILTDYEQNAKHIPQGFQTRFSSYRELN |
| | | DGDLVYYKTDSQGRVTDLAPVCLSRLADDRPLGKRLPEEYRPC |
| | | AHVCLEECDPCTGKDCPVPIYREGYPARGFCPACQLFGTQMYKG |
| | | RVRFSFGVPVNSTRSPQLKYVTLPSQERPRPTWVLPESCKGKEK |
| | | DVPGRKFYLRHDGWREMWGDDDKPDSRPSSEECQDIIEGIGPGE |
| | | KFHFRVAFENLDKNELGRLLYSLELDAGMNHHLGRGKAFGFGQ |
| | | VKIRVTKLERRLEPGQWRSEKICTDLPVTSSELVISSLKKVEERRK |
| | | LLRLVMTPYKGLTACYPGLERENGRPGYTDLKMLATYDPYREL |
| | | VVQIGSNQPLRPWYEPGKSFKPSPGNDCTGRGGSVSKSLISEPKV |
| | | VPAIAPFCEGVVKWFNSVKGFGFIETKEQRDIFVHFSAIRGEGYKI |
| | | LEPGEKVRFEIGEGRKGPQAINVIRIR |
| 17 | SybCas7- | MFPKGRQMRRQRLLGDAEYYGGTGREQPASIVISTDSDPDHKV |
| | 11 | YEWIITGQLKAETGFFFGTKAGAGGHTDLSILLGKDGHYRVPRS |
| | | VFRGALRRDLRVAFGAGCRVEVGRERPCECPVCKVMRQITVMD |
| | | TISSYREAPEIRQRIRLNPYTGTVDKGALFDMEVGPEGIEFPFVLR |
| | | FRGSKSFPSELAAVIGSWTKGTAWLGGAAATGKGRFSLLGLSIH |
| | | KWNLSTAEGRKSYLAAYGLRDAADKTVKRLSIDKGGKGDVGLP |
| | | AGLERDALPSSVREPLWKKLVCTVDFSSPLLLADPIAALLGVEG |
| | | DERIGFDNIAYEKRRYNGETNTTESIPAVKGETFRGIVRTALGKR |
| | | HGNLTRDHEDCRCRLCAVFGKEQEAGKIRFEDLMPVGAWTRKH |
| | | LDHVAIDRFHGGAEENMKFDTYALAASPTNPLRMKGLIWVRSD |
| | | LFETGHDGPTPPYVKDIIDALADVKRGLYPVGGKTGSGYGWIKD |
| | | VTIDGLPQGLSLPPAEERVDGVNEVPPYNYSAPPDLPSAAEGEYF |
| | | FPHVFIKPYDKVDRVSRLTGHDRFRQGRITGRITCTLKTLTPLIIPD |
| | | SEGIQTDATGHKMCKFFSVAGKPMIPGSEIRGMISSVYEALTNSC |
| | | FRVFDEEKYLTRRVQPKKGAKSSELVPGIIVWGQNGGLAVQQV |
| | | KNAYRVPLYDDPAVTSAIPTEAQKNKERWESVPSVNLQGALDW |
| | | NLTTANIARDNRTFLNSRPEEKDAILSGTKPISFELEGTNPNDMLV |
| | | RLVPDGVDGAHSGYLKFTGLNMVLKANKKTSRKLAPSEEDVRT |
| | | LAILHNDFDSRRDWRRPPNSQRYFPRSVLRFSLERSTYTIPKRCER |
| | | VFEGTCGEPYSVPSDVERQYNSIIDDISKNYGRISETYLTKTANRK |
| | | LTVGDLVYFIADLDKNMATHILPVFISRISDEKPLGELLPFSGKLIP |
| | | CEGEPPTILKKMAPSLLTEAWRTLISTHLEGFCPACRLFGTTSYK |
| | | GRIRFGFAEHTGTPKWLREELDWARPFLTLPIQERPRPTWSVPDD |
| | | KSEVPGRKFYLHHHGGNRIVESNLRNRPEVNQTKNNSSVEPISA |
| | | GNTFTFDVCFENLEAWELGLLLYCLELSPKLAHKLGRAKAFGFG |
| | | SVKIHVERIEERTTDGAYQDVTAVKKNGWITTGHDKLREWFHR |
| | | DDWEDVDHIRNLRTVLRFPDADQEHDVRYPELKANNGVSGYVE |
| | | LRDKMTASERQESLRTPWYRWFPQNGTGGSGRHEQAATSQEQD |
| | | TAKDESVLSATQRRQAVIDVSDPDERLSGTVESFDRQKGDGYIG |
| | | CGVRQFYVRLEDIRSRTALCEGQVVTFRARKEWEGHEAYDVEID |
| | | Q |
| 18 | gwCas7- | MTKKPGTEDKATLWGKESASKSVKTILEESIQGFTVEQKRSFFA |
| | 11 | NLADQLVSRAGEQGAKSVRSQGLIIGRKENYAKPSAQEPTRHHL |
| | | YRQPSNASAFLATGWLIAETPFFIGSGTEGQKQTDDQAESLHLRT |
| | | LRDGHGRFRIPFTTIRGVMDKELRDILQAGCAKGRSLRAPCPCQV |
| | | CTLMRRIQVRDAIAADILPPDLRMRTRIDPSHGTVAHLFSLEMAP |
| | | QGLKLPFFLKLKGVETIDPDKELLEILNDWSAGQCFLGGLWGTG |
| | | KGRFRLDDLQWHRLELDNADYYTPLLQDRFFAGETISDLRQGLQ |
| | | SINIQPERIPAQTPSRNMPYCRVDCILEFKSPVLSGDPVAALFESD |
| | | APDNVAYKKPVVQYDETGRLRTTDPGPVEMLTCLKGEGVRGV |
| | | VAYLAGKAYDQHDLSHDSCNCTFCQAFGNGQKAGSLRFDDFM |
| | | PVQFESDQAGNFSWSPHTPHAMRSDRVALDVFGGAMPEAKFDD |
| | | RPLAASPGKPLNFKSTIWYREDMGKEAGKALKRALIDLQNNMA |
| | | AIGSGGGIGRGWVSRVCFEGDIPDFLEDFPEPITVTEPEQDSQLLK |
| | | NQAVADETAVSACDTADAPHPLAVTLEPGARYFPRVIIPRAPTV |
| | | KRDECVTGQRYHTGRLSGKIFCELNTLGPLFVPDTDYSAGVPVPI |
| | | SDEQLAECQLQAVFENTSKFNEFFATYPEETVTKLKDLLCAADD |
| | | KWILAVKDITADLRQEIGEDTFQRIIRKAGHKTQRFHQINDEIGLP |
| | | GASLRGMVLSNYQILTNSCYRNLKATEEITRRMPADEAKYRKA |
| | | GRVTVSGDGAQKKYSIQEMEVLRLPIYDNMNTPDNMPDVAKQA |
| | | TTAKRCNNLMNEAAKTSRVELKARWREGQSKIKYQIIDALNKV |
| | | DPIIQVISSSKQINPNNGKTGWGYVKYTGANVFAKSLVAPIDCLR |
| | | KKDAGHVCCQVNLNPAWEASNFDILINEKCPVERQSGPRPTLRC |
| | | KGQDSAWYTLTKRSERIFTDKKPVPDPINIPPREVKRYNELRDSY |
| | | KKNTAHVPKPLQTFFNQESLANGDLVYFEVNQFGEASQLTPVSIS |
| | | RTTDLFPIGGRLPQGHKDLFPCTAMCLSECKNCVPASFCEFHSRS |
| | | HEKLCPACSLAGTTGNRGRIKFSEAWLSGLPKWHSVSQDNVGR |
| | | GLGVTMPRLERSRRTWHLPTKDAYLLGQSIYLNHPVPAILPSDQ |
| | | VPSENNQTVEPLGPKNIFSFQLAFDNLSIEELGLLLYSLELESGMA |
| | | HRLGRGRALGMGSVQISVKDIQIRDNKSFLFSSNISKKSEWIQCG |
| | | KDEFAQEAWFGESWDNIDHIQRLRQALTIPVKGDVGCIRYPKLE |
| | | AEGGMPDYIKLRKRLTPLCDREEPVRYRINPVQLARMILPFVPW |
| | | HGACPALLNEQVMIEAKRLTELXXXDRANWPC |

Table 2 below shows Cas7-11 guide sequences for trans-splicing.

TABLE 2

| SEQ | | |
|---|---|---|
| ID | | |
| NO | ID | Sequence |
| 19 | COL7A1_intron_ | GTTGATGTCACGGAACGGCCGTGGCCAGCAACTTCGCGG |
| | 4_6_1 | TGAGTGAC |
| 20 | COL7A1_intron_ | GTTGATGTCACGGAACGTGAGTGACGGGAGGATGGCGC |
| | 4_6_2 | TCTGAGCAC |
| 21 | COL7A1_intron_ | GTTGATGTCACGGAACTCTGAGCACAGCACAGCCCTTGA |
| | 4_6_3 | GCAGTGAC |
| 22 | COL7A1_intron_ | GTTGATGTCACGGAACAGCAGTGACCCTCCTATAGAACA |
| | 4_6_4 | CTATCTGG |
| 23 | COL7A1_intron_ | GTTGATGTCACGGAACACTATCTGGGCTGTGATTCCACA |
| | 4_6_5 | GTGCTGGG |
| 24 | COL7A1_intron_ | GTTGATGTCACGGAACAGTGCTGGGCCCGTGAGCAGGCT |
| | 4_6_6 | GGGAGCTC |
| 25 | COL7A1_intron_ | GTTGATGTCACGGAACTGGGAGCTCTGCGGCTCTCCTTC |
| | 4_6_7 | TGCTAGAA |
| 26 | COL7A1_intron_ | GTTGATGTCACGGAACCTGCTAGAACCTGCCCCCAGACT |
| | 4_6_8 | CTTGGCTA |
| 27 | COL7A1_intron_ | GTTGATGTCACGGAACTCTTGGCTATGATCCTGTGACCC |
| | 4_6_9 | CAAGACCG |
| 28 | COL7A1_intron_ | GTTGATGTCACGGAACCCAAGACCGCCATGCAGGTCATG |
| | 4_6_10 | AGCTCTTT |
| 29 | COL7A1_intron_ | GTTGATGTCACGGAACGAGCTCTTTGTGTCAGTCCATTTT |
| | 4_6_11 | GTATAAC |
| 30 | COL7A1_intron_ | GTTGATGTCACGGAACTTGTATAACCCCTTCCCTGCTGTC |
| | 4_6_12 | AGCGGTG |
| 31 | COL7A1_intron_ | GTTGATGTCACGGAACTCAGCGGTGACTCTGTGACTTCT |
| | 4_6_13 | GGGCGGGG |
| 32 | COL7A1_intron_ | GTTGATGTCACGGAACTGGGCGGGGACTGAGCTGTATGA |
| | 4_6_14 | CTTCCAAT |
| 33 | COL7A1_intron_ | GTTGATGTCACGGAACACTTCCAATTCCATGTGACCTCC |
| | 4_6_15 | ATTCCAAT |
| 34 | COL7A1_intron_ | GTTGATGTCACGGAACCATTCCAATGAAGACTTTGATCA |
| | 4_6_16 | TACAACCC |
| 35 | COL7A1_intron_ | GTTGATGTCACGGAACATACAACCCCAAGGCAGGGCCA |
| | 4_6_17 | AGCTGTATC |
| 36 | COL7A1_intron_ | GTTGATGTCACGGAACAGCTGTATCTGTCCTGTTTGTTTT |
| | 4_6_18 | CAGGGCA |
| 37 | COL7A1_intron_ | GTTGATGTCACGGAACTTCAGGGCAGTGGAGAGGGCAG |
| | 4_6_19 | AGGAAGTCT |
| 38 | COL7A1_intron_ | GTTGATGTCACGGAACAGGAAGTCTGCTAACATGCGGTG |
| | 4_6_20 | ACGTCGAG |
| 39 | COL7A1_intron_ | GTTGATGTCACGGAACGACGTCGAGGAGAATCCTGGCCC |
| | 4_6_21 | AATGCCCG |
| 40 | COL7A1_intron_ | GTTGATGTCACGGAACCAATGCCCGCCATGAAGATCGAG |
| | 4_6_22 | TGCCGCAT |
| 41 | COL7A1_intron_ | GTTGATGTCACGGAACGAAACCTCCCCTTGCCCCATACC |
| | 4_8_1 | AGGCTTAC |
| 42 | COL7A1_intron_ | GTTGATGTCACGGAACAGGCCCTATGACCTAGACCTCAA |
| | 4_8_2 | CCCTGTAG |
| 43 | COL7A1_intron_ | GTTGATGTCACGGAACAAGTCCTGTGACCCCCCAAGTCC |
| | 4_8_3 | CATAGATA |
| 44 | COL7A1_intron_ | GTTGATGTCACGGAACCCCAGGCTCCAGTTAACCCCCTG |
| | 4_8_4 | ACCCAGCA |
| 45 | COL7A1_intron_ | GTTGATGTCACGGAACCTGGAGGTGACAAAGACCATCA |
| | 4_8_5 | GTGCTAGTC |
| 46 | ANXA4_3TS_1 | GTTGATGTCACGGAACcagatagaaagtaccctcaatttatcatcaa |
| 47 | B4GALNT1_3TS_1 | GTTGATGTCACGGAACtagggctggccgttcctggccgcagegcccc |
| 48 | KRAS_3TS_1 | GTTGATGTCACGGAACctttttaaacagaaaccttgtatctctctca |
| 49 | MALAT_3TS_1 | GTTGATGTCACGGAACgttaaacaatggaaaagtatttctcctacac |
| 50 | NF2_3TS_1 | GTTGATGTCACGGAACtattggaggagcaactcagaaagctgcatga |
| 51 | PPARG_3TS_1 | GTTGATGTCACGGAACgtaatcacaggcaagttataacatctctaag |
| 52 | PPIA_3TS_1 | GTTGATGTCACGGAACtcccaaatgaagggagcaacccaaataaaat |
| 53 | RPS5_3TS_1 | GTTGATGTCACGGAACtgtccagacacacacacacacaggctgaagt |
| 54 | SMARCA1_3TS_1 | GTTGATGTCACGGAACtttggcaatgatacaagtaaatctgacccat |
| 55 | STAT3_3TS_1 | GTTGATGTCACGGAACtggctcacgcctgtaatgccagcactttgag |
| 56 | TERT_3TS_1 | GTTGATGTCACGGAACgacccctggctcaggactggggtgcaaggca |
| 57 | TUG1_3TS_1 | GTTGATGTCACGGAACaaaaaagaaaacacaaaagtctgattaacac |
| 58 | ANXA4_3TS_2 | GTTGATGTCACGGAACatgtttcatgaacataggcgatgctctatgt |
| 59 | B4GALNT1_3TS_2 | GTTGATGTCACGGAACcagttcccaggccccacttcgtggttctctc |
| 60 | KRAS_3TS_2 | GTTGATGTCACGGAACttccttccttcttctactaagttattttgtt |
| 61 | MALAT_3TS_2 | GTTGATGTCACGGAACcaaaatttttgaagcataccttaacatcttg |
| 62 | NF2_3TS_2 | GTTGATGTCACGGAACgtcacacagagagagggcgtgtaaataaggc |
| 63 | PPARG_3TS_2 | GTTGATGTCACGGAACaaaaaacactggagttaaggcaagaaaaaga |
| 64 | PPIA_3TS_2 | GTTGATGTCACGGAACcagttcagatatgtgtatcctgaaatattct |
| 65 | RPS5_3TS_2 | GTTGATGTCACGGAACgcctgatttgcaatcagatagagggtcacaa |
| 66 | SMARCA1_3TS_2 | GTTGATGTCACGGAACagatacttttttgtactgttatattttagag |
| 67 | STAT3_3TS_2 | GTTGATGTCACGGAACgtatccccaagagaaggctccctgttggcca |
| 68 | TERT_3TS_2 | GTTGATGTCACGGAACggggggctgtgtcccctctctgagcctcag |
| 69 | TUG1_3TS_2 | GTTGATGTCACGGAACaaagacaaatgataaatgaaaacaaacaaca |
| 70 | ANXA4_3TS_3 | GTTGATGTCACGGAACatcaatatttgctttgccagggaaattttag |
| 71 | B4GALNT1_3TS_3 | GTTGATGTCACGGAACgccccatcctcttccgcttcacccctgcagg |
| 72 | KRAS_3TS_3 | GTTGATGTCACGGAACgaaaacaatgtaattcctagtttccactaca |
| 73 | MALAT_3TS_3 | GTTGATGTCACGGAACacaatttacaaacagataagtttaaaataaa |
| 74 | NF2_3TS_3 | GTTGATGTCACGGAACagtaaagctatttttaaaaagctacacccag |
| 75 | PPARG_3TS_3 | GTTGATGTCACGGAACatttgtattgtttcagtgtaaaagcacagtg |
| 76 | PPIA_3TS_3 | GTTGATGTCACGGAACgaatagaagggttaaatagaaccgaaatggt |
| 77 | RPS5_3TS_3 | GTTGATGTCACGGAACacccataggcccactgagacaagaggtggtg |
| 78 | SMARCA1_3TS_3 | GTTGATGTCACGGAACaaatacaataaaatccatttatatggctggg |
| 79 | STAT3_3TS_3 | GTTGATGTCACGGAACcaaaacctcaaaaaagatacatgcaggacct |
| 80 | TERT_3TS_3 | GTTGATGTCACGGAACtctctctctgacccccaccactccagacccc |
| 81 | TUG1_3TS_3 | GTTGATGTCACGGAACcctgatggctgttaattcttgatgagcctgg |
| 82 | ANXA4_3TS_4 | GTTGATGTCACGGAACaagaaaagtgacagttgtttcctctgtttct |
| 83 | B4GALNT1_3TS_4 | GTTGATGTCACGGAACgtcaaggtctgcgctccggtgccttcggggg |
| 84 | KRAS_3TS_4 | GTTGATGTCACGGAACaacctgtccacaacttttgtcataaaatttg |
| 85 | MALAT_3TS_4 | GTTGATGTCACGGAACagacattcaagctgaactatcacaattctta |
| 86 | NF2_3TS_4 | GTTGATGTCACGGAACtttgccacttttataattatgcatcattttt |
| 87 | PPARG_3TS_4 | GTTGATGTCACGGAACgcaacagggcaagccaccatagtacaccttc |
| 88 | PPIA_3TS_4 | GTTGATGTCACGGAACaatctgcaaggttcaaactttaaacccaagt |
| 89 | RPS5_3TS_4 | GTTGATGTCACGGAACtggggccactcccaactgatgctgccagcca |
| 90 | SMARCA1_3TS_4 | GTTGATGTCACGGAACtacttatcttttactatttctgtgatatatg |
| 91 | STAT3_3TS_4 | GTTGATGTCACGGAACagaaaatataaagtttctgaggagaattcaa |
| 92 | TERT_3TS_4 | GTTGATGTCACGGAACcctctgccctcagggcctggcctggcggtgt |
| 93 | TUG1_3TS_4 | GTTGATGTCACGGAACcaggactctaagtgggtctgctgtcagcaca |
| 94 | ANXA4_3TS_5 | GTTGATGTCACGGAACagaagaaatgaaaagattacagataagaccc |
| 95 | B4GALNT1_3TS_5 | GTTGATGTCACGGAACttgcctccaggcgggcctgggataggggacc |
| 96 | KRAS_3TS_5 | GTTGATGTCACGGAACgttatagcacagtcattagtaacacaaatat |
| 97 | MALAT_3TS_5 | GTTGATGTCACGGAACgtttcccctccctcatcaacaaaagcccacc |
| 98 | NF2_3TS_5 | GTTGATGTCACGGAACaaaataaaaaacctacacatgaagtaaattt |
| 99 | PPARG_3TS_5 | GTTGATGTCACGGAACtgagaggataattatcccatgaaaacagtcc |
| 100 | PPIA_3TS_5 | GTTGATGTCACGGAACaaatgtcacttctgacaacataaccatgaag |
| 101 | RPS5_3TS_5 | GTTGATGTCACGGAACgtcaggctagaaggacagactgcggtcctcc |
| 102 | SMARCA1_3TS_5 | GTTGATGTCACGGAACgtattatatatacttcttttcagtacatgaa |
| 103 | STAT3_3TS_5 | GTTGATGTCACGGAACacaaaaaaacagaagtaaagaaagatttcct |
| 104 | TERT_3TS_5 | GTTGATGTCACGGAACaggagactgacagtggccacgcagaaactca |
| 105 | TUG1_3TS_5 | GTTGATGTCACGGAACaaaacaggtaaaataacaaatgcatggaatt |

Table 3 below shows trans-splicing cargo template sequences for human endogenous targets.

TABLE 3

| SEQ | | |
|---|---|---|
| ID | | |
| NO | ID | Sequence |
| 106 | ANXA4 | ctgtcaaagaaaaaaaaagaagaaatgaaaagattacagataagacccatgaaaaaagaaaagtgacag |
| | 3prime | ttgtttcctctgtttctacaaggtatcaatatttgctttgccagggaaattttagccaagcaatgtttcatgaaca |
| | cargo_1 | taggcaacgaggaattctcttcttttttttctgcagtaaaAtgcatgaggaacaaatctgcatattttgctgaa |
| | | aagctctataaatcgGACTACAAAGACGATGACGACAAGTAA |
| 107 | ANXA4 | gaaaaaagaaaagtgacagttgtttcctctgtttctacaaggtatcaatatttgctttgccagggaaattttagc |
| | 3prime | caagcaatgtttcatgaacataggcgatgctctatgtattttcacagatagaaagtaccctcaatttatcatca |
| | cargo_2 | aaacgaggaattctcttcttttttttctgcagtaaaAtgcatgaggaacaaatctgcatattttgctgaaaagc |
| | | tctataaatcgGACTACAAAGACGATGACGACAAGTAA |
| 108 | ANXA4 | tttgctttgccagggaaattttagccaagcaatgtttcatgaacataggcgatgctctatgtattttcacagata |
| | 3prime | gaaagtaccctcaatttatcatcaagttcctggagaagtaattgataaagtctatcaaagcaaagggtggta |
| | cargo_3 | tttaacgaggaattctcttcttttttttctgcagtaaaAtgcatgaggaacaaatctgcatattttgctgaaaag |
| | | ctctataaatcgGACTACAAAGACGATGACGACAAGTAA |
| 109 | B4GAL | ctgcagggagagggaggttgcctccaggcgggcctgggataggggacccgaaggggtcaaggtctgc |
| | NT1 | gctccggtgccttcgggggtacccctgccccatcctcttccgcttcacccctgcaggacccagacagttc |
| | 3prime | ccaggccccacttaacgaggaattctcttcttttttttctgcaggtCgggggcgcggtgcgcgagatctcc |
| | cargo_1 | ggctttgccaccacttatGACTACAAAGACGATGACGACAAGTAA |
| 110 | B4GAL | aaggggtcaaggtctgcgctccggtgccttcgggggtacccctgccccatcctcttccgcttcacccctgc |
| | NT1 | aggacccagacagttcccaggccccacttcgtggttctctctttgtcctagggctggccgttcctggccgc |
| | 3prime | agcgccccaacgaggaattctcttcttttttttctgcaggtCgggggcgcggtgcgcgagatctccggctt |
| | cargo_2 | tgccaccacttatGACTACAAAGACGATGACGACAAGTAA |
| 111 | B4GAL | cctcttccgcttcacccctgcaggacccagacagttcccaggccccacttcgtggttctctctttgtcctagg |
| | NT1 | gctggccgttcctggccgcagcgcccccgctgtggccttagcctgttcaaggcccttctccacccccaca |
| | 3prime | tcctgcaaacgaggaattctcttcttttttttctgcaggtCgggggcgcggtgcgcgagatctccggctttg |
| | cargo_3 | ccaccacttatGACTACAAAGACGATGACGACAAGTAA |
| 112 | KRAS | ctgggaaagaaaaaaaagttatagcacagtcattagtaacacaaatatctttcaaaacctgtccacaactttt |
| | 3prime | gtcataaaatttggctgaaagaaaacaatgtaattcctagtttccactacaccaaattttccttccttcttctact |
| | cargo_1 | aaacgaggaattctcttcttttttttctgcagagaGcaaattaaaagagttaaggactctgaagatgtaccta |
| | | tggtcctagtaggaaataaatgtGACTACAAAGACGATGACGACAAGTAA |
| 113 | KRAS | tttcaaaacctgtccacaacttttgtcataaaatttggctgaaagaaaacaatgtaattcctagtttccactaca |
| | 3prime | ccaaattttccttccttcttctactagttattttgtttctttacctttttaaacagaaaccttgtatctctctcaaacg |
| | cargo_2 | aggaattctcttcttttttttctgcagagaGcaaattaaaagagttaaggactctgaagatgtacctatggtc |
| | | ctagtaggaaataaatgtGACTACAAAGACGATGACGACAAGTAA |
| 114 | KRAS | aatgtaattcctagtttccactacaccaaattttccttccttcttctactagttattttgtttctttacctttttaaacag |
| | 3prime | aaaccttgtatctctctcaaaagatcaaatacctagaagtatagtaaaactataacctaataggttaataacg |
| | cargo_3 | aggaattctcttcttttttttctgcagagaGcaaattaaaagagttaaggactctgaagatgtacctatggtc |
| | | ctagtaggaaataaatgtGACTACAAAGACGATGACGACAAGTAA |
| 115 | MALAT | ctatagaaaaaaaaaaagtttcccctccctcatcaacaaaagcccaccctctaagagacattcaagctgaa |
| | 3prime | ctatcacaattcttaatcagttacaatttacaaacagataagtttaaaataaacaatttacaaaatttttgaagc |
| | cargo_1 | atacaacgaggaattctcttcttttttttctgcagacAtttttcagataacatcttctgagtcataaccagcctg |
| | | gcagtatgatggcctaGACTACAAAGACGATGACGACAAGTAA |
| 116 | MALAT | ctaagagacattcaagctgaactatcacaattcttaatcagttacaatttacaaacagataagtttaaaataaa |
| | 3prime | caatttacaaaatttttgaagcataccttaacatcttgttttgcagttaaacaatggaaaagtatttctcctacac |
| | cargo_2 | aacgaggaattctcttcttttttttctgcagacAtttttcagataacatcttctgagtcataaccagcctggca |
| | | gtatgatggcctaGACTACAAAGACGATGACGACAAGTAA |
| 117 | MALAT | acaatttacaaacagataagtttaaaataaacaatttacaaaatttttgaagcataccttaacatcttgttttgca |
| | 3prime | gttaaacaatggaaaagtatttctcctacactaaaaaaaaacttgcttacacacaactgaaaatagaatctta |
| | cargo_3 | caacgaggaattctcttcttttttttctgcagacAtttttcagataacatcttctgagtcataaccagcctggca |
| | | gtatgatggcctaGACTACAAAGACGATGACGACAAGTAA |
| 118 | NF2 | ctaccaaaaaatagagcaaaataaaaaacctacacatgaagtaaatttggtattgtttgccacttttataattat |
| | 3prime | gcatcatttttacaaaacagtaaagctatttttaaaaagctacacccagggagatagtcacacagagagag |
| | cargo_1 | ggcgaacgaggaattctcttcttttttttctgcaggtGataaatctgtatcagatgactccggaaatgtggg |
| | | aggagagaattactgcttggGACTACAAAGACGATGACGACAAGTAA |
| 119 | NF2 | tattgtttgccacttttataattatgcatcatttttacaaaacagtaaagctatttttaaaaagctacacccaggga |
| | 3prime | gatagtcacacagagagagggcgtgtaaataaggcaccattctattggaggagcaactcagaaagctg |
| | cargo_2 | catgaaacgaggaattctcttcttttttttctgcaggtGataaatctgtatcagatgactccggaaatgtggg |
| | | aggagagaattactgcttggGACTACAAAGACGATGACGACAAGTAA |
| 120 | NF2 | ctatttttaaaaagctacacccagggagatagtcacacagagagagggcgtgtaaataaggcaccattcta |
| | 3prime | ttggaggagcaactcagaaagctgcatgataactcagacctaggtgcaaccctcatctggcagtgcccct |
| | cargo_3 | gtccctgccaacgaggaattctcttcttttttttctgcaggtGataaatctgtatcagatgactccggaaatgt |
| | | gggaggagagaattactgcttggGACTACAAAGACGATGACGACAAGTAA |
| 121 | PPARG | ctgtgtatggagacatgtgagaggataattatcccatgaaaacagtcctaaaaaggcaacagggcaagcc |
| | 3prime | accatagtacaccttcatgctgtatttgtattgtttcagtgtaaaagcacagtggaacatgaaaaaacactgg |
| | cargo_1 | agttaagaacgaggaattctcttcttttttttctgcaggtgcTatcaaagtggagcctgcatctccaccttatt |
| | | attctgagaagactcagctctacaataagGACTACAAAGACGATGACGACAAGT |
| | | AA |
| 122 | PPARG | aaaaggcaacagggcaagccaccatagtacaccttcatgctgtatttgtattgtttcagtgtaaaagcacag |
| | 3prime | tggaacatgaaaaaacactggagttaaggcaagaaaaagaaaggtttgtaatcacaggcaagttataac |
| | cargo_2 | atctctaagaacgaggaattctcttcttttttttctgcaggtgcTatcaaagtggagcctgcatctccacctta |
| | | ttattctgagaagactcagctctacaataagGACTACAAAGACGATGACGACAAG |
| | | TAA |
| 123 | PPARG | ttgtttcagtgtaaaagcacagtggaacatgaaaaaacactggagttaaggcaagaaaaagaaaggtttgt |
| | 3prime | aatcacaggcaagttataacatctctaagcctcacctgtaaatataaaatgggaatgagaattaagtctgtg |
| | cargo_3 | gttctataacgaggaattctcttcttttttttctgcaggtgcTatcaaagtggagcctgcatctccaccttatta |
| | | ttctgagaagactcagctctacaataagGACTACAAAGACGATGACGACAAGT |
| | | AA |
| 124 | PPIA | ctgtcaacatataggaaaaatgtcacttctgacaacataaccatgaagtgtgccaaatctgcaaggttcaaa |
| | 3prime | ctttaaacccaagttcaaactgaatagaagggttaaatagaaccgaaatggtagagtaacagttcagatat |
| | cargo_1 | gtgtatcaacgaggaattctcttcttttttttctgcagggAggtgacttcacacgccataatggcactggtg |
| | | gcaagtccatctatggggagaaatttGACTACAAAGACGATGACGACAAGTAA |
| 125 | PPIA | tgccaaatctgcaaggttcaaactttaaacccaagttcaaactgaatagaagggttaaatagaaccgaaatg |
| | 3prime | gtagagtaacagttcagatatgtgtatcctgaaatattctggctcaatcccaaatgaagggagcaacccaa |
| | cargo_2 | ataaaataacgaggaattctcttcttttttttctgcagggAggtgacttcacacgccataatggcactggtg |
| | | gcaagtccatctatggggagaaatttGACTACAAAGACGATGACGACAAGTAA |
| 126 | PPIA | actttaaacccaagttcaaactgaatagaagggttaaatagaaccgaaatggtagagtaacagttcagatat |
| | 3prime | gtgtatcctgaaatattctggctcaatcccaaatgaagggagcaacccaaataaaataaaattcagtaaatt |
| | cargo_3 | tcgtacaacgaggaattctcttcttttttttctgcagggAggtgacttcacacgccataatggcactggtgg |
| | | caagtccatctatggggagaaatttGACTACAAAGACGATGACGACAAGTAA |
| 127 | RPS5 | ctaggaagacagcaggggtcaggctagaaggacagactgcggtcctccagcaccctggggccactcc |
| | 3prime | caactgatgctgccagccacgttgtcacccataggcccactgagacaagaggtggtggcatcatgcctg |
| | cargo_1 | atttgcaatcagataacgaggaattctcttcttttttttctgcaggcGatctggctgctgtgcacaggcgctc |
| | | gtgaggctgccttccggaacattaagaccGACTACAAAGACGATGACGACAAG |
| | | TAA |
| 128 | RPS5 | caccctggggccactcccaactgatgctgccagccacgttgtcacccataggcccactgagacaagagg |
| | 3prime | tggtggcatcatgcctgatttgcaatcagatagagggtcacaagagcaagtgtccagacacacacacac |
| | cargo_2 | acaggctgaagtaacgaggaattctcttcttttttttctgcaggcGatctggctgctgtgcacaggcgctcg |
| | | tgaggctgccttccggaacattaagaccGACTACAAAGACGATGACGACAAGT |
| | | AA |
| 129 | RPS5 | ggcccactgagacaagaggtggtggcatcatgcctgatttgcaatcagatagagggtcacaagagcaag |
| | 3prime | tgtccagacacacacacacacaggctgaagttgcgtccccagtgacaggagattgagacctgcctcaac |
| | cargo_3 | agcaaactgctaaacgaggaattctcttcttttttttctgcaggcGatctggctgctgtgcacaggcgctcg |
| | | tgaggctgccttccggaacattaagaccGACTACAAAGACGATGACGACAAGT |
| | | AA |
| 130 | SMARCA1 | ctatgagggaggaaaatgtattatatatacttcttttcagtacatgaagtaaacttacttatcttttactatttctgt |
| | 3prime | gatatatggaacaataaatacaataaaatccatttatatggctgggccaaatcagatacttttttgtactgtaa |
| | cargo_1 | cgaggaattctcttcttttttttctgcaggcAccacggcctccaaaacagccaaatgttcaggattttcaattt |
| | | ttcccaccacgcttaGACTACAAAGACGATGACGACAAGTAA |
| 131 | SMARCA1 | aaacttacttatcttttactatttctgtgatatatggaacaataaatacaataaaatccatttatatggctgggcca |
| | 3prime | aatcagatacttttttgtactgttatattttagagactatgatttggcaatgatacaagtaaatctgacccataac |
| | cargo 2 | gaggaattctcttcttttttttctgcaggcAccacggcctccaaaacagccaaatgttcaggattttcaatttt |
| | | tcccaccacgcttaGACTACAAAGACGATGACGACAAGTAA |
| 132 | SMARCA1 | ataaaatccatttatatggctgggccaaatcagatacttttttgtactgttatattttagagactatgatttggcaa |
| | 3prime | tgatacaagtaaatctgacccatagatcatgttgcaacagaaatctactttaggacataaaaactggccttg |
| | cargo_3 | taacgaggaattctcttcttttttttctgcaggcAccacggcctccaaaacagccaaatgttcaggattttca |
| | | atttttcccaccacgcttaGACTACAAAGACGATGACGACAAGTAA |
| 133 | STAT3 | ctgtttaaaataagcaaacaaaaaaacagaagtaaagaaagatttccttgggaacagaaaatataaagtttc |
| | 3prime | tgaggagaattcaaatgaagccaaaacctcaaaaaagatacatgcaggacctgcaggcagtatcccca |
| | cargo_1 | agagaaggctaacgaggaattctcttcttttttttctgcaggaCctagaacagaaaatgaaagtggtagag |
| | | aatctccaggatgactttgatttcaactatGACTACAAAGACGATGACGACAAGT |
| | | AA |
| 134 | STAT3 | ggaacagaaaatataaagtttctgaggagaattcaaatgaagccaaaacctcaaaaaagatacatgcagg |
| | 3prime | acctgcaggcagtatccccaagagaaggctccctgttggccaggtgcagtggctcacgcctgtaatgcc |
| | cargo_2 | agcactttgagaacgaggaattctcttcttttttttctgcaggaCctagaacagaaaatgaaagtggtagag |
| | | aatctccaggatgactttgatttcaactatGACTACAAAGACGATGACGACAAGT |
| | | AA |
| 135 | STAT3 | tcaaaaaagatacatgcaggacctgcaggcagtatccccaagagaaggctccctgttggccaggtgcag |
| | 3prime | tggctcacgcctgtaatgccagcactttgagaggctgagttgggaggatcacttgagcccaggagttcat |
| | cargo_3 | gatcagcctggaacgaggaattctcttcttttttttctgcaggaCctagaacagaaaatgaaagtggtaga |
| | | gaatctccaggatgactttgatttcaactatGACTACAAAGACGATGACGACAAGT |
| | | AA |
| 136 | TERT | ctgtgtgagtggaggcgaggagactgacagtggccacgcagaaactcagacatcacctctgccctcagg |
| | 3prime | gcctggcctggcggtgtcccccactctctctctgacccccaccactccagaccccaagggcagggcgg |
| | cargo_1 | gctgtgtcccctcaacgaggaattctcttcttttttttctgcaggtCgatgtgacgggcgcgtacgacacca |
| | | tcccccaggacaggctcacggaggtcatcgccGACTACAAAGACGATGACGACA |
| | | AGTAA |
| 137 | TERT | catcacctctgccctcagggcctggcctggcggtgtcccccactctctctctgacccccaccactccagac |
| | 3prime | cccaagggcaggggggctgtgtcccctctctgagcctcaggacaggagacccctggctcaggactg |
| | cargo_2 | gggtgcaaggcaaacgaggaattctcttcttttttttctgcaggtCgatgtgacgggcgcgtacgacacc |
| | | atcccccaggacaggctcacggaggtcatcgccGACTACAAAGACGATGACGAC |
| | | AAGTAA |
| 138 | TERT | ctgacccccaccactccagaccccaagggcaggggggctgtgtcccctctctgagcctcaggacagg |
| | 3prime | agacccctggctcaggactggggtgcaaggcaccggggcctggtggctgagccgttgcggttccttct |
| | cargo_3 | ctgacggaaactggaacgaggaattctcttcttttttttctgcaggtCgatgtgacgggcgcgtacgacac |
| | | catcccccaggacaggctcacggaggtcatcgccGACTACAAAGACGATGACGAC |
| | | AAGTAA |
| 139 | TUG1 | ctagaaggggcagggacaaaacaggtaaaataacaaatgcatggaattacaaacacaggactctaagtg |
| | 3prime | ggtctgctgtcagcacatcggcagcctgatggctgttaattcttgatgagcctggcttaggcaaagacaaa |
| | cargo_1 | tgataaatgaaacgaggaattctcttcttttttttctgcagtcctgtgcctcctgattgctgagtgttcacctgg |
| | | accttctgactaccttccctgtgctaGACTACAAAGACGATGACGACAAGTAA |
| 140 | TUG1 | aaacacaggactctaagtgggtctgctgtcagcacatcggcagcctgatggctgttaattcttgatgagcct |
| | 3prime | ggcttaggcaaagacaaatgataaatgaaaacaaacaacagcaatccaaaaaagaaaacacaaaagtc |
| | cargo_2 | tgattaacacaacgaggaattctcttcttttttttctgcagtcctgtgcctcctgattgctgagtgttcacctgg |
| | | accttctgactaccttccctgtgctaGACTACAAAGACGATGACGACAAGTAA |
| 141 | TUG1 | gctgttaattcttgatgagcctggcttaggcaaagacaaatgataaatgaaaacaaacaacagcaatccaa |
| | 3prime | aaaagaaaacacaaaagtctgattaacactctcgatttgtgggaactgtctcgcgaagcagcacacagaa |
| | cargo_3 | actaagcctaacgaggaattctcttcttttttttctgcagtcctgtgcctcctgattgctgagtgttcacctgga |
| | | ccttctgactaccttccctgtgctaGACTACAAAGACGATGACGACAAGTAA |

Table 4 below shows trans-splicing cargo template sequences for 5TS on COL7A1 intron48 (Gluc reporter).

TABLE 4

| SEQ | | |
|---|---|---|
| ID | | |
| NO | ID | Sequence |
| 142 | 5TS_1- | atgGGAGTCAAAGTTCTGTTTGCCCTGATCTGCATCGCTGTGG |
| | 76aa_int48_ | CCGAGGCCAAGCCCACCGAGAACAACGAAGACTTCAACAT |
| | 2_noBP | CGTGGCCGTGGCCAGCAACTTCGCGACCACGGATCTCGATG |
| | | CTGACCGCGGGAAGTTGCCCGGCAAGAAGCTGCCGCTGGA |
| | | GGTGCTCAAAGAGATGGAAGCCAATGCCCGGAAAGCTGGC |
| | | TGCACCAGGGGCTGTCTGATCTGCGTAAGCACGTTTGGGGT |
| | | TGAAAAAATCTATTGTACCCCAGGCTCCAGTTAACCCCCTG |
| | | ACCCAGCAAGTCCTGTGACCCCCCAAGTCCCATAGATAGGC |
| | | CCTATGACCTAGACCTC |
| 143 | 5TS_1- | atgGGAGTCAAAGTTCTGTTTGCCCTGATCTGCATCGCTGTGG |
| | 76aa_int48_ | CCGAGGCCAAGCCCACCGAGAACAACGAAGACTTCAACAT |
| | 2_BP | CGTGGCCGTGGCCAGCAACTTCGCGACCACGGATCTCGATG |
| | | CTGACCGCGGGAAGTTGCCCGGCAAGAAGCTGCCGCTGGA |
| | | GGTGCTCAAAGAGATGGAAGCCAATGCCCGGAAAGCTGGC |
| | | TGCACCAGGGGCTGTCTGATCTGCGTAAGCCCGCGGAACAT |
| | | TATTATAACGTTGCTCGAATACTAACACGTTTGGGGTTGAA |
| | | AAAATCTATTGTACCCCAGGCTCCAGTTAACCCCCTGACCC |
| | | AGCAAGTCCTGTGACCCCCCAAGTCCCATAGATAGGCCCTA |
| | | TGACCTAGACCTC |
| 144 | int48 1- | AACCCTGTAGAAACCTCCCCTTGCCCCATACCAGGCTTACA |
| | 40bp_5TS_ | ATCGGCTTCAACGTGCTCCACGGCTGGCGatgGGAGTCAAAG |
| | 1- | TTCTGTTTGCCCTGATCTGCATCGCTGTGGCCGAGGCCAAGC |
| | 76aa_int48_ | CCACCGAGAACAACGAAGACTTCAACATCGTGGCCGTGGCC |
| | 2_noBP | AGCAACTTCGCGACCACGGATCTCGATGCTGACCGCGGGAA |
| | | GTTGCCCGGCAAGAAGCTGCCGCTGGAGGTGCTCAAAGAG |
| | | ATGGAAGCCAATGCCCGGAAAGCTGGCTGCACCAGGGGCT |
| | | GTCTGATCTGCGTAAGCACGTTTGGGGTTGAAAAAATCTAT |
| | | TGTACCCCAGGCTCCAGTTAACCCCCTGACCCAGCAAGTCC |
| | | TGTGACCCCCCAAGTCCCATAGATAGGCCCTATGACCTAGA |
| | | CCTC |
| 145 | int48_1- | AACCCTGTAGAAACCTCCCCTTGCCCCATACCAGGCTTACA |
| | 40bp_5TS_ | ATCGGCTTCAACGTGCTCCACGGCTGGCGatgGGAGTCAAAG |
| | 1- | TTCTGTTTGCCCTGATCTGCATCGCTGTGGCCGAGGCCAAGC |
| | 76aa_int48_ | CCACCGAGAACAACGAAGACTTCAACATCGTGGCCGTGGCC |
| | 2_BP | AGCAACTTCGCGACCACGGATCTCGATGCTGACCGCGGGAA |
| | | GTTGCCCGGCAAGAAGCTGCCGCTGGAGGTGCTCAAAGAG |
| | | ATGGAAGCCAATGCCCGGAAAGCTGGCTGCACCAGGGGCT |
| | | GTCTGATCTGCGTAAGCCCGCGGAACATTATTATAACGTTG |
| | | CTCGAATACTAACACGTTTGGGGTTGAAAAAATCTATTGTA |
| | | CCCCAGGCTCCAGTTAACCCCCTGACCCAGCAAGTCCTGTG |
| | | ACCCCCCAAGTCCCATAGATAGGCCCTATGACCTAGACCTC |
| 146 | EF1a_1- | gaaataccagtgtgcagatcttggcccgcatttacaagactatcttgccagaaaaaaagcgtcgcag |
| | 80bp_5TS_ | caggtcatcaaaaAATCGGCTTCAACGTGCTCCACGGCTGGCGatgG |
| | 1- | GAGTCAAAGTTCTGTTTGCCCTGATCTGCATCGCTGTGGCCG |
| | 76aa_int48_ | AGGCCAAGCCCACCGAGAACAACGAAGACTTCAACATCGT |
| | 2_noBP | GGCCGTGGCCAGCAACTTCGCGACCACGGATCTCGATGCTG |
| | | ACCGCGGGAAGTTGCCCGGCAAGAAGCTGCCGCTGGAGGT |
| | | GCTCAAAGAGATGGAAGCCAATGCCCGGAAAGCTGGCTGC |
| | | ACCAGGGGCTGTCTGATCTGCGTAAGCACGTTTGGGGTTGA |
| | | AAAAATCTATTGTACCCCAGGCTCCAGTTAACCCCCTGACC |
| | | CAGCAAGTCCTGTGACCCCCCAAGTCCCATAGATAGGCCCT |
| | | ATGACCTAGACCTC |
| 147 | EF1a_1- | gaaataccagtgtgcagatcttggcccgcatttacaagactatcttgccagaaaaaaagcgtcgcag |
| | 80bp_5TS_ | caggtcatcaaaaAATCGGCTTCAACGTGCTCCACGGCTGGCGatgG |
| | 1- | GAGTCAAAGTTCTGTTTGCCCTGATCTGCATCGCTGTGGCCG |
| | 76aa_int48_ | AGGCCAAGCCCACCGAGAACAACGAAGACTTCAACATCGT |
| | 2_BP | GGCCGTGGCCAGCAACTTCGCGACCACGGATCTCGATGCTG |
| | | ACCGCGGGAAGTTGCCCGGCAAGAAGCTGCCGCTGGAGGT |
| | | GCTCAAAGAGATGGAAGCCAATGCCCGGAAAGCTGGCTGC |
| | | ACCAGGGGCTGTCTGATCTGCGTAAGCCCGCGGAACATTAT |
| | | TATAACGTTGCTCGAATACTAACACGTTTGGGGTTGAAAAA |
| | | ATCTATTGTACCCCAGGCTCCAGTTAACCCCCTGACCCAGC |
| | | AAGTCCTGTGACCCCCCAAGTCCCATAGATAGGCCCTATGA |
| | | CCTAGACCTC |
| 148 | EF1a_2- | cagggccgggaagcggccatctttccgctcacgcaactggtgccgaccgggccagccttgccgc |
| | 80bp_5TS_ | ccagggcggggcgataAATCGGCTTCAACGTGCTCCACGGCTGGCGa |
| | 1- | tgGGAGTCAAAGTTCTGTTTGCCCTGATCTGCATCGCTGTGG |
| | 76aa_int48_ | CCGAGGCCAAGCCCACCGAGAACAACGAAGACTTCAACAT |
| | 2_noBP | CGTGGCCGTGGCCAGCAACTTCGCGACCACGGATCTCGATG |
| | | CTGACCGCGGGAAGTTGCCCGGCAAGAAGCTGCCGCTGGA |
| | | GGTGCTCAAAGAGATGGAAGCCAATGCCCGGAAAGCTGGC |
| | | TGCACCAGGGGCTGTCTGATCTGCGTAAGCACGTTTGGGGT |
| | | TGAAAAAATCTATTGTACCCCAGGCTCCAGTTAACCCCCTG |
| | | ACCCAGCAAGTCCTGTGACCCCCCAAGTCCCATAGATAGGC |
| | | CCTATGACCTAGACCTC |
| 149 | EF1a_2- | cagggccgggaagcggccatctttccgctcacgcaactggtgccgaccgggccagccttgccgc |
| | 80bp_5TS_ | ccagggcggggcgataAATCGGCTTCAACGTGCTCCACGGCTGGCGa |
| | 1- | tgGGAGTCAAAGTTCTGTTTGCCCTGATCTGCATCGCTGTGG |
| | 76aa_int48_ | CCGAGGCCAAGCCCACCGAGAACAACGAAGACTTCAACAT |
| | 2_BP | CGTGGCCGTGGCCAGCAACTTCGCGACCACGGATCTCGATG |
| | | CTGACCGCGGGAAGTTGCCCGGCAAGAAGCTGCCGCTGGA |
| | | GGTGCTCAAAGAGATGGAAGCCAATGCCCGGAAAGCTGGC |
| | | TGCACCAGGGGCTGTCTGATCTGCGTAAGCCCGCGGAACAT |
| | | TATTATAACGTTGCTCGAATACTAACACGTTTGGGGTTGAA |
| | | AAAATCTATTGTACCCCAGGCTCCAGTTAACCCCCTGACCC |
| | | AGCAAGTCCTGTGACCCCCCAAGTCCCATAGATAGGCCCTA |
| | | TGACCTAGACCTC |
| 150 | EF1a_3- | tcacgacacctgaaatggaagaaaaaaactttgaaccactgtctgaggcttgagaatgaaccaagat |
| | 80bp_5TS_ | ccaaactcaaaaaAATCGGCTTCAACGTGCTCCACGGCTGGCGatgG |
| | 1- | GAGTCAAAGTTCTGTTTGCCCTGATCTGCATCGCTGTGGCCG |
| | 76aa_int48_ | AGGCCAAGCCCACCGAGAACAACGAAGACTTCAACATCGT |
| | 2_noBP | GGCCGTGGCCAGCAACTTCGCGACCACGGATCTCGATGCTG |
| | | ACCGCGGGAAGTTGCCCGGCAAGAAGCTGCCGCTGGAGGT |
| | | GCTCAAAGAGATGGAAGCCAATGCCCGGAAAGCTGGCTGC |
| | | ACCAGGGGCTGTCTGATCTGCGTAAGCACGTTTGGGGTTGA |
| | | AAAAATCTATTGTACCCCAGGCTCCAGTTAACCCCCTGACC |
| | | CAGCAAGTCCTGTGACCCCCCAAGTCCCATAGATAGGCCCT |
| | | ATGACCTAGACCTC |
| 151 | EF1a_3- | tcacgacacctgaaatggaagaaaaaaactttgaaccactgtctgaggcttgagaatgaaccaagat |
| | 80bp_5TS_ | ccaaactcaaaaaAATCGGCTTCAACGTGCTCCACGGCTGGCGatgG |
| | 1- | GAGTCAAAGTTCTGTTTGCCCTGATCTGCATCGCTGTGGCCG |
| | 76aa_int48_ | AGGCCAAGCCCACCGAGAACAACGAAGACTTCAACATCGT |
| | 2_BP | GGCCGTGGCCAGCAACTTCGCGACCACGGATCTCGATGCTG |
| | | ACCGCGGGAAGTTGCCCGGCAAGAAGCTGCCGCTGGAGGT |
| | | GCTCAAAGAGATGGAAGCCAATGCCCGGAAAGCTGGCTGC |
| | | ACCAGGGGCTGTCTGATCTGCGTAAGCCCGCGGAACATTAT |
| | | TATAACGTTGCTCGAATACTAACACGTTTGGGGTTGAAAAA |
| | | ATCTATTGTACCCCAGGCTCCAGTTAACCCCCTGACCCAGC |
| | | AAGTCCTGTGACCCCCCAAGTCCCATAGATAGGCCCTATGA |
| | | CCTAGACCTC |

Table 5 below shows trans-splicing cargo template sequences for 3′TS on COL7A1 intron46 (Gluc reporter).

TABLE 5

| SEQ | | |
|---|---|---|
| ID | | |
| NO | ID | Sequence |
| 152 | 3TS_ | TATCCCTATGATGTCCCCGATTATGCCGGTTCAAGAGCCCTGG |
| | intron46_ | TCGTGATTAGACTGAGCCGAGTGACAGACGCCACCACAACGA |
| | Gluc_ | GGAATTCTCTTCTTTTTTTTCTGCAGCTGTCCCACATCAAGTGC |
| | scrambled | ACGCCCAAGATGAAGAAGTTCATCCCAGGACGCTGCCACACC |
| | control | TACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 153 | 3TS_ | GTGAGTGACGGGAGGATGGCGCTCTGAGCACAGCACAGCCCT |
| | intron46_ | TGAGCAGTGACCCTCCTATAGAACACTATCTGGGCTGTAACGA |
| | Gluc_ | GGAATTCTCTTCTTTTTTTTCTGCAGCTGTCCCACATCAAGTGC |
| | cargo_1 | ACGCCCAAGATGAAGAAGTTCATCCCAGGACGCTGCCACACC |
| | | TACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 154 | 3TS_ | CTTGAGCAGTGACCCTCCTATAGAACACTATCTGGGCTGTGAT |
| | intron46_ | TCCACAGTGCTGGGCCCGTGAGCAGGCTGGGAGCTCTAACGA |
| | Gluc_ | GGAATTCTCTTCTTTTTTTTCTGCAGCTGTCCCACATCAAGTGC |
| | cargo_2 | ACGCCCAAGATGAAGAAGTTCATCCCAGGACGCTGCCACACC |
| | | TACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 155 | 3TS_ | GATTCCACAGTGCTGGGCCCGTGAGCAGGCTGGGAGCTCTGC |
| | intron46_ | GGCTCTCCTTCTGCTAGAACCTGCCCCCAGACTCTTGGAACGA |
| | Gluc_ | GGAATTCTCTTCTTTTTTTTCTGCAGCTGTCCCACATCAAGTGC |
| | cargo_3 | ACGCCCAAGATGAAGAAGTTCATCCCAGGACGCTGCCACACC |
| | | TACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 156 | 3TS_ | GCGGCTCTCCTTCTGCTAGAACCTGCCCCCAGACTCTTGGCTA |
| | intron46_ | TGATCCTGTGACCCCAAGACCGCCATGCAGGTCATGAAACGA |
| | G1uc_ | GGAATTCTCTTCTTTTTTTTCTGCAGCTGTCCCACATCAAGTGC |
| | cargo_4 | ACGCCCAAGATGAAGAAGTTCATCCCAGGACGCTGCCACACC |
| | | TACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 157 | 3TS_ | CTATGATCCTGTGACCCCAAGACCGCCATGCAGGTCATGAGCT |
| | intron46_ | CTTTGTGTCAGTCCATTTTGTATAACCCCTTCCCTGCAACGAGG |
| | Gluc_ | AATTCTCTTCTTTTTTTTCTGCAGCTGTCCCACATCAAGTGCAC |
| | cargo_5 | GCCCAAGATGAAGAAGTTCATCCCAGGACGCTGCCACACCTA |
| | | CGAAGGCGACAAAGAGTCCGCAcagGGCGGCATAGGCGAGGC |
| | | GATCGTCGACATTCCTGAGATTCCTGGGTTCAAGGACTTGGAG |
| | | CCCATGGAGCAGTTCATCGCACAGGTCGATCTGTGTGTGGACT |
| | | GCACAACTGGCTGCCTCAAAGGGCTTGCCAACGTGCAGTGTTC |
| | | TGACCTGCTCAAGAAGTGGCTGCCGCAACGCTGTGCGACCTTT |
| | | GCCAGCAAGATCCAGGGCCAGGTGGACAAGATCAAGGGGGCC |
| | | GGTGGTGACTAA |
| 158 | 3TS_ | TGTCAGCGGTGACTCTGTGACTTCTGGGCGGGGACTGAGCTGT |
| | intron46_ | ATGACTTCCAATTCCATGTGACCTCCATTCCAATGAAAACGAG |
| | Gluc_ | GAATTCTCTTCTTTTTTTTCTGCAGCTGTCCCACATCAAGTGCA |
| | cargo_6 | CGCCCAAGATGAAGAAGTTCATCCCAGGACGCTGCCACACCT |
| | | ACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 159 | 3TS_ | TGTATGACTTCCAATTCCATGTGACCTCCATTCCAATGAAGAC |
| | intron46_ | TTTGATCATACAACCCCAAGGCAGGGCCAAGCTGTATAACGA |
| | Gluc_ | GGAATTCTCTTCTTTTTTTTCTGCAGCTGTCCCACATCAAGTGC |
| | cargo_7 | ACGCCCAAGATGAAGAAGTTCATCCCAGGACGCTGCCACACC |
| | | TACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 160 | 3TS_ | GACTTTGATCATACAACCCCAAGGCAGGGCCAAGCTGTATCTG |
| | intron46_ | TCCTGTTTGTTTTCAGACaACGGATCTCGATGCTGACAACGAG |
| | Gluc_ | GAATTCTCTTCTTTTTTTTCTGCAGCTGTCCCACATCAAGTGCA |
| | cargo_8 | CGCCCAAGATGAAGAAGTTCATCCCAGGACGCTGCCACACCT |
| | | ACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 161 | 3TS_ | CAGGGCCAAGCTGTATCTGTCCTGTTTGTTTTCAGACaACGGAT |
| | intron46_ | CTCGATGCTGACCGCGGCAGTGGAGAGGGCAGAGGAAACGAG |
| | Gluc_ | GAATTCTCTTCTTTTTTTTCTGCAGCTGTCCCACATCAAGTGCA |
| | cargo_9 | CGCCCAAGATGAAGAAGTTCATCCCAGGACGCTGCCACACCT |
| | | ACGAAGGCGACAAAGAGTCCGCAcagGGCGGCATAGGCGAGG |
| | | CGATCGTCGACATTCCTGAGATTCCTGGGTTCAAGGACTTGGA |
| | | GCCCATGGAGCAGTTCATCGCACAGGTCGATCTGTGTGTGGAC |
| | | TGCACAACTGGCTGCCTCAAAGGGCTTGCCAACGTGCAGTGTT |
| | | CTGACCTGCTCAAGAAGTGGCTGCCGCAACGCTGTGCGACCTT |
| | | TGCCAGCAAGATCCAGGGCCAGGTGGACAAGATCAAGGGGGC |
| | | CGGTGGTGACTAA |
| 162 | 3TS_ | GGATCTCGATGCTGACCGCGGCAGTGGAGAGGGCAGAGGAAG |
| | intron46_ | TCTGCTAACATGCGGTGACGTCGAGGAGAATCCTGGCCAACG |
| | Gluc_ | AGGAATTCTCTTCTTTTTTTTCTGCAGCTGTCCCACATCAAGTG |
| | cargo_10 | CACGCCCAAGATGAAGAAGTTCATCCCAGGACGCTGCCACAC |
| | | CTACGAAGGCGACAAAGAGTCCGCAcagGGCGGCATAGGCGAG |
| | | GCGATCGTCGACATTCCTGAGATTCCTGGGTTCAAGGACTTGG |
| | | AGCCCATGGAGCAGTTCATCGCACAGGTCGATCTGTGTGTGGA |
| | | CTGCACAACTGGCTGCCTCAAAGGGCTTGCCAACGTGCAGTGT |
| | | TCTGACCTGCTCAAGAAGTGGCTGCCGCAACGCTGTGCGACCT |
| | | TTGCCAGCAAGATCCAGGGCCAGGTGGACAAGATCAAGGGGG |
| | | CCGGTGGTGACTAA |

Table 6 below shows cargo template sequences for internal trans-splicing on both COL7A1 intron46 and intron48 (Gluc reporter).

TABLE 6

| SEQ | | |
|---|---|---|
| ID | | |
| NO | ID | Sequence |
| 163 | internal 37-76 aa | CTGGGTGGGACGTGCTCCATTTATACCCTGCGCAGGCTG |
| | cargo, left | GACCGAGGACCGCAAGCTGCGACGGTGCACAAGTAATT |
| | scrambled right | GACAACGAGGAATTCTCTTCTTTTTTTTCTGCAGACCACG |
| | int48_2 | GATCTCGATGCTGACCGCGGGAAGTTGCCCGGCAAGAAG |
| | | CTGCCGCTGGAGGTGCTCAAAGAGATGGAAGCCAATGCC |
| | | CGGAAAGCTGGCTGCACCAGGGGCTGTCTGATCTGCGTA |
| | | AGCGGCGCGTAGGATCCAGGCTCCAGTTAACCCCCTGAC |
| | | CCAGCAAGTCCTGTGACCCCCCAAGTCCCATAGATAGGC |
| | | CCTATGACCTAGACCTC |
| 164 | internal 37-76 aa | CTGGGTGGGACGTGCTCCATTTATACCCTGCGCAGGCTG |
| | cargo, left | GACCGAGGACCGCAAGCTGCGACGGTGCACAAGTAATT |
| | scrambled right | GACAACGAGGAATTCTCTTCTTTTTTTTCTGCAGACCACG |
| | int48_3 | GATCTCGATGCTGACCGCGGGAAGTTGCCCGGCAAGAAG |
| | | CTGCCGCTGGAGGTGCTCAAAGAGATGGAAGCCAATGCC |
| | | CGGAAAGCTGGCTGCACCAGGGGCTGTCTGATCTGCGTA |
| | | AGCGGCGCGTAGGATTGGAGGTGACAAAGACCATCAGT |
| | | GCTAGTCCCAGGCTCCAGTTAACCCCCTGACCCAGCAAG |
| | | TCCTGTGACCCCCCAAGT |
| 165 | internal 37-76 aa | TCATGACCTGCATGGCGGTCTTGGGGTCACAGGATCATA |
| | cargo, left | GCCAAGAGTCTGGGGGCAGGTTCTAGCAGAAGGAGAGC |
| | int46_4 right | CGCAACGAGGAATTCTCTTCTTTTTTTTCTGCAGACCACG |
| | scframbled | GATCTCGATGCTGACCGCGGGAAGTTGCCCGGCAAGAAG |
| | | CTGCCGCTGGAGGTGCTCAAAGAGATGGAAGCCAATGCC |
| | | CGGAAAGCTGGCTGCACCAGGGGCTGTCTGATCTGCGTA |
| | | AGCGGCGCGTAGGATCTGGGTGGGACGTGCTCCATTTAT |
| | | ACCCTGCGCAGGCTGGACCGAGGACCGCAAGATGCGAC |
| | | GGTGCACAAGTAATTGAC |
| 166 | internal 37-76 aa | GCGGCTCTCCTTCTGCTAGAACCTGCCCCCAGACTCTTGG |
| | cargo, int46_4, | CTATGATCCTGTGACCCCAAGACCGCCATGCAGGTCATG |
| | int48_1 | AAACGAGGAATTCTCTTCTTTTTTTTCTGCAGACCACGGA |
| | | TCTCGATGCTGACCGCGGGAAGTTGCCCGGCAAGAAGCT |
| | | GCCGCTGGAGGTGCTCAAAGAGATGGAAGCCAATGCCC |
| | | GGAAAGCTGGCTGCACCAGGGGCTGTCTGATCTGCGTAA |
| | | GCGGCGCGTAGGATCCCCCCAAGTCCCATAGATAGGCCC |
| | | TATGACCTAGACCTCAACCCTGTAGAAACCTCCCCTTGC |
| | | CCCATACCAGGCTTAC |
| 167 | internal 37-76 aa | GCGGCTCTCCTTCTGCTAGAACCTGCCCCCAGACTCTTGG |
| | cargo, int46_4, | CTATGATCCTGTGACCCCAAGACCGCCATGCAGGTCATG |
| | int48_2 | AAACGAGGAATTCTCTTCTTTTTTTTCTGCAGACCACGGA |
| | | TCTCGATGCTGACCGCGGGAAGTTGCCCGGCAAGAAGCT |
| | | GCCGCTGGAGGTGCTCAAAGAGATGGAAGCCAATGCCC |
| | | GGAAAGCTGGCTGCACCAGGGGCTGTCTGATCTGCGTAA |
| | | GCGGCGCGTAGGATCCAGGCTCCAGTTAACCCCCTGACC |
| | | CAGCAAGTCCTGTGACCCCCCAAGTCCCATAGATAGGCC |
| | | CTATGACCTAGACCTC |
| 168 | internal 37-76 aa | GCGGCTCTCCTTCTGCTAGAACCTGCCCCCAGACTCTTGG |
| | cargo, int46_4, | CTATGATCCTGTGACCCCAAGACCGCCATGCAGGTCATG |
| | int48_3 | AAACGAGGAATTCTCTTCTTTTTTTTCTGCAGACCACGGA |
| | | TCTCGATGCTGACCGCGGGAAGTTGCCCGGCAAGAAGCT |
| | | GCCGCTGGAGGTGCTCAAAGAGATGGAAGCCAATGCCC |
| | | GGAAAGCTGGCTGCACCAGGGGCTGTCTGATCTGCGTAA |
| | | GCGGCGCGTAGGATTGGAGGTGACAAAGACCATCAGTG |
| | | CTAGTCCCAGGCTCCAGTTAACCCCCTGACCCAGCAAGT |
| | | CCTGTGACCCCCCAAGT |
| 169 | internal 37-76 aa | TGTCAGCGGTGACTCTGTGACTTCTGGGCGGGGACTGAG |
| | cargo, int46_4, | CTGTATGACTTCCAATTCCATGTGACCTCCATTCCAATGA |
| | int48_1 | AAACGAGGAATTCTCTTCTTTTTTTTCTGCAGACCACGGA |
| | | TCTCGATGCTGACCGCGGGAAGTTGCCCGGCAAGAAGCT |
| | | GCCGCTGGAGGTGCTCAAAGAGATGGAAGCCAATGCCC |
| | | GGAAAGCTGGCTGCACCAGGGGCTGTCTGATCTGCGTAA |
| | | GCGGCGCGTAGGATCCCCCCAAGTCCCATAGATAGGCCC |
| | | TATGACCTAGACCTCAACCCTGTAGAAACCTCCCCTTGC |
| | | CCCATACCAGGCTTAC |
| 170 | internal 37-76 aa | TGTCAGCGGTGACTCTGTGACTTCTGGGCGGGGACTGAG |
| | cargo, int46_4, | CTGTATGACTTCCAATTCCATGTGACCTCCATTCCAATGA |
| | int48_2 | AAACGAGGAATTCTCTTCTTTTTTTTCTGCAGACCACGGA |
| | | TCTCGATGCTGACCGCGGGAAGTTGCCCGGCAAGAAGCT |
| | | GCCGCTGGAGGTGCTCAAAGAGATGGAAGCCAATGCCC |
| | | GGAAAGCTGGCTGCACCAGGGGCTGTCTGATCTGCGTAA |
| | | GCGGCGCGTAGGATCCAGGCTCCAGTTAACCCCCTGACC |
| | | CAGCAAGTCCTGTGACCCCCCAAGTCCCATAGATAGGCC |
| | | CTATGACCTAGACCTC |
| 171 | internal 37-76 aa | TGTCAGCGGTGACTCTGTGACTTCTGGGCGGGGACTGAG |
| | cargo, int46_4, | CTGTATGACTTCCAATTCCATGTGACCTCCATTCCAATGA |
| | int48_3 | AAACGAGGAATTCTCTTCTTTTTTTTCTGCAGACCACGGA |
| | | TCTCGATGCTGACCGCGGGAAGTTGCCCGGCAAGAAGCT |
| | | GCCGCTGGAGGTGCTCAAAGAGATGGAAGCCAATGCCC |
| | | GGAAAGCTGGCTGCACCAGGGGCTGTCTGATCTGCGTAA |
| | | GCGGCGCGTAGGATTGGAGGTGACAAAGACCATCAGTG |
| | | CTAGTCCCAGGCTCCAGTTAACCCCCTGACCCAGCAAGT |
| | | CCTGTGACCCCCCAAGT |

Table 7 below shows the sequences of splicing proteins for fusion.

TABLE 7

| SEQ | | |
|---|---|---|
| ID | | |
| NO | ID | Sequence |
| 179 | RBM17 | MSLYDDLGVETSDSKTEGWSKNFKLLQSQLQVKKAALTQAKSQRTKQSTV |
| | | LAPVIDLKRGGSSDDRQIVDTPPHVAAGLKDPVPSGFSAGEVLIPLADEYDP |
| | | MFPNDYEKVVKRQREERQRQRELERQKEIEEREKRRKDRHEASGFARRPDP |
| | | DSDEDEDYERERRKRSMGGAAIAPPTSLVEKDKELPRDFPYEEDSRPRSQSS |
| | | KAAIPPPVYEEQDRPRSPTGPSNSFLANMGGTVAHKIMQKYGFREGQGLGK |
| | | HEQGLSTALSVEKTSKRGGKIIVGDATEKDASKKSDSNPLTEILKCPTKVVLL |
| | | RNMVGAGEVDEDLEVETKEECEKYGKVGKCVIFEIPGAPDDEAVRIFLEFER |
| | | VESAIKAVVDLNGRYFGGRVVKACFYNLDKFRVLDLAEQV |
| 180 | SF3B6 | MAMQAAKRANIRLPPEVNRILYIRNLPYKITAEEMYDIFGKYGPIRQIRVGNT |
| | | PETRGTAYVVYEDIFDAKNACDHLSGFNVCNRYLVVLYYNANRAFQKMDT |
| | | KKKEEQLKLLKEKYGINTDPPK |
| 181 | U2AF1 | MAEYLASIFGTEKDKVNCSFYFKIGACRHGDRCSRLHNKPTFSQTIALLNIYR |
| | | NPQNSSQSADGLRCAVSDVEMQEHYDEFFEEVFTEMEEKYGEVEEMNVCD |
| | | NLGDHLVGNVYVKFRREEDAEKAVIDLNNRWFNGQPIHAELSPVTDFREAC |
| | | CRQYEMGECTRGGFCNFMHLKPISRELRRELYGRRRKKHRSRSRSRERRSRS |
| | | RDRGRGGGGGGGGGGGGRERDRRRSRDRERSGRF |
| 182 | U2AF2 | MSDFDEFERQLNENKQERDKENRHRKRSHSRSRSRDRKRRSRSRDRRNRDQ |
| | | RSASRDRRRRSKPLTRGAKEEHGGLIRSPRHEKKKKVRKYWDVPPPGFEHIT |
| | | PMQYKAMQAAGQIPATALLPTMTPDGLAVTPTPVPVVGSQMTRQARRLYV |
| | | GNIPFGITEEAMMDFFNAQMRLGGLTQAPGNPVLAVQINQDKNFAFLEFRS |
| | | VDETTQAMAFDGIIFQGQSLKIRRPHDYQPLPGMSENPSVYVPGVVSTVVPD |
| | | SAHKLFIGGLPNYLNDDQVKELLTSFGPLKAFNLVKDSATGLSKGYAFCEY |
| | | VDINVTDQAIAGLNGMQLGDKKLLVQRASVGAKNATLVSPPSTINQTPVTL |
| | | QVPGLMSSQVQMGGHPTEVLCLMNMVLPEELLDDEEYEEIVEDVRDECSK |
| | | YGLVKSIEIPRPVDGVEVPGCGKIFVEFTSVFDCQKAMQGLTGRKFANRVVV |
| | | TKYCDPDSYHRRDFW |
| 183 | SF1 | MATGANATPLDFPSKKRKRSRWNQDTMEQKTVIPGMPTVIPPGLTREQERA |
| | | YIVQLQIEDLTRKLRTGDLGIPPNPEDRSPSPEPIYNSEGKRLNTREFRTRKKL |
| | | EEERHNLITEMVALNPDFKPPADYKPPATRVSDKVMIPQDEYPEINFVGLLIG |
| | | PRGNTLKNIEKECNAKIMIRGKGSVKEGKVGRKDGQMLPGEDEPLHALVTA |
| | | NTMENVKKAVEQIRNILKQGIETPEDQNDLRKMQLRELARLNGTLREDDNR |
| | | ILRPWQSSETRSITNTTVCTKCGGAGHIASDCKFQRPGDPQSAQDKARMDKE |
| | | YLSLMAELGEAPVPASVGSTSGPATTPLASAPRPAAPANNPPPPSLMSTTQSR |
| | | PPWMNSGPSESRPYHGMHGGGPGGPGGGPHSFPHPLPSLTGGHGGHPMQH |
| | | NPNGPPPPWMQPPPPPMNQGPHPPGHHGPPPMDQYLGSTPVGSGVYRLHQG |
| | | KGMMPPPPMGMMPPPPPPPSGQPPPPPSGPLPPWQQQQQQPPPPPPPSSSMAS |
| | | STPLPWQQNTTTTTTSAGTGSIPPWQQQQAAAAASPGAPQMQGNPTMVPLP |
| | | PGVQPPLPPGAPPPPPPPPPGSAGMMYAPPPPPPPPMDPSNFVTMMGMGVAG |
| | | MPPFGMPPAPPPPPPQN |
| 184 | SF3B1 | MAKIAKTHEDIEAQIREIQGKKAALDEAQGVGLDSTGYYDQEIYGGSDSRFA |
| | | GYVTSIAATELEDDDDDYSSSTSLLGQKKPGYHAPVALLNDIPQSTEQYDPF |
| | | AEHRPPKIADREDEYKKHRRTMIISPERLDPFADGGKTPDPKMNARTYMDV |
| | | MREQHLTKEEREIRQQLAEKAKAGELKVVNGAAASQPPSKRKRRWDQTAD |
| | | QTPGATPKKLSSWDQAETPGHTPSLRWDETPGRAKGSETPGATPGSKIWDP |
| | | TPSHTPAGAATPGRGDTPGHATPGHGGATSSARKNRWDETPKTERDTPGHG |
| | | SGWAETPRTDRGGDSIGETPTPGASKRKSRWDETPASQMGGSTPVLTPGKTP |
| | | IGTPAMNMATPTPGHIMSMTPEQLQAWRWEREIDERNRPLSDEELDAMFPE |
| | | GYKVLPPPAGYVPIRTPARKLTATPTPLGGMTGFHMQTEDRTMKSVNDQPS |
| | | GNLPFLKPDDIQYFDKLLVDVDESTLSPEEQKERKIMKLLLKIKNGTPPMRK |
| | | AALRQITDKAREFGAGPLFNQILPLLMSPTLEDQERHLLVKVIDRILYKLDDL |
| | | VRPYVHKILVVIEPLLIDEDYYARVEGREIISNLAKAAGLATMISTMRPDIDN |
| | | MDEYVRNTTARAFAVVASALGIPSLLPFLKAVCKSKKSWQARHTGIKIVQQI |
| | | AILMGCAILPHLRSLVEIIEHGLVDEQQKVRTISALAIAALAEAATPYGIESFD |
| | | SVLKPLWKGIRQHRGKGLAAFLKAIGYLIPLMDAEYANYYTREVMLILIREF |
| | | QSPDEEMKKIVLKVVKQCCGTDGVEANYIKTEILPPFFKHFWQHRMALDRR |
| | | NYRQLVDTTVELANKVGAAEIISRIVDDLKDEAEQYRKMVMETIEKIMGNL |
| | | GAADIDHKLEEQLIDGILYAFQEQTTEDSVMLNGFGTVVNALGKRVKPYLP |
| | | QICGTVLWRLNNKSAKVRQQAADLISRTAVVMKTCQEEKLMGHLGVVLYE |
| | | YLGEEYPEVLGSILGALKAIVNVIGMHKMTPPIKDLLPRLTPILKNRHEKVQE |
| | | NCIDLVGRIADRGAEYVSAREWMRICFELLELLKAHKKAIRRATVNTFGYIA |
| | | KAIGPHDVLATLLNNLKVQERQNRVCTTVAIAIVAETCSPFTVLPALMNEYR |
| | | VPELNVQNGVLKSLSFLFEYIGEMGKDYIYAVTPLLEDALMDRDLVHRQTA |
| | | SAVVQHMSLGVYGFGCEDSLNHLLNYVWPNVFETSPHVIQAVMGALEGLR |
| | | VAIGPCRMLQYCLQGLFHPARKVRDVYWKIYNSIYIGSQDALIAHYPRIYND |
| | | DKNTYIRYELDYIL |

Table 8 shows the codon optimized DNA sequences for the splicing proteins from Table 7.

TABLE 8

| SEQ | | |
|---|---|---|
| ID | | |
| NO | ID | Sequence |
| 185 | RBM17 | atgtccctgtacgatgacctaggagtggagaccagtgactcaaaaacagaaggctggtccaa |
| | | aaacttcaaacttctgcagtctcagcttcaggtgaagaaggcagctctcactcaggcaaagag |
| | | ccaaaggacgaaacaaagtacagtcctcgccccagtcattgacctgaagcgaggtggctcct |
| | | cagatgaccggcaaattgtggacactccaccgcatgtagcagctgggctgaaggatcctgttc |
| | | ccagtgggttttctgcaggggaagttctgattcccttagctgacgaatatgaccctatgtttccta |
| | | atgattatgagaaagtagtgaagcgccaaagagaggaacgacagagacagcgggagctgg |
| | | aaagacaaaaggaaatagaagaaagggaaaaaaggcgtaaagacagacatgaagcaagt |
| | | gggtttgcaaggagaccagatccagattctgatgaagatgaagattatgagcgagagaggag |
| | | gaaaagaagtatgggcggagctgccattgccccacccacttctctggtagagaaagacaaag |
| | | agttaccccgagattttccttatgaagaggactcaagacctcgatcacagtcttccaaagcagc |
| | | cattcctcccccagtgtacgaggaacaagacagaccgagatctccaaccggacctagcaact |
| | | ccttcctcgctaacatggggggcacggtggcgcacaagatcatgcagaagtacggcttccgg |
| | | gagggccagggtctggggaagcatgagcagggcctgagcactgccttgtcagtggagaag |
| | | accagcaagcgtggcggcaagatcatcgtgggcgacgccacagagaaagatgcatccaag |
| | | aagtcagattcaaatccgctgactgaaatacttaagtgtcctactaaagtggtcttactaaggaa |
| | | catggttggtgcgggagaggtggatgaagacttggaagttgaaaccaaggaagaatgtgaa |
| | | aaatatggcaaagttggaaaatgtgtgatatttgaaattcctggtgcccctgatgatgaagcagt |
| | | acggatatttttagaatttgagagagttgaatcagcaattaaagcggttgttgacttgaatggga |
| | | ggtattttggtggacgggtggtaaaagcatgtttctacaatttggacaaattcagggtcttggatt |
| | | tggcagaacaagtt |
| 186 | SF3B6 | atggcgatgcaagcggccaagagggcgaacattcgacttccacctgaagtaaatcggatatt |
| | | gtatataagaaatttgccatacaaaatcacagctgaagaaatgtatgatatatttgggaaatatg |
| | | gacctattcgtcaaatcagagtggggaacacacctgaaactagaggaacagcttatgtggtct |
| | | atgaggacatctttgatgccaagaatgcatgtgatcacctatcgggattcaatgtttgtaacagat |
| | | accttgtggttttgtactataatgccaacagggcatttcagaagatggacacaaagaagaagga |
| | | ggaacagttgaagcttctcaaggagaaatatggcatcaacacagatccaccaaaa |
| 187 | U2AF1 | atggcggagtatctggcctccatcttcggcaccgagaaagacaaagtcaactgttcattttattt |
| | | caaaattggagcatgtcgtcatggagacaggtgctctcggttgcacaataaaccgacgtttag |
| | | ccagaccattgccctcttgaacatttaccgtaaccctcaaaactcttcccagtctgctgacggttt |
| | | gcgctgtgccgtgagcgatgtggagatgcaggaacactatgatgagttttttgaggaggttttta |
| | | cagaaatggaggagaagtatggggaagtagaggagatgaacgtctgtgacaacctgggag |
| | | accacctggtggggaacgtgtacgtcaagtttcgccgtgaggaagatgcggaaaaggctgtg |
| | | attgacttgaataaccgttggtttaatggacagccgCtccacgccgagctgtcacccgtgacg |
| | | gacttcagagaagcctgctgccgtcagtatgagatgggagaatgcacacgaggcggcttctg |
| | | caacttcatgcatttgaagcccatttccagagagctgcggcgggagctgtatggccgccgtcg |
| | | caagaagcatagatcaagatcccgatcccgggagcgtcgttctcggtctagagaccgtggtc |
| | | gtggcggtggcggtggcggtggtggaggtggcggcggacgggagcgtgacaggaggcg |
| | | gtcgagagatcgtgaaagatctgggcgattc |
| 188 | U2AF2 | atgtcggacttcgacgagttcgagcggcagctcaacgagaataaacaagagcgggacaag |
| | | gagaaccggcatcggaagcgcagccacagccgctctcggagccgggaccgcaaacgccg |
| | | gagccggagccgcgaccggcgcaaccgggaccagcggagcgcctcccgggacaggcg |
| | | acgacgcagcaaacctttgaccagaggcgctaaagaggagcacggtggactgattcgttcc |
| | | ccccgccacgagaagaagaagaaggtccgtaaatactgggacgtgccacccccaggctttg |
| | | agcacatcaccccaatgcagtacaaggccatgcaagctgcgggtcagattccagccactgct |
| | | cttctccccaccatgacccctgacggtctggctgtgaccccaacgccggtgcccgtggtcgg |
| | | gagccagatgaccagacaagcccggcgcctctacgtgggcaacatcccctttggcatcactg |
| | | aggaggccatgatggatttcttcaacgcccagatgcgcctgggggggctgacccaggcccc |
| | | tggcaacccagtgttggctgtgcagattaaccaggacaagaattttgcctttttggagttccgct |
| | | cagtggacgagactacccaggctatggcctttgatggcatcatcttccagggccagtcactaa |
| | | agatccgcaggcctcacgactaccagccgcttcctggcatgtcagagaacccctccgtctatg |
| | | tgcctggggttgtgtccactgtggtccccgactctgcccacaagctgttcatcgggggcttacc |
| | | caactacctgaacgatgaccaggtcaaagagctgctgacatcctttgggcccctcaaggcctt |
| | | caacctggtcaaggacagtgccacggggctctccaagggctacgccttctgtgagtacgtgg |
| | | acatcaacgtcacggatcaggccattgcggggctgaacggcatgcagctgggggataagaa |
| | | gctgctggtccagagggcgagtgtgggagccaagaatgccacgctggtgagccccccgag |
| | | caccatcaatcagacgcctgtgaccctgcaagtgccgggcttgatgagctcccaggtgcaga |
| | | tgggcggccacccgactgaggtcctgtgcctcatgaacatggtgctgcctgaggagctgctg |
| | | gacgacgaggagtatgaggagatcgtggaggacgtgcgggacgagtgcagcaagtacgg |
| | | gcttgtcaagtccatcgagatcccccggcctgtggacggcgtcgaggtgcccggctgcgga |
| | | aagatctttgtggagttcacctctgtgtttgactgccagaaagccatgcagggcctgacgggcc |
| | | gcaagttcgccaacagagtggttgtcacaaaatactgtgaccccgactcttatcaccgccggg |
| | | acttctgg |
| 189 | SF1 | atggccaccggcgccaacgccacccccctggacttccccagcaagaagagaaagagaagc |
| | | agatggaaccaggacaccatggagcagaagaccgtgatccccggcatgcccaccgtgatc |
| | | ccccccggcctgaccagagagcaggagagagcctacatcgtgcagctgcagatcgaggac |
| | | ctgaccagaaagctgagaaccggcgacctgggcatcccccccaaccccgaggacagaag |
| | | ccccagccccgagcccatctacaacagcgagggcaagagactgaacaccagagagttcag |
| | | aaccagaaagaagctggaggaggagagacacaacctgatcaccgagatggtggccctgaa |
| | | ccccgacttcaagccccccgccgactacaagccccccgccaccagagtgagcgacaaggt |
| | | gatgatcccccaggacgagtaccccgagatcaacttcgtgggcctgctgatcggccccaga |
| | | ggcaacaccctgaagaacatcgagaaggagtgcaacgccaagatcatgatcagaggcaag |
| | | ggcagcgtgaaggagggcaaggtgggcagaaaggacggccagatgctgcccggcgagg |
| | | acgagcccctgcacgccctggtgaccgccaacaccatggagaacgtgaagaaggccgtgg |
| | | agcagatcagaaacatcctgaagcagggcatcgagacccccgaggaccagaacgacctga |
| | | gaaagatgcagctgagagagctggccagactgaacggcaccctgagagaggacgacaac |
| | | agaatcctgagaccctggcagagcagcgagaccagaagcatcaccaacaccaccgtgtgc |
| | | accaagtgcggcggcgccggccacatcgccagcgactgcaagttccagagacccggcga |
| | | cccccagagcgcccaggacaaggccagaatggacaaggagtacctgagcctgatggccg |
| | | agctgggcgaggcccccgtgcccgccagcgtgggcagcaccagcggccccgccaccacc |
| | | cccctggccagcgcccccagacccgccgcccccgccaacaacccccccccccccagcct |
| | | gatgagcaccacccagagcagacccccctggatgaacagcggccccagcgagagcagac |
| | | cctaccacggcatgcacggcggcggccccggcggccccggcggcggcccccacagcttc |
| | | ccccaccccctgcccagcctgaccggcggccacggcggccaccccatgcagcacaaccc |
| | | caacggccccccccccccctggatgcagcccccccccccccccatgaaccagggccccca |
| | | cccccccggccaccacggccccccccccatggaccagtacctgggcagcacccccgtggg |
| | | cagcggcgtgtacagactgcaccagggcaagggcatgatgccccccccccccatgggcat |
| | | gatgcccccccccccccccccccccagcggccagcccccccccccccccagcggccccct |
| | | gcccccctggcagcagcagcagcagcagcccccccccccccccccccccagcagcagca |
| | | tggccagcagcacccccctgccctggcagcagaacaccaccaccaccaccaccagcgccg |
| | | gcaccggcagcatccccccctggcagcagcagcaggccgccgccgccgccagccccgg |
| | | cgccccccagatgcagggcaaccccaccatggtgcccctgccccccggcgtgcagccccc |
| | | cctgccccccggcgcccccccccccccccccccccccccccccggcagcgccggcatgat |
| | | gtacgccccccccccccccccccccccccccatggaccccagcaacttcgtgaccatgatg |
| | | ggcatgggcgtggccggcatgccccccttcggcatgccccccgccccccccccccccccc |
| | | ccccagaac |
| 190 | SF3B1 | atggcgaagatcgccaagactcacgaagatattgaagcacagattcgagaaattcaaggcaa |
| | | gaaggcagctcttgatgaagctcaaggagtgggcctcgattctacaggttattatgaccagga |
| | | aatttatggtggaagtgacagcagatttgctggatacgtgacatcaattgctgcaactgaacttg |
| | | aagatgatgacgatgactattcatcatctacgagtttgcttggtcagaagaagccaggatatcat |
| | | gcccctgtggcattgcttaatgatataccacagtcaacagaacagtatgatccatttgctgagca |
| | | cagacctccaaagattgcagaccgggaagatgaatacaaaaagcataggcggaccatgata |
| | | atttccccagagcgtcttgatccttttgcagatggagggaaaacccctgatcctaaaatgaatgc |
| | | taggacttacatggatgtaatgcgagaacaacacttgactaaagaagaacgagaaattaggca |
| | | acagctagcagaaaaagctaaagctggagaactaaaagtcgtcaatggagcagcagcgtcc |
| | | cagcctccatcaaaacgaaaacggcgttgggatcaaacagctgatcagactcctggtgccac |
| | | tcccaaaaaactatcaagttgggatcaggcagagacccctgggcatactccttccttaagatg |
| | | ggatgagacaccaggtcgtgcaaagggaagcgagactcctggagcaaccccaggctcaaa |
| | | aatatgggatcctacacctagccacacaccagcgggagctgctactcctggacgaggtgata |
| | | caccaggccatgcgacaccaggccatggaggcgcaacttccagtgctcgtaaaaacagatg |
| | | ggatgaaacccccaaaacagagagagatactcctgggcatggaagtggatgggctgagact |
| | | cctcgaacagatcgaggtggagattctattggtgaaacaccgactcctggagccagtaaaag |
| | | aaaatcacggtgggatgaaacaccagctagtcagatgggtggaagcactccagttctgaccc |
| | | ctggaaagacaccaattggcacaccagccatgaacatggctacccctactccaggtcacata |
| | | atgagtatgactcctgaacagcttcaggcttggcggtgggaaagagaaattgatgagagaaat |
| | | cgcccactttctgatgaggaattagatgctatgttcccagaaggatataaggtacttcctcctcc |
| | | agctggttatgttcctattcgaactccagctcgaaagctgacagctactccaacacctttgggtg |
| | | gtatgactggtttccacatgcaaactgaagatcgaactatgaaaagtgttaatgaccagccatct |
| | | ggaaatcttccatttttaaaacctgatgatattcaatactttgataaactattggttgatgttgatgaa |
| | | tcaacacttagtccagaagagcaaaaagagagaaaaataatgaagttgcttttaaaaattaaga |
| | | atggaacaccaccaatgagaaaggctgcattgcgtcagattactgataaagctcgtgaatttgg |
| | | agctggtcctttgtttaatcagattcttcctctgctgatgtctcctacacttgaggatcaagagcgt |
| | | catttacttgtgaaagttattgataggatactgtacaaacttgatgacttagttcgtccatatgtgca |
| | | taagatcctcgtggtcattgaaccgctattgattgatgaagattactatgctagagtggaaggcc |
| | | gagagatcatttctaatttggcaaaggctgctggtctggctactatgatctctaccatgagacct |
| | | gatatagataacatggatgagtatgtccgtaacacaacagctagagcttttgctgttgtagcctct |
| | | gccctgggcattccttctttattgcccttcttaaaagctgtgtgcaaaagcaagaagtcctggca |
| | | agcgagacacactggtattaagattgtacaacagatagctattcttatgggctgtgccatcttgc |
| | | cacatcttagaagtttagttgaaatcattgaacatggtcttgtggatgagcagcagaaagttcgg |
| | | accatcagtgctttggccattgctgccttggctgaagcagcaactccttatggtatcgaatctttt |
| | | gattctgtgttaaagcctttatggaagggtatccgccaacacagaggaaagggtttggctgcttt |
| | | cttgaaggctattgggtatcttattcctcttatggatgcagaatatgccaactactatactagagaa |
| | | gtgatgttaatccttattcgagaattccagtctcctgatgaggaaatgaaaaaaattgtgctgaag |
| | | gtggtaaaacagtgttgtgggacagatggtgtagaagcaaactacattaaaacagagattcttc |
| | | ctcccttttttaaacacttctggcagcacaggatggctttggatagaagaaattaccgacagtta |
| | | gttgatactactgtggagttggcaaacaaagtaggtgcagcagaaattatatccaggattgtgg |
| | | atgatctgaaagatgaagccgaacagtacagaaaaatggtgatggagacaattgagaaaatt |
| | | atgggtaatttgggagcagcagatattgatcataaacttgaagaacaactgattgatggtattctt |
| | | tatgctttccaagaacagactacagaggactcagtaatgttgaacggctttggcacagtggtta |
| | | atgctcttggcaaacgagtcaaaccatacttgcctcagatctgtggtacagttttgtggcgtttaa |
| | | ataacaaatctgctaaagttaggcaacaggcagctgacttgatttctcgaactgctgttgtcatg |
| | | aagacttgtcaagaggaaaaattgatgggacacttgggtgttgtattgtatgagtatttgggtga |
| | | agagtaccctgaagtattgggcagcattcttggagcactgaaggccattgtaaatgtcataggt |
| | | atgcataagatgactccaccaattaaagatctgctgcctagactcacccccatcttaaagaaca |
| | | gacatgaaaaagtacaagagaattgtattgatcttgttggtcgtattgctgacaggggagctga |
| | | atatgtatctgcaagagagtggatgaggatttgctttgagcttttagagctcttaaaagcccaca |
| | | aaaaggctattcgtagagccacagtcaacacatttggttatattgcaaaggccattggccctcat |
| | | gatgtattggctacacttctgaacaacctcaaagttcaagaaaggcagaacagagtttgtacca |
| | | ctgtagcaatagctattgttgcagaaacatgttcaccctttacagtactccctgccttaatgaatg |
| | | aatacagagttcctgaactgaatgttcaaaatggagtgttaaaatcgctttccttcttgtttgaatat |
| | | attggtgaaatgggaaaagactacatttatgccgtaacaccgttacttgaagatgctttaatggat |
| | | agagaccttgtacacagacagacggctagtgcagtggtacagcacatgtcacttggggtttat |
| | | ggatttggttgtgaagattcgctgaatcacttgttgaactatgtatggcccaatgtatttgagacat |
| | | ctcctcatgtaattcaggcagttatgggagccctagagggcctgagagttgctattggaccatg |
| | | tagaatgttgcaatattgtttacagggtctgtttcacccagcccggaaagtcagagatgtatattg |
| | | gaaaatttacaactccatctacattggttcccaggacgctctcatagcacattacccaagaatct |
| | | acaacgatgataagaacacctatattcgttatgaacttgactatatctta |

Table 9 shows single, double, triple, and quadruple Cas7-11 mutations.

TABLE 9

| | | Mutation | Mutation | Mutation | Mutation |
|---|---|---|---|---|---|
| ID | Mutants | #1 | #2 | #3 | #4 |
| pDF0948 | Cas711_D1580R | D1580R | — | — | — |
| | (pDF0506 based) Singlemutant | | | | |
| pDF0949 | Cas711_D1580R_D988K | D1580R | D988K | — | — |
| | (pDF0506 based) Doublemutant | | | | |
| pDF0950 | Cas711_D1580R_D988K_D981K | D1580R | D988K | D981K | — |
| | (pDF0506 based) Triplemutant | | | | |
| pDF0951 | Cas711_D1580R_D988K_D981K_Y312K | D1580R | D988K | D981K | Y312K |
| | (pDF0506 based) Quadruplemutant | | | | |
| pDF0989 | Cas711_D1580R_D988K_Y312K | D1580R | D988K | — | Y312K |
| | (pDF0506 based) new_triplemutant | | | | |
| pDF0995 | Cas711_S1006-GGGS- | D1580R | D988K | — | Y312K |
| | R1294_D1580R_D988K_Y312K | | | | |

Table 10 shows the amino acid sequences of Cas7-11 mutants from Table 9.

TABLE 10

| SEQ | | |
|---|---|---|
| ID | | |
| NO | ID | Sequence |
| 191 | Cas711_D1580R | MKRTADGSEFESPKKKRKVTTTMKISIEFLEPFRMTKWQES |
| | (pDF0506 based) | TRRNKNNKEFVRGQAFARWHRNKKDNTKGRPYITGTLLR |
| | Singlemutant | SAVIRSAENLLTLSDGKISEKTCCPGKFDTEDKDRLLQLRQ |
| | | RSTLRWTDKNPCPDNAETYCPFCELLGRSGNDGKKAEKKD |
| | | WRFRIHFGNLSLPGKPDFDGPKAIGSQRVLNRVDFKSGKA |
| | | HDFFKAYEVDHTRFPRFEGEITIDNKVSAEARKLLCDSLKF |
| | | TDRLCGALCVIRFDEYTPAADSGKQTENVQAEPNANLAEK |
| | | TAEQIISILDDNKKTEYTRLLADAIRSLRRSSKLVAGLPKDH |
| | | DGKDDHYLWDIGKKKKDENSVTIRQILTTSADTKELKNAG |
| | | KWREFCEKLGEALYLKSKDMSGGLKITRRILGDAEFHGKP |
| | | DRLEKSRSVSIGSVLKETVVCGELVAKTPFFFGAIDEDAKQ |
| | | TDLQVLLTPDNKYRLPRSAVRGILRRDLQTYFDSPCNAELG |
| | | GRPCMCKTCRIMRGITVMDARSEYNAPPEIRHRTRINPFTG |
| | | TVAEGALFNMEVAPEGIVFPFQLRYRGSEDGLPDALKTVL |
| | | KWWAEGQAFMSGAASTGKGRFRMENAKYETLDLSDENQ |
| | | RNDYLKNWGWRDEKGLEELKKRLNSGLPEPGNYRDPKW |
| | | HEINVSIEMASPFINGDPIRAAVDKRGTDVVTFVKYKAEGE |
| | | EAKPVCAYKAESFRGVIRSAVARIHMEDGVPLTELTHSDCE |
| | | CLLCQIFGSEYEAGKIRFEDLVFESDPEPVTFDHVAIDRFTG |
| | | GAADKKKFDDSPLPGSPARPLMLKGSFWIRRDVLEDEEYC |
| | | KALGKALADVNNGLYPLGGKSAIGYGQVKSLGIKGDDKRI |
| | | SRLMNPAFDETDVAVPEKPKTDAEVRIEAEKVYYPHYFVE |
| | | PHKKVEREEKPCGHQKFHEGRLTGKIRCKLITKTPLIVPDTS |
| | | NDDFFRPADKEARKEKDEYHKSYAFFRLHKQIMIPGSELRG |
| | | MVSSVYETVTNSCFRIFDETKRLSWRMDADHQNVLQDFLP |
| | | GRVTADGKHIQKFSETARVPFYDKTQKHFDILDEQEIAGEK |
| | | PVRMWVKRFIKRLSLVDPAKHPQKKQDNKWKRRKEGIAT |
| | | FIEQKNGSYYFNVVTNNGCTSFHLWHKPDNFDQEKLEGIQ |
| | | NGEKLDCWVRDSRYQKAFQEIPENDPDGWECKEGYLHVV |
| | | GPSKVEFSDKKGDVINNFQGTLPSVPNDWKTIRTNDFKNR |
| | | KRKNEPVFCCEDDKGNYYTMAKYCETFFFDLKENEEYEIP |
| | | EKARIKYKELLRVYNNNPQAVPESVFQSRVARENVEKLKS |
| | | GDLVYFKHNEKYVEDIVPVRISRTVDDRMIGKRMSADLRP |
| | | CHGDWVEDGDLSALNAYPEKRLLLRHPKGLCPACRLFGT |
| | | GSYKGRVRFGFASLENDPEWLIPGKNPGDPFHGGPVMLSL |
| | | LERPRPTWSIPGSDNKFKVPGRKFYVHHHAWKTIKDGNHP |
| | | TTGKAIEQSPNNRTVEALAGGNSFSFEIAFENLKEWELGLLI |
| | | HSLQLEKGLAHKLGMAKSMGFGSVEIDVESVRLRKDWKQ |
| | | WRNGNSEIPNWLGKGFAKLKEWFRDELDFIENLKKLLWFP |
| | | EGDQAPRVCYPMLRKKDDPNGNSGYEELKRGEFKKEDRQ |
| | | KKLTTPWTPWAKRTADGSEFESPKKKRKV* |
| 192 | Cas711_D1580R_ | MKRTADGSEFESPKKKRKVTTTMKISIEFLEPFRMTKWQES |
| | D988K | TRRNKNNKEFVRGQAFARWHRNKKDNTKGRPYITGTLLR |
| | (pDF0506 based) | SAVIRSAENLLTLSDGKISEKTCCPGKFDTEDKDRLLQLRQ |
| | Doublemutant | RSTLRWTDKNPCPDNAETYCPFCELLGRSGNDGKKAEKKD |
| | | WRFRIHFGNLSLPGKPDFDGPKAIGSQRVLNRVDFKSGKA |
| | | HDFFKAYEVDHTRFPRFEGEITIDNKVSAEARKLLCDSLKF |
| | | TDRLCGALCVIRFDEYTPAADSGKQTENVQAEPNANLAEK |
| | | TAEQIISILDDNKKTEYTRLLADAIRSLRRSSKLVAGLPKDH |
| | | DGKDDHYLWDIGKKKKDENSVTIRQILTTSADTKELKNAG |
| | | KWREFCEKLGEALYLKSKDMSGGLKITRRILGDAEFHGKP |
| | | DRLEKSRSVSIGSVLKETVVCGELVAKTPFFFGAIDEDAKQ |
| | | TDLQVLLTPDNKYRLPRSAVRGILRRDLQTYFDSPCNAELG |
| | | GRPCMCKTCRIMRGITVMDARSEYNAPPEIRHRTRINPFTG |
| | | TVAEGALFNMEVAPEGIVFPFQLRYRGSEDGLPDALKTVL |
| | | KWWAEGQAFMSGAASTGKGRFRMENAKYETLDLSDENQ |
| | | RNDYLKNWGWRDEKGLEELKKRLNSGLPEPGNYRDPKW |
| | | HEINVSIEMASPFINGDPIRAAVDKRGTDVVTFVKYKAEGE |
| | | EAKPVCAYKAESFRGVIRSAVARIHMEDGVPLTELTHSDCE |
| | | CLLCQIFGSEYEAGKIRFEDLVFESDPEPVTFDHVAIDRFTG |
| | | GAADKKKFDDSPLPGSPARPLMLKGSFWIRRDVLEDEEYC |
| | | KALGKALADVNNGLYPLGGKSAIGYGQVKSLGIKGDDKRI |
| | | SRLMNPAFDETDVAVPEKPKTDAEVRIEAEKVYYPHYFVE |
| | | PHKKVEREEKPCGHQKFHEGRLTGKIRCKLITKTPLIVPDTS |
| | | NDDFFRPADKEARKEKDEYHKSYAFFRLHKQIMIPGSELRG |
| | | MVSSVYETVTNSCFRIFDETKRLSWRMDADHQNVLQKFLP |
| | | GRVTADGKHIQKFSETARVPFYDKTQKHFDILDEQEIAGEK |
| | | PVRMWVKRFIKRLSLVDPAKHPQKKQDNKWKRRKEGIAT |
| | | FIEQKNGSYYFNVVTNNGCTSFHLWHKPDNFDQEKLEGIQ |
| | | NGEKLDCWVRDSRYQKAFQEIPENDPDGWECKEGYLHVV |
| | | GPSKVEFSDKKGDVINNFQGTLPSVPNDWKTIRTNDFKNR |
| | | KRKNEPVFCCEDDKGNYYTMAKYCETFFFDLKENEEYEIP |
| | | EKARIKYKELLRVYNNNPQAVPESVFQSRVARENVEKLKS |
| | | GDLVYFKHNEKYVEDIVPVRISRTVDDRMIGKRMSADLRP |
| | | CHGDWVEDGDLSALNAYPEKRLLLRHPKGLCPACRLFGT |
| | | GSYKGRVRFGFASLENDPEWLIPGKNPGDPFHGGPVMLSL |
| | | LERPRPTWSIPGSDNKFKVPGRKFYVHHHAWKTIKDGNHP |
| | | TTGKAIEQSPNNRTVEALAGGNSFSFEIAFENLKEWELGLLI |
| | | HSLQLEKGLAHKLGMAKSMGFGSVEIDVESVRLRKDWKQ |
| | | WRNGNSEIPNWLGKGFAKLKEWFRDELDFIENLKKLLWFP |
| | | EGDQAPRVCYPMLRKKDDPNGNSGYEELKRGEFKKEDRQ |
| | | KKLTTPWTPWAKRTADGSEFESPKKKRKV* |
| 193 | Cas711_D1580R_ | MKRTADGSEFESPKKKRKVTTTMKISIEFLEPFRMTKWQES |
| | D988K_D981K | TRRNKNNKEFVRGQAFARWHRNKKDNTKGRPYITGTLLR |
| | (pDF0506 based) | SAVIRSAENLLTLSDGKISEKTCCPGKFDTEDKDRLLQLRQ |
| | Triplemutant | RSTLRWTDKNPCPDNAETYCPFCELLGRSGNDGKKAEKKD |
| | | WRFRIHFGNLSLPGKPDFDGPKAIGSQRVLNRVDFKSGKA |
| | | HDFFKAYEVDHTRFPRFEGEITIDNKVSAEARKLLCDSLKF |
| | | TDRLCGALCVIRFDEYTPAADSGKQTENVQAEPNANLAEK |
| | | TAEQIISILDDNKKTEYTRLLADAIRSLRRSSKLVAGLPKDH |
| | | DGKDDHYLWDIGKKKKDENSVTIRQILTTSADTKELKNAG |
| | | KWREFCEKLGEALYLKSKDMSGGLKITRRILGDAEFHGKP |
| | | DRLEKSRSVSIGSVLKETVVCGELVAKTPFFFGAIDEDAKQ |
| | | TDLQVLLTPDNKYRLPRSAVRGILRRDLQTYFDSPCNAELG |
| | | GRPCMCKTCRIMRGITVMDARSEYNAPPEIRHRTRINPFTG |
| | | TVAEGALFNMEVAPEGIVFPFQLRYRGSEDGLPDALKTVL |
| | | KWWAEGQAFMSGAASTGKGRFRMENAKYETLDLSDENQ |
| | | RNDYLKNWGWRDEKGLEELKKRLNSGLPEPGNYRDPKW |
| | | HEINVSIEMASPFINGDPIRAAVDKRGTDVVTFVKYKAEGE |
| | | EAKPVCAYKAESFRGVIRSAVARIHMEDGVPLTELTHSDCE |
| | | CLLCQIFGSEYEAGKIRFEDLVFESDPEPVTFDHVAIDRFTG |
| | | GAADKKKFDDSPLPGSPARPLMLKGSFWIRRDVLEDEEYC |
| | | KALGKALADVNNGLYPLGGKSAIGYGQVKSLGIKGDDKRI |
| | | SRLMNPAFDETDVAVPEKPKTDAEVRIEAEKVYYPHYFVE |
| | | PHKKVEREEKPCGHQKFHEGRLTGKIRCKLITKTPLIVPDTS |
| | | NDDFFRPADKEARKEKDEYHKSYAFFRLHKQIMIPGSELRG |
| | | MVSSVYETVTNSCFRIFDETKRLSWRMDAKHQNVLQKFLP |
| | | GRVTADGKHIQKFSETARVPFYDKTQKHFDILDEQEIAGEK |
| | | PVRMWVKRFIKRLSLVDPAKHPQKKQDNKWKRRKEGIAT |
| | | FIEQKNGSYYFNVVTNNGCTSFHLWHKPDNFDQEKLEGIQ |
| | | NGEKLDCWVRDSRYQKAFQEIPENDPDGWECKEGYLHVV |
| | | GPSKVEFSDKKGDVINNFQGTLPSVPNDWKTIRTNDFKNR |
| | | KRKNEPVFCCEDDKGNYYTMAKYCETFFFDLKENEEYEIP |
| | | EKARIKYKELLRVYNNNPQAVPESVFQSRVARENVEKLKS |
| | | GDLVYFKHNEKYVEDIVPVRISRTVDDRMIGKRMSADLRP |
| | | CHGDWVEDGDLSALNAYPEKRLLLRHPKGLCPACRLFGT |
| | | GSYKGRVRFGFASLENDPEWLIPGKNPGDPFHGGPVMLSL |
| | | LERPRPTWSIPGSDNKFKVPGRKFYVHHHAWKTIKDGNHP |
| | | TTGKAIEQSPNNRTVEALAGGNSFSFEIAFENLKEWELGLLI |
| | | HSLQLEKGLAHKLGMAKSMGFGSVEIDVESVRLRKDWKQ |
| | | WRNGNSEIPNWLGKGFAKLKEWFRDELDFIENLKKLLWFP |
| | | EGDQAPRVCYPMLRKKDDPNGNSGYEELKRGEFKKEDRQ |
| | | KKLTTPWTPWAKRTADGSEFESPKKKRKV* |
| 194 | Cas711_D1580R | MKRTADGSEFESPKKKRKVTTTMKISIEFLEPFRMTKWQES |
| | (pDF0506 based) | TRRNKNNKEFVRGQAFARWHRNKKDNTKGRPYITGTLLR |
| | Singlemutant | SAVIRSAENLLTLSDGKISEKTCCPGKFDTEDKDRLLQLRQ |
| | | RSTLRWTDKNPCPDNAETYCPFCELLGRSGNDGKKAEKKD |
| | | WRFRIHFGNLSLPGKPDFDGPKAIGSQRVLNRVDFKSGKA |
| | | HDFFKAYEVDHTRFPRFEGEITIDNKVSAEARKLLCDSLKF |
| | | TDRLCGALCVIRFDEYTPAADSGKQTENVQAEPNANLAEK |
| | | TAEQIISILDDNKKTEYTRLLADAIRSLRRSSKLVAGLPKDH |
| | | DGKDDHKLWDIGKKKKDENSVTIRQILTTSADTKELKNAG |
| | | KWREFCEKLGEALYLKSKDMSGGLKITRRILGDAEFHGKP |
| | | DRLEKSRSVSIGSVLKETVVCGELVAKTPFFFGAIDEDAKQ |
| | | TDLQVLLTPDNKYRLPRSAVRGILRRDLQTYFDSPCNAELG |
| | | GRPCMCKTCRIMRGITVMDARSEYNAPPEIRHRTRINPFTG |
| | | TVAEGALFNMEVAPEGIVFPFQLRYRGSEDGLPDALKTVL |
| | | KWWAEGQAFMSGAASTGKGRFRMENAKYETLDLSDENQ |
| | | RNDYLKNWGWRDEKGLEELKKRLNSGLPEPGNYRDPKW |
| | | HEINVSIEMASPFINGDPIRAAVDKRGTDVVTFVKYKAEGE |
| | | EAKPVCAYKAESFRGVIRSAVARIHMEDGVPLTELTHSDCE |
| | | CLLCQIFGSEYEAGKIRFEDLVFESDPEPVTFDHVAIDRFTG |
| | | GAADKKKFDDSPLPGSPARPLMLKGSFWIRRDVLEDEEYC |
| | | KALGKALADVNNGLYPLGGKSAIGYGQVKSLGIKGDDKRI |
| | | SRLMNPAFDETDVAVPEKPKTDAEVRIEAEKVYYPHYFVE |
| | | PHKKVEREEKPCGHQKFHEGRLTGKIRCKLITKTPLIVPDTS |
| | | NDDFFRPADKEARKEKDEYHKSYAFFRLHKQIMIPGSELRG |
| | | MVSSVYETVTNSCFRIFDETKRLSWRMDAKHQNVLQKFLP |
| | | GRVTADGKHIQKFSETARVPFYDKTQKHFDILDEQEIAGEK |
| | | PVRMWVKRFIKRLSLVDPAKHPQKKQDNKWKRRKEGIAT |
| | | FIEQKNGSYYFNVVTNNGCTSFHLWHKPDNFDQEKLEGIQ |
| | | NGEKLDCWVRDSRYQKAFQEIPENDPDGWECKEGYLHVV |
| | | GPSKVEFSDKKGDVINNFQGTLPSVPNDWKTIRTNDFKNR |
| | | KRKNEPVFCCEDDKGNYYTMAKYCETFFFDLKENEEYEIP |
| | | EKARIKYKELLRVYNNNPQAVPESVFQSRVARENVEKLKS |
| | | GDLVYFKHNEKYVEDIVPVRISRTVDDRMIGKRMSADLRP |
| | | CHGDWVEDGDLSALNAYPEKRLLLRHPKGLCPACRLFGT |
| | | GSYKGRVRFGFASLENDPEWLIPGKNPGDPFHGGPVMLSL |
| | | LERPRPTWSIPGSDNKFKVPGRKFYVHHHAWKTIKDGNHP |
| | | TTGKAIEQSPNNRTVEALAGGNSFSFEIAFENLKEWELGLLI |
| | | HSLQLEKGLAHKLGMAKSMGFGSVEIDVESVRLRKDWKQ |
| | | WRNGNSEIPNWLGKGFAKLKEWFRDELDFIENLKKLLWFP |
| | | EGDQAPRVCYPMLRKKDDPNGNSGYEELKRGEFKKEDRQ |
| | | KKLTTPWTPWAKRTADGSEFESPKKKRKV* |
| 195 | Cas711_D1580R_ | MKRTADGSEFESPKKKRKVTTTMKISIEFLEPFRMTKWQES |
| | D988K | TRRNKNNKEFVRGQAFARWHRNKKDNTKGRPYITGTLLR |
| | (pDF0506 based) | SAVIRSAENLLTLSDGKISEKTCCPGKFDTEDKDRLLQLRQ |
| | Doublemutant | RSTLRWTDKNPCPDNAETYCPFCELLGRSGNDGKKAEKKD |
| | | WRFRIHFGNLSLPGKPDFDGPKAIGSQRVLNRVDFKSGKA |
| | | HDFFKAYEVDHTRFPRFEGEITIDNKVSAEARKLLCDSLKF |
| | | TDRLCGALCVIRFDEYTPAADSGKQTENVQAEPNANLAEK |
| | | TAEQIISILDDNKKTEYTRLLADAIRSLRRSSKLVAGLPKDH |
| | | DGKDDHKLWDIGKKKKDENSVTIRQILTTSADTKELKNAG |
| | | KWREFCEKLGEALYLKSKDMSGGLKITRRILGDAEFHGKP |
| | | DRLEKSRSVSIGSVLKETVVCGELVAKTPFFFGAIDEDAKQ |
| | | TDLQVLLTPDNKYRLPRSAVRGILRRDLQTYFDSPCNAELG |
| | | GRPCMCKTCRIMRGITVMDARSEYNAPPEIRHRTRINPFTG |
| | | TVAEGALFNMEVAPEGIVFPFQLRYRGSEDGLPDALKTVL |
| | | KWWAEGQAFMSGAASTGKGRFRMENAKYETLDLSDENQ |
| | | RNDYLKNWGWRDEKGLEELKKRLNSGLPEPGNYRDPKW |
| | | HEINVSIEMASPFINGDPIRAAVDKRGTDVVTFVKYKAEGE |
| | | EAKPVCAYKAESFRGVIRSAVARIHMEDGVPLTELTHSDCE |
| | | CLLCQIFGSEYEAGKIRFEDLVFESDPEPVTFDHVAIDRFTG |
| | | GAADKKKFDDSPLPGSPARPLMLKGSFWIRRDVLEDEEYC |
| | | KALGKALADVNNGLYPLGGKSAIGYGQVKSLGIKGDDKRI |
| | | SRLMNPAFDETDVAVPEKPKTDAEVRIEAEKVYYPHYFVE |
| | | PHKKVEREEKPCGHQKFHEGRLTGKIRCKLITKTPLIVPDTS |
| | | NDDFFRPADKEARKEKDEYHKSYAFFRLHKQIMIPGSELRG |
| | | MVSSVYETVTNSCFRIFDETKRLSWRMDADHQNVLQKFLP |
| | | GRVTADGKHIQKFSETARVPFYDKTQKHFDILDEQEIAGEK |
| | | PVRMWVKRFIKRLSLVDPAKHPQKKQDNKWKRRKEGIAT |
| | | FIEQKNGSYYFNVVTNNGCTSFHLWHKPDNFDQEKLEGIQ |
| | | NGEKLDCWVRDSRYQKAFQEIPENDPDGWECKEGYLHVV |
| | | GPSKVEFSDKKGDVINNFQGTLPSVPNDWKTIRTNDFKNR |
| | | KRKNEPVFCCEDDKGNYYTMAKYCETFFFDLKENEEYEIP |
| | | EKARIKYKELLRVYNNNPQAVPESVFQSRVARENVEKLKS |
| | | GDLVYFKHNEKYVEDIVPVRISRTVDDRMIGKRMSADLRP |
| | | CHGDWVEDGDLSALNAYPEKRLLLRHPKGLCPACRLFGT |
| | | GSYKGRVRFGFASLENDPEWLIPGKNPGDPFHGGPVMLSL |
| | | LERPRPTWSIPGSDNKFKVPGRKFYVHHHAWKTIKDGNHP |
| | | TTGKAIEQSPNNRTVEALAGGNSFSFEIAFENLKEWELGLLI |
| | | HSLQLEKGLAHKLGMAKSMGFGSVEIDVESVRLRKDWKQ |
| | | WRNGNSEIPNWLGKGFAKLKEWFRDELDFIENLKKLLWFP |
| | | EGDQAPRVCYPMLRKKDDPNGNSGYEELKRGEFKKEDRQ |
| | | KKLTTPWTPWAKRTADGSEFESPKKKRKV* |
| 196 | Cas711_D1580R_ | MKRTADGSEFESPKKKRKVTTTMKISIEFLEPFRMTKWQES |
| | D988K_D981K | TRRNKNNKEFVRGQAFARWHRNKKDNTKGRPYITGTLLR |
| | (pDF0506 based) | SAVIRSAENLLTLSDGKISEKTCCPGKFDTEDKDRLLQLRQ |
| | Triplemutant | RSTLRWTDKNPCPDNAETYCPFCELLGRSGNDGKKAEKKD |
| | | WRFRIHFGNLSLPGKPDFDGPKAIGSQRVLNRVDFKSGKA |
| | | HDFFKAYEVDHTRFPRFEGEITIDNKVSAEARKLLCDSLKF |
| | | TDRLCGALCVIRFDEYTPAADSGKQTENVQAEPNANLAEK |
| | | TAEQIISILDDNKKTEYTRLLADAIRSLRRSSKLVAGLPKDH |
| | | DGKDDHKLWDIGKKKKDENSVTIRQILTTSADTKELKNAG |
| | | KWREFCEKLGEALYLKSKDMSGGLKITRRILGDAEFHGKP |
| | | DRLEKSRSVSIGSVLKETVVCGELVAKTPFFFGAIDEDAKQ |
| | | TDLQVLLTPDNKYRLPRSAVRGILRRDLQTYFDSPCNAELG |
| | | GRPCMCKTCRIMRGITVMDARSEYNAPPEIRHRTRINPFTG |
| | | TVAEGALFNMEVAPEGIVFPFQLRYRGSEDGLPDALKTVL |
| | | KWWAEGQAFMSGAASTGKGRFRMENAKYETLDLSDENQ |
| | | RNDYLKNWGWRDEKGLEELKKRLNSGLPEPGNYRDPKW |
| | | HEINVSIEMASPFINGDPIRAAVDKRGTDVVTFVKYKAEGE |
| | | EAKPVCAYKAESFRGVIRSAVARIHMEDGVPLTELTHSDCE |
| | | CLLCQIFGSEYEAGKIRFEDLVFESDPEPVTFDHVAIDRFTG |
| | | GAADKKKFDDSPLPGSPARPLMLKGSFWIRRDVLEDEEYC |
| | | KALGKALADVNNGLYPLGGKSAIGYGQVKSLGIKGDDKRI |
| | | SRLMNPAFDETDVAVPEKPKTDAEVRIEAEKVYYPHYFVE |
| | | PHKKVEREEKPCGHQKFHEGRLTGKIRCKLITKTPLIVPDTS |
| | | NDDFFRPADKEARKEKDEYHKSYAFFRLHKQIMIPGSELRG |
| | | MVSSVYETVTNSCFRIFDETKRLSWRMDADHQNVLQKFLP |
| | | GRVTADGKHIQKFSGGGSRTVDDRMIGKRMSADLRPCHG |
| | | DWVEDGDLSALNAYPEKRLLLRHPKGLCPACRLFGTGSYK |
| | | GRVRFGFASLENDPEWLIPGKNPGDPFHGGPVMLSLLERPR |
| | | PTWSIPGSDNKFKVPGRKFYVHHHAWKTIKDGNHPTTGKA |
| | | IEQSPNNRTVEALAGGNSFSFEIAFENLKEWELGLLIHSLQL |
| | | EKGLAHKLGMAKSMGFGSVEIDVESVRLRKDWKQWRNG |
| | | NSEIPNWLGKGFAKLKEWFRDELDFIENLKKLLWFPEGDQ |
| | | APRVCYPMLRKKDDPNGNSGYEELKRGEFKKEDRQKKLT |
| | | TPWTPWAKRTADGSEFESPKKKRKV* |

Table 11 shows the DNA sequences of the Cas7-11 mutants from Table 9.

TABLE 11

| SEQ | | |
|---|---|---|
| ID | | |
| NO | ID | Sequence |
| 197 | Cas711_D1580R | ATGaaacggacagccgacggaagcgagttcgagtcaccaaagaagaagcggaaagtc |
| | (pDF0506 based) | ACGACTACTATGAAGATTTCAATTGAATTCCTCGAGCCG |
| | Singlemutant | TTTCGGATGACCAAATGGCAGGAAAGCACTAGGCGAAA |
| | | CAAGAACAACAAGGAATTTGTTCGCGGCCAAGCTTTTGC |
| | | AAGATGGCATAGAAATAAGAAGGATAACACAAAAGGGC |
| | | GGCCTTACATAACTGGGACATTGCTGCGGTCCGCAGTAA |
| | | TTAGAAGCGCGGAAAACCTGCTGACATTGAGTGATGGG |
| | | AAAATTAGCGAAAAGACATGTTGTCCTGGGAAATTTGAT |
| | | ACGGAAGATAAAGACAGGTTGCTGCAATTGAGGCAGCG |
| | | GTCCACCCTTCGTTGGACCGATAAGAACCCCTGTCCAGA |
| | | TAACGCTGAAACTTACTGCCCGTTTTGCGAACTTCTCGG |
| | | GAGATCCGGTAACGATGGAAAGAAGGCTGAGAAGAAGG |
| | | ATTGGCGCTTTCGTATTCATTTTGGCAATCTTTCCCTTCC |
| | | AGGGAAGCCCGATTTCGACGGGCCAAAAGCTATAGGCA |
| | | GCCAACGGGTACTTAACAGGGTCGATTTCAAATCCGGTA |
| | | AGGCCCATGACTTCTTTAAGGCTTACGAAGTCGACCATA |
| | | CTAGGTTTCCCCGCTTCGAAGGGGAGATTACCATAGATA |
| | | ATAAAGTCAGCGCTGAAGCCAGGAAACTGCTCTGTGATT |
| | | CTCTCAAGTTTACGGATCGGCTGTGTGGAGCTCTGTGCG |
| | | TAATCAGGTTCGATGAATATACACCAGCAGCCGATAGTG |
| | | GGAAACAAACCGAAAATGTCCAAGCAGAACCGAATGCT |
| | | AATCTCGCTGAAAAGACCGCAGAGCAAATTATTAGCATT |
| | | CTGGACGATAATAAGAAAACCGAATATACCCGCCTGCTT |
| | | GCTGATGCTATTCGTTCACTCCGCCGCTCTAGTAAACTTG |
| | | TCGCTGGCTTGCCAAAGGATCATGACGGGAAGGATGAC |
| | | CATTACCTCTGGGATATCGGCAAGAAGAAGAAAGACGA |
| | | AAACTCTGTCACTATTAGGCAAATCCTTACGACCTCAGC |
| | | AGACACCAAGGAACTCAAGAATGCCGGAAAATGGAGAG |
| | | AATTCTGCGAGAAGCTGGGTGAAGCGCTGTACCTCAAG |
| | | AGTAAAGACATGAGTGGCGGCCTGAAAATTACTCGGAG |
| | | AATACTGGGCGATGCTGAATTCCATGGAAAGCCCGATCG |
| | | GCTTGAAAAGAGCCGGTCTGTGTCAATCGGATCTGTGTT |
| | | GAAAGAAACTGTGGTATGCGGCGAACTGGTCGCTAAAA |
| | | CGCCCTTCTTCTTCGGAGCGATAGACGAAGATGCAAAAC |
| | | AAACCgacCTGCAAGTACTCCTCACTCCCGATAACAAGTA |
| | | TAGACTGCCAAGAAGCGCCGTGCGAGGTATACTCCGTCG |
| | | AGATCTTCAAACCTATTTTGATAGCCCATGCAATGCTGA |
| | | GTTGGGTGGACGGCCATGCATGTGTAAGACGTGTAGGAT |
| | | TATGAGAGGGATCACGGTGATGGATGCGCGCAGTGAGT |
| | | ACAATGCCCCGCCAGAAATAAGGCATCGTACCCGCATTA |
| | | ATCCCTTCACAGGCACGGTTGCGGAAGGTGCCCTGTTTA |
| | | ATATGGAAGTAGCCCCCGAGGGGATTGTCTTTCCATTTC |
| | | AACTCCGGTACCGGGGCTCTGAAGATGGGCTGCCCGATG |
| | | CACTGAAAACGGTGTTGAAATGGTGGGCTGAGGGGCAG |
| | | GCATTCATGAGTGGCGCTGCCTCAACCGGGAAGGGCCG |
| | | ATTCCGGATGGAAAATGCTAAATATGAAACGCTGGATCT |
| | | GAGCGACGAGAATCAAAGGAATGACTATCTTAAGAATT |
| | | GGGGATGGCGTGACGAGAAGGGGCTCGAGGAACTGAAG |
| | | AAACGACTGAACTCAGGTCTGCCAGAGCCCGGTAATTAT |
| | | AGGGATCCAAAATGGCACGAGATTAACGTTTCCATTGAG |
| | | ATGGCAAGCCCTTTTATTAATGGCGACCCAATCCGCGCA |
| | | GCCGTGGACAAACGTGGTACAgatGTGGTTACCTTCGTTA |
| | | AGTATAAAGCTGAAGGGGAAGAGGCGAAACCCGTATGT |
| | | GCATACAAGGCCGAATCTTTTAGAGGGGTGATCAGAAG |
| | | TGCCGTGGCACGCATTCATATGGAAGATGGCGTCCCTTT |
| | | GACTGAGTTGACTCACAGTGACTGTGAATGTCTCCTGTG |
| | | CCAAATCTTTGGAAGTGAGTATGAAGCCGGCAAAATAA |
| | | GGTTTGAAGATCTCGTATTCGAAAGTGACCCGGAACCTG |
| | | TGACCTTCGATCATGTGGCCATCGATAGATTCACTGGCG |
| | | GTGCAGCTGATAAGAAGAAATTCGATGATTCCCCTCTGC |
| | | CCGGTAGCCCTGCAAGACCGCTCATGTTGAAAGGCTCCT |
| | | TCTGGATCCGCAGGGACGTTCTCGAGGACGAAGAGTACT |
| | | GTAAGGCACTCGGTAAGGCTCTTGCAGATGTGAATAATG |
| | | GCCTTTATCCCCTCGGTGGAAAGAGCGCCATCGGCTACG |
| | | GACAGGTCAAGAGTCTGGGTATAAAGGGAGATGATAAG |
| | | AGGATTTCTCGCCTCATGAATCCTGCCTTTGATGAGACA |
| | | GATGTAGCCGTTCCAGAAAAGCCCAAAACTGATGCCGA |
| | | GGTTCGCATCGAGGCAGAGAAAGTATATTACCCACACTA |
| | | TTTCGTCGAACCCCATAAGAAGGTGGAACGCGAGGAGA |
| | | AACCCTGTGGTCATCAAAAGTTCCACGAGGGGCGACTG |
| | | ACAGGTAAAATTCGGTGTAAGCTCATTACCAAGACACCC |
| | | CTCATCGTCCCAGATACTAGTAATGACGATTTCTTCAGA |
| | | CCTGCGGATAAAGAAGCTCGGAAGGAAAAGGACGAATA |
| | | TCATAAATCATATGCTTTCTTCAGACTTCATAAACAAAT |
| | | CATGATTCCCGGGAGCGAATTGAGAGGAATGGTGAGTA |
| | | GTGTCTACGAAACTGTGACAAATTCTTGCTTCAGGATAT |
| | | TTGATGAGACTAAACGGTTGTCATGGCGGATGGATGCTG |
| | | ATCACCAGAATGTGCTGCAAGACTTTCTCCCAGGTCGAG |
| | | TGACCGCCGATGGGAAACATATACAAAAGTTTTCCGAA |
| | | ACTGCAAGGGTGCCTTTCTATGACAAAACGCAGAAACAT |
| | | TTTGACATTCTTGATGAGCAAGAAATTGCTGGTGAGAAA |
| | | CCTGTTCGGATGTGGGTCAAGCGTTTTATTAAACGACTG |
| | | AGCCTCGTTGATCCTGCTAAACACCCCCAGAAGAAACAA |
| | | GACAATAAATGGAAAAGACGCAAAGAAGGCATCGCCAC |
| | | ATTTATAGAGCAAAAGAATGGCTCTTATTATTTTAACGT |
| | | GGTCACCAATAATGGCTGCACTTCTTTCCACTTGTGGCA |
| | | TAAACCTGACAATTTTGACCAGGAGAAACTCGAAGGCA |
| | | TACAGAATGGTGAGAAACTGGATTGCTGGGTAAGAGAT |
| | | AGTAGATACCAAAAGGCCTTTCAAGAGATACCCGAGAA |
| | | CGACCCAGACGGATGGGAGTGTAAAGAGGGCTACCTTC |
| | | ATGTCGTCGGCCCCAGCAAAGTAGAGTTTAGTGACAAG |
| | | AAAGGGGATGTGATCAATAACTTTCAAGGAACACTCCC |
| | | ATCAGTTCCCAACGACTGGAAAACAATTAGGACGAACG |
| | | ATTTTAAGAATAGGAAAAGGAAGAATGAACCTGTGTTTT |
| | | GTTGTGAGGATGATAAGGGCAATTACTATACCATGGCTA |
| | | AATATTGCGAAACCTTCTTCTTCGATCTGAAGGAGAACG |
| | | AGGAATACGAGATCCCCGAGAAAGCAAGAATCAAATAC |
| | | AAAGAACTGCTCAGGGTCTATAACAACAATCCTCAAGC |
| | | AGTGCCGGAGAGCGTATTTCAGTCTAGAGTTGCCCGGGA |
| | | AAACGTGGAAAAGCTGAAGTCCGGAGATCTTGTGTATTT |
| | | CAAACATAATGAAAAGTACGTAGAGGACATCGTCCCAG |
| | | TGCGGATTTCCCGAACTGTAGACGATAGGATGATCGGCA |
| | | AACGTATGAGCGCCGATCTGCGGCCGTGCCATGGAGATT |
| | | GGGTGGAAGATGGTGATCTCAGTGCCTTGAATGCATATC |
| | | CCGAGAAAAGACTCCTCTTGCGCCACCCCAAAGGACTCT |
| | | GCCCTGCTTGCCGGCTCTTTGGAACCGGATCTTACAAGG |
| | | GCAGAGTCAGGTTTGGATTCGCGTCACTCGAAAACGATC |
| | | CGGAGTGGCTGATCCCAGGCAAGAATCCCGGCGATCCG |
| | | TTTCACGGCGGGCCGGTGATGCTCTCATTGTTGGAACGG |
| | | CCTCGCCCGACTTGGAGTATACCGGGATCCGACAATAAG |
| | | TTTAAAGTGCCTGGCAGAAAGTTTTACGTCCACCACCAC |
| | | GCCTGGAAAACCATTAAGGACGGGAACCATCCCACAAC |
| | | AGGCAAAGCTATTGAACAAAGCCCTAATAACCGCACTG |
| | | TAGAAGCTCTCGCCGGCGGGAATTCCTTTAGCTTCGAAA |
| | | TTGCCTTTGAGAACCTGAAAGAATGGGAGCTGGGTTTGC |
| | | TCATCCACAGCCTGCAACTCGAAAAGGGTCTGGCGCATA |
| | | AACTTGGAATGGCAAAGTCTATGGGATTTGGTTCAGTTG |
| | | AAATTGACGTCGAATCAGTGCGCCTGAGAAAAGATTGG |
| | | AAGCAATGGCGGAATGGCAATTCCGAAATTCCCAACTG |
| | | GTTGGGAAAAGGATTTGCTAAACTGAAGGAATGGTTCC |
| | | GGGACGAGCTCGATTTTATAGAAAATCTTAAGAAACTTC |
| | | TTTGGTTTCCTGAGGGCGACCAAGCACCCCGGGTTTGCT |
| | | ACCCCATGCTGCGAAAGAAGGACGATCCTAATGGGAAT |
| | | AGCGGTTACGAAGAACTCAAAAGAGGGGAATTCAAGAA |
| | | AGAAGATCGGCAGAAGAAGCTGACCACGCCGTGGACAC |
| | | CGTGGGCAaaacggacagccgacggaagcgagttcgagtcaccaaagaagaagcg |
| | | gaaagtctaa |
| 198 | Cas711_D1580R_ | ATGaaacggacagccgacggaagcgagttcgagtcaccaaagaagaagcggaaagtc |
| | D988K | ACGACTACTATGAAGATTTCAATTGAATTCCTCGAGCCG |
| | (pDF0506 based) | TTTCGGATGACCAAATGGCAGGAAAGCACTAGGCGAAA |
| | Doublemutant | CAAGAACAACAAGGAATTTGTTCGCGGCCAAGCTTTTGC |
| | | AAGATGGCATAGAAATAAGAAGGATAACACAAAAGGGC |
| | | GGCCTTACATAACTGGGACATTGCTGCGGTCCGCAGTAA |
| | | TTAGAAGCGCGGAAAACCTGCTGACATTGAGTGATGGG |
| | | AAAATTAGCGAAAAGACATGTTGTCCTGGGAAATTTGAT |
| | | ACGGAAGATAAAGACAGGTTGCTGCAATTGAGGCAGCG |
| | | GTCCACCCTTCGTTGGACCGATAAGAACCCCTGTCCAGA |
| | | TAACGCTGAAACTTACTGCCCGTTTTGCGAACTTCTCGG |
| | | GAGATCCGGTAACGATGGAAAGAAGGCTGAGAAGAAGG |
| | | ATTGGCGCTTTCGTATTCATTTTGGCAATCTTTCCCTTCC |
| | | AGGGAAGCCCGATTTCGACGGGCCAAAAGCTATAGGCA |
| | | GCCAACGGGTACTTAACAGGGTCGATTTCAAATCCGGTA |
| | | AGGCCCATGACTTCTTTAAGGCTTACGAAGTCGACCATA |
| | | CTAGGTTTCCCCGCTTCGAAGGGGAGATTACCATAGATA |
| | | ATAAAGTCAGCGCTGAAGCCAGGAAACTGCTCTGTGATT |
| | | CTCTCAAGTTTACGGATCGGCTGTGTGGAGCTCTGTGCG |
| | | TAATCAGGTTCGATGAATATACACCAGCAGCCGATAGTG |
| | | GGAAACAAACCGAAAATGTCCAAGCAGAACCGAATGCT |
| | | AATCTCGCTGAAAAGACCGCAGAGCAAATTATTAGCATT |
| | | CTGGACGATAATAAGAAAACCGAATATACCCGCCTGCTT |
| | | GCTGATGCTATTCGTTCACTCCGCCGCTCTAGTAAACTTG |
| | | TCGCTGGCTTGCCAAAGGATCATGACGGGAAGGATGAC |
| | | CATTACCTCTGGGATATCGGCAAGAAGAAGAAAGACGA |
| | | AAACTCTGTCACTATTAGGCAAATCCTTACGACCTCAGC |
| | | AGACACCAAGGAACTCAAGAATGCCGGAAAATGGAGAG |
| | | AATTCTGCGAGAAGCTGGGTGAAGCGCTGTACCTCAAG |
| | | AGTAAAGACATGAGTGGCGGCCTGAAAATTACTCGGAG |
| | | AATACTGGGCGATGCTGAATTCCATGGAAAGCCCGATCG |
| | | GCTTGAAAAGAGCCGGTCTGTGTCAATCGGATCTGTGTT |
| | | GAAAGAAACTGTGGTATGCGGCGAACTGGTCGCTAAAA |
| | | CGCCCTTCTTCTTCGGAGCGATAGACGAAGATGCAAAAC |
| | | AAACCgacCTGCAAGTACTCCTCACTCCCGATAACAAGTA |
| | | TAGACTGCCAAGAAGCGCCGTGCGAGGTATACTCCGTCG |
| | | AGATCTTCAAACCTATTTTGATAGCCCATGCAATGCTGA |
| | | GTTGGGTGGACGGCCATGCATGTGTAAGACGTGTAGGAT |
| | | TATGAGAGGGATCACGGTGATGGATGCGCGCAGTGAGT |
| | | ACAATGCCCCGCCAGAAATAAGGCATCGTACCCGCATTA |
| | | ATCCCTTCACAGGCACGGTTGCGGAAGGTGCCCTGTTTA |
| | | ATATGGAAGTAGCCCCCGAGGGGATTGTCTTTCCATTTC |
| | | AACTCCGGTACCGGGGCTCTGAAGATGGGCTGCCCGATG |
| | | CACTGAAAACGGTGTTGAAATGGTGGGCTGAGGGGCAG |
| | | GCATTCATGAGTGGCGCTGCCTCAACCGGGAAGGGCCG |
| | | ATTCCGGATGGAAAATGCTAAATATGAAACGCTGGATCT |
| | | GAGCGACGAGAATCAAAGGAATGACTATCTTAAGAATT |
| | | GGGGATGGCGTGACGAGAAGGGGCTCGAGGAACTGAAG |
| | | AAACGACTGAACTCAGGTCTGCCAGAGCCCGGTAATTAT |
| | | AGGGATCCAAAATGGCACGAGATTAACGTTTCCATTGAG |
| | | ATGGCAAGCCCTTTTATTAATGGCGACCCAATCCGCGCA |
| | | GCCGTGGACAAACGTGGTACAgatGTGGTTACCTTCGTTA |
| | | AGTATAAAGCTGAAGGGGAAGAGGCGAAACCCGTATGT |
| | | GCATACAAGGCCGAATCTTTTAGAGGGGTGATCAGAAG |
| | | TGCCGTGGCACGCATTCATATGGAAGATGGCGTCCCTTT |
| | | GACTGAGTTGACTCACAGTGACTGTGAATGTCTCCTGTG |
| | | CCAAATCTTTGGAAGTGAGTATGAAGCCGGCAAAATAA |
| | | GGTTTGAAGATCTCGTATTCGAAAGTGACCCGGAACCTG |
| | | TGACCTTCGATCATGTGGCCATCGATAGATTCACTGGCG |
| | | GTGCAGCTGATAAGAAGAAATTCGATGATTCCCCTCTGC |
| | | CCGGTAGCCCTGCAAGACCGCTCATGTTGAAAGGCTCCT |
| | | TCTGGATCCGCAGGGACGTTCTCGAGGACGAAGAGTACT |
| | | GTAAGGCACTCGGTAAGGCTCTTGCAGATGTGAATAATG |
| | | GCCTTTATCCCCTCGGTGGAAAGAGCGCCATCGGCTACG |
| | | GACAGGTCAAGAGTCTGGGTATAAAGGGAGATGATAAG |
| | | AGGATTTCTCGCCTCATGAATCCTGCCTTTGATGAGACA |
| | | GATGTAGCCGTTCCAGAAAAGCCCAAAACTGATGCCGA |
| | | GGTTCGCATCGAGGCAGAGAAAGTATATTACCCACACTA |
| | | TTTCGTCGAACCCCATAAGAAGGTGGAACGCGAGGAGA |
| | | AACCCTGTGGTCATCAAAAGTTCCACGAGGGGCGACTG |
| | | ACAGGTAAAATTCGGTGTAAGCTCATTACCAAGACACCC |
| | | CTCATCGTCCCAGATACTAGTAATGACGATTTCTTCAGA |
| | | CCTGCGGATAAAGAAGCTCGGAAGGAAAAGGACGAATA |
| | | TCATAAATCATATGCTTTCTTCAGACTTCATAAACAAAT |
| | | CATGATTCCCGGGAGCGAATTGAGAGGAATGGTGAGTA |
| | | GTGTCTACGAAACTGTGACAAATTCTTGCTTCAGGATAT |
| | | TTGATGAGACTAAACGGTTGTCATGGCGGATGGATGCTG |
| | | ATCACCAGAATGTGCTGCAAAAGTTTCTCCCAGGTCGAG |
| | | TGACCGCCGATGGGAAACATATACAAAAGTTTTCCGAA |
| | | ACTGCAAGGGTGCCTTTCTATGACAAAACGCAGAAACAT |
| | | TTTGACATTCTTGATGAGCAAGAAATTGCTGGTGAGAAA |
| | | CCTGTTCGGATGTGGGTCAAGCGTTTTATTAAACGACTG |
| | | AGCCTCGTTGATCCTGCTAAACACCCCCAGAAGAAACAA |
| | | GACAATAAATGGAAAAGACGCAAAGAAGGCATCGCCAC |
| | | ATTTATAGAGCAAAAGAATGGCTCTTATTATTTTAACGT |
| | | GGTCACCAATAATGGCTGCACTTCTTTCCACTTGTGGCA |
| | | TAAACCTGACAATTTTGACCAGGAGAAACTCGAAGGCA |
| | | TACAGAATGGTGAGAAACTGGATTGCTGGGTAAGAGAT |
| | | AGTAGATACCAAAAGGCCTTTCAAGAGATACCCGAGAA |
| | | CGACCCAGACGGATGGGAGTGTAAAGAGGGCTACCTTC |
| | | ATGTCGTCGGCCCCAGCAAAGTAGAGTTTAGTGACAAG |
| | | AAAGGGGATGTGATCAATAACTTTCAAGGAACACTCCC |
| | | ATCAGTTCCCAACGACTGGAAAACAATTAGGACGAACG |
| | | ATTTTAAGAATAGGAAAAGGAAGAATGAACCTGTGTTTT |
| | | GTTGTGAGGATGATAAGGGCAATTACTATACCATGGCTA |
| | | AATATTGCGAAACCTTCTTCTTCGATCTGAAGGAGAACG |
| | | AGGAATACGAGATCCCCGAGAAAGCAAGAATCAAATAC |
| | | AAAGAACTGCTCAGGGTCTATAACAACAATCCTCAAGC |
| | | AGTGCCGGAGAGCGTATTTCAGTCTAGAGTTGCCCGGGA |
| | | AAACGTGGAAAAGCTGAAGTCCGGAGATCTTGTGTATTT |
| | | CAAACATAATGAAAAGTACGTAGAGGACATCGTCCCAG |
| | | TGCGGATTTCCCGAACTGTAGACGATAGGATGATCGGCA |
| | | AACGTATGAGCGCCGATCTGCGGCCGTGCCATGGAGATT |
| | | GGGTGGAAGATGGTGATCTCAGTGCCTTGAATGCATATC |
| | | CCGAGAAAAGACTCCTCTTGCGCCACCCCAAAGGACTCT |
| | | GCCCTGCTTGCCGGCTCTTTGGAACCGGATCTTACAAGG |
| | | GCAGAGTCAGGTTTGGATTCGCGTCACTCGAAAACGATC |
| | | CGGAGTGGCTGATCCCAGGCAAGAATCCCGGCGATCCG |
| | | TTTCACGGCGGGCCGGTGATGCTCTCATTGTTGGAACGG |
| | | CCTCGCCCGACTTGGAGTATACCGGGATCCGACAATAAG |
| | | TTTAAAGTGCCTGGCAGAAAGTTTTACGTCCACCACCAC |
| | | GCCTGGAAAACCATTAAGGACGGGAACCATCCCACAAC |
| | | AGGCAAAGCTATTGAACAAAGCCCTAATAACCGCACTG |
| | | TAGAAGCTCTCGCCGGCGGGAATTCCTTTAGCTTCGAAA |
| | | TTGCCTTTGAGAACCTGAAAGAATGGGAGCTGGGTTTGC |
| | | TCATCCACAGCCTGCAACTCGAAAAGGGTCTGGCGCATA |
| | | AACTTGGAATGGCAAAGTCTATGGGATTTGGTTCAGTTG |
| | | AAATTGACGTCGAATCAGTGCGCCTGAGAAAAGATTGG |
| | | AAGCAATGGCGGAATGGCAATTCCGAAATTCCCAACTG |
| | | GTTGGGAAAAGGATTTGCTAAACTGAAGGAATGGTTCC |
| | | GGGACGAGCTCGATTTTATAGAAAATCTTAAGAAACTTC |
| | | TTTGGTTTCCTGAGGGCGACCAAGCACCCCGGGTTTGCT |
| | | ACCCCATGCTGCGAAAGAAGGACGATCCTAATGGGAAT |
| | | AGCGGTTACGAAGAACTCAAAAGAGGGGAATTCAAGAA |
| | | AGAAGATCGGCAGAAGAAGCTGACCACGCCGTGGACAC |
| | | CGTGGGCAaaacggacagccgacggaagcgagttcgagtcaccaaagaagaagcg |
| | | gaaagtctaa |
| 199 | Cas711_D1580R_ | ATGaaacggacagccgacggaagcgagttcgagtcaccaaagaagaagcggaaagtc |
| | D988K_D981K | ACGACTACTATGAAGATTTCAATTGAATTCCTCGAGCCG |
| | (pDF0506 based) | TTTCGGATGACCAAATGGCAGGAAAGCACTAGGCGAAA |
| | Triplemutant | CAAGAACAACAAGGAATTTGTTCGCGGCCAAGCTTTTGC |
| | | AAGATGGCATAGAAATAAGAAGGATAACACAAAAGGGC |
| | | GGCCTTACATAACTGGGACATTGCTGCGGTCCGCAGTAA |
| | | TTAGAAGCGCGGAAAACCTGCTGACATTGAGTGATGGG |
| | | AAAATTAGCGAAAAGACATGTTGTCCTGGGAAATTTGAT |
| | | ACGGAAGATAAAGACAGGTTGCTGCAATTGAGGCAGCG |
| | | GTCCACCCTTCGTTGGACCGATAAGAACCCCTGTCCAGA |
| | | TAACGCTGAAACTTACTGCCCGTTTTGCGAACTTCTCGG |
| | | GAGATCCGGTAACGATGGAAAGAAGGCTGAGAAGAAGG |
| | | ATTGGCGCTTTCGTATTCATTTTGGCAATCTTTCCCTTCC |
| | | AGGGAAGCCCGATTTCGACGGGCCAAAAGCTATAGGCA |
| | | GCCAACGGGTACTTAACAGGGTCGATTTCAAATCCGGTA |
| | | AGGCCCATGACTTCTTTAAGGCTTACGAAGTCGACCATA |
| | | CTAGGTTTCCCCGCTTCGAAGGGGAGATTACCATAGATA |
| | | ATAAAGTCAGCGCTGAAGCCAGGAAACTGCTCTGTGATT |
| | | CTCTCAAGTTTACGGATCGGCTGTGTGGAGCTCTGTGCG |
| | | TAATCAGGTTCGATGAATATACACCAGCAGCCGATAGTG |
| | | GGAAACAAACCGAAAATGTCCAAGCAGAACCGAATGCT |
| | | AATCTCGCTGAAAAGACCGCAGAGCAAATTATTAGCATT |
| | | CTGGACGATAATAAGAAAACCGAATATACCCGCCTGCTT |
| | | GCTGATGCTATTCGTTCACTCCGCCGCTCTAGTAAACTTG |
| | | TCGCTGGCTTGCCAAAGGATCATGACGGGAAGGATGAC |
| | | CATTACCTCTGGGATATCGGCAAGAAGAAGAAAGACGA |
| | | AAACTCTGTCACTATTAGGCAAATCCTTACGACCTCAGC |
| | | AGACACCAAGGAACTCAAGAATGCCGGAAAATGGAGAG |
| | | AATTCTGCGAGAAGCTGGGTGAAGCGCTGTACCTCAAG |
| | | AGTAAAGACATGAGTGGCGGCCTGAAAATTACTCGGAG |
| | | AATACTGGGCGATGCTGAATTCCATGGAAAGCCCGATCG |
| | | GCTTGAAAAGAGCCGGTCTGTGTCAATCGGATCTGTGTT |
| | | GAAAGAAACTGTGGTATGCGGCGAACTGGTCGCTAAAA |
| | | CGCCCTTCTTCTTCGGAGCGATAGACGAAGATGCAAAAC |
| | | AAACCgacCTGCAAGTACTCCTCACTCCCGATAACAAGTA |
| | | TAGACTGCCAAGAAGCGCCGTGCGAGGTATACTCCGTCG |
| | | AGATCTTCAAACCTATTTTGATAGCCCATGCAATGCTGA |
| | | GTTGGGTGGACGGCCATGCATGTGTAAGACGTGTAGGAT |
| | | TATGAGAGGGATCACGGTGATGGATGCGCGCAGTGAGT |
| | | ACAATGCCCCGCCAGAAATAAGGCATCGTACCCGCATTA |
| | | ATCCCTTCACAGGCACGGTTGCGGAAGGTGCCCTGTTTA |
| | | ATATGGAAGTAGCCCCCGAGGGGATTGTCTTTCCATTTC |
| | | AACTCCGGTACCGGGGCTCTGAAGATGGGCTGCCCGATG |
| | | CACTGAAAACGGTGTTGAAATGGTGGGCTGAGGGGCAG |
| | | GCATTCATGAGTGGCGCTGCCTCAACCGGGAAGGGCCG |
| | | ATTCCGGATGGAAAATGCTAAATATGAAACGCTGGATCT |
| | | GAGCGACGAGAATCAAAGGAATGACTATCTTAAGAATT |
| | | GGGGATGGCGTGACGAGAAGGGGCTCGAGGAACTGAAG |
| | | AAACGACTGAACTCAGGTCTGCCAGAGCCCGGTAATTAT |
| | | AGGGATCCAAAATGGCACGAGATTAACGTTTCCATTGAG |
| | | ATGGCAAGCCCTTTTATTAATGGCGACCCAATCCGCGCA |
| | | GCCGTGGACAAACGTGGTACAgatGTGGTTACCTTCGTTA |
| | | AGTATAAAGCTGAAGGGGAAGAGGCGAAACCCGTATGT |
| | | GCATACAAGGCCGAATCTTTTAGAGGGGTGATCAGAAG |
| | | TGCCGTGGCACGCATTCATATGGAAGATGGCGTCCCTTT |
| | | GACTGAGTTGACTCACAGTGACTGTGAATGTCTCCTGTG |
| | | CCAAATCTTTGGAAGTGAGTATGAAGCCGGCAAAATAA |
| | | GGTTTGAAGATCTCGTATTCGAAAGTGACCCGGAACCTG |
| | | TGACCTTCGATCATGTGGCCATCGATAGATTCACTGGCG |
| | | GTGCAGCTGATAAGAAGAAATTCGATGATTCCCCTCTGC |
| | | CCGGTAGCCCTGCAAGACCGCTCATGTTGAAAGGCTCCT |
| | | TCTGGATCCGCAGGGACGTTCTCGAGGACGAAGAGTACT |
| | | GTAAGGCACTCGGTAAGGCTCTTGCAGATGTGAATAATG |
| | | GCCTTTATCCCCTCGGTGGAAAGAGCGCCATCGGCTACG |
| | | GACAGGTCAAGAGTCTGGGTATAAAGGGAGATGATAAG |
| | | AGGATTTCTCGCCTCATGAATCCTGCCTTTGATGAGACA |
| | | GATGTAGCCGTTCCAGAAAAGCCCAAAACTGATGCCGA |
| | | GGTTCGCATCGAGGCAGAGAAAGTATATTACCCACACTA |
| | | TTTCGTCGAACCCCATAAGAAGGTGGAACGCGAGGAGA |
| | | AACCCTGTGGTCATCAAAAGTTCCACGAGGGGCGACTG |
| | | ACAGGTAAAATTCGGTGTAAGCTCATTACCAAGACACCC |
| | | CTCATCGTCCCAGATACTAGTAATGACGATTTCTTCAGA |
| | | CCTGCGGATAAAGAAGCTCGGAAGGAAAAGGACGAATA |
| | | TCATAAATCATATGCTTTCTTCAGACTTCATAAACAAAT |
| | | CATGATTCCCGGGAGCGAATTGAGAGGAATGGTGAGTA |
| | | GTGTCTACGAAACTGTGACAAATTCTTGCTTCAGGATAT |
| | | TTGATGAGACTAAACGGTTGTCATGGCGGATGGATGCTA |
| | | AGCACCAGAATGTGCTGCAAAAGTTTCTCCCAGGTCGAG |
| | | TGACCGCCGATGGGAAACATATACAAAAGTTTTCCGAA |
| | | ACTGCAAGGGTGCCTTTCTATGACAAAACGCAGAAACAT |
| | | TTTGACATTCTTGATGAGCAAGAAATTGCTGGTGAGAAA |
| | | CCTGTTCGGATGTGGGTCAAGCGTTTTATTAAACGACTG |
| | | AGCCTCGTTGATCCTGCTAAACACCCCCAGAAGAAACAA |
| | | GACAATAAATGGAAAAGACGCAAAGAAGGCATCGCCAC |
| | | ATTTATAGAGCAAAAGAATGGCTCTTATTATTTTAACGT |
| | | GGTCACCAATAATGGCTGCACTTCTTTCCACTTGTGGCA |
| | | TAAACCTGACAATTTTGACCAGGAGAAACTCGAAGGCA |
| | | TACAGAATGGTGAGAAACTGGATTGCTGGGTAAGAGAT |
| | | AGTAGATACCAAAAGGCCTTTCAAGAGATACCCGAGAA |
| | | CGACCCAGACGGATGGGAGTGTAAAGAGGGCTACCTTC |
| | | ATGTCGTCGGCCCCAGCAAAGTAGAGTTTAGTGACAAG |
| | | AAAGGGGATGTGATCAATAACTTTCAAGGAACACTCCC |
| | | ATCAGTTCCCAACGACTGGAAAACAATTAGGACGAACG |
| | | ATTTTAAGAATAGGAAAAGGAAGAATGAACCTGTGTTTT |
| | | GTTGTGAGGATGATAAGGGCAATTACTATACCATGGCTA |
| | | AATATTGCGAAACCTTCTTCTTCGATCTGAAGGAGAACG |
| | | AGGAATACGAGATCCCCGAGAAAGCAAGAATCAAATAC |
| | | AAAGAACTGCTCAGGGTCTATAACAACAATCCTCAAGC |
| | | AGTGCCGGAGAGCGTATTTCAGTCTAGAGTTGCCCGGGA |
| | | AAACGTGGAAAAGCTGAAGTCCGGAGATCTTGTGTATTT |
| | | CAAACATAATGAAAAGTACGTAGAGGACATCGTCCCAG |
| | | TGCGGATTTCCCGAACTGTAGACGATAGGATGATCGGCA |
| | | AACGTATGAGCGCCGATCTGCGGCCGTGCCATGGAGATT |
| | | GGGTGGAAGATGGTGATCTCAGTGCCTTGAATGCATATC |
| | | CCGAGAAAAGACTCCTCTTGCGCCACCCCAAAGGACTCT |
| | | GCCCTGCTTGCCGGCTCTTTGGAACCGGATCTTACAAGG |
| | | GCAGAGTCAGGTTTGGATTCGCGTCACTCGAAAACGATC |
| | | CGGAGTGGCTGATCCCAGGCAAGAATCCCGGCGATCCG |
| | | TTTCACGGCGGGCCGGTGATGCTCTCATTGTTGGAACGG |
| | | CCTCGCCCGACTTGGAGTATACCGGGATCCGACAATAAG |
| | | TTTAAAGTGCCTGGCAGAAAGTTTTACGTCCACCACCAC |
| | | GCCTGGAAAACCATTAAGGACGGGAACCATCCCACAAC |
| | | AGGCAAAGCTATTGAACAAAGCCCTAATAACCGCACTG |
| | | TAGAAGCTCTCGCCGGCGGGAATTCCTTTAGCTTCGAAA |
| | | TTGCCTTTGAGAACCTGAAAGAATGGGAGCTGGGTTTGC |
| | | TCATCCACAGCCTGCAACTCGAAAAGGGTCTGGCGCATA |
| | | AACTTGGAATGGCAAAGTCTATGGGATTTGGTTCAGTTG |
| | | AAATTGACGTCGAATCAGTGCGCCTGAGAAAAGATTGG |
| | | AAGCAATGGCGGAATGGCAATTCCGAAATTCCCAACTG |
| | | GTTGGGAAAAGGATTTGCTAAACTGAAGGAATGGTTCC |
| | | GGGACGAGCTCGATTTTATAGAAAATCTTAAGAAACTTC |
| | | TTTGGTTTCCTGAGGGCGACCAAGCACCCCGGGTTTGCT |
| | | ACCCCATGCTGCGAAAGAAGGACGATCCTAATGGGAAT |
| | | AGCGGTTACGAAGAACTCAAAAGAGGGGAATTCAAGAA |
| | | AGAAGATCGGCAGAAGAAGCTGACCACGCCGTGGACAC |
| | | CGTGGGCAaaacggacagccgacggaagcgagttcgagtcaccaaagaagaagcg |
| | | gaaagtctaa |
| 200 | Cas711_D1580R_ | ATGaaacggacagccgacggaagcgagttcgagtcaccaaagaagaagcggaaagtc |
| | (pDF0506 based) | ACGACTACTATGAAGATTTCAATTGAATTCCTCGAGCCG |
| | Singlemutant | TTTCGGATGACCAAATGGCAGGAAAGCACTAGGCGAAA |
| | | CAAGAACAACAAGGAATTTGTTCGCGGCCAAGCTTTTGC |
| | | AAGATGGCATAGAAATAAGAAGGATAACACAAAAGGGC |
| | | GGCCTTACATAACTGGGACATTGCTGCGGTCCGCAGTAA |
| | | TTAGAAGCGCGGAAAACCTGCTGACATTGAGTGATGGG |
| | | AAAATTAGCGAAAAGACATGTTGTCCTGGGAAATTTGAT |
| | | ACGGAAGATAAAGACAGGTTGCTGCAATTGAGGCAGCG |
| | | GTCCACCCTTCGTTGGACCGATAAGAACCCCTGTCCAGA |
| | | TAACGCTGAAACTTACTGCCCGTTTTGCGAACTTCTCGG |
| | | GAGATCCGGTAACGATGGAAAGAAGGCTGAGAAGAAGG |
| | | ATTGGCGCTTTCGTATTCATTTTGGCAATCTTTCCCTTCC |
| | | AGGGAAGCCCGATTTCGACGGGCCAAAAGCTATAGGCA |
| | | GCCAACGGGTACTTAACAGGGTCGATTTCAAATCCGGTA |
| | | AGGCCCATGACTTCTTTAAGGCTTACGAAGTCGACCATA |
| | | CTAGGTTTCCCCGCTTCGAAGGGGAGATTACCATAGATA |
| | | ATAAAGTCAGCGCTGAAGCCAGGAAACTGCTCTGTGATT |
| | | CTCTCAAGTTTACGGATCGGCTGTGTGGAGCTCTGTGCG |
| | | TAATCAGGTTCGATGAATATACACCAGCAGCCGATAGTG |
| | | GGAAACAAACCGAAAATGTCCAAGCAGAACCGAATGCT |
| | | AATCTCGCTGAAAAGACCGCAGAGCAAATTATTAGCATT |
| | | CTGGACGATAATAAGAAAACCGAATATACCCGCCTGCTT |
| | | GCTGATGCTATTCGTTCACTCCGCCGCTCTAGTAAACTTG |
| | | TCGCTGGCTTGCCAAAGGATCATGACGGGAAGGATGAC |
| | | CATAAGCTCTGGGATATCGGCAAGAAGAAGAAAGACGA |
| | | AAACTCTGTCACTATTAGGCAAATCCTTACGACCTCAGC |
| | | AGACACCAAGGAACTCAAGAATGCCGGAAAATGGAGAG |
| | | AATTCTGCGAGAAGCTGGGTGAAGCGCTGTACCTCAAG |
| | | AGTAAAGACATGAGTGGCGGCCTGAAAATTACTCGGAG |
| | | AATACTGGGCGATGCTGAATTCCATGGAAAGCCCGATCG |
| | | GCTTGAAAAGAGCCGGTCTGTGTCAATCGGATCTGTGTT |
| | | GAAAGAAACTGTGGTATGCGGCGAACTGGTCGCTAAAA |
| | | CGCCCTTCTTCTTCGGAGCGATAGACGAAGATGCAAAAC |
| | | AAACCgacCTGCAAGTACTCCTCACTCCCGATAACAAGTA |
| | | TAGACTGCCAAGAAGCGCCGTGCGAGGTATACTCCGTCG |
| | | AGATCTTCAAACCTATTTTGATAGCCCATGCAATGCTGA |
| | | GTTGGGTGGACGGCCATGCATGTGTAAGACGTGTAGGAT |
| | | TATGAGAGGGATCACGGTGATGGATGCGCGCAGTGAGT |
| | | ACAATGCCCCGCCAGAAATAAGGCATCGTACCCGCATTA |
| | | ATCCCTTCACAGGCACGGTTGCGGAAGGTGCCCTGTTTA |
| | | ATATGGAAGTAGCCCCCGAGGGGATTGTCTTTCCATTTC |
| | | AACTCCGGTACCGGGGCTCTGAAGATGGGCTGCCCGATG |
| | | CACTGAAAACGGTGTTGAAATGGTGGGCTGAGGGGCAG |
| | | GCATTCATGAGTGGCGCTGCCTCAACCGGGAAGGGCCG |
| | | ATTCCGGATGGAAAATGCTAAATATGAAACGCTGGATCT |
| | | GAGCGACGAGAATCAAAGGAATGACTATCTTAAGAATT |
| | | GGGGATGGCGTGACGAGAAGGGGCTCGAGGAACTGAAG |
| | | AAACGACTGAACTCAGGTCTGCCAGAGCCCGGTAATTAT |
| | | AGGGATCCAAAATGGCACGAGATTAACGTTTCCATTGAG |
| | | ATGGCAAGCCCTTTTATTAATGGCGACCCAATCCGCGCA |
| | | GCCGTGGACAAACGTGGTACAgatGTGGTTACCTTCGTTA |
| | | AGTATAAAGCTGAAGGGGAAGAGGCGAAACCCGTATGT |
| | | GCATACAAGGCCGAATCTTTTAGAGGGGTGATCAGAAG |
| | | TGCCGTGGCACGCATTCATATGGAAGATGGCGTCCCTTT |
| | | GACTGAGTTGACTCACAGTGACTGTGAATGTCTCCTGTG |
| | | CCAAATCTTTGGAAGTGAGTATGAAGCCGGCAAAATAA |
| | | GGTTTGAAGATCTCGTATTCGAAAGTGACCCGGAACCTG |
| | | TGACCTTCGATCATGTGGCCATCGATAGATTCACTGGCG |
| | | GTGCAGCTGATAAGAAGAAATTCGATGATTCCCCTCTGC |
| | | CCGGTAGCCCTGCAAGACCGCTCATGTTGAAAGGCTCCT |
| | | TCTGGATCCGCAGGGACGTTCTCGAGGACGAAGAGTACT |
| | | GTAAGGCACTCGGTAAGGCTCTTGCAGATGTGAATAATG |
| | | GCCTTTATCCCCTCGGTGGAAAGAGCGCCATCGGCTACG |
| | | GACAGGTCAAGAGTCTGGGTATAAAGGGAGATGATAAG |
| | | AGGATTTCTCGCCTCATGAATCCTGCCTTTGATGAGACA |
| | | GATGTAGCCGTTCCAGAAAAGCCCAAAACTGATGCCGA |
| | | GGTTCGCATCGAGGCAGAGAAAGTATATTACCCACACTA |
| | | TTTCGTCGAACCCCATAAGAAGGTGGAACGCGAGGAGA |
| | | AACCCTGTGGTCATCAAAAGTTCCACGAGGGGCGACTG |
| | | ACAGGTAAAATTCGGTGTAAGCTCATTACCAAGACACCC |
| | | CTCATCGTCCCAGATACTAGTAATGACGATTTCTTCAGA |
| | | CCTGCGGATAAAGAAGCTCGGAAGGAAAAGGACGAATA |
| | | TCATAAATCATATGCTTTCTTCAGACTTCATAAACAAAT |
| | | CATGATTCCCGGGAGCGAATTGAGAGGAATGGTGAGTA |
| | | GTGTCTACGAAACTGTGACAAATTCTTGCTTCAGGATAT |
| | | TTGATGAGACTAAACGGTTGTCATGGCGGATGGATGCTA |
| | | AGCACCAGAATGTGCTGCAAAAGTTTCTCCCAGGTCGAG |
| | | TGACCGCCGATGGGAAACATATACAAAAGTTTTCCGAA |
| | | ACTGCAAGGGTGCCTTTCTATGACAAAACGCAGAAACAT |
| | | TTTGACATTCTTGATGAGCAAGAAATTGCTGGTGAGAAA |
| | | CCTGTTCGGATGTGGGTCAAGCGTTTTATTAAACGACTG |
| | | AGCCTCGTTGATCCTGCTAAACACCCCCAGAAGAAACAA |
| | | GACAATAAATGGAAAAGACGCAAAGAAGGCATCGCCAC |
| | | ATTTATAGAGCAAAAGAATGGCTCTTATTATTTTAACGT |
| | | GGTCACCAATAATGGCTGCACTTCTTTCCACTTGTGGCA |
| | | TAAACCTGACAATTTTGACCAGGAGAAACTCGAAGGCA |
| | | TACAGAATGGTGAGAAACTGGATTGCTGGGTAAGAGAT |
| | | AGTAGATACCAAAAGGCCTTTCAAGAGATACCCGAGAA |
| | | CGACCCAGACGGATGGGAGTGTAAAGAGGGCTACCTTC |
| | | ATGTCGTCGGCCCCAGCAAAGTAGAGTTTAGTGACAAG |
| | | AAAGGGGATGTGATCAATAACTTTCAAGGAACACTCCC |
| | | ATCAGTTCCCAACGACTGGAAAACAATTAGGACGAACG |
| | | ATTTTAAGAATAGGAAAAGGAAGAATGAACCTGTGTTTT |
| | | GTTGTGAGGATGATAAGGGCAATTACTATACCATGGCTA |
| | | AATATTGCGAAACCTTCTTCTTCGATCTGAAGGAGAACG |
| | | AGGAATACGAGATCCCCGAGAAAGCAAGAATCAAATAC |
| | | AAAGAACTGCTCAGGGTCTATAACAACAATCCTCAAGC |
| | | AGTGCCGGAGAGCGTATTTCAGTCTAGAGTTGCCCGGGA |
| | | AAACGTGGAAAAGCTGAAGTCCGGAGATCTTGTGTATTT |
| | | CAAACATAATGAAAAGTACGTAGAGGACATCGTCCCAG |
| | | TGCGGATTTCCCGAACTGTAGACGATAGGATGATCGGCA |
| | | AACGTATGAGCGCCGATCTGCGGCCGTGCCATGGAGATT |
| | | GGGTGGAAGATGGTGATCTCAGTGCCTTGAATGCATATC |
| | | CCGAGAAAAGACTCCTCTTGCGCCACCCCAAAGGACTCT |
| | | GCCCTGCTTGCCGGCTCTTTGGAACCGGATCTTACAAGG |
| | | GCAGAGTCAGGTTTGGATTCGCGTCACTCGAAAACGATC |
| | | CGGAGTGGCTGATCCCAGGCAAGAATCCCGGCGATCCG |
| | | TTTCACGGCGGGCCGGTGATGCTCTCATTGTTGGAACGG |
| | | CCTCGCCCGACTTGGAGTATACCGGGATCCGACAATAAG |
| | | TTTAAAGTGCCTGGCAGAAAGTTTTACGTCCACCACCAC |
| | | GCCTGGAAAACCATTAAGGACGGGAACCATCCCACAAC |
| | | AGGCAAAGCTATTGAACAAAGCCCTAATAACCGCACTG |
| | | TAGAAGCTCTCGCCGGCGGGAATTCCTTTAGCTTCGAAA |
| | | TTGCCTTTGAGAACCTGAAAGAATGGGAGCTGGGTTTGC |
| | | TCATCCACAGCCTGCAACTCGAAAAGGGTCTGGCGCATA |
| | | AACTTGGAATGGCAAAGTCTATGGGATTTGGTTCAGTTG |
| | | AAATTGACGTCGAATCAGTGCGCCTGAGAAAAGATTGG |
| | | AAGCAATGGCGGAATGGCAATTCCGAAATTCCCAACTG |
| | | GTTGGGAAAAGGATTTGCTAAACTGAAGGAATGGTTCC |
| | | GGGACGAGCTCGATTTTATAGAAAATCTTAAGAAACTTC |
| | | TTTGGTTTCCTGAGGGCGACCAAGCACCCCGGGTTTGCT |
| | | ACCCCATGCTGCGAAAGAAGGACGATCCTAATGGGAAT |
| | | AGCGGTTACGAAGAACTCAAAAGAGGGGAATTCAAGAA |
| | | AGAAGATCGGCAGAAGAAGCTGACCACGCCGTGGACAC |
| | | CGTGGGCAaaacggacagccgacggaagcgagttcgagtcaccaaagaagaagcg |
| | | gaaagtctaa |
| 201 | Cas711_D1580R_ | ATGaaacggacagccgacggaagcgagttcgagtcaccaaagaagaagcggaaagtc |
| | D988K | ACGACTACTATGAAGATTTCAATTGAATTCCTCGAGCCG |
| | (pDF0506 based) | TTTCGGATGACCAAATGGCAGGAAAGCACTAGGCGAAA |
| | Doublemutant | CAAGAACAACAAGGAATTTGTTCGCGGCCAAGCTTTTGC |
| | | AAGATGGCATAGAAATAAGAAGGATAACACAAAAGGGC |
| | | GGCCTTACATAACTGGGACATTGCTGCGGTCCGCAGTAA |
| | | TTAGAAGCGCGGAAAACCTGCTGACATTGAGTGATGGG |
| | | AAAATTAGCGAAAAGACATGTTGTCCTGGGAAATTTGAT |
| | | ACGGAAGATAAAGACAGGTTGCTGCAATTGAGGCAGCG |
| | | GTCCACCCTTCGTTGGACCGATAAGAACCCCTGTCCAGA |
| | | TAACGCTGAAACTTACTGCCCGTTTTGCGAACTTCTCGG |
| | | GAGATCCGGTAACGATGGAAAGAAGGCTGAGAAGAAGG |
| | | ATTGGCGCTTTCGTATTCATTTTGGCAATCTTTCCCTTCC |
| | | AGGGAAGCCCGATTTCGACGGGCCAAAAGCTATAGGCA |
| | | GCCAACGGGTACTTAACAGGGTCGATTTCAAATCCGGTA |
| | | AGGCCCATGACTTCTTTAAGGCTTACGAAGTCGACCATA |
| | | CTAGGTTTCCCCGCTTCGAAGGGGAGATTACCATAGATA |
| | | ATAAAGTCAGCGCTGAAGCCAGGAAACTGCTCTGTGATT |
| | | CTCTCAAGTTTACGGATCGGCTGTGTGGAGCTCTGTGCG |
| | | TAATCAGGTTCGATGAATATACACCAGCAGCCGATAGTG |
| | | GGAAACAAACCGAAAATGTCCAAGCAGAACCGAATGCT |
| | | AATCTCGCTGAAAAGACCGCAGAGCAAATTATTAGCATT |
| | | CTGGACGATAATAAGAAAACCGAATATACCCGCCTGCTT |
| | | GCTGATGCTATTCGTTCACTCCGCCGCTCTAGTAAACTTG |
| | | TCGCTGGCTTGCCAAAGGATCATGACGGGAAGGATGAC |
| | | CATAAGCTCTGGGATATCGGCAAGAAGAAGAAAGACGA |
| | | AAACTCTGTCACTATTAGGCAAATCCTTACGACCTCAGC |
| | | AGACACCAAGGAACTCAAGAATGCCGGAAAATGGAGAG |
| | | AATTCTGCGAGAAGCTGGGTGAAGCGCTGTACCTCAAG |
| | | AGTAAAGACATGAGTGGCGGCCTGAAAATTACTCGGAG |
| | | AATACTGGGCGATGCTGAATTCCATGGAAAGCCCGATCG |
| | | GCTTGAAAAGAGCCGGTCTGTGTCAATCGGATCTGTGTT |
| | | GAAAGAAACTGTGGTATGCGGCGAACTGGTCGCTAAAA |
| | | CGCCCTTCTTCTTCGGAGCGATAGACGAAGATGCAAAAC |
| | | AAACCgacCTGCAAGTACTCCTCACTCCCGATAACAAGTA |
| | | TAGACTGCCAAGAAGCGCCGTGCGAGGTATACTCCGTCG |
| | | AGATCTTCAAACCTATTTTGATAGCCCATGCAATGCTGA |
| | | GTTGGGTGGACGGCCATGCATGTGTAAGACGTGTAGGAT |
| | | TATGAGAGGGATCACGGTGATGGATGCGCGCAGTGAGT |
| | | ACAATGCCCCGCCAGAAATAAGGCATCGTACCCGCATTA |
| | | ATCCCTTCACAGGCACGGTTGCGGAAGGTGCCCTGTTTA |
| | | ATATGGAAGTAGCCCCCGAGGGGATTGTCTTTCCATTTC |
| | | AACTCCGGTACCGGGGCTCTGAAGATGGGCTGCCCGATG |
| | | CACTGAAAACGGTGTTGAAATGGTGGGCTGAGGGGCAG |
| | | GCATTCATGAGTGGCGCTGCCTCAACCGGGAAGGGCCG |
| | | ATTCCGGATGGAAAATGCTAAATATGAAACGCTGGATCT |
| | | GAGCGACGAGAATCAAAGGAATGACTATCTTAAGAATT |
| | | GGGGATGGCGTGACGAGAAGGGGCTCGAGGAACTGAAG |
| | | AAACGACTGAACTCAGGTCTGCCAGAGCCCGGTAATTAT |
| | | AGGGATCCAAAATGGCACGAGATTAACGTTTCCATTGAG |
| | | ATGGCAAGCCCTTTTATTAATGGCGACCCAATCCGCGCA |
| | | GCCGTGGACAAACGTGGTACAgatGTGGTTACCTTCGTTA |
| | | AGTATAAAGCTGAAGGGGAAGAGGCGAAACCCGTATGT |
| | | GCATACAAGGCCGAATCTTTTAGAGGGGTGATCAGAAG |
| | | TGCCGTGGCACGCATTCATATGGAAGATGGCGTCCCTTT |
| | | GACTGAGTTGACTCACAGTGACTGTGAATGTCTCCTGTG |
| | | CCAAATCTTTGGAAGTGAGTATGAAGCCGGCAAAATAA |
| | | GGTTTGAAGATCTCGTATTCGAAAGTGACCCGGAACCTG |
| | | TGACCTTCGATCATGTGGCCATCGATAGATTCACTGGCG |
| | | GTGCAGCTGATAAGAAGAAATTCGATGATTCCCCTCTGC |
| | | CCGGTAGCCCTGCAAGACCGCTCATGTTGAAAGGCTCCT |
| | | TCTGGATCCGCAGGGACGTTCTCGAGGACGAAGAGTACT |
| | | GTAAGGCACTCGGTAAGGCTCTTGCAGATGTGAATAATG |
| | | GCCTTTATCCCCTCGGTGGAAAGAGCGCCATCGGCTACG |
| | | GACAGGTCAAGAGTCTGGGTATAAAGGGAGATGATAAG |
| | | AGGATTTCTCGCCTCATGAATCCTGCCTTTGATGAGACA |
| | | GATGTAGCCGTTCCAGAAAAGCCCAAAACTGATGCCGA |
| | | GGTTCGCATCGAGGCAGAGAAAGTATATTACCCACACTA |
| | | TTTCGTCGAACCCCATAAGAAGGTGGAACGCGAGGAGA |
| | | AACCCTGTGGTCATCAAAAGTTCCACGAGGGGCGACTG |
| | | ACAGGTAAAATTCGGTGTAAGCTCATTACCAAGACACCC |
| | | CTCATCGTCCCAGATACTAGTAATGACGATTTCTTCAGA |
| | | CCTGCGGATAAAGAAGCTCGGAAGGAAAAGGACGAATA |
| | | TCATAAATCATATGCTTTCTTCAGACTTCATAAACAAAT |
| | | CATGATTCCCGGGAGCGAATTGAGAGGAATGGTGAGTA |
| | | GTGTCTACGAAACTGTGACAAATTCTTGCTTCAGGATAT |
| | | TTGATGAGACTAAACGGTTGTCATGGCGGATGGATGCTG |
| | | ATCACCAGAATGTGCTGCAAAAGTTTCTCCCAGGTCGAG |
| | | TGACCGCCGATGGGAAACATATACAAAAGTTTTCCGAA |
| | | ACTGCAAGGGTGCCTTTCTATGACAAAACGCAGAAACAT |
| | | TTTGACATTCTTGATGAGCAAGAAATTGCTGGTGAGAAA |
| | | CCTGTTCGGATGTGGGTCAAGCGTTTTATTAAACGACTG |
| | | AGCCTCGTTGATCCTGCTAAACACCCCCAGAAGAAACAA |
| | | GACAATAAATGGAAAAGACGCAAAGAAGGCATCGCCAC |
| | | ATTTATAGAGCAAAAGAATGGCTCTTATTATTTTAACGT |
| | | GGTCACCAATAATGGCTGCACTTCTTTCCACTTGTGGCA |
| | | TAAACCTGACAATTTTGACCAGGAGAAACTCGAAGGCA |
| | | TACAGAATGGTGAGAAACTGGATTGCTGGGTAAGAGAT |
| | | AGTAGATACCAAAAGGCCTTTCAAGAGATACCCGAGAA |
| | | CGACCCAGACGGATGGGAGTGTAAAGAGGGCTACCTTC |
| | | ATGTCGTCGGCCCCAGCAAAGTAGAGTTTAGTGACAAG |
| | | AAAGGGGATGTGATCAATAACTTTCAAGGAACACTCCC |
| | | ATCAGTTCCCAACGACTGGAAAACAATTAGGACGAACG |
| | | ATTTTAAGAATAGGAAAAGGAAGAATGAACCTGTGTTTT |
| | | GTTGTGAGGATGATAAGGGCAATTACTATACCATGGCTA |
| | | AATATTGCGAAACCTTCTTCTTCGATCTGAAGGAGAACG |
| | | AGGAATACGAGATCCCCGAGAAAGCAAGAATCAAATAC |
| | | AAAGAACTGCTCAGGGTCTATAACAACAATCCTCAAGC |
| | | AGTGCCGGAGAGCGTATTTCAGTCTAGAGTTGCCCGGGA |
| | | AAACGTGGAAAAGCTGAAGTCCGGAGATCTTGTGTATTT |
| | | CAAACATAATGAAAAGTACGTAGAGGACATCGTCCCAG |
| | | TGCGGATTTCCCGAACTGTAGACGATAGGATGATCGGCA |
| | | AACGTATGAGCGCCGATCTGCGGCCGTGCCATGGAGATT |
| | | GGGTGGAAGATGGTGATCTCAGTGCCTTGAATGCATATC |
| | | CCGAGAAAAGACTCCTCTTGCGCCACCCCAAAGGACTCT |
| | | GCCCTGCTTGCCGGCTCTTTGGAACCGGATCTTACAAGG |
| | | GCAGAGTCAGGTTTGGATTCGCGTCACTCGAAAACGATC |
| | | CGGAGTGGCTGATCCCAGGCAAGAATCCCGGCGATCCG |
| | | TTTCACGGCGGGCCGGTGATGCTCTCATTGTTGGAACGG |
| | | CCTCGCCCGACTTGGAGTATACCGGGATCCGACAATAAG |
| | | TTTAAAGTGCCTGGCAGAAAGTTTTACGTCCACCACCAC |
| | | GCCTGGAAAACCATTAAGGACGGGAACCATCCCACAAC |
| | | AGGCAAAGCTATTGAACAAAGCCCTAATAACCGCACTG |
| | | TAGAAGCTCTCGCCGGCGGGAATTCCTTTAGCTTCGAAA |
| | | TTGCCTTTGAGAACCTGAAAGAATGGGAGCTGGGTTTGC |
| | | TCATCCACAGCCTGCAACTCGAAAAGGGTCTGGCGCATA |
| | | AACTTGGAATGGCAAAGTCTATGGGATTTGGTTCAGTTG |
| | | AAATTGACGTCGAATCAGTGCGCCTGAGAAAAGATTGG |
| | | AAGCAATGGCGGAATGGCAATTCCGAAATTCCCAACTG |
| | | GTTGGGAAAAGGATTTGCTAAACTGAAGGAATGGTTCC |
| | | GGGACGAGCTCGATTTTATAGAAAATCTTAAGAAACTTC |
| | | TTTGGTTTCCTGAGGGCGACCAAGCACCCCGGGTTTGCT |
| | | ACCCCATGCTGCGAAAGAAGGACGATCCTAATGGGAAT |
| | | AGCGGTTACGAAGAACTCAAAAGAGGGGAATTCAAGAA |
| | | AGAAGATCGGCAGAAGAAGCTGACCACGCCGTGGACAC |
| | | CGTGGGCAaaacggacagccgacggaagcgagttcgagtcaccaaagaagaagcg |
| | | gaaagtctaa |
| 202 | Cas711_D1580R_ | ATGaaacggacagccgacggaagcgagttcgagtcaccaaagaagaagcggaaagtc |
| | D988K_D981K | ACGACTACTATGAAGATTTCAATTGAATTCCTCGAGCCG |
| | (pDF0506 based) | TTTCGGATGACCAAATGGCAGGAAAGCACTAGGCGAAA |
| | Triplemutant | CAAGAACAACAAGGAATTTGTTCGCGGCCAAGCTTTTGC |
| | | AAGATGGCATAGAAATAAGAAGGATAACACAAAAGGGC |
| | | GGCCTTACATAACTGGGACATTGCTGCGGTCCGCAGTAA |
| | | TTAGAAGCGCGGAAAACCTGCTGACATTGAGTGATGGG |
| | | AAAATTAGCGAAAAGACATGTTGTCCTGGGAAATTTGAT |
| | | ACGGAAGATAAAGACAGGTTGCTGCAATTGAGGCAGCG |
| | | GTCCACCCTTCGTTGGACCGATAAGAACCCCTGTCCAGA |
| | | TAACGCTGAAACTTACTGCCCGTTTTGCGAACTTCTCGG |
| | | GAGATCCGGTAACGATGGAAAGAAGGCTGAGAAGAAGG |
| | | ATTGGCGCTTTCGTATTCATTTTGGCAATCTTTCCCTTCC |
| | | AGGGAAGCCCGATTTCGACGGGCCAAAAGCTATAGGCA |
| | | GCCAACGGGTACTTAACAGGGTCGATTTCAAATCCGGTA |
| | | AGGCCCATGACTTCTTTAAGGCTTACGAAGTCGACCATA |
| | | CTAGGTTTCCCCGCTTCGAAGGGGAGATTACCATAGATA |
| | | ATAAAGTCAGCGCTGAAGCCAGGAAACTGCTCTGTGATT |
| | | CTCTCAAGTTTACGGATCGGCTGTGTGGAGCTCTGTGCG |
| | | TAATCAGGTTCGATGAATATACACCAGCAGCCGATAGTG |
| | | GGAAACAAACCGAAAATGTCCAAGCAGAACCGAATGCT |
| | | AATCTCGCTGAAAAGACCGCAGAGCAAATTATTAGCATT |
| | | CTGGACGATAATAAGAAAACCGAATATACCCGCCTGCTT |
| | | GCTGATGCTATTCGTTCACTCCGCCGCTCTAGTAAACTTG |
| | | TCGCTGGCTTGCCAAAGGATCATGACGGGAAGGATGAC |
| | | CATAAGCTCTGGGATATCGGCAAGAAGAAGAAAGACGA |
| | | AAACTCTGTCACTATTAGGCAAATCCTTACGACCTCAGC |
| | | AGACACCAAGGAACTCAAGAATGCCGGAAAATGGAGAG |
| | | AATTCTGCGAGAAGCTGGGTGAAGCGCTGTACCTCAAG |
| | | AGTAAAGACATGAGTGGCGGCCTGAAAATTACTCGGAG |
| | | AATACTGGGCGATGCTGAATTCCATGGAAAGCCCGATCG |
| | | GCTTGAAAAGAGCCGGTCTGTGTCAATCGGATCTGTGTT |
| | | GAAAGAAACTGTGGTATGCGGCGAACTGGTCGCTAAAA |
| | | CGCCCTTCTTCTTCGGAGCGATAGACGAAGATGCAAAAC |
| | | AAACCgacCTGCAAGTACTCCTCACTCCCGATAACAAGTA |
| | | TAGACTGCCAAGAAGCGCCGTGCGAGGTATACTCCGTCG |
| | | AGATCTTCAAACCTATTTTGATAGCCCATGCAATGCTGA |
| | | GTTGGGTGGACGGCCATGCATGTGTAAGACGTGTAGGAT |
| | | TATGAGAGGGATCACGGTGATGGATGCGCGCAGTGAGT |
| | | ACAATGCCCCGCCAGAAATAAGGCATCGTACCCGCATTA |
| | | ATCCCTTCACAGGCACGGTTGCGGAAGGTGCCCTGTTTA |
| | | ATATGGAAGTAGCCCCCGAGGGGATTGTCTTTCCATTTC |
| | | AACTCCGGTACCGGGGCTCTGAAGATGGGCTGCCCGATG |
| | | CACTGAAAACGGTGTTGAAATGGTGGGCTGAGGGGCAG |
| | | GCATTCATGAGTGGCGCTGCCTCAACCGGGAAGGGCCG |
| | | ATTCCGGATGGAAAATGCTAAATATGAAACGCTGGATCT |
| | | GAGCGACGAGAATCAAAGGAATGACTATCTTAAGAATT |
| | | GGGGATGGCGTGACGAGAAGGGGCTCGAGGAACTGAAG |
| | | AAACGACTGAACTCAGGTCTGCCAGAGCCCGGTAATTAT |
| | | AGGGATCCAAAATGGCACGAGATTAACGTTTCCATTGAG |
| | | ATGGCAAGCCCTTTTATTAATGGCGACCCAATCCGCGCA |
| | | GCCGTGGACAAACGTGGTACAgatGTGGTTACCTTCGTTA |
| | | AGTATAAAGCTGAAGGGGAAGAGGCGAAACCCGTATGT |
| | | GCATACAAGGCCGAATCTTTTAGAGGGGTGATCAGAAG |
| | | TGCCGTGGCACGCATTCATATGGAAGATGGCGTCCCTTT |
| | | GACTGAGTTGACTCACAGTGACTGTGAATGTCTCCTGTG |
| | | CCAAATCTTTGGAAGTGAGTATGAAGCCGGCAAAATAA |
| | | GGTTTGAAGATCTCGTATTCGAAAGTGACCCGGAACCTG |
| | | TGACCTTCGATCATGTGGCCATCGATAGATTCACTGGCG |
| | | GTGCAGCTGATAAGAAGAAATTCGATGATTCCCCTCTGC |
| | | CCGGTAGCCCTGCAAGACCGCTCATGTTGAAAGGCTCCT |
| | | TCTGGATCCGCAGGGACGTTCTCGAGGACGAAGAGTACT |
| | | GTAAGGCACTCGGTAAGGCTCTTGCAGATGTGAATAATG |
| | | GCCTTTATCCCCTCGGTGGAAAGAGCGCCATCGGCTACG |
| | | GACAGGTCAAGAGTCTGGGTATAAAGGGAGATGATAAG |
| | | AGGATTTCTCGCCTCATGAATCCTGCCTTTGATGAGACA |
| | | GATGTAGCCGTTCCAGAAAAGCCCAAAACTGATGCCGA |
| | | GGTTCGCATCGAGGCAGAGAAAGTATATTACCCACACTA |
| | | TTTCGTCGAACCCCATAAGAAGGTGGAACGCGAGGAGA |
| | | AACCCTGTGGTCATCAAAAGTTCCACGAGGGGCGACTG |
| | | ACAGGTAAAATTCGGTGTAAGCTCATTACCAAGACACCC |
| | | CTCATCGTCCCAGATACTAGTAATGACGATTTCTTCAGA |
| | | CCTGCGGATAAAGAAGCTCGGAAGGAAAAGGACGAATA |
| | | TCATAAATCATATGCTTTCTTCAGACTTCATAAACAAAT |
| | | CATGATTCCCGGGAGCGAATTGAGAGGAATGGTGAGTA |
| | | GTGTCTACGAAACTGTGACAAATTCTTGCTTCAGGATAT |
| | | TTGATGAGACTAAACGGTTGTCATGGCGGATGGATGCTG |
| | | ATCACCAGAATGTGCTGCAAAAGTTTCTCCCAGGTCGAG |
| | | TGACCGCCGATGGGAAACATATACAAAAGTTTTCCgggggt |
| | | gggtcaCGAACTGTAGACGATAGGATGATCGGCAAACGTA |
| | | TGAGCGCCGATCTGCGGCCGTGCCATGGAGATTGGGTGG |
| | | AAGATGGTGATCTCAGTGCCTTGAATGCATATCCCGAGA |
| | | AAAGACTCCTCTTGCGCCACCCCAAAGGACTCTGCCCTG |
| | | CTTGCCGGCTCTTTGGAACCGGATCTTACAAGGGCAGAG |
| | | TCAGGTTTGGATTCGCGTCACTCGAAAACGATCCGGAGT |
| | | GGCTGATCCCAGGCAAGAATCCCGGCGATCCGTTTCACG |
| | | GCGGGCCGGTGATGCTCTCATTGTTGGAACGGCCTCGCC |
| | | CGACTTGGAGTATACCGGGATCCGACAATAAGTTTAAAG |
| | | TGCCTGGCAGAAAGTTTTACGTCCACCACCACGCCTGGA |
| | | AAACCATTAAGGACGGGAACCATCCCACAACAGGCAAA |
| | | GCTATTGAACAAAGCCCTAATAACCGCACTGTAGAAGCT |
| | | CTCGCCGGCGGGAATTCCTTTAGCTTCGAAATTGCCTTT |
| | | GAGAACCTGAAAGAATGGGAGCTGGGTTTGCTCATCCA |
| | | CAGCCTGCAACTCGAAAAGGGTCTGGCGCATAAACTTG |
| | | GAATGGCAAAGTCTATGGGATTTGGTTCAGTTGAAATTG |
| | | ACGTCGAATCAGTGCGCCTGAGAAAAGATTGGAAGCAA |
| | | TGGCGGAATGGCAATTCCGAAATTCCCAACTGGTTGGGA |
| | | AAAGGATTTGCTAAACTGAAGGAATGGTTCCGGGACGA |
| | | GCTCGATTTTATAGAAAATCTTAAGAAACTTCTTTGGTTT |
| | | CCTGAGGGCGACCAAGCACCCCGGGTTTGCTACCCCATG |
| | | CTGCGAAAGAAGGACGATCCTAATGGGAATAGCGGTTA |
| | | CGAAGAACTCAAAAGAGGGGAATTCAAGAAAGAAGATC |
| | | GGCAGAAGAAGCTGACCACGCCGTGGACACCGTGGGCA |
| | | aaacggacagccgacggaagcgagttcgagtcaccaaagaagaagcggaaagtctaa |

Table 12 shows DNA sequences used as linkers.

| SEQ | | |
|---|---|---|
| ID NO | ID | Sequence |
| 203 | XTEN | tctggcagcgagacaccaggaacaagcgagtcag |
| | | caacaccagagagc |
| 204 | GS | ggatccggtgggtccggtagtggtggttccgggt |
| | | ccggtggaagt |

Table 13 shows guide sequences.

| SEQ | | |
|---|---|---|
| ID | | |
| NO | ID | Sequence |
| 91 | pDF0866 STAT3 | GTTGATGTCACGGAACagaaaatataaagtttctgaggagaattcaa |
| | guide 4 | |
| 205 | pDF0222 NT guide | GTTGATGTCACGGAACggtaatgcctggcttgtcgacgcatagtctg |
| | (for all sequences | |
| 206 | Lwa Guide 1 | ATTTAGACTACCCCAAAAACGAAGGGGACTtggctcacgcctgtaatg |
| | Cas13 | ccagcactttgag |
| 207 | Lwa Guide 2 | ATTTAGACTACCCCAAAAACGAAGGGGACTgtatccccaagagaaggc |
| | Cas13 | tccctgttggcca |
| 208 | Lwa Guide 3 | ATTTAGACTACCCCAAAAACGAAGGGGACTcaaaacctcaaaaaagat |
| | Cas13 | acatgcaggacct |
| 209 | Lwa Guide 4 | ATTTAGACTACCCCAAAAACGAAGGGGACTagaaaatataaagtttct |
| | Cas13 | gaggagaattcaa |
| 210 | Lwa Guide 5 | ATTTAGACTACCCCAAAAACGAAGGGGACTacaaaaaaacagaagtaa |
| | Cas13 | agaaagatttcct |
| 211 | Lwa Guide NT | ATTTAGACTACCCCAAAAACGAAGGGGACTggtccgctgccgttcgct |
| | Cas13 | tgggacatcctgt |
| 212 | Psp Guide 1 Cas13 | GTTGTGGAAGGTCCAGTTTTGAGGGGCTATtggctcacgcctgtaatg |
| | | ccagcactttgag |
| 213 | Psp Guide 2 Cas13 | GTTGTGGAAGGTCCAGTTTTGAGGGGCTATgtatccccaagagaaggc |
| | | tccctgttggcca |
| 214 | Psp Guide 3 Cas13 | GTTGTGGAAGGTCCAGTTTTGAGGGGCTATcaaaacctcaaaaaagat |
| | | acatgcaggacct |
| 215 | Psp Guide 4 Cas13 | GTTGTGGAAGGTCCAGTTTTGAGGGGCTATagaaaatataaagtttct |
| | | gaggagaattcaa |
| 216 | Psp Guide 5 Cas13 | GTTGTGGAAGGTCCAGTTTTGAGGGGCTATacaaaaaaacagaagtaa |
| | | agaaagatttcct |
| 217 | Psp Guide NT | GTTGTGGAAGGTCCAGTTTTGAGGGGCTATggtccgctgccgttcgct |
| | Cas13 | tgggacatcctgt |
| 218 | Rfx Guide 1 Cas13 | AACCCCTACCAACTGGTCGGGGTTTGAAACtggctcacgcctgtaatg |
| | | ccagcactttgag |
| 219 | Rfx Guide 2 Cas13 | AACCCCTACCAACTGGTCGGGGTTTGAAACgtatccccaagagaaggc |
| | | tccctgttggcca |
| 220 | Rfx Guide 3 Cas13 | AACCCCTACCAACTGGTCGGGGTTTGAAACcaaaacctcaaaaaagat |
| | | acatgcaggacct |
| 221 | Rfx Guide 4 Cas13 | AACCCCTACCAACTGGTCGGGGTTTGAAACagaaaatataaagtttct |
| | | gaggagaattcaa |
| 222 | Rfx Guide 5 Cas13 | AACCCCTACCAACTGGTCGGGGTTTGAAACacaaaaaaacagaagtaa |
| | | agaaagatttcct |
| 223 | Rfx Guide NT | AACCCCTACCAACTGGTCGGGGTTTGAAACggtccgctgccgttcgct |
| | Cas13 | tgggacatcctgt |
| 224 | PABPC1 targeting | GTTGATGTCACGGAACgtttcttccctcaaatgaaagtataaattgt |
| | guide | |
| 225 | pDF0868 PPIB | GTTGATGTCACGGAACgccaagggtgaggaggaggaagagggtgacc |
| | guide 4 | |
| 226 | TOP2A targeting | GTTGATGTCACGGAACtttaacaatatttattgagcacttgctatgt |
| | guide | |
| 227 | SHANK3 guide h | GTTGATGTCACGGAACaggcgccgggttggcaagtgggcagggaaca |
| 228 | PABPC1_5TS_guide_1 | GTTGATGTCACGGAACagtgtgtgatacttgaaaggtctagccatct |
| 229 | PABPC1_5TS_guide_2 | GTTGATGTCACGGAACctttatacaacttaggtcccacactagtgtg |
| 230 | PABPC1_5TS_guide_3 | GTTGATGTCACGGAACcgggggcttctggtatttgtctttgctttat |
| 231 | PABPC1_5TS_guide_4 | GTTGATGTCACGGAACgaattcttttatatgtgagaaatttcggggg |
| 232 | PPIB_5TS_guide_1 | GTTGATGTCACGGAACctcataggatttttaccgtcaccaaaatcag |
| 233 | PPIB_5TS_guide_2 | GTTGATGTCACGGAACgaaaagggtctggagctttcattagattctc |
| 234 | PPIB_5TS_guide_3 | GTTGATGTCACGGAACgggtatagataagcatgttttccaagaaaag |
| 235 | PPIB_5TS_guide_4 | GTTGATGTCACGGAACatattgttatcctgtagtccaaggagggtat |
| 236 | RPL41_5TS_guide_1 | GTTGATGTCACGGAACgaaaaacgtttgagtgttttctccctggagc |
| 237 | RPL41_5TS_guide_2 | GTTGATGTCACGGAACgaaatcttaaaagatctttaggagaaaaacg |
| 238 | RPL41_5TS_guide_3 | GTTGATGTCACGGAACcaaaacttaggagaaacatttggtttggaaa |
| 239 | RPL41_5TS_guide_4 | GTTGATGTCACGGAACcccaggaggagggaagttccttggacaaaac |
| 224 | pDF0874_PABPC1_ | GTTGATGTCACGGAACgtttcttccctcaaatgaaagtataaattgt |
| | 3TS_guide3 | |
| | (pDF0114 based) | |
| 240 | pDF0909 USF1 | GTTGATGTCACGGAACccaaaagtaggttcacactttggacctcatt |
| | guide | |
| 241 | AAV T single | GTTGATGTCACGGAACtgacagccattggtgaactcgtgccctgtgc |
| | vector HTT | |
| 205 | AAV NT single | GTTGATGTCACGGAACggtaatgcctggcttgtcgacgcatagtctg |
| | vector HTT | |
| 242 | AAV T single | GTTGATGTCACGGAACagaaggcgccgggttggcaagtgggcaggga |
| | vector SHANK3 | |
| 205 | AAV NT single | GTTGATGTCACGGAACggtaatgcctggcttgtcgacgcatagtctg |
| | vector SHANK3 | |
| 243 | SHANK3_guide_v2_1 | GTTGATGTCACGGAACgagcctagaaggcgccgggttggcaagtggg |
| 244 | SHANK3_guide_v2_2 | GTTGATGTCACGGAACcctagaaggcgccgggttggcaagtgggcag |
| 242 | SHANK3_guide_v2_3 | GTTGATGTCACGGAACagaaggcgccgggttggcaagtgggcaggga |
| 227 | SHANK3_guide_h | GTTGATGTCACGGAACaggcgccgggttggcaagtgggcagggaaca |
| 245 | SHANK3_guide_v2_4 | GTTGATGTCACGGAACcgccgggttggcaagtgggcagggaacagag |
| 246 | SHANK3_guide_v2_5 | GTTGATGTCACGGAACcgggttggcaagtgggcagggaacagagaca |
| 247 | SHANK3_guide_v2_6 | GTTGATGTCACGGAACgttggcaagtgggcagggaacagagacatgc |
| 248 | HTT_guide_2 | GTTGATGTCACGGAACcatggagtataacggtttattcatagtagtc |
| 249 | HTT_guide_v2_1 | GTTGATGTCACGGAACaccgttatactccatgttgcgggcagaatgg |
| 250 | HTT_guide_v2_2 | GTTGATGTCACGGAACtgttgcgggcagaatggggatctggacaggg |
| 251 | HTT_guide_v2_3 | GTTGATGTCACGGAACgatctggacagggaagcacagggcacgagtt |
| 241 | pDF0944 | GTTGATGTCACGGAACtgacagccattggtgaactcgtgccctgtgc |
| | HTT_guide_3 | |
| 252 | HTT_guide_v2_4 | GTTGATGTCACGGAACgagttcaccaatggctgtcaagctacgctgc |
| 253 | HTT_guide_v2_5 | GTTGATGTCACGGAACctgtcaagctacgctgctcacagaaaaaaca |
| 254 | HTT_guide_v2_6 | GTTGATGTCACGGAACctgctcacagaaaaaacagatgatgttacta |
| 255 | HTT_guide_4 | GTTGATGTCACGGAACtaaaatgggggaaatgaactgctttagtaac |
| 91 | Guides on Cargo | GTTGATGTCACGGAACagaaaatataaagtttctgaggagaattcaa |
| | plasmid STAT3 | |
| 225 | Guides on Cargo | GTTGATGTCACGGAACgccaagggtgaggaggaggaagagggtgacc |
| | plasmid PPIB | |
| 242 | SHANK3 g and c | GTTGATGTCACGGAACagaaggcgccgggttggcaagtgggcaggga |
| | lenti | |
| 256 | pDF0987_RPL41_ | GTTGATGTCACGGAACtccctcccacattaaatcaaacgtccacata |
| | Guide2 | |
| 241 | HTT g and c lenti | GTTGATGTCACGGAACtgacagccattggtgaactcgtgccctgtgc |
| 241 | HTT_guide3_cargo13_ | GTTGATGTCACGGAACtgacagccattggtgaactcgtgccctgtgc |
| | single vector | |
| | aRY1599 A1-3-5-7- | |
| | 10-11-12 | |
| 257 | 5ts and its | GTTGATGTCACGGAACctttactctgcaagataaggtcaacaaaatg |
| | USF1_g1 | |
| 258 | 5ts and its | GTTGATGTCACGGAACagaactaggatttcagatacccagcttgctt |
| | USF1_g2 | |
| 259 | 5ts and its | GTTGATGTCACGGAACggttcacactttggacctcattttcatctaa |
| | USF1_g3 | |
| 260 | 5ts and its | GTTGATGTCACGGAACgatacaggaacctcagggagagataagacta |
| | USF1_g4 | |
| 261 | 5ts and its | GTTGATGTCACGGAACaataccaggaggcagaattcaggcatcctgc |
| | USF1_g5 | |
| 205 | 5ts and its | GTTGATGTCACGGAACggtaatgcctggcttgtcgacgcatagtctg |
| | USF1_g6 | |

Table 14 shows cargo sequences.

| SEQ | | | |
|---|---|---|---|
| ID NO | ID | Sequence | Length |
| 262 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsAT | ctgcaggTtctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 263 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsAC | ctgcaggCtctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 264 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsAG | ctgcaggGtctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 265 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsTA | ctgcaggaActagaacagaaaatgaaagtggtagagaatctccaggatgactttgat | |
| | | ttcaactataaaaccctcaagagtcaaggaGACTACAAAGACGATGACGAC | |
| | | AAGTAA | |
| 266 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsTC | ctgcaggaCctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 267 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsTG | ctgcaggaGctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 268 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsCT | ctgcaggatTtagaacagaaaatgaaagtggtagagaatctccaggatgactttgat | |
| | | ttcaactataaaaccctcaagagtcaaggaGACTACAAAGACGATGACGAC | |
| | | AAGTAA | |
| 269 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsCG | ctgcaggatGtagaacagaaaatgaaagtggtagagaatctccaggatgactttgat | |
| | | ttcaactataaaaccctcaagagtcaaggaGACTACAAAGACGATGACGAC | |
| | | AAGTAA | |
| 270 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsCA | ctgcaggatAtagaacagaaaatgaaagtggtagagaatctccaggatgactttgat | |
| | | ttcaactataaaaccctcaagagtcaaggaGACTACAAAGACGATGACGAC | |
| | | AAGTAA | |
| 271 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsGA | ctgcagAatctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 272 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsGT | ctgcagTatctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 273 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | subsGC | ctgcagCatctagaacagaaaatgaaagtggtagagaatctccaggatgactttgat | |
| | | ttcaactataaaaccctcaagagtcaaggaGACTACAAAGACGATGACGAC | |
| | | AAGTAA | |
| 274 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | 1aachange | ctgcaggatAAGgaacagaaaatgaaagtggtagagaatctccaggatgactttga | |
| | | tttcaactataaaaccctcaagagtcaaggaGACTACAAAGACGATGACGA | |
| | | CAAGTAA | |
| 275 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | 2aachange | ctgcaggatAAGTCAcagaaaatgaaagtggtagagaatctccaggatgactttg | |
| | | atttcaactataaaaccctcaagagtcaaggaGACTACAAAGACGATGACG | |
| | | ACAAGTAA | |
| 276 | STAT3_3TS_ | gaacagaaaatataaagtttctgaggagaattcaaatgaagccaaaacctcaaaaa | 288 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | 3aachange | ctgcaggatAAGTCAGCAaaaatgaaagtggtagagaatctccaggatgactttg | |
| | | atttcaactataaaaccctcaagagtcaaggaGACTACAAAGACGATGACG | |
| | | ACAAGTAA | |
| 277 | STAT3_3TS_ | gaacagaaaatataaagtttctgaggagaattcaaatgaagccaaaacctcaaaaa | 289 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | frameshiftcorr1bp | ctgcaggaAtctagaacagaaaatgaaagtggtagagaatctccaggatgactttga | |
| | | tttcaactataaaaccctcaagagtcaaggaGACTACAAAGACGATGACGA | |
| | | CAAGTAA | |
| 278 | STAT3_3TS_ | gaacagaaaatataaagtttctgaggagaattcaaatgaagccaaaacctcaaaaa | 290 |
| | CARGO2_ | agatacatgcaggacctgcaggcagtatccccaagagaaggctccctgttggccagg | |
| | primelike_ | tgcagtggctcacgcctgtaatgccagcactttgagaacgaggaattctcttctttttttt | |
| | frameshiftcorr2bp | ctgcaggaAGtctagaacagaaaatgaaagtggtagagaatctccaggatgacttt | |
| | | gatttcaactataaaaccctcaagagtcaaggaGACTACAAAGACGATGAC | |
| | | GACAAGTAA | |
| 279 | STAT3_3TS_ | gaacagaaaatataaagtttctgaggagaattcaaatgaagccaaaacctcaaaaa | 294 |
| | CARGO2_ | agatacatgcaggacctgcaggcagtatccccaagagaaggctccctgttggccagg | |
| | primelike_6ins | tgcagtggctcacgcctgtaatgccagcactttgagaacgaggaattctcttctttttttt | |
| | | ctgcaggatTTGCACctagaacagaaaatgaaagtggtagagaatctccaggatg | |
| | | actttgatttcaactataaaaccctcaagagtcaaggaGACTACAAAGACGAT | |
| | | GACGACAAGTAA | |
| 280 | STAT3_3TS_ | gaacagaaaatataaagtttctgaggagaattcaaatgaagccaaaacctcaaaaa | 300 |
| | CARGO2_ | agatacatgcaggacctgcaggcagtatccccaagagaaggctccctgttggccagg | |
| | primelike_12ins | tgcagtggctcacgcctgtaatgccagcactttgagaacgaggaattctcttctttttttt | |
| | | ctgcaggatTTGCACATTGCGctagaacagaaaatgaaagtggtagagaatctc | |
| | | caggatgactttgatttcaactataaaaccctcaagagtcaaggaGACTACAAAG | |
| | | ACGATGACGACAAGTAA | |
| 281 | STAT3_3TS_ | gaacagaaaatataaagtttctgaggagaattcaaatgaagccaaaacctcaaaaa | 312 |
| | CARGO2_ | agatacatgcaggacctgcaggcagtatccccaagagaaggctccctgttggccagg | |
| | primelike_24ins | tgcagtggctcacgcctgtaatgccagcactttgagaacgaggaattctcttctttttttt | |
| | | ctgcaggatTTGCACATTGCGTCAACTCATAAGctagaacagaaaatgaa | |
| | | agtggtagagaatctccaggatgactttgatttcaactataaaaccctcaagagtcaa | |
| | | ggaGACTACAAAGACGATGACGACAAGTAA | |
| 282 | STAT3_3TS_ | gaacagaaaatataaagtttctgaggagaattcaaatgaagccaaaacctcaaaaa | 336 |
| | CARGO2_ | agatacatgcaggacctgcaggcagtatccccaagagaaggctccctgttggccagg | |
| | primelike_48ins | tgcagtggctcacgcctgtaatgccagcactttgagaacgaggaattctcttctttttttt | |
| | | ctgcaggatTTGCACATTGCGTCAACTCATAAGATGTCTCAACGGCA | |
| | | TGCGCAACTTctagaacagaaaatgaaagtggtagagaatctccaggatgacttt | |
| | | gatttcaactataaaaccctcaagagtcaaggaGACTACAAAGACGATGAC | |
| | | GACAAGTAA | |
| 283 | STAT3_3TS_ | gaacagaaaatataaagtttctgaggagaattcaaatgaagccaaaacctcaaaaa | 384 |
| | CARGO2_ | agatacatgcaggacctgcaggcagtatccccaagagaaggctccctgttggccagg | |
| | primelike_96ins | tgcagtggctcacgcctgtaatgccagcactttgagaacgaggaattctcttctttttttt | |
| | | ctgcaggatTTGCACATTGCGTCAACTCATAAGATGTCTCAACGGCA | |
| | | TGCGCAACTTGTGAAGTGTCTACTATCCTTAAACGCATATCTCGC | |
| | | ACAGTATCTCCCGctagaacagaaaatgaaagtggtagagaatctccaggatg | |
| | | actttgatttcaactataaaaccctcaagagtcaaggaGACTACAAAGACGAT | |
| | | GACGACAAGTAA | |
| 284 | STAT3_3TS_ | gaacagaaaatataaagtttctgaggagaattcaaatgaagccaaaacctcaaaaa | 276 |
| | CARGO2_ | agatacatgcaggacctgcaggcagtatccccaagagaaggctccctgttggccagg | |
| | primelike_12del | tgcagtggctcacgcctgtaatgccagcactttgagaacgaggaattctcttctttttttt | |
| | | ctgcagaaaatgaaagtggtagagaatctccaggatgactttgatttcaactataaaa | |
| | | ccctcaagagtcaaggaGACTACAAAGACGATGACGACAAGTAA | |
| 285 | STAT3_primelike_ | gaacagaaaatataaagtttctgaggagaattcaaatgaagccaaaacctcaaaaa | 288 |
| | v2_v10 | agatacatgcaggacctgcaggcagtatccccaagagaaggctccctgttggccagg | |
| | | tgcagtggctcacgcctgtaatgccagcactttgagaacgaggaattctcttctttttttt | |
| | | ctgcaggatctaAaacagaaaatgaaagtggtagagaatctccaggatgactttgat | |
| | | ttcaactataaaaccctcaagagtcaaggaGACTACAAAGACGATGACGAC | |
| | | AAGTAA | |
| 286 | STAT3_primelike_ | gaacagaaaatataaagtttctgaggagaattcaaatgaagccaaaacctcaaaaa | 288 |
| | v2_v11 | agatacatgcaggacctgcaggcagtatccccaagagaaggctccctgttggccagg | |
| | | tgcagtggctcacgcctgtaatgccagcactttgagaacgaggaattctcttctttttttt | |
| | | ctgcaggatctaTaacagaaaatgaaagtggtagagaatctccaggatgactttgat | |
| | | ttcaactataaaaccctcaagagtcaaggaGACTACAAAGACGATGACGAC | |
| | | AAGTAA | |
| 287 | STAT3_primelike_ | gaacagaaaatataaagtttctgaggagaattcaaatgaagccaaaacctcaaaaa | 288 |
| | v2_v12 | agatacatgcaggacctgcaggcagtatccccaagagaaggctccctgttggccagg | |
| | | tgcagtggctcacgcctgtaatgccagcactttgagaacgaggaattctcttctttttttt | |
| | | ctgcaggatctaCaacagaaaatgaaagtggtagagaatctccaggatgactttgat | |
| | | ttcaactataaaaccctcaagagtcaaggaGACTACAAAGACGATGACGAC | |
| | | AAGTAA | |
| 288 | PPIB_primelike_ | atgtggcttctcagggacattgcgttcagctgcactctgtatacctcaggggtgggacc | 328 |
| | v1 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcagAaggtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 289 | PPIB_primelike_ | atgtggcttctcagggacattgcgttcagctgcactctgtatacctcaggggtgggacc | 328 |
| | v2 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcagTaggtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 290 | PPIB_primelike_ | atgtggcttctcagggacattgcgttcagctgcactctgtatacctcaggggtgggacc | 328 |
| | v3 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcagCaggtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 291 | PPIB_primelike_ | atgtggcttctcagggacattgcgttcagctgcactctgtatacctcaggggtgggacc | 328 |
| | v4 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggTggtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 292 | PPIB_primelike_ | atgtggcttctcagggacattgcgttcagctgcactctgtatacctcaggggtgggacc | 328 |
| | v5 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggCggtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 293 | PPIB_primelike_ | atgtggcttctcagggacattgcgttcagctgcactctgtatacctcaggggtgggacc | 328 |
| | v6 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggGggtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 294 | PPIB_primelike_ | atgtggcttctcagggacattgcgttcagctgcactctgtatacctcaggggtgggacc | 328 |
| | v7 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggAggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 295 | PPIB_primelike_ | atgtggcttctcagggacattgcgttcagctgcactctgtatacctcaggggtgggacc | 328 |
| | v8 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggCggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 296 | PPIB_primelike_ | atgtggcttctcagggacattgcgttcagctgcactctgtatacctcaggggtgggacc | 328 |
| | v9 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggGggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 297 | PPIB_primelike_ | atgtggcttctcagggacattgcgttcagctgcactctgtatacctcaggggtgggacc | 328 |
| | v10 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggtggtgTggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 298 | PPIB_primelike_ | atgtggcttctcagggacattgcgttcagctgcactctgtatacctcaggggtgggacc | 328 |
| | v11 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggtggtgGggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 299 | PPIB_primelike_ | atgtggcttctcagggacattgcgttcagctgcactctgtatacctcaggggtgggacc | 328 |
| | v12 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggtggtgAggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 300 | PPIB_primelike_ | atgtggcttctcagggacattgcgttcagctgcactctgtatacctcaggggtgggacc | 328 |
| | v13 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaAgtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 301 | PPIB_primelike_ | atgtggcttctcagggacattgcgttcagctgcactctgtatacctcaggggtgggacc | 328 |
| | v14 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaTgtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 302 | PPIB_primelike_ | atgtggcttctcagggacattgcgttcagctgcactctgtatacctcaggggtgggacc | 328 |
| | v15 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaCgtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 303 | PPIB_primelike_ | atgtggcttctcagggacattgcgttcagctgcactctgtatacctcaggggtgggacc | 328 |
| | v16 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcagGCAgtggtgcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 304 | PPIB_primelike_ | atgtggcttctcagggacattgcgttcagctgcactctgtatacctcaggggtgggacc | 328 |
| | v17 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcagGCACCGgtgcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 305 | PPIB_primelike_ | atgtggcttctcagggacattgcgttcagctgcactctgtatacctcagggggggacc | 328 |
| | v18 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcagGCACCGACCcggaaggtggagagcaccaagacagacagccgggataaa | |
| | | cccctgaaggatgtgatcatcgcagactgcggcaagatcgaggtggagaagcccttt | |
| | | gccatcgccaaggagGACTACAAAGACGATGACGACAAGTAA | |
| 306 | PPIB_primelike_ | atgtggcttctcagggacattgcgttcagctgcactctgtatacctcaggggtgggacc | 329 |
| | v19 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagAgtggtgcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 307 | PPIB_primelike_ | atgtggcttctcagggacattgcgttcagctgcactctgtatacctcaggggtgggacc | 330 |
| | v20 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagAGgtggtgcggaaggtggagagcaccaagacagacagccgggataaa | |
| | | cccctgaaggatgtgatcatcgcagactgcggcaagatcgaggtggagaagcccttt | |
| | | gccatcgccaaggagGACTACAAAGACGATGACGACAAGTAA | |
| 308 | PPIB_primelike_ | atgtggcttctcagggacattgcgttcagctgcactctgtatacctcaggggtgggacc | 334 |
| | v21 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagTTGCACgtggtgcggaaggtggagagcaccaagacagacagccggg | |
| | | ataaacccctgaaggatgtgatcatcgcagactgcggcaagatcgaggtggagaagc | |
| | | cctttgccatcgccaaggagGACTACAAAGACGATGACGACAAGTAA | |
| 309 | PPIB_primelike_ | atgtggcttctcagggacattgcgttcagctgcactctgtatacctcaggggtgggacc | 340 |
| | v22 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagTTGCACATTGCGgtggtgcggaaggtggagagcaccaagacagac | |
| | | agccgggataaacccctgaaggatgtgatcatcgcagactgcggcaagatcgaggtg | |
| | | gagaagccctttgccatcgccaaggagGACTACAAAGACGATGACGACAA | |
| | | GTAA | |
| 310 | PPIB_primelike_ | atgtggcttctcagggacattgcgttcagctgcactctgtatacctcaggggtgggacc | 352 |
| | v23 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagTTGCACATTGCGTCAACTCATAAGgtggtgcggaaggtggag | |
| | | agcaccaagacagacagccgggataaacccctgaaggatgtgatcatcgcagactg | |
| | | cggcaagatcgaggtggagaagccctttgccatcgccaaggagGACTACAAAG | |
| | | ACGATGACGACAAGTAA | |
| 311 | PPIB_primelike_ | atgtggcttctcagggacattgcgttcagctgcactctgtatacctcaggggtgggacc | 376 |
| | v24 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagTTGCACATTGCGTCAACTCATAAGATGTCTCAACGGCAT | |
| | | GCGCAACTTgtggtgcggaaggtggagagcaccaagacagacagccgggataa | |
| | | acccctgaaggatgtgatcatcgcagactgcggcaagatcgaggtggagaagccctt | |
| | | tgccatcgccaaggagGACTACAAAGACGATGACGACAAGTAA | |
| 312 | PPIB_primelike_ | atgtggcttctcagggacattgcgttcagctgcactctgtatacctcaggggtgggacc | 424 |
| | v25 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagTTGCACATTGCGTCAACTCATAAGATGTCTCAACGGCAT | |
| | | GCGCAACTTGTGAAGTGTCTACTATCCTTAAACGCATATCTCGCA | |
| | | CAGTATCTCCCGgtggtgcggaaggtggagagcaccaagacagacagccggga | |
| | | taaacccctgaaggatgtgatcatcgcagactgcggcaagatcgaggtggagaagcc | |
| | | ctttgccatcgccaaggagGACTACAAAGACGATGACGACAAGTAA | |
| 313 | PPIB_primelike_ | atgtggcttctcagggacattgcgttcagctgcactctgtatacctcaggggtgggacc | 322 |
| | v26 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagcggaaggtggagagcaccaagacagacagccgggataaacccctgaa | |
| | | ggatgtgatcatcgcagactgcggcaagatcgaggtggagaagccctttgccatcgc | |
| | | caaggagGACTACAAAGACGATGACGACAAGTAA | |
| 314 | PPIB_primelike_ | atgtggcttctcagggacattgcgttcagctgcactctgtatacctcaggggtgggacc | 316 |
| | v27 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaggtggagagcaccaagacagacagccgggataaacccctgaaggatgtg | |
| | | atcatcgcagactgcggcaagatcgaggtggagaagccctttgccatcgccaaggag | |
| | | GACTACAAAGACGATGACGACAAGTAA | |
| 315 | PPIB_primelike_ | atgtggcttctcagggacattgcgttcagctgcactctgtatacctcagggggggacc | 304 |
| | v28 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggagaagacagacagccgggataaacccctgaaggatgtgatcatcgcagac | |
| | | tgcggcaagatcgaggtggagaagccctttgccatcgccaaggagGACTACAAA | |
| | | GACGATGACGACAAGTAA | |
| 316 | branchpoint | ggaacagaaaatataaagtttctgaggagaattcaaatgaagccaaaacctcaaaa | 289 |
| | STAT3 Var1 | aagatacatgcaggacctgcaggcagtatccccaagagaaggctccctgttggccag | |
| | | gtgcagtggctcacgcctgtaatgccagcactttgagaacgacTaActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 317 | branchpoint | ggaacagaaaatataaagtttctgaggagaattcaaatgaagccaaaacctcaaaa | 289 |
| | STAT3 Var2 | aagatacatgcaggacctgcaggcagtatccccaagagaaggctccctgttggccag | |
| | | gtgcagtggctcacgcctgtaatgccagcactttgagaacgatTaActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 318 | branchpoint | ggaacagaaaatataaagtttctgaggagaattcaaatgaagccaaaacctcaaaa | 289 |
| | STAT3 Var3 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 319 | branchpoint | ggaacagaaaatataaagtttctgaggagaattcaaatgaagccaaaacctcaaaa | 289 |
| | STAT3 Var4 | aagatacatgcaggacctgcaggcagtatccccaagagaaggctccctgttggccag | |
| | | gtgcagtggctcacgcctgtaatgccagcactttgagaacgacTaAttctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 320 | branchpoint | ggaacagaaaatataaagtttctgaggagaattcaaatgaagccaaaacctcaaaa | 289 |
| | STAT3 Var5 | aagatacatgcaggacctgcaggcagtatccccaagagaaggctccctgttggccag | |
| | | gtgcagtggctcacgcctgtaatgccagcactttgagaacgacTgActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 321 | branchpoint | ggaacagaaaatataaagtttctgaggagaattcaaatgaagccaaaacctcaaaa | 289 |
| | STAT3 Var6 | aagatacatgcaggacctgcaggcagtatccccaagagaaggctccctgttggccag | |
| | | gtgcagtggctcacgcctgtaatgccagcactttgagaacgatTgActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 322 | branchpoint | ggaacagaaaatataaagtttctgaggagaattcaaatgaagccaaaacctcaaaa | 289 |
| | STAT3 Var7 | aagatacatgcaggacctgcaggcagtatccccaagagaaggctccctgttggccag | |
| | | gtgcagtggctcacgcctgtaatgccagcactttgagaacgatTgAttctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 323 | branchpoint | ggaacagaaaatataaagtttctgaggagaattcaaatgaagccaaaacctcaaaa | 289 |
| | STAT3 Var8 | aagatacatgcaggacctgcaggcagtatccccaagagaaggctccctgttggccag | |
| | | gtgcagtggctcacgcctgtaatgccagcactttgagaacgacTgAttctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 324 | branchpoint | ggaacagaaaatataaagtttctgaggagaattcaaatgaagccaaaacctcaaaa | 289 |
| | STAT3 Var9 | aagatacatgcaggacctgcaggcagtatccccaagagaaggctccctgttggccag | |
| | | gtgcagtggctcacgcctgtaatgccagcactttgagaacgacTcActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 325 | branchpoint | ggaacagaaaatataaagtttctgaggagaattcaaatgaagccaaaacctcaaaa | 289 |
| | STAT3 | aagatacatgcaggacctgcaggcagtatccccaagagaaggctccctgttggccag | |
| | Var10 | gtgcagtggctcacgcctgtaatgccagcactttgagaacgatTcActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 326 | branchpoint | ggaacagaaaatataaagtttctgaggagaattcaaatgaagccaaaacctcaaaa | 289 |
| | STAT3 | aagatacatgcaggacctgcaggcagtatccccaagagaaggctccctgttggccag | |
| | Var11 | gtgcagtggctcacgcctgtaatgccagcactttgagaacgatTcAttctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 327 | branchpoint | ggaacagaaaatataaagtttctgaggagaattcaaatgaagccaaaacctcaaaa | 289 |
| | STAT3 | aagatacatgcaggacctgcaggcagtatccccaagagaaggctccctgttggccag | |
| | Var13 | gtgcagtggctcacgcctgtaatgccagcactttgagaacgacTtActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 328 | branchpoint | ggaacagaaaatataaagtttctgaggagaattcaaatgaagccaaaacctcaaaa | 289 |
| | STAT3 | aagatacatgcaggacctgcaggcagtatccccaagagaaggctccctgttggccag | |
| | Var14 | gtgcagtggctcacgcctgtaatgccagcactttgagaacgatTtActctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 329 | branchpoint | ggaacagaaaatataaagtttctgaggagaattcaaatgaagccaaaacctcaaaa | 289 |
| | STAT3 | aagatacatgcaggacctgcaggcagtatccccaagagaaggctccctgttggccag | |
| | Var15 | gtgcagtggctcacgcctgtaatgccagcactttgagaacgatTtAttctcttcttttttt | |
| | | tctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 330 | branchpoint | ggaacagaaaatataaagtttctgaggagaattcaaatgaagccaaaacctcaaaa | 289 |
| | STAT3 | aagatacatgcaggacctgcaggcagtatccccaagagaaggctccctgttggccag | |
| | Var16 | gtgcagtggctcacgcctgtaatgccagcactttgagaacgacTtAttctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 331 | branchpoint | atgtggcttctcagggacattgcgttcagctgcactctgtatacctcaggggtgggacc | 328 |
| | PPIB Var1 | agcacgtcactgagtgaaggaggggagggaggctctggcagttgtgcagccttcctg | |
| | | gctgggctctgagggggctggaagaatttagaacaacgacTaActctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 332 | branchpoint | atgtggcttctcagggacattgcgttcagctgcactctgtatacctcaggggtgggacc | 328 |
| | PPIB Var2 | agcacgtcactgagtgaaggaggggagggaggctctggcagttgtgcagccttcctg | |
| | | gctgggctctgagggggctggaagaatttagaacaacgatTaActctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 333 | branchpoint | atgtggcttctcagggacattgcgttcagctgcactctgtatacctcagggggggacc | 328 |
| | PPIB Var3 | agcacgtcactgagtgaaggaggggagggaggctctggcagttgtgcagccttcctg | |
| | | gctgggctctgagggggctggaagaatttagaacaacgatTaAttctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 334 | branchpoint | atgtggcttctcagggacattgcgttcagctgcactctgtatacctcaggggtgggacc | 328 |
| | PPIB Var4 | agcacgtcactgagtgaaggaggggagggaggctctggcagttgtgcagccttcctg | |
| | | gctgggctctgagggggctggaagaatttagaacaacgacTaAttctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 335 | branchpoint | atgtggcttctcagggacattgcgttcagctgcactctgtatacctcaggggtgggacc | 328 |
| | PPIB Var5 | agcacgtcactgagtgaaggaggggagggaggctctggcagttgtgcagccttcctg | |
| | | gctgggctctgagggggctggaagaatttagaacaacgacTgActctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 336 | branchpoint | atgtggcttctcagggacattgcgttcagctgcactctgtatacctcaggggtgggacc | 328 |
| | PPIB Var6 | agcacgtcactgagtgaaggaggggagggaggctctggcagttgtgcagccttcctg | |
| | | gctgggctctgagggggctggaagaatttagaacaacgatTgActctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 337 | branchpoint | atgtggcttctcagggacattgcgttcagctgcactctgtatacctcaggggtgggacc | 328 |
| | PPIB Var7 | agcacgtcactgagtgaaggaggggagggaggctctggcagttgtgcagccttcctg | |
| | | gctgggctctgagggggctggaagaatttagaacaacgatTgAttctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 338 | branchpoint | atgtggcttctcagggacattgcgttcagctgcactctgtatacctcaggggtgggacc | 328 |
| | PPIB Var8 | agcacgtcactgagtgaaggaggggagggaggctctggcagttgtgcagccttcctg | |
| | | gctgggctctgagggggctggaagaatttagaacaacgacTgAttctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 339 | branchpoint | atgtggcttctcagggacattgcgttcagctgcactctgtatacctcaggggtgggacc | 328 |
| | PPIB Var9 | agcacgtcactgagtgaaggaggggagggaggctctggcagttgtgcagccttcctg | |
| | | gctgggctctgagggggctggaagaatttagaacaacgacTcActctcttctttttttt | |
| | | ctgcaggaAgtTgtTcggaaggtggagagcaccaagacagacagccgggataaac | |
| | | ccctgaaggatgtgatcatcgcagactgcggcaagatcgaggtggagaagccctttg | |
| | | ccatcgccaaggagGACTACAAAGACGATGACGACAAGTAA | |
| 340 | branchpoint | atgtggcttctcagggacattgcgttcagctgcactctgtatacctcaggggtgggacc | 328 |
| | PPIB Var10 | agcacgtcactgagtgaaggaggggagggaggctctggcagttgtgcagccttcctg | |
| | | gctgggctctgagggggctggaagaatttagaacaacgatTcActctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 341 | branchpoint | atgtggcttctcagggacattgcgttcagctgcactctgtatacctcaggggtgggacc | 328 |
| | PPIB Var11 | agcacgtcactgagtgaaggaggggagggaggctctggcagttgtgcagccttcctg | |
| | | gctgggctctgagggggctggaagaatttagaacaacgatTcAttctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 342 | branchpoint | atgtggcttctcagggacattgcgttcagctgcactctgtatacctcagggggggacc | 328 |
| | PPIB Var13 | agcacgtcactgagtgaaggaggggagggaggctctggcagttgtgcagccttcctg | |
| | | gctgggctctgagggggctggaagaatttagaacaacgacTtActctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 343 | branchpoint | atgtggcttctcagggacattgcgttcagctgcactctgtatacctcaggggtgggacc | 328 |
| | PPIB Var14 | agcacgtcactgagtgaaggaggggagggaggctctggcagttgtgcagccttcctg | |
| | | gctgggctctgagggggctggaagaatttagaacaacgatTtActctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 344 | branchpoint | atgtggcttctcagggacattgcgttcagctgcactctgtatacctcaggggtgggacc | 328 |
| | PPIB Var15 | agcacgtcactgagtgaaggaggggagggaggctctggcagttgtgcagccttcctg | |
| | | gctgggctctgagggggctggaagaatttagaacaacgatTtAttctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 345 | branchpoint | atgtggcttctcagggacattgcgttcagctgcactctgtatacctcaggggtgggacc | 328 |
| | PPIB Var16 | agcacgtcactgagtgaaggaggggagggaggctctggcagttgtgcagccttcctg | |
| | | gctgggctctgagggggctggaagaatttagaacaacgacTtAttctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 346 | USF1_5TS_ | agCaaGggAgggattctatccaaagcttgtgattatatccaggagcttcggcagagt | 330 |
| | 150bp | aaccaccgcttgtctgaagaactgcagggacttgaccaactgcagctggacaatgac | |
| | | gtgctCcgTcaGcagGTAAGTttgggcTGCATGacTGCATGgtTGCATGa | |
| | | acacaTATTAATttccGTAGTAAAGCTGGCACTTCCAAGCCCCTGAA | |
| | | TGTATTCAGACATCCACTGGTGAGGGGGAAAAGATGAAGCCTTC | |
| | | TCCATGGAGAACAAAGTAGAGGGTGTCAAACTGGGTCAGTGGCT | |
| | | AGCAGAACTGAGAAGGGCTGCACTGGGGGTA | |
| 347 | USF1_5TS_ | agCaaGggAgggattctatccaaagcttgtgattatatccaggagcttcggcagagt | 260 |
| | 80bp_left | aaccaccgcttgtctgaagaactgcagggacttgaccaactgcagctggacaatgac | |
| | | gtgctCcgTcaGcagGTAAGTttgggcTGCATGacTGCATGgtTGCATGa | |
| | | acacaTATTAATttccCCTTCTCCATGGAGAACAAAGTAGAGGGTGT | |
| | | CAAACTGGGTCAGTGGCTAGCAGAACTGAGAAGGGCTGCACTG | |
| | | GGGGTA | |
| 348 | USF1_5TS_ | agCaaGggAgggattctatccaaagcttgtgattatatccaggagcttcggcagagt | 260 |
| | 80bp_midle | aaccaccgcttgtctgaagaactgcagggacttgaccaactgcagctggacaatgac | |
| | | gtgctCcgTcaGcagGTAAGTttgggcTGCATGacTGCATGgtTGCATGa | |
| | | acacaTATTAATttccATGTATTCAGACATCCACTGGTGAGGGGGAA | |
| | | AAGATGAAGCCTTCTCCATGGAGAACAAAGTAGAGGGTGTCAAA | |
| | | CTGGG | |
| 349 | USF1_5TS_ | agCaaGggAgggattctatccaaagcttgtgattatatccaggagcttcggcagagt | 260 |
| | 80bp_right | aaccaccgcttgtctgaagaactgcagggacttgaccaactgcagctggacaatgac | |
| | | gtgctCcgTcaGcagGTAAGTttgggcTGCATGacTGCATGgtTGCATGa | |
| | | acacaTATTAATttccAAGGGAATGGGTAGTAAAGCTGGCACTTCCA | |
| | | AGCCCCTGAATGTATTCAGACATCCACTGGTGAGGGGGAAAAGA | |
| | | TGAAG | |
| 350 | USF1_5TS_ | agCaaGggAgggattctatccaaagcttgtgattatatccaggagcttcggcagagt | 330 |
| | 150bp_NT | aaccaccgcttgtctgaagaactgcagggacttgaccaactgcagctggacaatgac | |
| | | gtgctCcgTcaGcagGTAAGTttgggcTGCATGacTGCATGgtTGCATGa | |
| | | acacaTATTAATttccTTGACCAAGTGGAGGGTGCTCTTCCAGCTCT | |
| | | TGAACAGGACCTAGAGAGTTGGATGTATTAGATGGGCGTACGCA | |
| | | gTATGTGCCCAGTTGTATGATTGTGCGTTTTCAAGGAAGGGAGTG | |
| | | TGCGTCGATTCGTTCAGTATCGACAgGGGG | |
| 351 | HTT_opt_ | GCCACCATGGACTACAAAGACGATGACGACAAGggcccggctgtggc | 225 |
| | hyb2_noGURAGU_ | tgaggagccCctCcaccgaccGTTGAAttgggcTGCATGacTGCATGgtTG | |
| | ISE_BP_ | CATGaacacaTATTAATttcctccacttagttctacacctcattcattcattcagtg | |
| | cargo10 | agtgtttctcgactactatgaataaaccgttatactccatgttgcgggcagaatgggga | |
| | | tctggacaggg | |
| 352 | HTT_opt_ | GCCACCATGGACTACAAAGACGATGACGACAAGggcccggctgtggc | 203 |
| | hyb2_noGURAGU_ | tgaggagccCctCcaccgaccGTGAGTttgggcaacacaTATTAATttcctcca | |
| | noISE_ | cttagttctacacctcattcattcattcagtgagtgtttctcgactactatgaataaaccg | |
| | BP_cargo11 | ttatactccatgttgcgggcagaatggggatctggacaggg | |
| 353 | HTT_opt_ | GCCACCATGGACTACAAAGACGATGACGACAAGggcccggctgtggc | 214 |
| | hyb2_noGURAGU_ | tgaggagccCctCcaccgaccGTTGAAttgggcTGCATGacTGCATGgtTG | |
| | ISE_noBP_ | CATGaacacatccacttagttctacacctcattcattcattcagtgagtgtttctcgac | |
| | cargo12 | tactatgaataaaccgttatactccatgttgcgggcagaatggggatctggacaggg | |
| 354 | HTT_opt_ | GCCACCATGGACTACAAAGACGATGACGACAAGggcccggctgtggc | 225 |
| | hyb2_natGURAGU_ | tgaggagccCctCcaccgaccGTGAGTttgggcTGCATGacTGCATGgtTG | |
| | ISE_BP_ | CATGaacacaTATTAATttcctccacttagttctacacctcattcattcattcagtg | |
| | cargo13 | agtgtttctcgactactatgaataaaccgttatactccatgttgcgggcagaatgggga | |
| | | tctggacaggg | |
| 355 | HTT_opt_ | GCCACCATGGACTACAAAGACGATGACGACAAGggcccggctgtggc | 203 |
| | hyb2_GUAAGU_ | tgaggagccCctCcaccgaccGTAAGTttgggcaacacaTATTAATttcctcca | |
| | noISE_BP_ | cttagttctacacctcattcattcattcagtgagtgtttctcgactactatgaataaaccg | |
| | cargo14 | ttatactccatgttgcgggcagaatggggatctggacaggg | |
| 356 | HTT_opt_ | GCCACCATGGACTACAAAGACGATGACGACAAGggcccggctgtggc | 192 |
| | hyb2_GUAAGU_ | tgaggagccCctCcaccgaccGTAAGTttgggcaacacatccacttagttctacac | |
| | noISE_noBP_ | ctcattcattcattcagtgagtgtttctcgactactatgaataaaccgttatactccatgt | |
| | cargo15 | tgcgggcagaatggggatctggacaggg | |
| 357 | HTT_opt_ | GCCACCATGGACTACAAAGACGATGACGACAAGggcccggctgtggc | 280 |
| | hyb2_ | tgaggagccCctCcaccgaccGTAAGTttgggcTGCATGacTGCATGgtTG | |
| | 100bpdown_ | CATGaacacaTATTAATttcctccacttagttctacacctcattcattcattcagtg | |
| | GUAAGU_ISE_BP_ | agtgtttctcgactactatgaataaaccgttatactccatgttgcgggcagaatgggga | |
| | cargo16 | tctggctggcggccgctcgagcatgcatctagagggccctattctatagtgtcacctaa | |
| | | atgctag | |
| 358 | HTT_opt_hyb2_ | GCCACCATGGACTACAAAGACGATGACGACAAGggcccggctgtggc | 175 |
| | 100bpup_GUAAGU_ | tgaggagccCctCcaccgaccGTAAGTttgggcTGCATGacTGCATGgtTG | |
| | ISE_BP_ | CATGaacacaTATTAATttccactatgaataaaccgttatactccatgttgcgggc | |
| | cargo17 | agaatggggatctggacaggg | |
| 359 | HTT_opt_hyb2_ | GCCACCATGGACTACAAAGACGATGACGACAAGggcccggctgtggc | 395 |
| | U1snRNA_ | tgaggagccCctCcaccgaccGTAAGTttgggcatacttacctggcaggggagat | |
| | FL_ISE_BP_ | accatgatcacgaaggtggttttcccagggcgaggcttatccattgcactccggatgtg | |
| | cargo18 | ctgacccctgcgatttccccaaatgtgggaaactcgactgcataatttgtggtagtggg | |
| | | ggactgcgttcgcgctttcccctgggtttcTGCATGacTGCATGgtTGCATGaa | |
| | | cacaTATTAATttcctccacttagttctacacctcattcattcattcagtgagtgtttct | |
| | | cgactactatgaataaaccgttatactccatgttgcgggcagaatggggatctggaca | |
| | | ggg | |
| 360 | HTT_opt_hyb2_ | GCCACCATGGACTACAAAGACGATGACGACAAGggcccggctgtggc | 260 |
| | U1snRNA_ | tgaggagccCctCcaccgaccGTAAGTttgggctgcgatttccccaaatgtgggaa | |
| | SL3_ISE_ | actcggggtttcTGCATGacTGCATGgtTGCATGaacacaTATTAATttcct | |
| | BP_cargo19 | ccacttagttctacacctcattcattcattcagtgagtgtttctcgactactatgaataaa | |
| | | ccgttatactccatgttgcgggcagaatggggatctggacaggg | |
| 361 | HTT_opt_hyb2_ | GCCACCATGGACTACAAAGACGATGACGACAAGggcccggctgtggc | 273 |
| | U1snRNA_ | tgaggagccCctCcaccgaccGTAAGTttgggcataatttgtggtagtgggggact | |
| | smSL4_ISE_ | gcgttcgcgctttcccctgggtttcTGCATGacTGCATGgtTGCATGaacacaT | |
| | BP_cargo20 | ATTAATttcctccacttagttctacacctcattcattcattcagtgagtgtttctcgact | |
| | | actatgaataaaccgttatactccatgttgcgggcagaatggggatctggacaggg | |
| 362 | HTT_opt_hyb2_ | GCCACCATGGACTACAAAGACGATGACGACAAGggcccggctgtggc | 260 |
| | ISE_U1snRNA_ | tgaggagccCctCcaccgaccGTAAGTttgggcTGCATGacTGCATGgtTG | |
| | SL3_BP_ | CATGtgcgatttccccaaatgtgggaaactcggggtttcaacacaTATTAATttcc | |
| | cargo21 | tccacttagttctacacctcattcattcattcagtgagtgtttctcgactactatgaataa | |
| | | accgttatactccatgttgcgggcagaatggggatctggacaggg | |
| 363 | HTT_opt_hyb2_ | GCCACCATGGACTACAAAGACGATGACGACAAGggcccggctgtggc | 225 |
| | GUAAGU_ | tgaggagccCctCcaccgaccGTAAGTttgggcTTTGGGacTTTGGGgtTTT | |
| | altISE_BP_ | GGGaacacaTATTAATttcctccacttagttctacacctcattcattcattcagtga | |
| | cargo22 | gtgtttctcgactactatgaataaaccgttatactccatgttgcgggcagaatggggat | |
| | | ctggacaggg | |
| 364 | HTT_opt_hyb2_ | GCCACCATGGACTACAAAGACGATGACGACAAGggcccggctgtggc | 225 |
| | GUAAGU_ | tgaggagccCctCcaccgaccGTAAGTttgggcTTTGGGacGAGGGGgtT | |
| | mixISE_BP_ | GCATGaacacaTATTAATttcctccacttagttctacacctcattcattcattcagt | |
| | cargo23 | gagtgtttctcgactactatgaataaaccgttatactccatgttgcgggcagaatgggg | |
| | | atctggacaggg | |
| 365 | HTT_opt_hyb2_ | GCCACCATGGACTACAAAGACGATGACGACAAGggcccggctgtggc | 246 |
| | GUAAGU_ | tgaggagccCctCcaccgaccGTAAGTttgggcTTTGGGcTTTGGGacGAG | |
| | mixdoubleISE_ | GGGcGAGGGGgtTGCATGcTGCATGaacacaTATTAATttcctccactta | |
| | BP_cargo24 | gttctacacctcattcattcattcagtgagtgtttctcgactactatgaataaaccgttat | |
| | | actccatgttgcgggcagaatggggatctggacaggg | |
| 366 | HTT_opt_hyb2_ | GCAAGGCGGAGGAAGGCCACCATGGACTACAAAGACGATGACG | 240 |
| | ESE_Ax1_ | ACAAGggcccggctgtggctgaggagccCctCcaccgaccGTAAGTttgggcT | |
| | GUAAGU_ISE_ | GCATGacTGCATGgtTGCATGaacacaTATTAATttcctccacttagttcta | |
| | BP_cargo25 | cacctcattcattcattcagtgagtgtttctcgactactatgaataaaccgttatactcc | |
| | | atgttgcgggcagaatggggatctggacaggg | |
| 367 | HTT_opt_hyb2_ | GCAAGGCGGAGGAAAGGCAAGGCGGAGGAAGGCAAGGCGGAG | 271 |
| | ESE_Ax3_ | GAAGGCCACCATGGACTACAAAGACGATGACGACAAGggcccggct | |
| | GUAAGU_ISE_ | gtggctgaggagccCctCcaccgaccGTAAGTttgggcTGCATGacTGCATG | |
| | BP_cargo26 | gtTGCATGaacacaTATTAATttcctccacttagttctacacctcattcattcattc | |
| | | agtgagtgtttctcgactactatgaataaaccgttatactccatgttgcgggcagaatg | |
| | | gggatctggacaggg | |
| 368 | HTT_opt_hyb2_ | GCACACAGGACCACACAGGACGCACACAGGACCACACAGGACG | 267 |
| | ESE_Bx4_ | CCACCATGGACTACAAAGACGATGACGACAAGggcccggctgtggctg | |
| | GUAAGU_ISE_ | aggagccCctCcaccgaccGTAAGTttgggcTGCATGacTGCATGgtTGCA | |
| | BP_cargo27 | TGaacacaTATTAATttcctccacttagttctacacctcattcattcattcagtgagt | |
| | | gtttctcgactactatgaataaaccgttatactccatgttgcgggcagaatggggatct | |
| | | ggacaggg | |
| 369 | HTT_opt_hyb2_ | GAAAAAGAAAGAAAAAAAGAAAGAAGCCACCATGGACTACAAA | 250 |
| | ESE_Cx2_ | GACGATGACGACAAGggcccggctgtggctgaggagccCctCcaccgaccG | |
| | GUAAGU_ISE_ | TAAGTttgggcTGCATGacTGCATGgtTGCATGaacacaTATTAATttcc | |
| | BP_cargo28 | tccacttagttctacacctcattcattcattcagtgagtgtttctcgactactatgaataa | |
| | | accgttatactccatgttgcgggcagaatggggatctggacaggg | |
| 370 | HTT_opt_hyb2_ | GTCAGAGGATCAGAGGAGTCAGAGGATCAGAGGAGCCACCATG | 259 |
| | ESE_Dx4_ | GACTACAAAGACGATGACGACAAGggcccggctgtggctgaggagccCct | |
| | GUAAGU_ISE_ | CcaccgaccGTAAGTttgggcTGCATGacTGCATGgtTGCATGaacacaT | |
| | BP_cargo29 | ATTAATttcctccacttagttctacacctcattcattcattcagtgagtgtttctcgact | |
| | | actatgaataaaccgttatactccatgttgcgggcagaatggggatctggacaggg | |
| 371 | pDF0945_ | gcccggctgtggctgaggagccCctCcaccgaccGTAAGTttgggcTGCATGac | 235 |
| | HTT_cargo3 | TGCATGgtTGCATGaacacaTATTAATttcctccacttagttctacacctcattc | |
| | | attcattcagtgagtgtttctcgactactatgaataaaccgttatactccatgttgcggg | |
| | | cagaatggggatctggacagggaagcacagggcacgagttcaccaatggctgtcaa | |
| | | gctacgctgc | |
| 372 | HTT cargo13 | tcacactttggacctcattttcatctaaggaaggtggtataatatctcccagggataca | 379 |
| | aRY1584 | ggaacctcagggagagataagactactgtcatgtgtgcccctctctctaccatttctgg | |
| | ABCD2 | aaacaataccaggaggcagaattcaggcatccaacgacTtActctcttcttttttttct | |
| | | gcagagCaaGggAgggattctatccaaagcttgtgattatatccaggagcttcggca | |
| | | gagtaaccaccgcttgtctgaagaactgcagggacttgaccaactgcagctggacaa | |
| | | tgacgtgcttcgacaacagGACTACAAGGACCACGACGGTGACTACAA | |
| | | GGACCACGACATCGACTACAAGGACGACGACGACAAGTAA | |
| 373 | USF1 NT | TTGACCAAGTGGAGGGTGCTCTTCCAGCTCTTGAACAGGACCTA | 379 |
| | cargo | GAGAGTTGGATGTATTAGATGGGCGTACGCAgTATGTGCCCAGT | |
| | 3xFLAG_ | TGTATGATTGTGCGTTTTCAAGGAAGGGAGTGTGCGTCGATTCGT | |
| | ary1852_E1 | TCAGTATCGACAgGGGGaacgacTtActctcttcttttttttctgcagagCaaG | |
| | | ggAgggattctatccaaagcttgtgattatatccaggagcttcggcagagtaaccacc | |
| | | gcttgtctgaagaactgcagggacttgaccaactgcagctggacaatgacgtgcttcg | |
| | | acaacagGACTACAAGGACCACGACGGTGACTACAAGGACCACGA | |
| | | CATCGACTACAAGGACGACGACGACAAGTAA | |
| 374 | USF1 T | agacccaagcttggtaccgagctcggatcctcacactttggacctcattttcatctaag | 409 |
| | cargo xten | gaaggtggtataatatctcccagggatacaggaacctcagggagagataagactact | |
| | 3xFLAG- | gtcatgtgtgcccctctctctaccatttctggaaacaataccaggaggcagaattcagg | |
| | ary1852_D1 | catccaacgacTtActctcttcttttttttctgcagagCaaGggAgggattctatccaa | |
| | | agcttgtgattatatccaggagcttcggcagagtaaccaccgcttgtctgaagaactgc | |
| | | agggacttgaccaactgcagctggacaatgacgtgcttcgacaacagGACTACAA | |
| | | GGACCACGACGGTGACTACAAGGACCACGACATCGACTACAAGG | |
| | | ACGACGACGACAAGTAA | |
| 375 | pDF0978_ | taaatcaaacgtccacataaagaatgaggtggtaaaatgaacaagcactacggttct | 248 |
| | RPL41 | atcgttctctgttctgttaaatcctggctccagggagaaaacactcaaacgtttttctcc | |
| | branchpoint | taaagatcttttaagatttccaaaccaaatgttaacgacTtActctcttcttttttttctg | |
| | v13 cargo 2 | caggctCaagcgcaaaagaagaaagatgaggcagaggtccaagGACTACAAA | |
| | | GACGATGACGACAAGTAA | |
| 376 | pDF0865_ | ggaacagaaaatataaagtttctgaggagaattcaaatgaagccaaaacctcaaaa | 289 |
| | STAT3_3TS_ | aagatacatgcaggacctgcaggcagtatccccaagagaaggctccctgttggccag | |
| | Cargo2 | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAA | |
| 377 | pDF0867 | atgtggcttctcagggacattgcgttcagctgcactctgtatacctcaggggtgggacc | 328 |
| | PPIB cargo 3 | agcacgtcactgagtgaaggaggggagggaggctctggcagttgtgcagccttcctg | |
| | | gctgggctctgagggggctggaagaatttagaacaacgaggaattctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 378 | SHANK3 | GAGGAGCCTAGAAGGCGCCGGGTTGGCAAGTGGGCAGGGAAC | 289 |
| | Cargo 3 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgacTtActctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| 379 | pDF0986 | GCCACCATGGACTACAAAGACGATGACGACAAGggcccggctgtggc | 269 |
| | HTT cargo | tgaggagccCctCcaccgaccGTGAGTttgggcTGCATGacTGCATGgtTG | |
| | | CATGaacacaTATTAATttcctccacttagttctacacctcattcattcattcagtg | |
| | | agtgtttctcgactactatgaataaaccgttatactccatgttgcgggcagaatgggga | |
| | | tctggacagggaagcacagggcacgagttcaccaatggctgtcaagctacgctgc | |
| 384 | PABPC1_5TS_ | GCCACCATGGACTACAAAGACGATGACGACAAGaaccccagtgcccc | 424 |
| | cargo_1 | cagctaccccatggcctcgctctacgtgggggacctccaccccgacgtgaccgaggcg | |
| | | atgctctacgagaagttcagcccggccgggcccatcctctccatccgggtctgcaggg | |
| | | acatgatcacccgccgctccttgggctacgcgtatgtgaacttccagcagccAgcTga | |
| | | TgGTGAGTttgggcTGCATGacTGCATGgtTGCATGaacacaTATTAAT | |
| | | ttccCAATATTACTTCAAAATTTTTGCTGGCTACTTAAGATTATATA | |
| | | AACTATGGTGACTGGAGTGGGAGGACACATGGTCTCACAGTTGA | |
| | | ACGCTTCCTCTTTAAGCTTCAAGATGGCTAGACCTTTCAAGTATCA | |
| | | CACACTAGTGTGGGACC | |
| 385 | PABPC1_5TS_ | GCCACCATGGACTACAAAGACGATGACGACAAGaaccccagtgcccc | 424 |
| | cargo_2 | cagctaccccatggcctcgctctacgtgggggacctccaccccgacgtgaccgaggcg | |
| | | atgctctacgagaagttcagcccggccgggcccatcctctccatccgggtctgcaggg | |
| | | acatgatcacccgccgctccttgggctacgcgtatgtgaacttccagcagccAgcTga | |
| | | TgGTGAGTttgggcTGCATGacTGCATGgtTGCATGaacacaTATTAAT | |
| | | ttccGCTACTTAAGATTATATAAACTATGGTGACTGGAGTGGGAG | |
| | | GACACATGGTCTCACAGTTGAACGCTTCCTCTTTAAGCTTCAAGA | |
| | | TGGCTAGACCTTTCAAGTATCACACACTAGTGTGGGACCTAAGTT | |
| | | GTATAAAGCAAAGACAAAT | |
| 386 | PABPC1_5TS_ | GCCACCATGGACTACAAAGACGATGACGACAAGaaccccagtgcccc | 424 |
| | cargo_3 | cagctaccccatggcctcgctctacgtgggggacctccaccccgacgtgaccgaggcg | |
| | | atgctctacgagaagttcagcccggccgggcccatcctctccatccgggtctgcaggg | |
| | | acatgatcacccgccgctccttgggctacgcgtatgtgaacttccagcagccAgcTga | |
| | | TgGTGAGTttgggcTGCATGacTGCATGgtTGCATGaacacaTATTAAT | |
| | | ttccGTGACTGGAGTGGGAGGACACATGGTCTCACAGTTGAACGC | |
| | | TTCCTCTTTAAGCTTCAAGATGGCTAGACCTTTCAAGTATCACACA | |
| | | CTAGTGTGGGACCTAAGTTGTATAAAGCAAAGACAAATACCAGA | |
| | | AGCCCCCGAAATTTCTCAC | |
| 387 | PABPC1_5TS_ | GCCACCATGGACTACAAAGACGATGACGACAAGaaccccagtgcccc | 424 |
| | cargo_4 | cagctaccccatggcctcgctctacgtgggggacctccaccccgacgtgaccgaggcg | |
| | | atgctctacgagaagttcagcccggccgggcccatcctctccatccgggtctgcaggg | |
| | | acatgatcacccgccgctccttgggctacgcgtatgtgaacttccagcagccAgcTga | |
| | | TgGTGAGTttgggcTGCATGacTGCATGgtTGCATGaacacaTATTAAT | |
| | | ttccTCTCACAGTTGAACGCTTCCTCTTTAAGCTTCAAGATGGCTAG | |
| | | ACCTTTCAAGTATCACACACTAGTGTGGGACCTAAGTTGTATAAA | |
| | | GCAAAGACAAATACCAGAAGCCCCCGAAATTTCTCACATATAAAA | |
| | | GAATTCCATATTGCTAA | |
| 388 | PPIB_5TS_ | GCCACCATGGACTACAAAGACGATGACGACAAGctgcgcctctccgaa | 366 |
| | cargo_1 | cgcaacatgaaggtgctccttgccgccgccctcatcgcggggtccgtcttcttcctgctg | |
| | | ctgccgggaccttctgcggccgatgagaagaagaaggggcccaaagtTacAgtGa | |
| | | agGTGAGTttgggcTGCATGacTGCATGgtTGCATGaacacaTATTAAT | |
| | | ttccCTTAGCTTCTTTAAAGGGGCGTTGCTAGGGGAGGGAAGGTA | |
| | | CAAGAAGCTAACCTGAGGATGGGAGAGAGAATAGAGCCATATTT | |
| | | TTAGAGAAGTGGTTCTGAATCTGATTTTGGTGACGGTAAAAATCC | |
| | | TATGAGAATCTAATGAAAGC | |
| 389 | PPIB_5TS_ | GCCACCATGGACTACAAAGACGATGACGACAAGctgcgcctctccgaa | 366 |
| | cargo_2 | cgcaacatgaaggtgctccttgccgccgccctcatcgcggggtccgtcttcttcctgctg | |
| | | ctgccgggaccttctgcggccgatgagaagaagaaggggcccaaagtTacAgtGa | |
| | | agGTGAGTttgggcTGCATGacTGCATGgtTGCATGaacacaTATTAAT | |
| | | ttccTAGGGGAGGGAAGGTACAAGAAGCTAACCTGAGGATGGGA | |
| | | GAGAGAATAGAGCCATATTTTTAGAGAAGTGGTTCTGAATCTGA | |
| | | TTTTGGTGACGGTAAAAATCCTATGAGAATCTAATGAAAGCTCCA | |
| | | GACCCTTTTCTTGGAAAACAT | |
| 390 | PPIB_5TS_ | GCCACCATGGACTACAAAGACGATGACGACAAGctgcgcctctccgaa | 366 |
| | cargo_3 | cgcaacatgaaggtgctccttgccgccgccctcatcgcggggtccgtcttcttcctgctg | |
| | | ctgccgggaccttctgcggccgatgagaagaagaaggggcccaaagtTacAgtGa | |
| | | agGTGAGTttgggcTGCATGacTGCATGgtTGCATGaacacaTATTAAT | |
| | | ttccAACCTGAGGATGGGAGAGAGAATAGAGCCATATTTTTAGAG | |
| | | AAGTGGTTCTGAATCTGATTTTGGTGACGGTAAAAATCCTATGAG | |
| | | AATCTAATGAAAGCTCCAGACCCTTTTCTTGGAAAACATGCTTATC | |
| | | TATACCCTCCTTGGACTA | |
| 391 | PPIB_5TS_ | GCCACCATGGACTACAAAGACGATGACGACAAGctgcgcctctccgaa | 366 |
| | cargo_4 | cgcaacatgaaggtgctccttgccgccgccctcatcgcggggtccgtcttcttcctgctg | |
| | | ctgccgggaccttctgcggccgatgagaagaagaaggggcccaaagtTacAgtGa | |
| | | agGTGAGTttgggcTGCATGacTGCATGgtTGCATGaacacaTATTAAT | |
| | | ttccAGCCATATTTTTAGAGAAGTGGTTCTGAATCTGATTTTGGTG | |
| | | ACGGTAAAAATCCTATGAGAATCTAATGAAAGCTCCAGACCCTTT | |
| | | TCTTGGAAAACATGCTTATCTATACCCTCCTTGGACTACAGGATA | |
| | | ACAATATTTGCTCTAAAC | |
| 392 | RPL41_5TS_ | GCCACCATGGACTACAAAGACGATGACGACAAGagagccaagtgga | 266 |
| | cargo_1 | ggaagaagcgaatgcgGCgGTGAGTttgggcTGCATGacTGCATGgtTGC | |
| | | ATGaacacaTATTAATttccCCAGAGTTGCCTTTCCCTCCCACATTAA | |
| | | ATCAAACGTCCACATAAAGAATGAGGTGGTAAAATGAACAAGCA | |
| | | CTACGGTTCTATCGTTCTCTGTTCTGTTAAATCCTGGCTCCAGGGA | |
| | | GAAAACACTCAAACGTTTTTCTCCTAAAGATC | |
| 393 | RPL41_5TS_ | GCCACCATGGACTACAAAGACGATGACGACAAGagagccaagtgga | 266 |
| | cargo_2 | ggaagaagcgaatgcgGCgGTGAGTttgggcTGCATGacTGCATGgtTGC | |
| | | ATGaacacaTATTAATttccTAAATCAAACGTCCACATAAAGAATGA | |
| | | GGTGGTAAAATGAACAAGCACTACGGTTCTATCGTTCTCTGTTCT | |
| | | GTTAAATCCTGGCTCCAGGGAGAAAACACTCAAACGTTTTTCTCC | |
| | | TAAAGATCTTTTAAGATTTCCAAACCAAATGTT | |
| 394 | RPL41_5TS_ | GCCACCATGGACTACAAAGACGATGACGACAAGagagccaagtgga | 266 |
| | cargo_3 | ggaagaagcgaatgcgGCgGTGAGTttgggcTGCATGacTGCATGgtTGC | |
| | | ATGaacacaTATTAATttccGAGGTGGTAAAATGAACAAGCACTACG | |
| | | GTTCTATCGTTCTCTGTTCTGTTAAATCCTGGCTCCAGGGAGAAA | |
| | | ACACTCAAACGTTTTTCTCCTAAAGATCTTTTAAGATTTCCAAACC | |
| | | AAATGTTTCTCCTAAGTTTTGTCCAAGGAACT | |
| 395 | RPL41_5TS_ | GCCACCATGGACTACAAAGACGATGACGACAAGagagccaagtgga | 266 |
| | cargo_4 | ggaagaagcgaatgcgGCgGTGAGTttgggcTGCATGacTGCATGgtTGC | |
| | | ATGaacacaTATTAATttccCGGTTCTATCGTTCTCTGTTCTGTTAAAT | |
| | | CCTGGCTCCAGGGAGAAAACACTCAAACGTTTTTCTCCTAAAGAT | |
| | | CTTTTAAGATTTCCAAACCAAATGTTTCTCCTAAGTTTTGTCCAAG | |
| | | GAACTTCCCTCCTCCTGGGCTGGCAAAGTC | |
| 396 | pDF0907_ | tcacactttggacctcattttcatctaaggaaggtggtataatatctcccagggataca | 337 |
| | USF1 Cargo 2 | ggaacctcagggagagataagactactgtcatgtgtgcccctctctctaccatttctgg | |
| | (pdf0114 | aaacaataccaggaggcagaattcaggcatccaacgaggaattctcttcttttttttct | |
| | based) | gcagagCaaGggAgggattctatccaaagcttgtgattatatccaggagcttcggca | |
| | | gagtaaccaccgcttgtctgaagaactgcagggacttgaccaactgcagctggacaa | |
| | | tgacgtgcttcgacaacagGACTACAAAGACGATGACGACAAGTAA | |
| 397 | PPIB 3x | atgtggcttctcagggacattgcgttcagctgcactctgtatacctcaggggtgggacc | 367 |
| | FLAG | agcacgtcactgagtgaaggaggggagggaggctctggcagttgtgcagccttcctg | |
| | | gctgggctctgagggggctggaagaatttagaacaacgacTtActctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAGGACCACGACGGTGACTACAAGGAC | |
| | | CACGACATCGACTACAAGGACGACGACGACAAG | |
| 398 | PPIB XTEN | atgtggcttctcagggacattgcgttcagctgcactctgtatacctcagggggggacc | 610 |
| | 3x FLAG | agcacgtcactgagtgaaggaggggagggaggctctggcagttgtgcagccttcctg | |
| | | gctgggctctgagggggctggaagaatttagaacaacgacTtActctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagggagggccgagctctggcgcacccccaccaagtggagggtctcc | |
| | | tgccgggtccccaacatctactgaagaaggcaccagcgaatccgcaacgcccgagtc | |
| | | aggccctggtacctccacagaaccatctgaaggtagtgcgcctggttccccagctgga | |
| | | agccctacttccaccgaagaaggcacgtcaaccgaaccaagtgaaggatctgcccct | |
| | | gggaccagcactgaaccatctgagGACTACAAGGACCACGACGGTGACT | |
| | | ACAAGGACCACGACATCGACTACAAGGACGACGACGACAAGTAA | |
| 399 | aRY1596 FL | ggaacagaaaatataaagtttctgaggagaattcaaatgaagccaaaacctcaaaa | 2047 |
| | cargo | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacaaagactctggggacgttgcagctctcagaggatcccggaaatt | |
| | | taacattctgggcacaaacacaaaagtgatgaacatggaagaatccaacaacggca | |
| | | gcctctctgcagaattcaaacacttgaccctgagggagcagagatgtgggaatgggg | |
| | | gccgagccaattgtgatgcttccctgattgtgactgaggagctgcacctgatcaccttt | |
| | | gagaccgaggtgtatcaccaaggcctcaagattgacctagagacccactccttgcca | |
| | | gttgtggtgatctccaacatctgtcagatgccaaatgcctgggcgtccatcctgtggta | |
| | | caacatgctgaccaacaatcccaagaatgtaaacttttttaccaagcccccaattgga | |
| | | acctgggatcaagtggccgaggtcctgagctggcagttctcctccaccaccaagcgag | |
| | | gactgagcatcgagcagctgactacactggcagagaaactcttgggacctggtgtga | |
| | | attattcagggtgtcagatcacatgggctaaattttgcaaagaaaacatggctggcaa | |
| | | gggcttctccttctgggtctggctggacaatatcattgaccttgtgaaaaagtacatcct | |
| | | ggccctttggaacgaagggtacatcatgggctttatcagtaaggagcgggagcgggc | |
| | | catcttgagcactaagcctccaggcaccttcctgctaagattcagtgaaagcagcaaa | |
| | | gaaggaggcgtcactttcacttgggtggagaaggacatcagcggtaagacccagatc | |
| | | cagtccgtggaaccatacacaaagcagcagctgaacaacatgtcatttgctgaaatc | |
| | | atcatgggctataagatcatggatgctaccaatatcctggtgtctccactggtctatctc | |
| | | tatcctgacattcccaaggaggaggcattcggaaagtattgtcggccagagagccag | |
| | | gagcatcctgaagctgacccaggcgctgccccatacctgaagaccaagtttatctgtg | |
| | | tgacaccaacgacctgcagcaataccattgacctgccgatgtccccccgcactttaga | |
| | | ttcattgatgcagtttggaaataatggtgaaggtgctgaaccctcagcaggagggcag | |
| | | tttgagtccctcacctttgacatggagttgacctcggagtgcgctacctcccccatgGA | |
| | | CTACAAAGACGATGACGACAAGTAA | |
| 400 | aRY1596 FL | ggaacagaaaatataaagtttctgaggagaattcaaatgaagccaaaacctcaaaa | 447 |
| | cargo -1600 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacGACTACAAAGACGATGACGACAAGTAA | |
| 401 | aRY1596 FL | ggaacagaaaatataaagtttctgaggagaattcaaatgaagccaaaacctcaaaa | 647 |
| | cargo -1400 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggcGACTACAA | |
| | | AGACGATGACGACAAGTAA | |
| 402 | aRY1596 FL | ggaacagaaaatataaagtttctgaggagaattcaaatgaagccaaaacctcaaaa | 847 |
| | cargo -1200 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacGACTACAAAGACGATGACGACAAGTAA | |
| 403 | aRY1596 FL | ggaacagaaaatataaagtttctgaggagaattcaaatgaagccaaaacctcaaaa | 1047 |
| | cargo -1000 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacaaagactctggggacgttgcagctctcagaggatcccggaaatt | |
| | | taacattctgggcacaaacacaaaagtgatgaacatggaagaatccaacaacggca | |
| | | gcctctctgcagaattcaaacacttgaccctgagggagcagagatgtgggaatgggg | |
| | | gccgagccaattgtgatgcttccctgattgtgactgaggagctGACTACAAAGAC | |
| | | GATGACGACAAGTAA | |
| 404 | aRY1596 FL | ggaacagaaaatataaagtttctgaggagaattcaaatgaagccaaaacctcaaaa | 1247 |
| | cargo -800 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacaaagactctggggacgttgcagctctcagaggatcccggaaatt | |
| | | taacattctgggcacaaacacaaaagtgatgaacatggaagaatccaacaacggca | |
| | | gcctctctgcagaattcaaacacttgaccctgagggagcagagatgtgggaatgggg | |
| | | gccgagccaattgtgatgcttccctgattgtgactgaggagctgcacctgatcaccttt | |
| | | gagaccgaggtgtatcaccaaggcctcaagattgacctagagacccactccttgcca | |
| | | gttgtggtgatctccaacatctgtcagatgccaaatgcctgggcgtccatcctgtggta | |
| | | caacatgctgaccaacaatcccaagaatgtaaacttttttaccaagcccccaattgga | |
| | | acctgggatcGACTACAAAGACGATGACGACAAGTAA | |
| 405 | aRY1596 FL | ggaacagaaaatataaagtttctgaggagaattcaaatgaagccaaaacctcaaaa | 1447 |
| | cargo -600 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacaaagactctggggacgttgcagctctcagaggatcccggaaatt | |
| | | taacattctgggcacaaacacaaaagtgatgaacatggaagaatccaacaacggca | |
| | | gcctctctgcagaattcaaacacttgaccctgagggagcagagatgtgggaatgggg | |
| | | gccgagccaattgtgatgcttccctgattgtgactgaggagctgcacctgatcaccttt | |
| | | gagaccgaggtgtatcaccaaggcctcaagattgacctagagacccactccttgcca | |
| | | gttgtggtgatctccaacatctgtcagatgccaaatgcctgggcgtccatcctgtggta | |
| | | caacatgctgaccaacaatcccaagaatgtaaacttttttaccaagcccccaattgga | |
| | | acctgggatcaagtggccgaggtcctgagctggcagttctcctccaccaccaagcgag | |
| | | gactgagcatcgagcagctgactacactggcagagaaactcttgggacctggtgtga | |
| | | attattcagggtgtcagatcacatgggctaaattttgcaaagaaaacatggctggcaa | |
| | | gggcttctccttctgggtctggctggacaatatcattGACTACAAAGACGATGA | |
| | | CGACAAGTAA | |
| 406 | aRY1596 FL | ggaacagaaaatataaagtttctgaggagaattcaaatgaagccaaaacctcaaaa | 1647 |
| | cargo -400 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacaaagactctggggacgttgcagctctcagaggatcccggaaatt | |
| | | taacattctgggcacaaacacaaaagtgatgaacatggaagaatccaacaacggca | |
| | | gcctctctgcagaattcaaacacttgaccctgagggagcagagatgtgggaatgggg | |
| | | gccgagccaattgtgatgcttccctgattgtgactgaggagctgcacctgatcaccttt | |
| | | gagaccgaggtgtatcaccaaggcctcaagattgacctagagacccactccttgcca | |
| | | gttgtggtgatctccaacatctgtcagatgccaaatgcctgggcgtccatcctgtggta | |
| | | caacatgctgaccaacaatcccaagaatgtaaacttttttaccaagcccccaattgga | |
| | | acctgggatcaagtggccgaggtcctgagctggcagttctcctccaccaccaagcgag | |
| | | gactgagcatcgagcagctgactacactggcagagaaactcttgggacctggtgtga | |
| | | attattcagggtgtcagatcacatgggctaaattttgcaaagaaaacatggctggcaa | |
| | | gggcttctccttctgggtctggctggacaatatcattgaccttgtgaaaaagtacatcct | |
| | | ggccctttggaacgaagggtacatcatgggctttatcagtaaggagcgggagcgggc | |
| | | catcttgagcactaagcctccaggcaccttcctgctaagattcagtgaaagcagcaaa | |
| | | gaaggaggcgtcactttcacttgggtggagaaggacatcagcggtaagacccagatc | |
| | | cagtcGACTACAAAGACGATGACGACAAGTAA | |
| 407 | aRY1596 FL | ggaacagaaaatataaagtttctgaggagaattcaaatgaagccaaaacctcaaaa | 1847 |
| | cargo -200 | aagatacatgcaggacctgcaggcagtatccccaagagaaggctccctgttggccag | |
| | | gtgcagtggctcacgcctgtaatgccagcactttgagaacgatTaAttctcttctttttt | |
| | | ttctgcaggatctagaacagaaaatgaaagtggtagagaatctccaggatgactttg | |
| | | atttcaactataaaaccctcaagagtcaaggagacatgcaagatctgaatggaaaca | |
| | | accagtcagtgaccaggcagaagatgcagcagctggaacagatgctcactgcgctgg | |
| | | accagatgcggagaagcatcgtgagtgagctggcggggcttttgtcagcgatggagt | |
| | | acgtgcagaaaactctcacggacgaggagctggctgactggaagaggcggcaacag | |
| | | attgcctgcattggaggcccgcccaacatctgcctagatcggctagaaaactggataa | |
| | | cgtcattagcagaatctcaacttcagacccgtcaacaaattaagaaactggaggagtt | |
| | | gcagcaaaaagtttcctacaaaggggaccccattgtacagcaccggccgatgctgga | |
| | | ggagagaatcgtggagctgtttagaaacttaatgaaaagtgcctttgtggtggagcgg | |
| | | cagccctgcatgcccatgcatcctgaccggcccctcgtcatcaagaccggcgtccagtt | |
| | | cactactaaagtcaggttgctggtcaaattccctgagttgaattatcagcttaaaatta | |
| | | aagtgtgcattgacaaagactctggggacgttgcagctctcagaggatcccggaaatt | |
| | | taacattctgggcacaaacacaaaagtgatgaacatggaagaatccaacaacggca | |
| | | gcctctctgcagaattcaaacacttgaccctgagggagcagagatgtgggaatgggg | |
| | | gccgagccaattgtgatgcttccctgattgtgactgaggagctgcacctgatcaccttt | |
| | | gagaccgaggtgtatcaccaaggcctcaagattgacctagagacccactccttgcca | |
| | | gttgtggtgatctccaacatctgtcagatgccaaatgcctgggcgtccatcctgtggta | |
| | | caacatgctgaccaacaatcccaagaatgtaaacttttttaccaagcccccaattgga | |
| | | acctgggatcaagtggccgaggtcctgagctggcagttctcctccaccaccaagcgag | |
| | | gactgagcatcgagcagctgactacactggcagagaaactcttgggacctggtgtga | |
| | | attattcagggtgtcagatcacatgggctaaattttgcaaagaaaacatggctggcaa | |
| | | gggcttctccttctgggtctggctggacaatatcattgaccttgtgaaaaagtacatcct | |
| | | ggccctttggaacgaagggtacatcatgggctttatcagtaaggagcgggagcgggc | |
| | | catcttgagcactaagcctccaggcaccttcctgctaagattcagtgaaagcagcaaa | |
| | | gaaggaggcgtcactttcacttgggtggagaaggacatcagcggtaagacccagatc | |
| | | cagtccgtggaaccatacacaaagcagcagctgaacaacatgtcatttgctgaaatc | |
| | | atcatgggctataagatcatggatgctaccaatatcctggtgtctccactggtctatctc | |
| | | tatcctgacattcccaaggaggaggcattcggaaagtattgtcggccagagagccag | |
| | | gagcatcctgaagctgacccaggcgctgcccGACTACAAAGACGATGACGA | |
| | | CAAGTAA | |
| 408 | pDF0873_PABPC1_ | ttgagttctattacaccactattctagaattatgaatcgctcccctgcactactctttcct | 298 |
| | 3TS_Cargo2 | tgtcctccccacactcgaaaaatatttctctttctccactagagaaagcagcagcagttg | |
| | (pDY0088 based) | agagtatggctgttggagctgatgggattaacgaggaattctcttcttttttttctgcag | |
| | 3 mutations | gtCgaCgaGgctgtagctgtactacaagcccaccaagctaaagaggctgcccagaa | |
| | in cargo | agcagttaacagtgccaccggtgttccaactgttGACTACAAAGACGATGAC | |
| | | GACAAGTAA | |
| 409 | TOP2A | caatatttattgagcacttgctatgtgtcacgcacatggacataaagtctcaatcctca | 362 |
| | cargo | aggagctcacagtccagtagaagtttgcaattaacacatattttgttaggtggtgggat | |
| | | aaacaagagaagaaaaagtgggaaagtgactgaacgaggaattctcttcttttttttc | |
| | | tgcagctcCttAgcAcgattgttatttccaccaaaagatgatcacacgttgaagttttt | |
| | | atatgatgacaaccagcgtgttgagcctgaatggtacattcctattattcccatggtgct | |
| | | gataaatggtgctgaaggaatcggtactgggtggtcctgcaaaGACTACAAAGA | |
| | | CGATGACGACAAG | |
| 410 | PPIB Hyb 50 | gaacgaggaattctcttcttttttttctgcaggaAgtTgtTcggaaggtggagagcac | 179 |
| | 1 | caagacagacagccgggataaacccctgaaggatgtgatcatcgcagactgcggca | |
| | | agatcgaggtggagaagccctttgccatcgccaaggagGACTACAAAGACGA | |
| | | TGACGACAAGTAA | |
| 411 | PPIB Hyb 50 | taatacgactcactataggggtgggaccagcacgtcactgagtgaaggaggggagg | 247 |
| | 2 | gaggctctggcagaacgaggaattctcttcttttttttctgcaggaAgtTgtTcggaag | |
| | | gtggagagcaccaagacagacagccgggataaacccctgaaggatgtgatcatcgc | |
| | | agactgcggcaagatcgaggtggagaagccctttgccatcgccaaggagGACTAC | |
| | | AAAGACGATGACGACAAGTAA | |
| 412 | PPIB Hyb 50 | taatacgactcactataggttgtgcagccttcctggctgggctctgagggggctggaa | 247 |
| | 3 | gaatttagaacaacgaggaattctcttcttttttttctgcaggaAgtTgtTcggaaggt | |
| | | ggagagcaccaagacagacagccgggataaacccctgaaggatgtgatcatcgcag | |
| | | actgcggcaagatcgaggtggagaagccctttgccatcgccaaggagGACTACA | |
| | | AAGACGATGACGACAAGTAA | |
| 413 | PPIB Hyb | gggtgggaccagcacgtcactgagtgaaggaggggagggaggctctggcagaacga | 229 |
| | 100 1 | ggaattctcttcttttttttctgcaggaAgtTgtTcggaaggtggagagcaccaagac | |
| | | agacagccgggataaacccctgaaggatgtgatcatcgcagactgcggcaagatcg | |
| | | aggtggagaagccctttgccatcgccaaggagGACTACAAAGACGATGACG | |
| | | ACAAGTAA | |
| 414 | PPIB Hyb | taatacgactcactataggggtgggaccagcacgtcactgagtgaaggaggggagg | 297 |
| | 100 2 | gaggctctggcagttgtgcagccttcctggctgggctctgagggggctggaagaattt | |
| | | agaacaacgaggaattctcttcttttttttctgcaggaAgtTgtTcggaaggtggaga | |
| | | gcaccaagacagacagccgggataaacccctgaaggatgtgatcatcgcagactgc | |
| | | ggcaagatcgaggtggagaagccctttgccatcgccaaggagGACTACAAAGA | |
| | | CGATGACGACAAGTAA | |
| 415 | STAT3_ | acagaagtaaagaaagatttccttgggaacagaaaatataaagtttctgaggagaat | 366 |
| | HYBPLUS_ | tcaaatgaagccaaaacctcaaaaaagatacatgcaggacctgcaggcagtatcccc | |
| | 25SIDES | aagagaaggctccctgttggccaggtgcagtggctcacgcctgtaatgccagcacttt | |
| | | gagaggctgagttgggaggatcacttgaaacgaggaattctcttcttttttttctgcag | |
| | | gaCctCgaGcagaaaatgaaagtggtagagaatctccaggatgactttgatttcaac | |
| | | tataaaaccctcaagagtcaaggaGACTACAAAGACGATGACGACAAGT | |
| | | AAGACTACAAAGACGATGACGACAAGTAA | |
| 416 | STAT3_ | ctgtttaaaataagcaaacaaaaaaacagaagtaaagaaagatttccttgggaaca | 416 |
| | HYBPLUS_ | gaaaatataaagtttctgaggagaattcaaatgaagccaaaacctcaaaaaagata | |
| | 50SIDES | catgcaggacctgcaggcagtatccccaagagaaggctccctgttggccaggtgcag | |
| | | tggctcacgcctgtaatgccagcactttgagaggctgagttgggaggatcacttgagc | |
| | | ccaggagttcatgatcagcctggaacgaggaattctcttcttttttttctgcaggaCctC | |
| | | gaGcagaaaatgaaagtggtagagaatctccaggatgactttgatttcaactataaa | |
| | | accctcaagagtcaaggaGACTACAAAGACGATGACGACAAGTAAGA | |
| | | CTACAAAGACGATGACGACAAGTAA | |
| 417 | STAT3_ | ggaacagaaaatataaagtttctgaggagaattcaaatgaagccaaaacctcaaaa | 366 |
| | HYBPLUS_50_ | aagatacatgcaggacctgcaggcagtatccccaagagaaggctccctgttggccag | |
| | 5PRIME | gtgcagtggctcacgcctgtaatgccagcactttgagaggctgagttgggaggatcac | |
| | | ttgagcccaggagttcatgatcagcctggaacgaggaattctcttcttttttttctgcag | |
| | | gaCctCgaGcagaaaatgaaagtggtagagaatctccaggatgactttgatttcaac | |
| | | tataaaaccctcaagagtcaaggaGACTACAAAGACGATGACGACAAGT | |
| | | AAGACTACAAAGACGATGACGACAAGTAA | |
| 418 | STAT3_ | ggaacagaaaatataaagtttctgaggagaattcaaatgaagccaaaacctcaaaa | 416 |
| | HYBPLUS_100_ | aagatacatgcaggacctgcaggcagtatccccaagagaaggctccctgttggccag | |
| | 5PRIME | gtgcagtggctcacgcctgtaatgccagcactttgagaggctgagttgggaggatcac | |
| | | ttgagcccaggagttcatgatcagcctggacaacacagggagacccccatctctaca | |
| | | aattttttttttttaattagctaacgaggaattctcttcttttttttctgcaggaCctCg | |
| | | aGcagaaaatgaaagtggtagagaatctccaggatgactttgatttcaactataaaaccc | |
| | | tcaagagtcaaggaGACTACAAAGACGATGACGACAAGTAAGACTAC | |
| | | AAAGACGATGACGACAAGTAA | |
| 419 | STAT3_ | ggaacagaaaatataaagtttctgaggagaattcaaatgaagccaaaacctcaaaa | 466 |
| | HYBPLUS_150_ | aagatacatgcaggacctgcaggcagtatccccaagagaaggctccctgttggccag | |
| | 5PRIME | gtgcagtggctcacgcctgtaatgccagcactttgagaggctgagttgggaggatcac | |
| | | ttgagcccaggagttcatgatcagcctggacaacacagggagacccccatctctaca | |
| | | aattttttttttttaattagctgggcgtggtggtgcatgcctgtggtcccggctacttggg | |
| | | aggatgaggtaaacgaggaattctcttcttttttttctgcaggaCctCgaGcagaaaa | |
| | | tgaaagtggtagagaatctccaggatgactttgatttcaactataaaaccctcaagag | |
| | | tcaaggaGACTACAAAGACGATGACGACAAGTAAGACTACAAAGA | |
| | | CGATGACGACAAGTAA | |
| 420 | STAT3_ | ctgtttaaaataagcaaacaaaaaaacagaagtaaagaaagatttccttgggaaca | 366 |
| | HYBPLUS_50_ | gaaaatataaagtttctgaggagaattcaaatgaagccaaaacctcaaaaaagata | |
| | 3PRIME | catgcaggacctgcaggcagtatccccaagagaaggctccctgttggccaggtgcag | |
| | | tggctcacgcctgtaatgccagcactttgagaacgaggaattctcttcttttttttctgca | |
| | | ggaCctCgaGcagaaaatgaaagtggtagagaatctccaggatgactttgatttcaa | |
| | | ctataaaaccctcaagagtcaaggaGACTACAAAGACGATGACGACAAG | |
| | | TAAGACTACAAAGACGATGACGACAAGTAA | |
| 421 | PPIB_wider | ATGTGGCTTCTCAGGGACATTGCGTTCAGCTGCACTCTGTATACC | 478 |
| | Cargos_150left | TCAGGGGTGGGACCAGCACGTCACTGAGTGAAGGAGGGGAGG | |
| | | GAGGCTCTGGCAGTTGTGCAGCCTTCCTGGCTGGGCTCTGAGGG | |
| | | GGCTGGAAGAATTTAGAACCTTGGAGGCATGGAGGTACAGGGT | |
| | | TTATTCTGGACAGGAGCACTGGGCTGCATCTGTGGGTTGGGTCCT | |
| | | TTTGGGAAAGGGATGGACACATGGAGCTCCTGCCCTGGGGTCTG | |
| | | TGTTGAATCCCCGGTGAGGATTGCCCAGTAGTAGCCCaacgaggaa | |
| | | ttctcttcttttttttctgcaggaAgtTgtTcggaaggtggagagcaccaagacagaca | |
| | | gccgggataaacccctgaaggatgtgatcatcgcagactgcggcaagatcgaggtgg | |
| | | agaagccctttgccatcgccaaggagGACTACAAAGACGATGACGACAAG | |
| | | TAA | |
| 422 | PPIB_wider | ATGTGGCTTCTCAGGGACATTGCGTTCAGCTGCACTCTGTATACC | 428 |
| | Cargos_100left | TCAGGGGTGGGACCAGCACGTCACTGAGTGAAGGAGGGGAGG | |
| | | GAGGCTCTGGCAGTTGTGCAGCCTTCCTGGCTGGGCTCTGAGGG | |
| | | GGCTGGAAGAATTTAGAACCTTGGAGGCATGGAGGTACAGGGT | |
| | | TTATTCTGGACAGGAGCACTGGGCTGCATCTGTGGGTTGGGTCCT | |
| | | TTTGGGAAAGGGATGGACACATGGAGCTCCTaacgaggaattctcttct | |
| | | tttttttctgcaggaAgtTgtTcggaaggtggagagcaccaagacagacagccggga | |
| | | taaacccctgaaggatgtgatcatcgcagactgcggcaagatcgaggtggagaagcc | |
| | | ctttgccatcgccaaggagGACTACAAAGACGATGACGACAAGTAA | |
| 423 | PPIB_wider | ATGTGGCTTCTCAGGGACATTGCGTTCAGCTGCACTCTGTATACC | 378 |
| | Cargos_50left | TCAGGGGTGGGACCAGCACGTCACTGAGTGAAGGAGGGGAGG | |
| | | GAGGCTCTGGCAGTTGTGCAGCCTTCCTGGCTGGGCTCTGAGGG | |
| | | GGCTGGAAGAATTTAGAACCTTGGAGGCATGGAGGTACAGGGT | |
| | | TTATTCTGGACAGGAGCACTGGGCTGaacgaggaattctcttcttttttttc | |
| | | tgcaggaAgtTgtTcggaaggtggagagcaccaagacagacagccgggataaacc | |
| | | cctgaaggatgtgatcatcgcagactgcggcaagatcgaggtggagaagccctttgc | |
| | | catcgccaaggagGACTACAAAGACGATGACGACAAGTAA | |
| 424 | PPIB_wider | CACACAAAACTGGAGGCACCAAAATTCTAACAGACTCCTGGCCA | 478 |
| | Cargos_150right | GAGCAGGGAGAATGCAGATTTGACGAGGGGGTACAGGAATTTT | |
| | | GTTCCTTTGAAGTAAGACCCAGGTTGGGCCAAGGGTGAGGAGG | |
| | | AGGAAGAGGGTGACCAGGGCATGTGGCTTCTCAGGGACATTGC | |
| | | GTTCAGCTGCACTCTGTATACCTCAGGGGTGGGACCAGCACGTC | |
| | | ACTGAGTGAAGGAGGGGAGGGAGGCTCTGGCAGTTGTGCAGCC | |
| | | TTCCTGGCTGGGCTCTGAGGGGGCTGGAAGAATTTAGAACaacga | |
| | | ggaattctcttcttttttttctgcaggaAgtTgtTcggaaggtggagagcaccaagac | |
| | | agacagccgggataaacccctgaaggatgtgatcatcgcagactgcggcaagatcg | |
| | | aggtggagaagccctttgccatcgccaaggagGACTACAAAGACGATGACG | |
| | | ACAAGTAA | |
| 425 | PPIB_wider | GGAGAATGCAGATTTGACGAGGGGGTACAGGAATTTTGTTCCTT | 428 |
| | Cargos_100right | TGAAGTAAGACCCAGGTTGGGCCAAGGGTGAGGAGGAGGAAGA | |
| | | GGGTGACCAGGGCATGTGGCTTCTCAGGGACATTGCGTTCAGCT | |
| | | GCACTCTGTATACCTCAGGGGTGGGACCAGCACGTCACTGAGTG | |
| | | AAGGAGGGGAGGGAGGCTCTGGCAGTTGTGCAGCCTTCCTGGC | |
| | | TGGGCTCTGAGGGGGCTGGAAGAATTTAGAACaacgaggaattctctt | |
| | | cttttttttctgcaggaAgtTgtTcggaaggtggagagcaccaagacagacagccgg | |
| | | gataaacccctgaaggatgtgatcatcgcagactgcggcaagatcgaggtggagaa | |
| | | gccctttgccatcgccaaggagGACTACAAAGACGATGACGACAAGTAA | |
| 426 | PPIB_wider | AAGACCCAGGTTGGGCCAAGGGTGAGGAGGAGGAAGAGGGTG | 378 |
| | Cargos_50right | ACCAGGGCATGTGGCTTCTCAGGGACATTGCGTTCAGCTGCACTC | |
| | | TGTATACCTCAGGGGTGGGACCAGCACGTCACTGAGTGAAGGAG | |
| | | GGGAGGGAGGCTCTGGCAGTTGTGCAGCCTTCCTGGCTGGGCTC | |
| | | TGAGGGGGCTGGAAGAATTTAGAACaacgaggaattctcttcttttttttct | |
| | | gcaggaAgtTgtTcggaaggtggagagcaccaagacagacagccgggataaaccc | |
| | | ctgaaggatgtgatcatcgcagactgcggcaagatcgaggtggagaagccctttgcc | |
| | | atcgccaaggagGACTACAAAGACGATGACGACAAGTAA | |
| 427 | PPIB_wider | TACAGGAATTTTGTTCCTTTGAAGTAAGACCCAGGTTGGGCCAAG | 478 |
| | Cargos_7575 | GGTGAGGAGGAGGAAGAGGGTGACCAGGGCATGTGGCTTCTCA | |
| | | GGGACATTGCGTTCAGCTGCACTCTGTATACCTCAGGGGTGGGA | |
| | | CCAGCACGTCACTGAGTGAAGGAGGGGAGGGAGGCTCTGGCAG | |
| | | TTGTGCAGCCTTCCTGGCTGGGCTCTGAGGGGGCTGGAAGAATT | |
| | | TAGAACCTTGGAGGCATGGAGGTACAGGGTTTATTCTGGACAGG | |
| | | AGCACTGGGCTGCATCTGTGGGTTGGGTCCTTTTGGGaacgaggaa | |
| | | ttctcttcttttttttctgcaggaAgtTgtTcggaaggtggagagcaccaagacagaca | |
| | | gccgggataaacccctgaaggatgtgatcatcgcagactgcggcaagatcgaggtgg | |
| | | agaagccctttgccatcgccaaggagGACTACAAAGACGATGACGACAAG | |
| | | TAA | |
| 428 | PPIB_wider | AAGACCCAGGTTGGGCCAAGGGTGAGGAGGAGGAAGAGGGTG | 428 |
| | Cargos_5050 | ACCAGGGCATGTGGCTTCTCAGGGACATTGCGTTCAGCTGCACTC | |
| | | TGTATACCTCAGGGGTGGGACCAGCACGTCACTGAGTGAAGGAG | |
| | | GGGAGGGAGGCTCTGGCAGTTGTGCAGCCTTCCTGGCTGGGCTC | |
| | | TGAGGGGGCTGGAAGAATTTAGAACCTTGGAGGCATGGAGGTA | |
| | | CAGGGTTTATTCTGGACAGGAGCACTGGGCTGaacgaggaattctctt | |
| | | cttttttttctgcaggaAgtTgtTcggaaggtggagagcaccaagacagacagccgg | |
| | | gataaacccctgaaggatgtgatcatcgcagactgcggcaagatcgaggtggagaa | |
| | | gccctttgccatcgccaaggagGACTACAAAGACGATGACGACAAGTAA | |
| 429 | PPIB_wider | GGAGGAGGAAGAGGGTGACCAGGGCATGTGGCTTCTCAGGGAC | 378 |
| | Cargos_2525 | ATTGCGTTCAGCTGCACTCTGTATACCTCAGGGGGGGACCAGCA | |
| | | CGTCACTGAGTGAAGGAGGGGAGGGAGGCTCTGGCAGTTGTGC | |
| | | AGCCTTCCTGGCTGGGCTCTGAGGGGGCTGGAAGAATTTAGAAC | |
| | | CTTGGAGGCATGGAGGTACAGGGTTaacgaggaattctcttcttttttttct | |
| | | gcaggaAgtTgtTcggaaggtggagagcaccaagacagacagccgggataaaccc | |
| | | ctgaaggatgtgatcatcgcagactgcggcaagatcgaggtggagaagccctttgcc | |
| | | atcgccaaggagGACTACAAAGACGATGACGACAAGTAA | |
| 430 | STAT3 | ggaacagaaaatataaagtttctgaggagaattcaaatgaagccaaaacctcaaaa | 304 |
| | ESE_Ax1 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGCAAGGCGGAGGAAG | |
| 431 | STAT3 | ggaacagaaaatataaagtttctgaggagaattcaaatgaagccaaaacctcaaaa | 318 |
| | ESE_Ax2 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGCAAGGCGGAGGAAGCAAGGCGGAGGAAG | |
| 432 | STAT3 | ggaacagaaaatataaagtttctgaggagaattcaaatgaagccaaaacctcaaaa | 334 |
| | ESE_Ax3 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGCAAGGCGGAGGAAAGCAAGGCGGAGGAAGGCAA | |
| | | GGCGGAGGAAG | |
| 433 | STAT3 | ggaacagaaaatataaagtttctgaggagaattcaaatgaagccaaaacctcaaaa | 350 |
| | ESE_Ax4 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGCAAGGCGGAGGAAAGCAAGGCGGAGGAAGAGCA | |
| | | AGGCGGAGGAAGGCAAGGCGGAGGAAG | |
| 434 | STAT3 | ggaacagaaaatataaagtttctgaggagaattcaaatgaagccaaaacctcaaaa | 310 |
| | ESE_Bx2 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGCACACAGGACCACACAGGAC | |
| 435 | STAT3 | ggaacagaaaatataaagtttctgaggagaattcaaatgaagccaaaacctcaaaa | 314 |
| | ESE_Cx2 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGAAAAAGAAAGAAAAAAAGAAAGAA | |
| 436 | STAT3 | ggaacagaaaatataaagtttctgaggagaattcaaatgaagccaaaacctcaaaa | 306 |
| | ESE_Dx2 | aagatacatgcaggacctgcaggcagtatccccaagagaaggctccctgttggccag | |
| | | gtgcagtggctcacgcctgtaatgccagcactttgagaacgaggaattctcttctttttt | |
| | | ttctgcaggaCctCgaGcagaaaatgaaagtggtagagaatctccaggatgactttg | |
| | | atttcaactataaaaccctcaagagtcaaggaGACTACAAAGACGATGACG | |
| | | ACAAGTAAGTCAGAGGATCAGAGGA | |
| 437 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v0 | ctgaaggcagaggcaccaaaagctacaagagcaaacaacgaggaAttctcttctttt | |
| | | ttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 438 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v1 | ctgaaggcagaggcaccaaaagctacaagagcaaacaacgacTaActctcttctttt | |
| | | ttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 439 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v3 | ctgaaggcagaggcaccaaaagctacaagagcaaacaacgatTaAttctcttctttt | |
| | | ttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 440 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v6 | ctgaaggcagaggcaccaaaagctacaagagcaaacaacgatTgActctcttctttt | |
| | | ttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 441 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v12 | ctgaaggcagaggcaccaaaagctacaagagcaaacaacgacTcAttctcttctttt | |
| | | ttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 378 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v13 | ctgaaggcagaggcaccaaaagctacaagagcaaacaacgacTtActctcttctttt | |
| | | ttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 442 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | v15 | ctgaaggcagaggcaccaaaagctacaagagcaaacaacgatTtAttctcttcttttt | |
| | | tttctgcagtcCCtgttCgaacgccagggcctcccaggcccagagaagctgccgggc | |
| | | tccttgcggaaggggattccacggaccaagtctGACTACAAAGACGATGACG | |
| | | ACAAGTAA | |
| 443 | SHANK3 | gaggagcctagaaggcgccgggttggcaagtgggcagggaacagagacatgctttg | 289 |
| | branchpoint | tcgtgttctcagcggagctggggtaccttggtggtctcaggcgtgagacagggactgc | |
| | yeast | ctgaaggcagaggcaccaaaagctacaagagcaaacaacTACTAACtctcttcttt | |
| | | tttttctgcagtcCCtgttCgaacgccagggcctcccaggcccagagaagctgccggg | |
| | | ctccttgcggaaggggattccacggaccaagtctGACTACAAAGACGATGAC | |
| | | GACAAGTAA | |
| 444 | SHANK3_ | GAGGAGCCTAGAAGGCGCCGGGTTGGCAAGTGGGCAGGGAAC | 304 |
| | ESE_Ax1 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GCAAGGCGGAGGAAG | |
| 445 | SHANK3_ | GAGGAGCCTAGAAGGCGCCGGGTTGGCAAGTGGGCAGGGAAC | 318 |
| | ESE_Ax2 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GCAAGGCGGAGGAAGCAAGGCGGAGGAAG | |
| 446 | SHANK3_ | GAGGAGCCTAGAAGGCGCCGGGTTGGCAAGTGGGCAGGGAAC | 334 |
| | ESE_Ax3 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GCAAGGCGGAGGAAAGCAAGGCGGAGGAAGGCAAGGCGGAGG | |
| | | AAG | |
| 447 | SHANK3_ | GAGGAGCCTAGAAGGCGCCGGGTTGGCAAGTGGGCAGGGAAC | 350 |
| | ESE_Ax4 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GCAAGGCGGAGGAAAGCAAGGCGGAGGAAGAGCAAGGCGGAG | |
| | | GAAGGCAAGGCGGAGGAAG | |
| 448 | SHANK3_ | GAGGAGCCTAGAAGGCGCCGGGTTGGCAAGTGGGCAGGGAAC | 310 |
| | ESE_Bx2 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GCACACAGGACCACACAGGAC | |
| 449 | SHANK3_ | GAGGAGCCTAGAAGGCGCCGGGTTGGCAAGTGGGCAGGGAAC | 331 |
| | ESE_Bx4 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GCACACAGGACCACACAGGACGCACACAGGACCACACAGGAC | |
| 450 | SHANK3_ | GAGGAGCCTAGAAGGCGCCGGGTTGGCAAGTGGGCAGGGAAC | 314 |
| | ESE_Cx2 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GAAAAAGAAAGAAAAAAAGAAAGAA | |
| 612 | SHANK3_ | GAGGAGCCTAGAAGGCGCCGGGTTGGCAAGTGGGCAGGGAAC | 339 |
| | ESE_Cx4 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GAAAAAGAAAGAAAAAAAGAAAGAAGAAAAAGAAAGAAAAAA | |
| | | AGAAAGAA | |
| 451 | SHANK3_ | GAGGAGCCTAGAAGGCGCCGGGTTGGCAAGTGGGCAGGGAAC | 306 |
| | ESE_Dx2 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GTCAGAGGATCAGAGGA | |
| 452 | SHANK3_ | GAGGAGCCTAGAAGGCGCCGGGTTGGCAAGTGGGCAGGGAAC | 323 |
| | ESE_Dx4 | AGAGACATGCTTTGTCGTGTTCTCAGCGGAGCTGGGGTACCTTG | |
| | | GTGGTCTCAGGCGTGAGACAGGGACTGCCTGAAGGCAGAGGCA | |
| | | CCAAAAGCTACAAGAGCAAACaacgaggaattctcttcttttttttctgcagtc | |
| | | CCtgttCgaacgccagggcctcccaggcccagagaagctgccgggctccttgcggaa | |
| | | ggggattccacggaccaagtctGACTACAAAGACGATGACGACAAGTAA | |
| | | GTCAGAGGATCAGAGGAGTCAGAGGATCAGAGGA | |
| 453 | USF1 ITS | tcacactttggacctcattttcatctaaggaaggtggtataatatctcccagggataca | 511 |
| | cargo 1 | ggaacctcagggagagataagactactgtcatgtgtgcccctctctctaccatttctgg | |
| | | aaacaataccaggaggcagaattcaggcatccaacgacTaActctcttcttttttttct | |
| | | gcagagCaaGggAgggattctatccaaagcttgtgattatatccaggagcttcggca | |
| | | gagtaaccaccgcttgtctgaagaactgcagggacttgaccaactgcagctggacaa | |
| | | tgacgtgctCcgTcaGcagGTAAGTttgggcTGCATGacTGCATGgtTGCA | |
| | | TGaacacaTATTAATttccGTAGTAAAGCTGGCACTTCCAAGCCCCT | |
| | | GAATGTATTCAGACATCCACTGGTGAGGGGGAAAAGATGAAGCC | |
| | | TTCTCCATGGAGAACAAAGTAGAGGGTGTCAAACTGGGTCAGTG | |
| | | GCTAGCAGAACTGAGAAGGGCTGCACTGGGGGTA | |
| 454 | USF1 ITS | TTGACCAAGTGGAGGGTGCTCTTCCAGCTCTTGAACAGGACCTA | 511 |
| | cargo 2 | GAGAGTTGGATGTATTAGATGGGCGTACGCAgTATGTGCCCAGT | |
| | | TGTATGATTGTGCGTTTTCAAGGAAGGGAGTGTGCGTCGATTCGT | |
| | | TCAGTATCGACAgGGGGaacgacTaActctcttcttttttttctgcagagCaa | |
| | | GggAgggattctatccaaagcttgtgattatatccaggagcttcggcagagtaacca | |
| | | ccgcttgtctgaagaactgcagggacttgaccaactgcagctggacaatgacgtgctC | |
| | | cgTcaGcagGTAAGTttgggcTGCATGacTGCATGgtTGCATGaacacaT | |
| | | ATTAATttccGTAGTAAAGCTGGCACTTCCAAGCCCCTGAATGTAT | |
| | | TCAGACATCCACTGGTGAGGGGGAAAAGATGAAGCCTTCTCCAT | |
| | | GGAGAACAAAGTAGAGGGTGTCAAACTGGGTCAGTGGCTAGCA | |
| | | GAACTGAGAAGGGCTGCACTGGGGGTA | |
| 455 | USF1 ITS | tcacactttggacctcattttcatctaaggaaggtggtataatatctcccagggataca | 511 |
| | cargo 3 | ggaacctcagggagagataagactactgtcatgtgtgcccctctctctaccatttctgg | |
| | | aaacaataccaggaggcagaattcaggcatccaacgacTaActctcttcttttttttct | |
| | | gcagagCaaGggAgggattctatccaaagcttgtgattatatccaggagcttcggca | |
| | | gagtaaccaccgcttgtctgaagaactgcagggacttgaccaactgcagctggacaa | |
| | | tgacgtgctCcgTcaGcagGTAAGTttgggcTGCATGacTGCATGgtTGCA | |
| | | TGaacacaTATTAATttccTTGACCAAGTGGAGGGTGCTCTTCCAGC | |
| | | TCTTGAACAGGACCTAGAGAGTTGGATGTATTAGATGGGCGTAC | |
| | | GCAgTATGTGCCCAGTTGTATGATTGTGCGTTTTCAAGGAAGGGA | |
| | | GTGTGCGTCGATTCGTTCAGTATCGACAgGGGG | |
| 456 | USF1 ITS | TTGACCAAGTGGAGGGTGCTCTTCCAGCTCTTGAACAGGACCTA | 511 |
| | cargo 4 | GAGAGTTGGATGTATTAGATGGGCGTACGCAgTATGTGCCCAGT | |
| | | TGTATGATTGTGCGTTTTCAAGGAAGGGAGTGTGCGTCGATTCGT | |
| | | TCAGTATCGACAgGGGGaacgacTaActctcttcttttttttctgcagagCaa | |
| | | GggAgggattctatccaaagcttgtgattatatccaggagcttcggcagagtaacca | |
| | | ccgcttgtctgaagaactgcagggacttgaccaactgcagctggacaatgacgtgctC | |
| | | cgTcaGcagGTAAGTttgggcTGCATGacTGCATGgtTGCATGaacacaT | |
| | | ATTAATttccCTATTCAGGGATTGACTGATACCGGAAGACATCTCA | |
| | | GTTGAAGTGGTCTATACGACAGAGACCGTGCACCTACCAAATCTC | |
| | | CTTAGTGTAAGTTCAGACCAATTGGTAGTTTGTCCAGAACTCAGA | |
| | | TTTTAACAGCAGAGGACGCATGCT | |
| 457 | HTT_opt_ | gcccggctgtggctgaggagccCctCcaccgaccGTAAGTttgggcTGCATGac | 235 |
| | hyb1_ISE_BP_ | TGCATGgtTGCATGaacacaTATTAATttccatatgtgttgaattacccactcc | |
| | cargo1 | acttagttctacacctcattcattcattcagtgagtgtttctcgactactatgaataaacc | |
| | | gttatactccatgttgcgggcagaatggggatctggacagggaagcacagggcacga | |
| | | gttcaccaa | |
| 458 | HTT_opt_ | gcccggctgtggctgaggagccCctCcaccgaccGTAAGTttgggcTGCATGac | 224 |
| | hyb1_ISE_noBP_ | TGCATGgtTGCATGaacacaatatgtgttgaattacccactccacttagttctaca | |
| | cargo2 | cctcattcattcattcagtgagtgtttctcgactactatgaataaaccgttatactccatg | |
| | | ttgcgggcagaatggggatctggacagggaagcacagggcacgagttcaccaa | |
| 371 | HTT_opt_ | gcccggctgtggctgaggagccCctCcaccgaccGTAAGTttgggcTGCATGac | 235 |
| | hyb2_ISE_BP_ | TGCATGgtTGCATGaacacaTATTAATttcctccacttagttctacacctcattc | |
| | cargo3 | attcattcagtgagtgtttctcgactactatgaataaaccgttatactccatgttgcggg | |
| | | cagaatggggatctggacagggaagcacagggcacgagttcaccaatggctgtcaa | |
| | | gctacgctgc | |
| 459 | HTT_opt_ | gcccggctgtggctgaggagccCctCcaccgaccGTAAGTttgggcTGCATGac | 224 |
| | hyb2_ISE_noBP_ | TGCATGgtTGCATGaacacatccacttagttctacacctcattcattcattcagtg | |
| | cargo4 | agtgtttctcgactactatgaataaaccgttatactccatgttgcgggcagaatgggga | |
| | | tctggacagggaagcacagggcacgagttcaccaatggctgtcaagctacgctgc | |
| 460 | HTT_opt_ | gcccggctgtggctgaggagccCctCcaccgaccGTAAGTttgggcTGCATGac | 251 |
| | hyb3_ISE_BP_ | TGCATGgtTGCATGaacacaTATTAATttccttcttttttttattttaaaataaa | |
| | PPT_cargo5 | aaaaagaaggaaattaatatgtgttgaattacccactccacttagttctacacctcatt | |
| | | cattcattcagtgagtgtttctcgactactatgaataaaccgttatactccatgttgcgg | |
| | | gcagaatggggatctggacaggg | |
| 461 | HTT_opt_ | gcccggctgtggctgaggagccCctCcaccgaccGTAAGTttgggcaacacaTA | 213 |
| | hyb1_noISE_ | TTAATttccatatgtgttgaattacccactccacttagttctacacctcattcattcatt | |
| | BP_cargo6 | cagtgagtgtttctcgactactatgaataaaccgttatactccatgttgcgggcagaat | |
| | | ggggatctggacagggaagcacagggcacgagttcaccaa | |
| 462 | HTT_opt_ | gcccggctgtggctgaggagccCctCcaccgaccGTAAGTttgggcTGCATGac | 185 |
| | hyb1(100 bp)_ | TGCATGgtTGCATGaacacaTATTAATttccatatgtgttgaattacccactcc | |
| | ISE_BP_cargo7 | acttagttctacacctcattcattcattcagtgagtgtttctcgactactatgaataaacc | |
| | | gttatactccatgttg | |
| 463 | HTT_opt_ | gcccggctgtggctgaggagccCctCcaccgaccGTAAGTttggatacttacctgg | 401 |
| | hyb1_ | caggggagataccatgatcacgaaggtggttttcccagggcgaggcttatccattgca | |
| | U1snRNA(FL)_ | ctccggatgtgctgacccctgcgatttccccaaatgtgggaaactcgactgcataattt | |
| | ISE_BP_ | gtggtagtgggggactgcgttcgcgctttcccctggccaTGCATGacTGCATGgt | |
| | cargo8 | TGCATGaacacaTATTAATttccatatgtgttgaattacccactccacttagttct | |
| | | acacctcattcattcattcagtgagtgtttctcgactactatgaataaaccgttatactc | |
| | | catgttgcgggcagaatggggatctggacagggaagcacagggcacgagttcacca | |
| | | a | |
| 464 | HTT_opt_ | gcccggctgtggctgaggagccCctCcaccgaccGTAAGTttggataatttgtggt | 279 |
| | hyb1_ | agtgggggactgcgttcgcgctttcccctggccaTGCATGacTGCATGgtTGCA | |
| | U1snRNA(sm&SL4)_ | TGaacacaTATTAATttccatatgtgttgaattacccactccacttagttctacacct | |
| | ISE_BP_cargo9 | cattcattcattcagtgagtgtttctcgactactatgaataaaccgttatactccatgttg | |
| | | cgggcagaatggggatctggacagggaagcacagggcacgagttcaccaa | |

Table 15 shows the protein for nuclease/reporter constructs.

| SEQ | | | |
|---|---|---|---|
| ID NO | ID | Sequence | Length |
| 465 | SMALL | MTTTMKISIEFLEPFRMTKWQESTRRNKNNKEFVRGQA | 1529 |
| | CAS | FARWHRNKKDNTKGRPYITGTLLRSAVIRSAENLLTLS | |
| | R1045- | DGKISEKTCCPGKFDTEDKDRLLQLRQRSTLRWTDKNP | |
| | GGGS- | CPDNAETYCPFCELLGRSGNDGKKAEKKDWRFRIHFGN | |
| | R1122 | LSLPGKPDFDGPKAIGSQRVLNRVDFKSGKAHDFFKAY | |
| | | EVDHTRFPRFEGEITIDNKVSAEARKLLCDSLKFTDRL | |
| | | CGALCVIRFDEYTPAADSGKQTENVQAEPNANLAEKTA | |
| | | EQIISILDDNKKTEYTRLLADAIRSLRRSSKLVAGLPK | |
| | | DHDGKDDHYLWDIGKKKKDENSVTIRQILTTSADTKEL | |
| | | KNAGKWREFCEKLGEALYLKSKDMSGGLKITRRILGDA | |
| | | EFHGKPDRLEKSRSVSIGSVLKETVVCGELVAKTPFFF | |
| | | GAIDEDAKQTDLQVLLTPDNKYRLPRSAVRGILRRDLQ | |
| | | TYFDSPCNAELGGRPCMCKTCRIMRGITVMDARSEYNA | |
| | | PPEIRHRTRINPFTGTVAEGALFNMEVAPEGIVEPFQL | |
| | | RYRGSEDGLPDALKTVLKWWAEGQAFMSGAASTGKGRF | |
| | | RMENAKYETLDLSDENQRNDYLKNWGWRDEKGLEELKK | |
| | | RLNSGLPEPGNYRDPKWHEINVSIEMASPFINGDPIRA | |
| | | AVDKRGTDVVTFVKYKAEGEEAKPVCAYKAESFRGVIR | |
| | | SAVARIHMEDGVPLTELTHSDCECLLCQIFGSEYEAGK | |
| | | IRFEDLVFESDPEPVTFDHVAIDRFTGGAADKKKFDDS | |
| | | PLPGSPARPLMLKGSFWIRRDVLEDEEYCKALGKALAD | |
| | | VNNGLYPLGGKSAIGYGQVKSLGIKGDDKRISRLMNPA | |
| | | FDETDVAVPEKPKTDAEVRIEAEKVYYPHYFVEPHKKV | |
| | | EREEKPCGHQKFHEGRLTGKIRCKLITKTPLIVPDTSN | |
| | | DDFFRPADKEARKEKDEYHKSYAFFRLHKQIMIPGSEL | |
| | | RGMVSSVYETVTNSCFRIFDETKRLSWRMDADHQNVLQ | |
| | | DFLPGRVTADGKHIQKFSETARVPFYDKTQKHFDILDE | |
| | | QEIAGEKPVRMWVKRFIKRGGGSRDSRYQKAFQEIPEN | |
| | | DPDGWECKEGYLHVVGPSKVEFSDKKGDVINNFQGTLP | |
| | | SVPNDWKTIRTNDFKNRKRKNEPVFCCEDDKGNYYTMA | |
| | | KYCETFFFDLKENEEYEIPEKARIKYKELLRVYNNNPQ | |
| | | AVPESVFQSRVARENVEKLKSGDLVYFKHNEKYVEDIV | |
| | | PVRISRTVDDRMIGKRMSADLRPCHGDWVEDGDLSALN | |
| | | AYPEKRLLLRHPKGLCPACRLFGTGSYKGRVRFGFASL | |
| | | ENDPEWLIPGKNPGDPFHGGPVMLSLLERPRPTWSIPG | |
| | | SDNKFKVPGRKFYVHHHAWKTIKDGNHPTTGKAIEQSP | |
| | | NNRTVEALAGGNSFSFEIAFENLKEWELGLLIHSLQLE | |
| | | KGLAHKLGMAKSMGFGSVEIDVESVRLRKDWKQWRNGN | |
| | | SEIPNWLGKGFAKLKEWFRDELDFIENLKKLLWFPEGD | |
| | | QAPRVCYPMLRKKDDPNGNSGYEELKDGEFKKEDRQKK | |
| | | LTTPWTPWA | |
| 466 | SMALL | MKRTADGSEFESPKKKRKVTTTMKISIEFLEPFRMTKW | 1354 |
| | CAS | QESTRRNKNNKEFVRGQAFARWHRNKKDNTKGRPYITG | |
| | S1006- | TLLRSAVIRSAENLLTLSDGKISEKTCCPGKFDTEDKD | |
| | GGGS- | RLLQLRQRSTLRWTDKNPCPDNAETYCPFCELLGRSGN | |
| | D1221 | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAKRTADGSEFESPKKKRKV | |
| 467 | SMALL | MKRTADGSEFESPKKKRKVTTTMKISIEFLEPFRMTKW | 1326 |
| | CAS | QESTRRNKNNKEFVRGQAFARWHRNKKDNTKGRPYITG | |
| | R978- | TLLRSAVIRSAENLLTLSDGKISEKTCCPGKFDTEDKD | |
| | GGGS- | RLLQLRQRSTLRWTDKNPCPDNAETYCPFCELLGRSGN | |
| | R1294 | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSESFEIAFENLKEWELGLL | |
| | | IHSLQLEKGLAHKLGMAKSMGFGSVEIDVESVRLRKDW | |
| | | KQWRNGNSEIPNWLGKGFAKLKEWERDELDFIENLKKL | |
| | | LWFPEGDQAPRVCYPMLRKKDDPNGNSGYEELKDGEFK | |
| | | KEDRQKKLTTPWTPWAKRTADGSEFESPKKKRKV | |
| 2 | WT | MKRTADGSEFESPKKKRKVTTTMKISIEFLEPFRMTKW | 1637 |
| | CAS7-11 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSESFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAKRTADGSEFESPKKK | |
| | | RKV | |
| 468 | PDF0610 | MKRTADGSEFESPKKKRKVTTTMKISIEFLEPFRMTKW | 1637 |
| | dead | QESTRRNKNNKEFVRGQAFARWHRNKKDNTKGRPYITG | |
| | Cas7-11 | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDEDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTALQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTAVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSESFEIAFENLK | |
| | | EWELGLLIHSLQLEKGLAHKLGMAKSMGFGSVEIDVES | |
| | | VRLRKDWKQWRNGNSEIPNWLGKGFAKLKEWERDELDF | |
| | | IENLKKLLWFPEGDQAPRVCYPMLRKKDDPNGNSGYEE | |
| | | LKDGEFKKEDRQKKLTTPWTPWAKRTADGSEFESPKKK | |
| | | RKV | |
| 469 | LwaCas13 | MKVTKVDGISHKKYIEEGKLVKSTSEENRTSERLSELL | 1458 |
| | | SIRLDIYIKNPDNASEEENRIRRENLKKFFSNKVLHLK | |
| | | DSVLYLKNRKEKNAVQDKNYSEEDISEYDLKNKNSFSV | |
| | | LKKILLNEDVNSEELEIFRKDVEAKLNKINSLKYSFEE | |
| | | NKANYQKINENNVEKVGGKSKRNIIYDYYRESAKRNDY | |
| | | INNVQEAFDKLYKKEDIEKLFFLIENSKKHEKYKIREY | |
| | | YHKIIGRKNDKENFAKIIYEEIQNVNNIKELIEKIPDM | |
| | | SELKKSQVFYKYYLDKEELNDKNIKYAFCHFVEIEMSQ | |
| | | LLKNYVYKRLSNISNDKIKRIFEYQNLKKLIENKLLNK | |
| | | LDTYVRNCGKYNYYLQVGEIATSDFIARNRQNEAFLRN | |
| | | IIGVSSVAYFSLRNILETENENGITGRMRGKTVKNNKG | |
| | | EEKYVSGEVDKIYNENKQNEVKENLKMFYSYDFNMDNK | |
| | | NEIEDFFANIDEAISSIRHGIVHFNLELEGKDIFAFKN | |
| | | IAPSEISKKMFQNEINEKKLKLKIFKQLNSANVFNYYE | |
| | | KDVIIKYLKNTKFNFVNKNIPFVPSFTKLYNKIEDLRN | |
| | | TLKFFWSVPKDKEEKDAQIYLLKNIYYGEFLNKFVKNS | |
| | | KVFFKITNEVIKINKQRNQKTGHYKYQKFENIEKTVPV | |
| | | EYLAIIQSREMINNQDKEEKNTYIDFIQQIFLKGFIDY | |
| | | LNKNNLKYIESNNNNDNNDIFSKIKIKKDNKEKYDKIL | |
| | | KNYEKHNRNKEIPHEINEFVREIKLGKILKYTENLNMF | |
| | | YLILKLLNHKELTNLKGSLEKYQSANKEETFSDELELI | |
| | | NLLNLDNNRVTEDFELEANEIGKFLDFNENKIKDRKEL | |
| | | KKFDTNKIYFDGENIIKHRAFYNIKKYGMLNLLEKIAD | |
| | | KAKYKISLKELKEYSNKKNEIEKNYTMQQNLHRKYARP | |
| | | KKDEKFNDEDYKEYEKAIGNIQKYTHLKNKVEFNELNL | |
| | | LQGLLLKILHRLVGYTSIWERDLRFRLKGEFPENHYIE | |
| | | EIFNFDNSKNVKYKSGQIVEKYINFYKELYKDNVEKRS | |
| | | IYSDKKVKKLKQEKKDLYIRNYIAHFNYIPHAEISLLE | |
| | | VLENLRKLLSYDRKLKNAIMKSIVDILKEYGFVATFKI | |
| | | GADKKIEIQTLESEKIVHLKNLKKKKLMTDRNSEELCE | |
| | | LVKVMFEYKALEGGGGSGGGGSGGGGSVSKGEELFTGV | |
| | | VPILVELDGDVNGHKFSVRGEGEGDATNGKLTLKFICT | |
| | | TGKLPVPWPTLVTTLTYGVQCFSRYPDHMKQHDFFKSA | |
| | | MPEGYVQERTISFKDDGTYKTRAEVKFEGDTLVNRIEL | |
| | | KGIDFKEDGNILGHKLEYNFNSHNVYITADKQKNGIKA | |
| | | NFKIRHNVEDGSVQLADHYQQNTPIGDGPVLLPDNHYL | |
| | | STQSKLSKDPNEKRDHMVLLEFVTAAGITLGMDELYKG | |
| | | SEGAPKKKRKVGSSYPYDVPDYAYPYDVPDYAYPYDVP | |
| | | DYAKRTADGSEFES | |
| 470 | PspCas13 | MNIPALVENQKKYFGTYSVMAMLNAQTVLDHIQKVADI | 1133 |
| | | EGEQNENNENLWFHPVMSHLYNAKNGYDKQPEKTMFII | |
| | | ERLQSYFPFLKIMAENQREYSNGKYKQNRVEVNSNDIF | |
| | | EVLKRAFGVLKMYRDLTNHYKTYEEKLNDGCEFLTSTE | |
| | | QPLSGMINNYYTVALRNMNERYGYKTEDLAFIQDKRFK | |
| | | FVKDAYGKKKSQVNTGFFLSLQDYNGDTQKKLHLSGVG | |
| | | IALLICLFLDKQYINIFLSRLPIFSSYNAQSEERRIII | |
| | | RSFGINSIKLPKDRIHSEKSNKSVAMDMLNEVKRCPDE | |
| | | LFTTLSAEKQSRFRIISDDHNEVLMKRSSDRFVPLLLQ | |
| | | YIDYGKLFDHIRFHVNMGKLRYLLKADKTCIDGQTRVR | |
| | | VIEQPLNGFGRLEEAETMRKQENGTFGNSGIRIRDFEN | |
| | | MKRDDANPANYPYIVDTYTHYILENNKVEMFINDKEDS | |
| | | APLLPVIEDDRYVVKTIPSCRMSTLEIPAMAFHMFLFG | |
| | | SKKTEKLIVDVHNRYKRLFQAMQKEEVTAENIASFGIA | |
| | | ESDLPQKILDLISGNAHGKDVDAFIRLTVDDMLTDTER | |
| | | RIKRFKDDRKSIRSADNKMGKRGFKQISTGKLADFLAK | |
| | | DIVLFQPSVNDGENKITGLNYRIMQSAIAVYDSGDDYE | |
| | | AKQQFKLMFEKARLIGKGTTEPHPFLYKVFARSIPANA | |
| | | VEFYERYLIERKFYLTGLSNEIKKGNRVDVPFIRRDQN | |
| | | KWKTPAMKTLGRIYSEDLPVELPRQMFDNEIKSHLKSL | |
| | | PQMEGIDFNNANVTYLIAEYMKRVLDDDFQTFYQWNRN | |
| | | YRYMDMLKGEYDRKGSLQHCFTSVEEREGLWKERASRT | |
| | | ERYRKQASNKIRSNRQMRNASSEEIETILDKRLSNSRN | |
| | | EYQKSEKVIRRYRVQDALLFLLAKKTLTELADEDGERF | |
| | | KLKEIMPDAEKGILSEIMPMSFTFEKGGKKYTITSEGM | |
| | | KLKNYGDFFVLASDKRIGNLLELVGSDIVSKEDIMEEF | |
| | | NKYDQCRPEISSIVFNLEKWAFDTYPELSARVDREEKV | |
| | | DFKSILKILLNNKNINKEQSDILRKIRNAFDHNNYPDK | |
| | | GVVEIKALPEIAMSIKKAFGEYAIMKGSLQLPPLERLT | |
| | | LGSSYPYDVPDYAYPYDVPDYAYPYDVPDYA | |
| 471 | RfxCas13 | MSPKKKRKVEASIEKKKSFAKGMGVKSTLVSGSKVYMT | 1016 |
| | | TFAEGSDARLEKIVEGDSIRSVNEGEAFSAEMADKNAG | |
| | | YKIGNAKFSHPKGYAVVANNPLYTGPVQQDMLGLKETL | |
| | | EKRYFGESADGNDNICIQVIHNILDIEKILAEYITNAA | |
| | | YAVNNISGLDKDIIGFGKFSTVYTYDEFKDPEHHRAAF | |
| | | NNNDKLINAIKAQYDEFDNFLDNPRLGYFGQAFFSKEG | |
| | | RNYIINYGNECYDILALLSGLRHWVVHNNEEESRISRT | |
| | | WLYNLDKNLDNEYISTLNYLYDRITNELTNSFSKNSAA | |
| | | NVNYIAETLGINPAEFAEQYFRFSIMKEQKNLGFNITK | |
| | | LREVMLDRKDMSEIRKNHKVFDSIRTKVYTMMDFVIYR | |
| | | YYIEEDAKVAAANKSLPDNEKSLSEKDIFVINLRGSFN | |
| | | DDQKDALYYDEANRIWRKLENIMHNIKEFRGNKTREYK | |
| | | KKDAPRLPRILPAGRDVSAFSKLMYALTMFLDGKEIND | |
| | | LLTTLINKFDNIQSFLKVMPLIGVNAKFVEEYAFFKDS | |
| | | AKIADELRLIKSFARMGEPIADARRAMYIDAIRILGTN | |
| | | LSYDELKALADTFSLDENGNKLKKGKHGMRNFIINNVI | |
| | | SNKRFHYLIRYGDPAHLHEIAKNEAVVKFVLGRIADIQ | |
| | | KKQGQNGKNQIDRYYETCIGKDKGKSVSEKVDALTKII | |
| | | TGMNYDQFDKKRSVIEDTGRENAEREKFKKIISLYLTV | |
| | | IYHILKNIVNINARYVIGFHCVERDAQLYKEKGYDINL | |
| | | KKLEEKGFSSVTKLCAGIDETAPDKRKDVEKEMAERAK | |
| | | ESIDSLESANPKLYANYIKYSDEKKAEEFTRQINREKA | |
| | | KTALNAYLRNTKWNVIIREDLLRIDNKTCTLFRNKAVH | |
| | | LEVARYVHAYINDIAEVNSYFQLYHYIMQRIIMNERYE | |
| | | KSSGKVSEYFDAVNDEKKYNDRLLKLLCVPFGYCIPRF | |
| | | KNLSIEALFDRNEAAKFDKEKKKVSGNSGSGPKKKRKV | |
| | | AAAYPYDVPDYAASGSGKRTADGSEFES | |
| 472 | LwaCas13 | MKRTADGSEFESPKKKRKVKVTKVDGISHKKYIEEGKL | 1483 |
| | with | VKSTSEENRTSERLSELLSIRLDIYIKNPDNASEEENR | |
| | NLS | IRRENLKKFFSNKVLHLKDSVLYLKNRKEKNAVQDKNY | |
| | | SEEDISEYDLKNKNSFSVLKKILLNEDVNSEELEIFRK | |
| | | DVEAKLNKINSLKYSFEENKANYQKINENNVEKVGGKS | |
| | | KRNIIYDYYRESAKRNDYINNVQEAFDKLYKKEDIEKL | |
| | | FFLIENSKKHEKYKIREYYHKIIGRKNDKENFAKIIYE | |
| | | EIQNVNNIKELIEKIPDMSELKKSQVFYKYYLDKEELN | |
| | | DKNIKYAFCHFVEIEMSQLLKNYVYKRLSNISNDKIKR | |
| | | IFEYQNLKKLIENKLLNKLDTYVRNCGKYNYYLQVGEI | |
| | | ATSDFIARNRQNEAFLRNIIGVSSVAYFSLRNILETEN | |
| | | ENGITGRMRGKTVKNNKGEEKYVSGEVDKIYNENKQNE | |
| | | VKENLKMFYSYDFNMDNKNEIEDFFANIDEAISSIRHG | |
| | | IVHFNLELEGKDIFAFKNIAPSEISKKMFQNEINEKKL | |
| | | KLKIFKQLNSANVENYYEKDVIIKYLKNTKFNFVNKNI | |
| | | PFVPSFTKLYNKIEDLRNTLKFFWSVPKDKEEKDAQIY | |
| | | LLKNIYYGEFLNKFVKNSKVFFKITNEVIKINKQRNQK | |
| | | TGHYKYQKFENIEKTVPVEYLAIIQSREMINNQDKEEK | |
| | | NTYIDFIQQIFLKGFIDYLNKNNLKYIESNNNNDNNDI | |
| | | FSKIKIKKDNKEKYDKILKNYEKHNRNKEIPHEINEFV | |
| | | REIKLGKILKYTENLNMFYLILKLLNHKELTNLKGSLE | |
| | | KYQSANKEETFSDELELINLLNLDNNRVTEDFELEANE | |
| | | IGKFLDFNENKIKDRKELKKFDINKIYFDGENIIKHRA | |
| | | FYNIKKYGMLNLLEKIADKAKYKISLKELKEYSNKKNE | |
| | | IEKNYTMQQNLHRKYARPKKDEKFNDEDYKEYEKAIGN | |
| | | IQKYTHLKNKVEFNELNLLQGLLLKILHRLVGYTSIWE | |
| | | RDLRFRLKGEFPENHYIEEIFNFDNSKNVKYKSGQIVE | |
| | | KYINFYKELYKDNVEKRSIYSDKKVKKLKQEKKDLYIR | |
| | | NYIAHFNYIPHAEISLLEVLENLRKLLSYDRKLKNAIM | |
| | | KSIVDILKEYGFVATFKIGADKKIEIQTLESEKIVHLK | |
| | | NLKKKKLMTDRNSEELCELVKVMFEYKALEGGGGSGGG | |
| | | GSGGGGSVSKGEELFTGVVPILVELDGDVNGHKFSVRG | |
| | | EGEGDATNGKLTLKFICTTGKLPVPWPTLVTTLTYGVQ | |
| | | CFSRYPDHMKQHDFFKSAMPEGYVQERTISFKDDGTYK | |
| | | TRAEVKFEGDTLVNRIELKGIDFKEDGNILGHKLEYNF | |
| | | NSHNVYITADKQKNGIKANFKIRHNVEDGSVQLADHYQ | |
| | | QNTPIGDGPVLLPDNHYLSTQSKLSKDPNEKRDHMVLL | |
| | | EFVTAAGITLGMDELYKGSEGAPKKKRKVGSSYPYDVP | |
| | | DYAYPYDVPDYAYPYDVPDYAKRTADGSEFESPKKKRK | |
| | | V | |
| 473 | PspCas13 | MKRTADGSEFESPKKKRKVNIPALVENQKKYFGTYSVM | 1169 |
| | with | AMLNAQTVLDHIQKVADIEGEQNENNENLWFHPVMSHL | |
| | NLS | YNAKNGYDKQPEKTMFIIERLQSYFPFLKIMAENQREY | |
| | | SNGKYKQNRVEVNSNDIFEVLKRAFGVLKMYRDLTNHY | |
| | | KTYEEKLNDGCEFLTSTEQPLSGMINNYYTVALRNMNE | |
| | | RYGYKTEDLAFIQDKRFKFVKDAYGKKKSQVNTGFFLS | |
| | | LQDYNGDTQKKLHLSGVGIALLICLFLDKQYINIFLSR | |
| | | LPIFSSYNAQSEERRIIIRSFGINSIKLPKDRIHSEKS | |
| | | NKSVAMDMLNEVKRCPDELFTTLSAEKQSRFRIISDDH | |
| | | NEVLMKRSSDRFVPLLLQYIDYGKLFDHIRFHVNMGKL | |
| | | RYLLKADKTCIDGQTRVRVIEQPLNGFGRLEEAETMRK | |
| | | QENGTFGNSGIRIRDFENMKRDDANPANYPYIVDTYTH | |
| | | YILENNKVEMFINDKEDSAPLLPVIEDDRYVVKTIPSC | |
| | | RMSTLEIPAMAFHMFLFGSKKTEKLIVDVHNRYKRLFQ | |
| | | AMQKEEVTAENIASFGIAESDLPQKILDLISGNAHGKD | |
| | | VDAFIRLTVDDMLTDTERRIKRFKDDRKSIRSADNKMG | |
| | | KRGFKQISTGKLADFLAKDIVLFQPSVNDGENKITGLN | |
| | | YRIMQSAIAVYDSGDDYEAKQQFKLMFEKARLIGKGTT | |
| | | EPHPFLYKVFARSIPANAVEFYERYLIERKFYLTGLSN | |
| | | EIKKGNRVDVPFIRRDQNKWKTPAMKTLGRIYSEDLPV | |
| | | ELPRQMFDNEIKSHLKSLPQMEGIDFNNANVTYLIAEY | |
| | | MKRVLDDDFQTFYQWNRNYRYMDMLKGEYDRKGSLQHC | |
| | | FTSVEEREGLWKERASRTERYRKQASNKIRSNRQMRNA | |
| | | SSEEIETILDKRLSNSRNEYQKSEKVIRRYRVQDALLF | |
| | | LLAKKTLTELADFDGERFKLKEIMPDAEKGILSEIMPM | |
| | | SFTFEKGGKKYTITSEGMKLKNYGDFFVLASDKRIGNL | |
| | | LELVGSDIVSKEDIMEEFNKYDQCRPEISSIVFNLEKW | |
| | | AFDTYPELSARVDREEKVDFKSILKILLNNKNINKEQS | |
| | | DILRKIRNAFDHNNYPDKGVVEIKALPEIAMSIKKAFG | |
| | | EYAIMKGSLQLPPLERLTLGSSYPYDVPDYAYPYDVPD | |
| | | YAYPYDVPDYAKRTADGSEFESPKKKRKV | |
| 474 | RfxCas13 | MKRTADGSEFESPKKKRKVSPKKKRKVEASIEKKKSFA | 1041 |
| | with | KGMGVKSTLVSGSKVYMTTFAEGSDARLEKIVEGDSIR | |
| | NLS | SVNEGEAFSAEMADKNAGYKIGNAKFSHPKGYAVVANN | |
| | | PLYTGPVQQDMLGLKETLEKRYFGESADGNDNICIQVI | |
| | | HNILDIEKILAEYITNAAYAVNNISGLDKDIIGFGKFS | |
| | | TVYTYDEFKDPEHHRAAFNNNDKLINAIKAQYDEFDNF | |
| | | LDNPRLGYFGQAFFSKEGRNYIINYGNECYDILALLSG | |
| | | LRHWVVHNNEEESRISRTWLYNLDKNLDNEYISTLNYL | |
| | | YDRITNELTNSFSKNSAANVNYIAETLGINPAEFAEQY | |
| | | FRFSIMKEQKNLGFNITKLREVMLDRKDMSEIRKNHKV | |
| | | FDSIRTKVYTMMDFVIYRYYIEEDAKVAAANKSLPDNE | |
| | | KSLSEKDIFVINLRGSFNDDQKDALYYDEANRIWRKLE | |
| | | NIMHNIKEFRGNKTREYKKKDAPRLPRILPAGRDVSAF | |
| | | SKLMYALTMFLDGKEINDLLTTLINKFDNIQSFLKVMP | |
| | | LIGVNAKFVEEYAFFKDSAKIADELRLIKSFARMGEPI | |
| | | ADARRAMYIDAIRILGTNLSYDELKALADTFSLDENGN | |
| | | KLKKGKHGMRNFIINNVISNKRFHYLIRYGDPAHLHEI | |
| | | AKNEAVVKFVLGRIADIQKKQGQNGKNQIDRYYETCIG | |
| | | KDKGKSVSEKVDALTKIITGMNYDQFDKKRSVIEDTGR | |
| | | ENAEREKFKKIISLYLTVIYHILKNIVNINARYVIGFH | |
| | | CVERDAQLYKEKGYDINLKKLEEKGFSSVTKLCAGIDE | |
| | | TAPDKRKDVEKEMAERAKESIDSLESANPKLYANYIKY | |
| | | SDEKKAEEFTRQINREKAKTALNAYLRNTKWNVIIRED | |
| | | LLRIDNKTCTLFRNKAVHLEVARYVHAYINDIAEVNSY | |
| | | FQLYHYIMQRIIMNERYEKSSGKVSEYFDAVNDEKKYN | |
| | | DRLLKLLCVPFGYCIPRFKNLSIEALFDRNEAAKFDKE | |
| | | KKKVSGNSGSGPKKKRKVAAAYPYDVPDYAASGSGKRT | |
| | | ADGSEFESPKKKRKV | |
| 475 | SMALL | MKRTADGSEFESPKKKRKVTTTMKISIEFLEPFRMTKW | 1326 |
| | CAS | QESTRRNKNNKEFVRGQAFARWHRNKKDNTKGRPYITG | |
| | Cas711S_ | TLLRSAVIRSAENLLTLSDGKISEKTCCPGKFDTEDKD | |
| | D1580R | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAKRTADGSEFESPKKKRKV | |
| 476 | D1580R | MKRTADGSEFESPKKKRKVTTTMKISIEFLEPFRMTKW | 1637 |
| | | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 477 | Doublemut | MKRTADGSEFESPKKKRKVTTTMKISIEFLEPFRMTKW | 1637 |
| | 6 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 478 | Triplemut | MKRTADGSEFESPKKKRKVTTTMKISIEFLEPFRMTKW | 1637 |
| | 4 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 479 | Quadruplemut | MKRTADGSEFESPKKKRKVTTTMKISIEFLEPFRMTKW | 1637 |
| | 3 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHKLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 480 | Y280K- | MKRTADGSEFESPKKKRKVTTTMKISIEFLEPFRMTKW | 1354 |
| | D1580R- | QESTRRNKNNKEFVRGQAFARWHRNKKDNTKGRPYITG | |
| | Cas711S | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKESGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 481 | E279R- | MKRTADGSEFESPKKKRKVTTTMKISIEFLEPFRMTKW | 1354 |
| | D1580R- | QESTRRNKNNKEFVRGQAFARWHRNKKDNTKGRPYITG | |
| | Cas711S | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 466 | NewSmallCas- | MKRTADGSEFESPKKKRKVTTTMKISIEFLEPFRMTKW | 1354 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKESGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAKRTADGSEFESPKKKRKV | |
| 482 | NewSmallCas- | MKRTADGSEFESPKKKRKVTTTMKISIEFLEPFRMTKW | 1354 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | D1580R- | DGKKAEKKDWRFRIHFGNLSLPGKPDFDGPKAIGSQRV | |
| | D988K | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 483 | NewSmallCas- | MKRTADGSEFESPKKKRKVTTTMKISIEFLEPFRMTKW | 1354 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | D1580R- | DGKKAEKKDWRFRIHFGNLSLPGKPDFDGPKAIGSQRV | |
| | D988K- | LNRVDFKSGKAHDFFKAYEVDHTRFPRFEGEITIDNKV | |
| | D981K | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 484 | NewSmallCas- | MKRTADGSEFESPKKKRKVTTTMKISIEFLEPFRMTKW | 1354 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | D1580R- | DGKKAEKKDWRFRIHFGNLSLPGKPDFDGPKAIGSQRV | |
| | D988K- | LNRVDFKSGKAHDFFKAYEVDHTRFPRFEGEITIDNKV | |
| | D981K- | SAEARKLLCDSLKFTDRLCGALCVIRFDEYTPAADSGK | |
| | Y312K | QTENVQAEPNANLAEKTAEQIISILDDNKKTEYTRLLA | |
| | | DAIRSLRRSSKLVAGLPKDHDGKDDHKLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 485 | NewSmallCas- | MKRTADGSEFESPKKKRKVTTTMKISIEFLEPFRMTKW | 1479 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | SF3B6- | DGKKAEKKDWRFRIHFGNLSLPGKPDFDGPKAIGSQRV | |
| | fusion | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAMAMQAAKRANIRLPPEVNRILYIRNLPYKITA | |
| | | EEMYDIFGKYGPIRQIRVGNTPETRGTAYVVYEDIFDA | |
| | | KNACDHLSGFNVCNRYLVVLYYNANRAFQKMDTKKKEE | |
| | | QLKLLKEKYGINTDPPKKRTADGSEFESPKKKRKV | |
| 486 | NewSmallCas- | MKRTADGSEFESPKKKRKVTTTMKISIEFLEPFRMTKW | 1594 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | U2AF1- | DGKKAEKKDWRFRIHFGNLSLPGKPDFDGPKAIGSQRV | |
| | fusion | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWERDELDFIENLKKLLWFPEGDQAP | |
| | | RVCYPMLRKKDDPNGNSGYEELKDGEFKKEDRQKKLTT | |
| | | PWTPWAMAEYLASIFGTEKDKVNCSFYFKIGACRHGDR | |
| | | CSRLHNKPTFSQTIALLNIYRNPQNSSQSADGLRCAVS | |
| | | DVEMQEHYDEFFEEVFTEMEEKYGEVEEMNVCDNLGDH | |
| | | LVGNVYVKFRREEDAEKAVIDLNNRWFNGQPLHAELSP | |
| | | VTDFREACCRQYEMGECTRGGFCNFMHLKPISRELRRE | |
| | | LYGRRRKKHRSRSRSRERRSRSRDRGRGGGGGGGGGGG | |
| | | GRERDRRRSRDRERSGRFKRTADGSEFESPKKKRKV | |
| 487 | NewSmallCas- | MKRTADGSEFESPKKKRKVTTTMKISIEFLEPFRMTKW | 1755 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | RBM17- | DGKKAEKKDWRFRIHFGNLSLPGKPDFDGPKAIGSQRV | |
| | fusion | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAMSLYDDLGVETSDSKTEGWSKNFKLLQSQLQV | |
| | | KKAALTQAKSQRTKQSTVLAPVIDLKRGGSSDDRQIVD | |
| | | TPPHVAAGLKDPVPSGFSAGEVLIPLADEYDPMFPNDY | |
| | | EKVVKRQREERQRQRELERQKEIEEREKRRKDRHEASG | |
| | | FARRPDPDSDEDEDYERERRKRSMGGAAIAPPTSLVEK | |
| | | DKELPRDFPYEEDSRPRSQSSKAAIPPPVYEEQDRPRS | |
| | | PTGPSNSFLANMGGTVAHKIMQKYGFREGQGLGKHEQG | |
| | | LSTALSVEKTSKRGGKIIVGDATEKDASKKSDSNPLTE | |
| | | ILKCPTKVVLLRNMVGAGEVDEDLEVETKEECEKYGKV | |
| | | GKCVIFEIPGAPDDEAVRIFLEFERVESAIKAVVDLNG | |
| | | RYFGGRVVKACFYNLDKFRVLDLAEQVKRTADGSEFES | |
| | | PKKKRKV | |
| 488 | NewSmallCas- | MKRTADGSEFESPKKKRKVTTTMKISIEFLEPFRMTKW | 1829 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | U2AF2- | DGKKAEKKDWRFRIHFGNLSLPGKPDFDGPKAIGSQRV | |
| | fusion | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAMSDFDEFERQLNENKQERDKENRHRKRSHSRS | |
| | | RSRDRKRRSRSRDRRNRDQRSASRDRRRRSKPLTRGAK | |
| | | EEHGGLIRSPRHEKKKKVRKYWDVPPPGFEHITPMQYK | |
| | | AMQAAGQIPATALLPTMTPDGLAVTPTPVPVVGSQMTR | |
| | | QARRLYVGNIPFGITEEAMMDFFNAQMRLGGLTQAPGN | |
| | | PVLAVQINQDKNFAFLEFRSVDETTQAMAFDGIIFQGQ | |
| | | SLKIRRPHDYQPLPGMSENPSVYVPGVVSTVVPDSAHK | |
| | | LFIGGLPNYLNDDQVKELLTSFGPLKAFNLVKDSATGL | |
| | | SKGYAFCEYVDINVTDQAIAGLNGMQLGDKKLLVQRAS | |
| | | VGAKNATLVSPPSTINQTPVTLQVPGLMSSQVQMGGHP | |
| | | TEVLCLMNMVLPEELLDDEEYEEIVEDVRDECSKYGLV | |
| | | KSIEIPRPVDGVEVPGCGKIFVEFTSVFDCQKAMQGLT | |
| | | GRKFANRVVVTKYCDPDSYHRRDFWKRTADGSEFESPK | |
| | | KKRKV | |
| 489 | NewSmallCas- | MKRTADGSEFESPKKKRKVTTTMKISIEFLEPFRMTKW | 1354 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | D1580R- | DGKKAEKKDWRFRIHFGNLSLPGKPDFDGPKAIGSQRV | |
| | D988K- | LNRVDFKSGKAHDFFKAYEVDHTRFPRFEGEITIDNKV | |
| | E279R | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 490 | NewSmallCas- | MKRTADGSEFESPKKKRKVTTTMKISIEFLEPFRMTKW | 1354 |
| | S1006- | QESTRRNKNNKEFVRGQAFARWHRNKKDNTKGRPYITG | |
| | R1294- | TLLRSAVIRSAENLLTLSDGKISEKTCCPGKFDTEDKD | |
| | Cas711S- | RLLQLRQRSTLRWTDKNPCPDNAETYCPFCELLGRSGN | |
| | D1580R- | DGKKAEKKDWRFRIHFGNLSLPGKPDFDGPKAIGSQRV | |
| | D988K- | LNRVDFKSGKAHDFFKAYEVDHTRFPRFEGEITIDNKV | |
| | Y312K | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHKLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSGG | |
| | | GSRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKRGEFKKEDRQKKLTT | |
| | | PWTPWAKRTADGSEFESPKKKRKV | |
| 491 | NewTriple- | MKRTADGSEFESPKKKRKVTTTMKISIEFLEPFRMTKW | 1637 |
| | Mutant- | QESTRRNKNNKEFVRGQAFARWHRNKKDNTKGRPYITG | |
| | Cas711- | TLLRSAVIRSAENLLTLSDGKISEKTCCPGKFDTEDKD | |
| | D1580R- | RLLQLRQRSTLRWTDKNPCPDNAETYCPFCELLGRSGN | |
| | D988K- | DGKKAEKKDWRFRIHFGNLSLPGKPDFDGPKAIGSQRV | |
| | Y312K | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHKLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 492 | pDF0910_ | MKRTADGSEFESPKKKRKVTTTMKISIEFLEPFRMTKW | 1762 |
| | Cas711_ | QESTRRNKNNKEFVRGQAFARWHRNKKDNTKGRPYITG | |
| | SF3B6_ | TLLRSAVIRSAENLLTLSDGKISEKTCCPGKFDTEDKD | |
| | Ct_ | RLLQLRQRSTLRWTDKNPCPDNAETYCPFCELLGRSGN | |
| | directfusion | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKKRTA | |
| | | DGSEFESPKKKRKV | |
| 493 | pDF0947_ | MKRTADGSEFESPKKKRKVTTTMKISIEFLEPFRMTKW | 2112 |
| | Cas711_ | QESTRRNKNNKEFVRGQAFARWHRNKKDNTKGRPYITG | |
| | U2AF2_ | TLLRSAVIRSAENLLTLSDGKISEKTCCPGKFDTEDKD | |
| | Ct_ | RLLQLRQRSTLRWTDKNPCPDNAETYCPFCELLGRSGN | |
| | directfusion | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMSDEDEFERQLNENK | |
| | | QERDKENRHRKRSHSRSRSRDRKRRSRSRDRRNRDQRS | |
| | | ASRDRRRRSKPLTRGAKEEHGGLIRSPRHEKKKKVRKY | |
| | | WDVPPPGFEHITPMQYKAMQAAGQIPATALLPTMTPDG | |
| | | LAVTPTPVPVVGSQMTRQARRLYVGNIPFGITEEAMMD | |
| | | FFNAQMRLGGLTQAPGNPVLAVQINQDKNFAFLEFRSV | |
| | | DETTQAMAFDGIIFQGQSLKIRRPHDYQPLPGMSENPS | |
| | | VYVPGVVSTVVPDSAHKLFIGGLPNYLNDDQVKELLTS | |
| | | FGPLKAFNLVKDSATGLSKGYAFCEYVDINVTDQAIAG | |
| | | LNGMQLGDKKLLVQRASVGAKNATLVSPPSTINQTPVT | |
| | | LQVPGLMSSQVQMGGHPTEVLCLMNMVLPEELLDDEEY | |
| | | EEIVEDVRDECSKYGLVKSIEIPRPVDGVEVPGCGKIF | |
| | | VEFTSVFDCQKAMQGLTGRKFANRVVVTKYCDPDSYHR | |
| | | RDFWKRTADGSEFESPKKKRKV | |
| 477 | PDF0949 | MKRTADGSEFESPKKKRKVTTTMKISIEFLEPFRMTKW | 1637 |
| | double | QESTRRNKNNKEFVRGQAFARWHRNKKDNTKGRPYITG | |
| | mutant | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVEPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 478 | PDF0950 | MKRTADGSEFESPKKKRKVTTTMKISIEFLEPFRMTKW | 1637 |
| | triple | QESTRRNKNNKEFVRGQAFARWHRNKKDNTKGRPYITG | |
| | mutant | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 494 | double | MKRTADGSEFESPKKKRKVTTTMKISIEFLEPFRMTKW | 1762 |
| | mutant | QESTRRNKNNKEFVRGQAFARWHRNKKDNTKGRPYITG | |
| | fusion | TLLRSAVIRSAENLLTLSDGKISEKTCCPGKFDTEDKD | |
| | 1 | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSESFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKKRTA | |
| | | DGSEFESPKKKRKV | |
| 495 | triple | MKRTADGSEFESPKKKRKVTTTMKISIEFLEPFRMTKW | 2112 |
| | mutant | QESTRRNKNNKEFVRGQAFARWHRNKKDNTKGRPYITG | |
| | fusion | TLLRSAVIRSAENLLTLSDGKISEKTCCPGKFDTEDKD | |
| | 1 | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVEPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMSDFDEFERQLNENK | |
| | | QERDKENRHRKRSHSRSRSRDRKRRSRSRDRRNRDQRS | |
| | | ASRDRRRRSKPLTRGAKEEHGGLIRSPRHEKKKKVRKY | |
| | | WDVPPPGFEHITPMQYKAMQAAGQIPATALLPTMTPDG | |
| | | LAVTPTPVPVVGSQMTRQARRLYVGNIPFGITEEAMMD | |
| | | FFNAQMRLGGLTQAPGNPVLAVQINQDKNFAFLEFRSV | |
| | | DETTQAMAFDGIIFQGQSLKIRRPHDYQPLPGMSENPS | |
| | | VYVPGVVSTVVPDSAHKLFIGGLPNYLNDDQVKELLTS | |
| | | FGPLKAFNLVKDSATGLSKGYAFCEYVDINVTDQAIAG | |
| | | LNGMQLGDKKLLVQRASVGAKNATLVSPPSTINQTPVT | |
| | | LQVPGLMSSQVQMGGHPTEVLCLMNMVLPEELLDDEEY | |
| | | EEIVEDVRDECSKYGLVKSIEIPRPVDGVEVPGCGKIF | |
| | | VEFTSVFDCQKAMQGLTGRKFANRVVVTKYCDPDSYHR | |
| | | RDFWKRTADGSEFESPKKKRKV | |
| 496 | double | MKRTADGSEFESPKKKRKVTTTMKISIEFLEPFRMTKW | 1762 |
| | mutant | QESTRRNKNNKEFVRGQAFARWHRNKKDNTKGRPYITG | |
| | fusion | TLLRSAVIRSAENLLTLSDGKISEKTCCPGKFDTEDKD | |
| | 2 | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKKRTA | |
| | | DGSEFESPKKKRKV | |
| 497 | triple | MKRTADGSEFESPKKKRKVTTTMKISIEFLEPFRMTKW | 2112 |
| | mutant | QESTRRNKNNKEFVRGQAFARWHRNKKDNTKGRPYITG | |
| | fusion | TLLRSAVIRSAENLLTLSDGKISEKTCCPGKFDTEDKD | |
| | 2 | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMSDFDEFERQLNENK | |
| | | QERDKENRHRKRSHSRSRSRDRKRRSRSRDRRNRDQRS | |
| | | ASRDRRRRSKPLTRGAKEEHGGLIRSPRHEKKKKVRKY | |
| | | WDVPPPGFEHITPMQYKAMQAAGQIPATALLPTMTPDG | |
| | | LAVTPTPVPVVGSQMTRQARRLYVGNIPFGITEEAMMD | |
| | | FFNAQMRLGGLTQAPGNPVLAVQINQDKNFAFLEFRSV | |
| | | DETTQAMAFDGIIFQGQSLKIRRPHDYQPLPGMSENPS | |
| | | VYVPGVVSTVVPDSAHKLFIGGLPNYLNDDQVKELLTS | |
| | | FGPLKAFNLVKDSATGLSKGYAFCEYVDINVTDQAIAG | |
| | | LNGMQLGDKKLLVQRASVGAKNATLVSPPSTINQTPVT | |
| | | LQVPGLMSSQVQMGGHPTEVLCLMNMVLPEELLDDEEY | |
| | | EEIVEDVRDECSKYGLVKSIEIPRPVDGVEVPGCGKIF | |
| | | VEFTSVFDCQKAMQGLTGRKFANRVVVTKYCDPDSYHR | |
| | | RDFWKRTADGSEFESPKKKRKV | |
| 498 | RBM17 | MKRTADGSEFESPKKKRKVMSLYDDLGVETSDSKTEGW | 2038 |
| | Direct_ | SKNFKLLQSQLQVKKAALTQAKSQRTKQSTVLAPVIDL | |
| | Nt | KRGGSSDDRQIVDTPPHVAAGLKDPVPSGFSAGEVLIP | |
| | | LADEYDPMFPNDYEKVVKRQREERQRQRELERQKEIEE | |
| | | REKRRKDRHEASGFARRPDPDSDEDEDYERERRKRSMG | |
| | | GAAIAPPTSLVEKDKELPRDFPYEEDSRPRSQSSKAAI | |
| | | PPPVYEEQDRPRSPTGPSNSFLANMGGTVAHKIMQKYG | |
| | | FREGQGLGKHEQGLSTALSVEKTSKRGGKIIVGDATEK | |
| | | DASKKSDSNPLTEILKCPTKVVLLRNMVGAGEVDEDLE | |
| | | VETKEECEKYGKVGKCVIFEIPGAPDDEAVRIFLEFER | |
| | | VESAIKAVVDLNGRYFGGRVVKACFYNLDKFRVLDLAE | |
| | | QVTTTMKISIEFLEPFRMTKWQESTRRNKNNKEFVRGQ | |
| | | AFARWHRNKKDNTKGRPYITGTLLRSAVIRSAENLLTL | |
| | | SDGKISEKTCCPGKFDTEDKDRLLQLRQRSTLRWTDKN | |
| | | PCPDNAETYCPFCELLGRSGNDGKKAEKKDWRFRIHFG | |
| | | NLSLPGKPDFDGPKAIGSQRVLNRVDFKSGKAHDFFKA | |
| | | YEVDHTRFPRFEGEITIDNKVSAEARKLLCDSLKFTDR | |
| | | LCGALCVIRFDEYTPAADSGKQTENVQAEPNANLAEKT | |
| | | AEQIISILDDNKKTEYTRLLADAIRSLRRSSKLVAGLP | |
| | | KDHDGKDDHYLWDIGKKKKDENSVTIRQILTTSADTKE | |
| | | LKNAGKWREFCEKLGEALYLKSKDMSGGLKITRRILGD | |
| | | AEFHGKPDRLEKSRSVSIGSVLKETVVCGELVAKTPFF | |
| | | FGAIDEDAKQTDLQVLLTPDNKYRLPRSAVRGILRRDL | |
| | | QTYFDSPCNAELGGRPCMCKTCRIMRGITVMDARSEYN | |
| | | APPEIRHRTRINPFTGTVAEGALFNMEVAPEGIVFPFQ | |
| | | LRYRGSEDGLPDALKTVLKWWAEGQAFMSGAASTGKGR | |
| | | FRMENAKYETLDLSDENQRNDYLKNWGWRDEKGLEELK | |
| | | KRLNSGLPEPGNYRDPKWHEINVSIEMASPFINGDPIR | |
| | | AAVDKRGTDVVTFVKYKAEGEEAKPVCAYKAESFRGVI | |
| | | RSAVARIHMEDGVPLTELTHSDCECLLCQIFGSEYEAG | |
| | | KIRFEDLVFESDPEPVTFDHVAIDRFTGGAADKKKFDD | |
| | | SPLPGSPARPLMLKGSFWIRRDVLEDEEYCKALGKALA | |
| | | DVNNGLYPLGGKSAIGYGQVKSLGIKGDDKRISRLMNP | |
| | | AFDETDVAVPEKPKTDAEVRIEAEKVYYPHYFVEPHKK | |
| | | VEREEKPCGHQKFHEGRLTGKIRCKLITKTPLIVPDTS | |
| | | NDDFFRPADKEARKEKDEYHKSYAFFRLHKQIMIPGSE | |
| | | LRGMVSSVYETVTNSCFRIFDETKRLSWRMDADHQNVL | |
| | | QDFLPGRVTADGKHIQKFSETARVPFYDKTQKHFDILD | |
| | | EQEIAGEKPVRMWVKRFIKRLSLVDPAKHPQKKQDNKW | |
| | | KRRKEGIATFIEQKNGSYYFNVVTNNGCTSFHLWHKPD | |
| | | NFDQEKLEGIQNGEKLDCWVRDSRYQKAFQEIPENDPD | |
| | | GWECKEGYLHVVGPSKVEFSDKKGDVINNFQGTLPSVP | |
| | | NDWKTIRTNDFKNRKRKNEPVFCCEDDKGNYYTMAKYC | |
| | | ETFFFDLKENEEYEIPEKARIKYKELLRVYNNNPQAVP | |
| | | ESVFQSRVARENVEKLKSGDLVYFKHNEKYVEDIVPVR | |
| | | ISRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAKRTADGSEFESPKKKRKV | |
| 499 | RBM17 | MKRTADGSEFESPKKKRKVTTTMKISIEFLEPFRMTKW | 2038 |
| | Direct_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMSLYDDLGVETSDSK | |
| | | TEGWSKNFKLLQSQLQVKKAALTQAKSQRTKQSTVLAP | |
| | | VIDLKRGGSSDDRQIVDTPPHVAAGLKDPVPSGFSAGE | |
| | | VLIPLADEYDPMFPNDYEKVVKRQREERQRQRELERQK | |
| | | EIEEREKRRKDRHEASGFARRPDPDSDEDEDYERERRK | |
| | | RSMGGAAIAPPTSLVEKDKELPRDFPYEEDSRPRSQSS | |
| | | KAAIPPPVYEEQDRPRSPTGPSNSFLANMGGTVAHKIM | |
| | | QKYGFREGQGLGKHEQGLSTALSVEKTSKRGGKIIVGD | |
| | | ATEKDASKKSDSNPLTEILKCPTKVVLLRNMVGAGEVD | |
| | | EDLEVETKEECEKYGKVGKCVIFEIPGAPDDEAVRIFL | |
| | | EFERVESAIKAVVDLNGRYFGGRVVKACFYNLDKFRVL | |
| | | DLAEQVKRTADGSEFESPKKKRKV | |
| 500 | RBM17 | MKRTADGSEFESPKKKRKVTTTMKISIEFLEPFRMTKW | 2053 |
| | XTENLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWASGSETPGTSESATPE | |
| | | SSLYDDLGVETSDSKTEGWSKNFKLLQSQLQVKKAALT | |
| | | QAKSQRTKQSTVLAPVIDLKRGGSSDDRQIVDTPPHVA | |
| | | AGLKDPVPSGFSAGEVLIPLADEYDPMFPNDYEKVVKR | |
| | | QREERQRQRELERQKEIEEREKRRKDRHEASGFARRPD | |
| | | PDSDEDEDYERERRKRSMGGAAIAPPTSLVEKDKELPR | |
| | | DFPYEEDSRPRSQSSKAAIPPPVYEEQDRPRSPTGPSN | |
| | | SFLANMGGTVAHKIMQKYGFREGQGLGKHEQGLSTALS | |
| | | VEKTSKRGGKIIVGDATEKDASKKSDSNPLTEILKCPT | |
| | | KVVLLRNMVGAGEVDEDLEVETKEECEKYGKVGKCVIF | |
| | | EIPGAPDDEAVRIFLEFERVESAIKAVVDLNGRYFGGR | |
| | | VVKACFYNLDKFRVLDLAEQVKRTADGSEFESPKKKRK | |
| | | V | |
| 501 | SF3B6 | MKRTADGSEFESPKKKRKVMAMQAAKRANIRLPPEVNR | 1762 |
| | Direct_ | ILYIRNLPYKITAEEMYDIFGKYGPIRQIRVGNTPETR | |
| | Nt | GTAYVVYEDIFDAKNACDHLSGFNVCNRYLVVLYYNAN | |
| | | RAFQKMDTKKKEEQLKLLKEKYGINTDPPKTTTMKISI | |
| | | EFLEPFRMTKWQESTRRNKNNKEFVRGQAFARWHRNKK | |
| | | DNTKGRPYITGTLLRSAVIRSAENLLTLSDGKISEKTC | |
| | | CPGKFDTEDKDRLLQLRQRSTLRWTDKNPCPDNAETYC | |
| | | PFCELLGRSGNDGKKAEKKDWRFRIHFGNLSLPGKPDF | |
| | | DGPKAIGSQRVLNRVDFKSGKAHDFFKAYEVDHTRFPR | |
| | | FEGEITIDNKVSAEARKLLCDSLKFTDRLCGALCVIRF | |
| | | DEYTPAADSGKQTENVQAEPNANLAEKTAEQIISILDD | |
| | | NKKTEYTRLLADAIRSLRRSSKLVAGLPKDHDGKDDHY | |
| | | LWDIGKKKKDENSVTIRQILTTSADTKELKNAGKWREF | |
| | | CEKLGEALYLKSKDMSGGLKITRRILGDAEFHGKPDRL | |
| | | EKSRSVSIGSVLKETVVCGELVAKTPFFFGAIDEDAKQ | |
| | | TDLQVLLTPDNKYRLPRSAVRGILRRDLQTYFDSPCNA | |
| | | ELGGRPCMCKTCRIMRGITVMDARSEYNAPPEIRHRTR | |
| | | INPFTGTVAEGALFNMEVAPEGIVFPFQLRYRGSEDGL | |
| | | PDALKTVLKWWAEGQAFMSGAASTGKGRFRMENAKYET | |
| | | LDLSDENQRNDYLKNWGWRDEKGLEELKKRLNSGLPEP | |
| | | GNYRDPKWHEINVSIEMASPFINGDPIRAAVDKRGTDV | |
| | | VTFVKYKAEGEEAKPVCAYKAESFRGVIRSAVARIHME | |
| | | DGVPLTELTHSDCECLLCQIFGSEYEAGKIRFEDLVFE | |
| | | SDPEPVTFDHVAIDRFTGGAADKKKFDDSPLPGSPARP | |
| | | LMLKGSFWIRRDVLEDEEYCKALGKALADVNNGLYPLG | |
| | | GKSAIGYGQVKSLGIKGDDKRISRLMNPAFDETDVAVP | |
| | | EKPKTDAEVRIEAEKVYYPHYFVEPHKKVEREEKPCGH | |
| | | QKFHEGRLTGKIRCKLITKTPLIVPDTSNDDFFRPADK | |
| | | EARKEKDEYHKSYAFFRLHKQIMIPGSELRGMVSSVYE | |
| | | TVTNSCFRIFDETKRLSWRMDADHQNVLQDFLPGRVTA | |
| | | DGKHIQKFSETARVPFYDKTQKHFDILDEQEIAGEKPV | |
| | | RMWVKRFIKRLSLVDPAKHPQKKQDNKWKRRKEGIATF | |
| | | IEQKNGSYYFNVVTNNGCTSFHLWHKPDNFDQEKLEGI | |
| | | QNGEKLDCWVRDSRYQKAFQEIPENDPDGWECKEGYLH | |
| | | VVGPSKVEFSDKKGDVINNFQGTLPSVPNDWKTIRTND | |
| | | FKNRKRKNEPVFCCEDDKGNYYTMAKYCETFFFDLKEN | |
| | | EEYEIPEKARIKYKELLRVYNNNPQAVPESVFQSRVAR | |
| | | ENVEKLKSGDLVYFKHNEKYVEDIVPVRISRTVDDRMI | |
| | | GKRMSADLRPCHGDWVEDGDLSALNAYPEKRLLLRHPK | |
| | | GLCPACRLFGTGSYKGRVRFGFASLENDPEWLIPGKNP | |
| | | GDPFHGGPVMLSLLERPRPTWSIPGSDNKFKVPGRKFY | |
| | | VHHHAWKTIKDGNHPTTGKAIEQSPNNRTVEALAGGNS | |
| | | FSFEIAFENLKEWELGLLIHSLQLEKGLAHKLGMAKSM | |
| | | GFGSVEIDVESVRLRKDWKQWRNGNSEIPNWLGKGFAK | |
| | | LKEWFRDELDFIENLKKLLWFPEGDQAPRVCYPMLRKK | |
| | | DDPNGNSGYEELKDGEFKKEDRQKKLTTPWTPWAKRTA | |
| | | DGSEFESPKKKRKV | |
| 492 | SF3B6 | MKRTADGSEFESPKKKRKVTTTMKISIEFLEPFRMTKW | 1762 |
| | Direct_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKKRTA | |
| | | DGSEFESPKKKRKV | |
| 502 | SF3B6 | MKRTADGSEFESPKKKRKVTTTMKISIEFLEPFRMTKW | 1777 |
| | XTENLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWASGSETPGTSESATPE | |
| | | SAMQAAKRANIRLPPEVNRILYIRNLPYKITAEEMYDI | |
| | | FGKYGPIRQIRVGNTPETRGTAYVVYEDIFDAKNACDH | |
| | | LSGFNVCNRYLVVLYYNANRAFQKMDTKKKEEQLKLLK | |
| | | EKYGINTDPPKKRTADGSEFESPKKKRKV | |
| 503 | U2AF1 | MKRTADGSEFESPKKKRKVMAEYLASIFGTEKDKVNCS | 1877 |
| | Direct_ | FYFKIGACRHGDRCSRLHNKPTFSQTIALLNIYRNPQN | |
| | Nt | SSQSADGLRCAVSDVEMQEHYDEFFEEVETEMEEKYGE | |
| | | VEEMNVCDNLGDHLVGNVYVKFRREEDAEKAVIDLNNR | |
| | | WFNGQPIHAELSPVTDFREACCRQYEMGECTRGGFCNF | |
| | | MHLKPISRELRRELYGRRRKKHRSRSRSRERRSRSRDR | |
| | | GRGGGGGGGGGGGGRERDRRRSRDRERSGRFTTTMKIS | |
| | | IEFLEPFRMTKWQESTRRNKNNKEFVRGQAFARWHRNK | |
| | | KDNTKGRPYITGTLLRSAVIRSAENLLTLSDGKISEKT | |
| | | CCPGKFDTEDKDRLLQLRQRSTLRWTDKNPCPDNAETY | |
| | | CPFCELLGRSGNDGKKAEKKDWRFRIHFGNLSLPGKPD | |
| | | FDGPKAIGSQRVLNRVDFKSGKAHDFFKAYEVDHTRFP | |
| | | RFEGEITIDNKVSAEARKLLCDSLKFTDRLCGALCVIR | |
| | | FDEYTPAADSGKQTENVQAEPNANLAEKTAEQIISILD | |
| | | DNKKTEYTRLLADAIRSLRRSSKLVAGLPKDHDGKDDH | |
| | | YLWDIGKKKKDENSVTIRQILTTSADTKELKNAGKWRE | |
| | | FCEKLGEALYLKSKDMSGGLKITRRILGDAEFHGKPDR | |
| | | LEKSRSVSIGSVLKETVVCGELVAKTPFFFGAIDEDAK | |
| | | QTDLQVLLTPDNKYRLPRSAVRGILRRDLQTYFDSPCN | |
| | | AELGGRPCMCKTCRIMRGITVMDARSEYNAPPEIRHRT | |
| | | RINPFTGTVAEGALFNMEVAPEGIVFPFQLRYRGSEDG | |
| | | LPDALKTVLKWWAEGQAFMSGAASTGKGRFRMENAKYE | |
| | | TLDLSDENQRNDYLKNWGWRDEKGLEELKKRLNSGLPE | |
| | | PGNYRDPKWHEINVSIEMASPFINGDPIRAAVDKRGTD | |
| | | VVTFVKYKAEGEEAKPVCAYKAESFRGVIRSAVARIHM | |
| | | EDGVPLTELTHSDCECLLCQIFGSEYEAGKIRFEDLVF | |
| | | ESDPEPVTFDHVAIDRFTGGAADKKKFDDSPLPGSPAR | |
| | | PLMLKGSFWIRRDVLEDEEYCKALGKALADVNNGLYPL | |
| | | GGKSAIGYGQVKSLGIKGDDKRISRLMNPAFDETDVAV | |
| | | PEKPKTDAEVRIEAEKVYYPHYFVEPHKKVEREEKPCG | |
| | | HQKFHEGRLIGKIRCKLITKTPLIVPDTSNDDFFRPAD | |
| | | KEARKEKDEYHKSYAFFRLHKQIMIPGSELRGMVSSVY | |
| | | ETVTNSCFRIFDETKRLSWRMDADHQNVLQDFLPGRVT | |
| | | ADGKHIQKFSETARVPFYDKTQKHFDILDEQEIAGEKP | |
| | | VRMWVKRFIKRLSLVDPAKHPQKKQDNKWKRRKEGIAT | |
| | | FIEQKNGSYYFNVVTNNGCTSFHLWHKPDNFDQEKLEG | |
| | | IQNGEKLDCWVRDSRYQKAFQEIPENDPDGWECKEGYL | |
| | | HVVGPSKVEFSDKKGDVINNFQGTLPSVPNDWKTIRTN | |
| | | DFKNRKRKNEPVFCCEDDKGNYYTMAKYCETFFFDLKE | |
| | | NEEYEIPEKARIKYKELLRVYNNNPQAVPESVFQSRVA | |
| | | RENVEKLKSGDLVYFKHNEKYVEDIVPVRISRTVDDRM | |
| | | IGKRMSADLRPCHGDWVEDGDLSALNAYPEKRLLLRHP | |
| | | KGLCPACRLFGTGSYKGRVRFGFASLENDPEWLIPGKN | |
| | | PGDPFHGGPVMLSLLERPRPTWSIPGSDNKFKVPGRKF | |
| | | YVHHHAWKTIKDGNHPTTGKAIEQSPNNRTVEALAGGN | |
| | | SFSFEIAFENLKEWELGLLIHSLQLEKGLAHKLGMAKS | |
| | | MGFGSVEIDVESVRLRKDWKQWRNGNSEIPNWLGKGFA | |
| | | KLKEWFRDELDFIENLKKLLWFPEGDQAPRVCYPMLRK | |
| | | KDDPNGNSGYEELKDGEFKKEDRQKKLTTPWTPWAKRT | |
| | | ADGSEFESPKKKRKV | |
| 504 | U2AF1 | MKRTADGSEFESPKKKRKVTTTMKISIEFLEPFRMTKW | 1877 |
| | Direct_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAEYLASIFGTEKDK | |
| | | VNCSFYFKIGACRHGDRCSRLHNKPTFSQTIALLNIYR | |
| | | NPQNSSQSADGLRCAVSDVEMQEHYDEFFEEVFTEMEE | |
| | | KYGEVEEMNVCDNLGDHLVGNVYVKFRREEDAEKAVID | |
| | | LNNRWFNGQPIHAELSPVTDFREACCRQYEMGECTRGG | |
| | | FCNFMHLKPISRELRRELYGRRRKKHRSRSRSRERRSR | |
| | | SRDRGRGGGGGGGGGGGGRERDRRRSRDRERSGRFKRT | |
| | | ADGSEFESPKKKRKV | |
| 505 | U2AF1 | MKRTADGSEFESPKKKRKVTTTMKISIEFLEPFRMTKW | 1892 |
| | XTENLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWASGSETPGTSESATPE | |
| | | SAEYLASIFGTEKDKVNCSFYFKIGACRHGDRCSRLHN | |
| | | KPTFSQTIALLNIYRNPQNSSQSADGLRCAVSDVEMQE | |
| | | HYDEFFEEVFTEMEEKYGEVEEMNVCDNLGDHLVGNVY | |
| | | VKFRREEDAEKAVIDLNNRWFNGQPIHAELSPVTDFRE | |
| | | ACCRQYEMGECTRGGFCNFMHLKPISRELRRELYGRRR | |
| | | KKHRSRSRSRERRSRSRDRGRGGGGGGGGGGGGRERDR | |
| | | RRSRDRERSGRFKRTADGSEFESPKKKRKV | |
| 493 | U2AF2 | MKRTADGSEFESPKKKRKVTTTMKISIEFLEPFRMTKW | 2112 |
| | Direct_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMSDFDEFERQLNENK | |
| | | QERDKENRHRKRSHSRSRSRDRKRRSRSRDRRNRDQRS | |
| | | ASRDRRRRSKPLTRGAKEEHGGLIRSPRHEKKKKVRKY | |
| | | WDVPPPGFEHITPMQYKAMQAAGQIPATALLPTMTPDG | |
| | | LAVTPTPVPVVGSQMTRQARRLYVGNIPFGITEEAMMD | |
| | | FFNAQMRLGGLTQAPGNPVLAVQINQDKNFAFLEFRSV | |
| | | DETTQAMAFDGIIFQGQSLKIRRPHDYQPLPGMSENPS | |
| | | VYVPGVVSTVVPDSAHKLFIGGLPNYLNDDQVKELLTS | |
| | | FGPLKAFNLVKDSATGLSKGYAFCEYVDINVTDQAIAG | |
| | | LNGMQLGDKKLLVQRASVGAKNATLVSPPSTINQTPVT | |
| | | LQVPGLMSSQVQMGGHPTEVLCLMNMVLPEELLDDEEY | |
| | | EEIVEDVRDECSKYGLVKSIEIPRPVDGVEVPGCGKIF | |
| | | VEFTSVFDCQKAMQGLTGRKFANRVVVTKYCDPDSYHR | |
| | | RDFWKRTADGSEFESPKKKRKV | |
| 506 | U2AF2 | MKRTADGSEFESPKKKRKVTTTMKISIEFLEPFRMTKW | 2127 |
| | XTENLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWASGSETPGTSESATPE | |
| | | SSDFDEFERQLNENKQERDKENRHRKRSHSRSRSRDRK | |
| | | RRSRSRDRRNRDQRSASRDRRRRSKPLTRGAKEEHGGL | |
| | | IRSPRHEKKKKVRKYWDVPPPGFEHITPMQYKAMQAAG | |
| | | QIPATALLPTMTPDGLAVTPTPVPVVGSQMTRQARRLY | |
| | | VGNIPFGITEEAMMDFFNAQMRLGGLTQAPGNPVLAVQ | |
| | | INQDKNFAFLEFRSVDETTQAMAFDGIIFQGQSLKIRR | |
| | | PHDYQPLPGMSENPSVYVPGVVSTVVPDSAHKLFIGGL | |
| | | PNYLNDDQVKELLTSFGPLKAFNLVKDSATGLSKGYAF | |
| | | CEYVDINVTDQAIAGLNGMQLGDKKLLVQRASVGAKNA | |
| | | TLVSPPSTINQTPVTLQVPGLMSSQVQMGGHPTEVLCL | |
| | | MNMVLPEELLDDEEYEEIVEDVRDECSKYGLVKSIEIP | |
| | | RPVDGVEVPGCGKIFVEFTSVFDCQKAMQGLTGRKFAN | |
| | | RVVVTKYCDPDSYHRRDFWKRTADGSEFESPKKKRKV | |
| 179 | RBM17 | MSLYDDLGVETSDSKTEGWSKNFKLLQSQLQVKKAALT | 401 |
| | splicing | QAKSQRTKQSTVLAPVIDLKRGGSSDDRQIVDTPPHVA | |
| | factor | AGLKDPVPSGFSAGEVLIPLADEYDPMFPNDYEKVVKR | |
| | expression | QREERQRQRELERQKEIEEREKRRKDRHEASGFARRPD | |
| | | PDSDEDEDYERERRKRSMGGAAIAPPTSLVEKDKELPR | |
| | | DFPYEEDSRPRSQSSKAAIPPPVYEEQDRPRSPTGPSN | |
| | | SFLANMGGTVAHKIMQKYGFREGQGLGKHEQGLSTALS | |
| | | VEKTSKRGGKIIVGDATEKDASKKSDSNPLTEILKCPT | |
| | | KVVLLRNMVGAGEVDEDLEVETKEECEKYGKVGKCVIF | |
| | | EIPGAPDDEAVRIFLEFERVESAIKAVVDLNGRYFGGR | |
| | | VVKACFYNLDKFRVLDLAEQV | |
| 507 | RBM17_G | MKRTADGSEFESPKKKRKVTTTMKISIEFLEPFRMTKW | 2052 |
| | GSLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAGSGGSGSGGSGSGGS | |
| | | SLYDDLGVETSDSKTEGWSKNFKLLQSQLQVKKAALTQ | |
| | | AKSQRTKQSTVLAPVIDLKRGGSSDDRQIVDTPPHVAA | |
| | | GLKDPVPSGFSAGEVLIPLADEYDPMFPNDYEKVVKRQ | |
| | | REERQRQRELERQKEIEEREKRRKDRHEASGFARRPDP | |
| | | DSDEDEDYERERRKRSMGGAAIAPPTSLVEKDKELPRD | |
| | | FPYEEDSRPRSQSSKAAIPPPVYEEQDRPRSPTGPSNS | |
| | | FLANMGGTVAHKIMQKYGFREGQGLGKHEQGLSTALSV | |
| | | EKTSKRGGKIIVGDATEKDASKKSDSNPLTEILKCPTK | |
| | | VVLLRNMVGAGEVDEDLEVETKEECEKYGKVGKCVIFE | |
| | | IPGAPDDEAVRIFLEFERVESAIKAVVDLNGRYFGGRV | |
| | | VKACFYNLDKFRVLDLAEQVKRTADGSEFESPKKKRKV | |
| 180 | SF3B6 | MAMQAAKRANIRLPPEVNRILYIRNLPYKITAEEMYDI | 125 |
| | splicing | FGKYGPIRQIRVGNTPETRGTAYVVYEDIFDAKNACDH | |
| | factor | LSGFNVCNRYLVVLYYNANRAFQKMDTKKKEEQLKLLK | |
| | expression | EKYGINTDPPK | |
| 508 | SF3B6_G | MKRTADGSEFESPKKKRKVTTTMKISIEFLEPFRMTKW | 1776 |
| | GSLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSESFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAGSGGSGSGGSGSGGS | |
| | | AMQAAKRANIRLPPEVNRILYIRNLPYKITAEEMYDIF | |
| | | GKYGPIRQIRVGNTPETRGTAYVVYEDIFDAKNACDHL | |
| | | SGFNVCNRYLVVLYYNANRAFQKMDTKKKEEQLKLLKE | |
| | | KYGINTDPPKKRTADGSEFESPKKKRKV | |
| 181 | U2AF1 | MAEYLASIFGTEKDKVNCSFYFKIGACRHGDRCSRLHN | 240 |
| | splicing | KPTFSQTIALLNIYRNPQNSSQSADGLRCAVSDVEMQE | |
| | factor | HYDEFFEEVFTEMEEKYGEVEEMNVCDNLGDHLVGNVY | |
| | expression | VKFRREEDAEKAVIDLNNRWFNGQPIHAELSPVTDFRE | |
| | | ACCRQYEMGECTRGGFCNFMHLKPISRELRRELYGRRR | |
| | | KKHRSRSRSRERRSRSRDRGRGGGGGGGGGGGGRERDR | |
| | | RRSRDRERSGRF | |
| 509 | U2AF1_ | MKRTADGSEFESPKKKRKVTTTMKISIEFLEPFRMTKW | 1891 |
| | GSLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAGSGGSGSGGSGSGGS | |
| | | AEYLASIFGTEKDKVNCSFYFKIGACRHGDRCSRLHNK | |
| | | PTFSQTIALLNIYRNPQNSSQSADGLRCAVSDVEMQEH | |
| | | YDEFFEEVFTEMEEKYGEVEEMNVCDNLGDHLVGNVYV | |
| | | KFRREEDAEKAVIDLNNRWFNGQPIHAELSPVTDFREA | |
| | | CCRQYEMGECTRGGFCNFMHLKPISRELRRELYGRRRK | |
| | | KHRSRSRSRERRSRSRDRGRGGGGGGGGGGGGRERDRR | |
| | | RSRDRERSGRFKRTADGSEFESPKKKRKV | |
| 182 | U2AF2 | MSDFDEFERQLNENKQERDKENRHRKRSHSRSRSRDRK | 475 |
| | splicing | RRSRSRDRRNRDQRSASRDRRRRSKPLTRGAKEEHGGL | |
| | factor | IRSPRHEKKKKVRKYWDVPPPGFEHITPMQYKAMQAAG | |
| | expression | QIPATALLPTMTPDGLAVTPTPVPVVGSQMTRQARRLY | |
| | | VGNIPFGITEEAMMDFFNAQMRLGGLTQAPGNPVLAVQ | |
| | | INQDKNFAFLEFRSVDETTQAMAFDGIIFQGQSLKIRR | |
| | | PHDYQPLPGMSENPSVYVPGVVSTVVPDSAHKLFIGGL | |
| | | PNYLNDDQVKELLTSFGPLKAFNLVKDSATGLSKGYAF | |
| | | CEYVDINVTDQAIAGLNGMQLGDKKLLVQRASVGAKNA | |
| | | TLVSPPSTINQTPVTLQVPGLMSSQVQMGGHPTEVLCL | |
| | | MNMVLPEELLDDEEYEEIVEDVRDECSKYGLVKSIEIP | |
| | | RPVDGVEVPGCGKIFVEFTSVEDCQKAMQGLTGRKFAN | |
| | | RVVVTKYCDPDSYHRRDFW | |
| 510 | U2AF2_ | MKRTADGSEFESPKKKRKVTTTMKISIEFLEPFRMTKW | 2126 |
| | GSLinker_ | QESTRRNKNNKEFVRGQAFARWHRNKKDNTKGRPYITG | |
| | Ct | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAGSGGSGSGGSGSGGS | |
| | | SDFDEFERQLNENKQERDKENRHRKRSHSRSRSRDRKR | |
| | | RSRSRDRRNRDQRSASRDRRRRSKPLTRGAKEEHGGLI | |
| | | RSPRHEKKKKVRKYWDVPPPGFEHITPMQYKAMQAAGQ | |
| | | IPATALLPTMTPDGLAVTPTPVPVVGSQMTRQARRLYV | |
| | | GNIPFGITEEAMMDFFNAQMRLGGLTQAPGNPVLAVQI | |
| | | NQDKNFAFLEFRSVDETTQAMAFDGIIFQGQSLKIRRP | |
| | | HDYQPLPGMSENPSVYVPGVVSTVVPDSAHKLFIGGLP | |
| | | NYLNDDQVKELLTSFGPLKAFNLVKDSATGLSKGYAFC | |
| | | EYVDINVTDQAIAGLNGMQLGDKKLLVQRASVGAKNAT | |
| | | LVSPPSTINQTPVTLQVPGLMSSQVQMGGHPTEVLCLM | |
| | | NMVLPEELLDDEEYEEIVEDVRDECSKYGLVKSIEIPR | |
| | | PVDGVEVPGCGKIFVEFTSVEDCQKAMQGLTGRKFANR | |
| | | VVVTKYCDPDSYHRRDFWKRTADGSEFESPKKKRKV | |
| 511 | Ct- | MKRTADGSEFESPKKKRKVTTTMKISIEFLEPFRMTKW | 2002 |
| | SF3B6- | QESTRRNKNNKEFVRGQAFARWHRNKKDNTKGRPYITG | |
| | U2AF1 | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKEDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKMAEY | |
| | | LASIFGTEKDKVNCSFYFKIGACRHGDRCSRLHNKPTF | |
| | | SQTIALLNIYRNPQNSSQSADGLRCAVSDVEMQEHYDE | |
| | | FFEEVFTEMEEKYGEVEEMNVCDNLGDHLVGNVYVKFR | |
| | | REEDAEKAVIDLNNRWFNGQPLHAELSPVTDFREACCR | |
| | | QYEMGECTRGGFCNEMHLKPISRELRRELYGRRRKKHR | |
| | | SRSRSRERRSRSRDRGRGGGGGGGGGGGGRERDRRRSR | |
| | | DRERSGRFKRTADGSEFESPKKKRKV | |
| 512 | Ct- | MKRTADGSEFESPKKKRKVTTTMKISIEFLEPFRMTKW | 2278 |
| | U2AF1- | QESTRRNKNNKEFVRGQAFARWHRNKKDNTKGRPYITG | |
| | RBM17 | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAEYLASIFGTEKDK | |
| | | VNCSFYFKIGACRHGDRCSRLHNKPTFSQTIALLNIYR | |
| | | NPQNSSQSADGLRCAVSDVEMQEHYDEFFEEVFTEMEE | |
| | | KYGEVEEMNVCDNLGDHLVGNVYVKFRREEDAEKAVID | |
| | | LNNRWFNGQPLHAELSPVTDFREACCRQYEMGECTRGG | |
| | | FCNEMHLKPISRELRRELYGRRRKKHRSRSRSRERRSR | |
| | | SRDRGRGGGGGGGGGGGGRERDRRRSRDRERSGREMSL | |
| | | YDDLGVETSDSKTEGWSKNFKLLQSQLQVKKAALTQAK | |
| | | SQRTKQSTVLAPVIDLKRGGSSDDRQIVDTPPHVAAGL | |
| | | KDPVPSGFSAGEVLIPLADEYDPMFPNDYEKVVKRQRE | |
| | | ERQRQRELERQKEIEEREKRRKDRHEASGFARRPDPDS | |
| | | DEDEDYERERRKRSMGGAAIAPPTSLVEKDKELPRDFP | |
| | | YEEDSRPRSQSSKAAIPPPVYEEQDRPRSPTGPSNSFL | |
| | | ANMGGTVAHKIMQKYGFREGQGLGKHEQGLSTALSVEK | |
| | | TSKRGGKIIVGDATEKDASKKSDSNPLTEILKCPTKVV | |
| | | LLRNMVGAGEVDEDLEVETKEECEKYGKVGKCVIFEIP | |
| | | GAPDDEAVRIFLEFERVESAIKAVVDLNGRYFGGRVVK | |
| | | ACFYNLDKFRVLDLAEQVKRTADGSEFESPKKKRKV | |
| 513 | Ct- | MKRTADGSEFESPKKKRKVTTTMKISIEFLEPFRMTKW | 2002 |
| | U2AF1- | QESTRRNKNNKEFVRGQAFARWHRNKKDNTKGRPYITG | |
| | SF3B6 | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAEYLASIFGTEKDK | |
| | | VNCSFYFKIGACRHGDRCSRLHNKPTFSQTIALLNIYR | |
| | | NPQNSSQSADGLRCAVSDVEMQEHYDEFFEEVFTEMEE | |
| | | KYGEVEEMNVCDNLGDHLVGNVYVKFRREEDAEKAVID | |
| | | LNNRWFNGQPLHAELSPVTDFREACCRQYEMGECTRGG | |
| | | FCNEMHLKPISRELRRELYGRRRKKHRSRSRSRERRSR | |
| | | SRDRGRGGGGGGGGGGGGRERDRRRSRDRERSGRFMAM | |
| | | QAAKRANIRLPPEVNRILYIRNLPYKITAEEMYDIFGK | |
| | | YGPIRQIRVGNTPETRGTAYVVYEDIFDAKNACDHLSG | |
| | | FNVCNRYLVVLYYNANRAFQKMDTKKKEEQLKLLKEKY | |
| | | GINTDPPKKRTADGSEFESPKKKRKV | |
| 514 | Ct- | MKRTADGSEFESPKKKRKVTTTMKISIEFLEPFRMTKW | 2352 |
| | U2AF1- | QESTRRNKNNKEFVRGQAFARWHRNKKDNTKGRPYITG | |
| | U2AF2 | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQDFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKDGEFKKEDRQKKLTTPWTPWAMAEYLASIFGTEKDK | |
| | | VNCSFYFKIGACRHGDRCSRLHNKPTFSQTIALLNIYR | |
| | | NPQNSSQSADGLRCAVSDVEMQEHYDEFFEEVFTEMEE | |
| | | KYGEVEEMNVCDNLGDHLVGNVYVKFRREEDAEKAVID | |
| | | LNNRWFNGQPLHAELSPVTDFREACCRQYEMGECTRGG | |
| | | FCNFMHLKPISRELRRELYGRRRKKHRSRSRSRERRSR | |
| | | SRDRGRGGGGGGGGGGGGRERDRRRSRDRERSGRFMSD | |
| | | FDEFERQLNENKQERDKENRHRKRSHSRSRSRDRKRRS | |
| | | RSRDRRNRDQRSASRDRRRRSKPLTRGAKEEHGGLIRS | |
| | | PRHEKKKKVRKYWDVPPPGFEHITPMQYKAMQAAGQIP | |
| | | ATALLPTMTPDGLAVTPTPVPVVGSQMTRQARRLYVGN | |
| | | IPFGITEEAMMDFFNAQMRLGGLTQAPGNPVLAVQINQ | |
| | | DKNFAFLEFRSVDETTQAMAFDGIIFQGQSLKIRRPHD | |
| | | YQPLPGMSENPSVYVPGVVSTVVPDSAHKLFIGGLPNY | |
| | | LNDDQVKELLTSFGPLKAFNLVKDSATGLSKGYAFCEY | |
| | | VDINVTDQAIAGLNGMQLGDKKLLVQRASVGAKNATLV | |
| | | SPPSTINQTPVTLQVPGLMSSQVQMGGHPTEVLCLMNM | |
| | | VLPEELLDDEEYEEIVEDVRDECSKYGLVKSIEIPRPV | |
| | | DGVEVPGCGKIFVEFTSVEDCQKAMQGLTGRKFANRVV | |
| | | VTKYCDPDSYHRRDFWKRTADGSEFESPKKKRKV | |
| 515 | Nt-Ct- | MKRTADGSEFESPKKKRKVMAMQAAKRANIRLPPEVNR | 1887 |
| | SF3B6 | ILYIRNLPYKITAEEMYDIFGKYGPIRQIRVGNTPETR | |
| | | GTAYVVYEDIFDAKNACDHLSGFNVCNRYLVVLYYNAN | |
| | | RAFQKMDTKKKEEQLKLLKEKYGINTDPPKTTTMKISI | |
| | | EFLEPFRMTKWQESTRRNKNNKEFVRGQAFARWHRNKK | |
| | | DNTKGRPYITGTLLRSAVIRSAENLLTLSDGKISEKTC | |
| | | CPGKFDTEDKDRLLQLRQRSTLRWTDKNPCPDNAETYC | |
| | | PFCELLGRSGNDGKKAEKKDWRFRIHFGNLSLPGKPDF | |
| | | DGPKAIGSQRVLNRVDFKSGKAHDFFKAYEVDHTRFPR | |
| | | FEGEITIDNKVSAEARKLLCDSLKFTDRLCGALCVIRF | |
| | | DEYTPAADSGKQTENVQAEPNANLAEKTAEQIISILDD | |
| | | NKKTEYTRLLADAIRSLRRSSKLVAGLPKDHDGKDDHY | |
| | | LWDIGKKKKDENSVTIRQILTTSADTKELKNAGKWREF | |
| | | CEKLGEALYLKSKDMSGGLKITRRILGDAEFHGKPDRL | |
| | | EKSRSVSIGSVLKETVVCGELVAKTPFFFGAIDEDAKQ | |
| | | TDLQVLLTPDNKYRLPRSAVRGILRRDLQTYFDSPCNA | |
| | | ELGGRPCMCKTCRIMRGITVMDARSEYNAPPEIRHRTR | |
| | | INPFTGTVAEGALFNMEVAPEGIVFPFQLRYRGSEDGL | |
| | | PDALKTVLKWWAEGQAFMSGAASTGKGRFRMENAKYET | |
| | | LDLSDENQRNDYLKNWGWRDEKGLEELKKRLNSGLPEP | |
| | | GNYRDPKWHEINVSIEMASPFINGDPIRAAVDKRGTDV | |
| | | VTFVKYKAEGEEAKPVCAYKAESFRGVIRSAVARIHME | |
| | | DGVPLTELTHSDCECLLCQIFGSEYEAGKIRFEDLVFE | |
| | | SDPEPVTFDHVAIDRFTGGAADKKKFDDSPLPGSPARP | |
| | | LMLKGSFWIRRDVLEDEEYCKALGKALADVNNGLYPLG | |
| | | GKSAIGYGQVKSLGIKGDDKRISRLMNPAFDETDVAVP | |
| | | EKPKTDAEVRIEAEKVYYPHYFVEPHKKVEREEKPCGH | |
| | | QKFHEGRLTGKIRCKLITKTPLIVPDTSNDDFFRPADK | |
| | | EARKEKDEYHKSYAFFRLHKQIMIPGSELRGMVSSVYE | |
| | | TVTNSCFRIFDETKRLSWRMDADHQNVLQDFLPGRVTA | |
| | | DGKHIQKFSETARVPFYDKTQKHFDILDEQEIAGEKPV | |
| | | RMWVKRFIKRLSLVDPAKHPQKKQDNKWKRRKEGIATF | |
| | | IEQKNGSYYFNVVTNNGCTSFHLWHKPDNFDQEKLEGI | |
| | | QNGEKLDCWVRDSRYQKAFQEIPENDPDGWECKEGYLH | |
| | | VVGPSKVEFSDKKGDVINNFQGTLPSVPNDWKTIRTND | |
| | | FKNRKRKNEPVFCCEDDKGNYYTMAKYCETFFFDLKEN | |
| | | EEYEIPEKARIKYKELLRVYNNNPQAVPESVFQSRVAR | |
| | | ENVEKLKSGDLVYFKHNEKYVEDIVPVRISRTVDDRMI | |
| | | GKRMSADLRPCHGDWVEDGDLSALNAYPEKRLLLRHPK | |
| | | GLCPACRLFGTGSYKGRVRFGFASLENDPEWLIPGKNP | |
| | | GDPFHGGPVMLSLLERPRPTWSIPGSDNKFKVPGRKFY | |
| | | VHHHAWKTIKDGNHPTTGKAIEQSPNNRTVEALAGGNS | |
| | | FSFEIAFENLKEWELGLLIHSLQLEKGLAHKLGMAKSM | |
| | | GFGSVEIDVESVRLRKDWKQWRNGNSEIPNWLGKGFAK | |
| | | LKEWFRDELDFIENLKKLLWFPEGDQAPRVCYPMLRKK | |
| | | DDPNGNSGYEELKDGEFKKEDRQKKLTTPWTPWAMAMQ | |
| | | AAKRANIRLPPEVNRILYIRNLPYKITAEEMYDIFGKY | |
| | | GPIRQIRVGNTPETRGTAYVVYEDIFDAKNACDHLSGE | |
| | | NVCNRYLVVLYYNANRAFQKMDTKKKEEQLKLLKEKYG | |
| | | INTDPPKKRTADGSEFESPKKKRKV | |
| 516 | Nt-Ct- | MKRTADGSEFESPKKKRKVMAEYLASIFGTEKDKVNCS | 2117 |
| | U2AF1 | FYFKIGACRHGDRCSRLHNKPTFSQTIALLNIYRNPQN | |
| | | SSQSADGLRCAVSDVEMQEHYDEFFEEVFTEMEEKYGE | |
| | | VEEMNVCDNLGDHLVGNVYVKFRREEDAEKAVIDLNNR | |
| | | WFNGQPIHAELSPVTDFREACCRQYEMGECTRGGFCNF | |
| | | MHLKPISRELRRELYGRRRKKHRSRSRSRERRSRSRDR | |
| | | GRGGGGGGGGGGGGRERDRRRSRDRERSGRFTTTMKIS | |
| | | IEFLEPFRMTKWQESTRRNKNNKEFVRGQAFARWHRNK | |
| | | KDNTKGRPYITGTLLRSAVIRSAENLLTLSDGKISEKT | |
| | | CCPGKFDTEDKDRLLQLRQRSTLRWTDKNPCPDNAETY | |
| | | CPFCELLGRSGNDGKKAEKKDWRFRIHFGNLSLPGKPD | |
| | | FDGPKAIGSQRVLNRVDFKSGKAHDFFKAYEVDHTRFP | |
| | | RFEGEITIDNKVSAEARKLLCDSLKFTDRLCGALCVIR | |
| | | FDEYTPAADSGKQTENVQAEPNANLAEKTAEQIISILD | |
| | | DNKKTEYTRLLADAIRSLRRSSKLVAGLPKDHDGKDDH | |
| | | YLWDIGKKKKDENSVTIRQILTTSADTKELKNAGKWRE | |
| | | FCEKLGEALYLKSKDMSGGLKITRRILGDAEFHGKPDR | |
| | | LEKSRSVSIGSVLKETVVCGELVAKTPFFFGAIDEDAK | |
| | | QTDLQVLLTPDNKYRLPRSAVRGILRRDLQTYFDSPCN | |
| | | AELGGRPCMCKTCRIMRGITVMDARSEYNAPPEIRHRT | |
| | | RINPFTGTVAEGALFNMEVAPEGIVFPFQLRYRGSEDG | |
| | | LPDALKTVLKWWAEGQAFMSGAASTGKGRFRMENAKYE | |
| | | TLDLSDENQRNDYLKNWGWRDEKGLEELKKRLNSGLPE | |
| | | PGNYRDPKWHEINVSIEMASPFINGDPIRAAVDKRGTD | |
| | | VVTFVKYKAEGEEAKPVCAYKAESFRGVIRSAVARIHM | |
| | | EDGVPLTELTHSDCECLLCQIFGSEYEAGKIRFEDLVF | |
| | | ESDPEPVTFDHVAIDRFTGGAADKKKFDDSPLPGSPAR | |
| | | PLMLKGSFWIRRDVLEDEEYCKALGKALADVNNGLYPL | |
| | | GGKSAIGYGQVKSLGIKGDDKRISRLMNPAFDETDVAV | |
| | | PEKPKTDAEVRIEAEKVYYPHYFVEPHKKVEREEKPCG | |
| | | HQKFHEGRLTGKIRCKLITKTPLIVPDTSNDDFFRPAD | |
| | | KEARKEKDEYHKSYAFFRLHKQIMIPGSELRGMVSSVY | |
| | | ETVTNSCFRIFDETKRLSWRMDADHQNVLQDFLPGRVT | |
| | | ADGKHIQKFSETARVPFYDKTQKHFDILDEQEIAGEKP | |
| | | VRMWVKRFIKRLSLVDPAKHPQKKQDNKWKRRKEGIAT | |
| | | FIEQKNGSYYFNVVTNNGCTSFHLWHKPDNFDQEKLEG | |
| | | IQNGEKLDCWVRDSRYQKAFQEIPENDPDGWECKEGYL | |
| | | HVVGPSKVEFSDKKGDVINNFQGTLPSVPNDWKTIRTN | |
| | | DFKNRKRKNEPVFCCEDDKGNYYTMAKYCETFFFDLKE | |
| | | NEEYEIPEKARIKYKELLRVYNNNPQAVPESVFQSRVA | |
| | | RENVEKLKSGDLVYFKHNEKYVEDIVPVRISRTVDDRM | |
| | | IGKRMSADLRPCHGDWVEDGDLSALNAYPEKRLLLRHP | |
| | | KGLCPACRLFGTGSYKGRVRFGFASLENDPEWLIPGKN | |
| | | PGDPFHGGPVMLSLLERPRPTWSIPGSDNKFKVPGRKF | |
| | | YVHHHAWKTIKDGNHPTTGKAIEQSPNNRTVEALAGGN | |
| | | SFSFEIAFENLKEWELGLLIHSLQLEKGLAHKLGMAKS | |
| | | MGFGSVEIDVESVRLRKDWKQWRNGNSEIPNWLGKGFA | |
| | | KLKEWFRDELDFIENLKKLLWFPEGDQAPRVCYPMLRK | |
| | | KDDPNGNSGYEELKDGEFKKEDRQKKLTTPWTPWAMAE | |
| | | YLASIFGTEKDKVNCSFYFKIGACRHGDRCSRLHNKPT | |
| | | FSQTIALLNIYRNPQNSSQSADGLRCAVSDVEMQEHYD | |
| | | EFFEEVFTEMEEKYGEVEEMNVCDNLGDHLVGNVYVKF | |
| | | RREEDAEKAVIDLNNRWFNGQPIHAELSPVTDFREACC | |
| | | RQYEMGECTRGGFCNEMHLKPISRELRRELYGRRRKKH | |
| | | RSRSRSRERRSRSRDRGRGGGGGGGGGGGGRERDRRRS | |
| | | RDRERSGRFKRTADGSEFESPKKKRKV | |
| 517 | Nt- | MKRTADGSEFESPKKKRKVMSLYDDLGVETSDSKTEGW | 2278 |
| | RBM17- | SKNFKLLQSQLQVKKAALTQAKSQRTKQSTVLAPVIDL | |
| | Ct- | KRGGSSDDRQIVDTPPHVAAGLKDPVPSGFSAGEVLIP | |
| | U2AF1 | LADEYDPMFPNDYEKVVKRQREERQRQRELERQKEIEE | |
| | | REKRRKDRHEASGFARRPDPDSDEDEDYERERRKRSMG | |
| | | GAAIAPPTSLVEKDKELPRDFPYEEDSRPRSQSSKAAI | |
| | | PPPVYEEQDRPRSPTGPSNSFLANMGGTVAHKIMQKYG | |
| | | FREGQGLGKHEQGLSTALSVEKTSKRGGKIIVGDATEK | |
| | | DASKKSDSNPLTEILKCPTKVVLLRNMVGAGEVDEDLE | |
| | | VETKEECEKYGKVGKCVIFEIPGAPDDEAVRIFLEFER | |
| | | VESAIKAVVDLNGRYFGGRVVKACFYNLDKFRVLDLAE | |
| | | QVITTMKISIEFLEPFRMTKWQESTRRNKNNKEFVRGQ | |
| | | AFARWHRNKKDNTKGRPYITGTLLRSAVIRSAENLLTL | |
| | | SDGKISEKTCCPGKFDTEDKDRLLQLRQRSTLRWTDKN | |
| | | PCPDNAETYCPFCELLGRSGNDGKKAEKKDWRFRIHFG | |
| | | NLSLPGKPDFDGPKAIGSQRVLNRVDFKSGKAHDFFKA | |
| | | YEVDHTRFPRFEGEITIDNKVSAEARKLLCDSLKFTDR | |
| | | LCGALCVIRFDEYTPAADSGKQTENVQAEPNANLAEKT | |
| | | AEQIISILDDNKKTEYTRLLADAIRSLRRSSKLVAGLP | |
| | | KDHDGKDDHYLWDIGKKKKDENSVTIRQILTTSADTKE | |
| | | LKNAGKWREFCEKLGEALYLKSKDMSGGLKITRRILGD | |
| | | AEFHGKPDRLEKSRSVSIGSVLKETVVCGELVAKTPFF | |
| | | FGAIDEDAKQTDLQVLLTPDNKYRLPRSAVRGILRRDL | |
| | | QTYFDSPCNAELGGRPCMCKTCRIMRGITVMDARSEYN | |
| | | APPEIRHRTRINPFTGTVAEGALFNMEVAPEGIVFPFQ | |
| | | LRYRGSEDGLPDALKTVLKWWAEGQAFMSGAASTGKGR | |
| | | FRMENAKYETLDLSDENQRNDYLKNWGWRDEKGLEELK | |
| | | KRLNSGLPEPGNYRDPKWHEINVSIEMASPFINGDPIR | |
| | | AAVDKRGTDVVTFVKYKAEGEEAKPVCAYKAESFRGVI | |
| | | RSAVARIHMEDGVPLTELTHSDCECLLCQIFGSEYEAG | |
| | | KIRFEDLVFESDPEPVTFDHVAIDRFTGGAADKKKFDD | |
| | | SPLPGSPARPLMLKGSFWIRRDVLEDEEYCKALGKALA | |
| | | DVNNGLYPLGGKSAIGYGQVKSLGIKGDDKRISRLMNP | |
| | | AFDETDVAVPEKPKTDAEVRIEAEKVYYPHYFVEPHKK | |
| | | VEREEKPCGHQKFHEGRLTGKIRCKLITKTPLIVPDTS | |
| | | NDDFFRPADKEARKEKDEYHKSYAFFRLHKQIMIPGSE | |
| | | LRGMVSSVYETVTNSCFRIFDETKRLSWRMDADHQNVL | |
| | | QDFLPGRVTADGKHIQKFSETARVPFYDKTQKHFDILD | |
| | | EQEIAGEKPVRMWVKRFIKRLSLVDPAKHPQKKQDNKW | |
| | | KRRKEGIATFIEQKNGSYYFNVVTNNGCTSFHLWHKPD | |
| | | NFDQEKLEGIQNGEKLDCWVRDSRYQKAFQEIPENDPD | |
| | | GWECKEGYLHVVGPSKVEFSDKKGDVINNFQGTLPSVP | |
| | | NDWKTIRTNDFKNRKRKNEPVFCCEDDKGNYYTMAKYC | |
| | | ETFFFDLKENEEYEIPEKARIKYKELLRVYNNNPQAVP | |
| | | ESVFQSRVARENVEKLKSGDLVYFKHNEKYVEDIVPVR | |
| | | ISRTVDDRMIGKRMSADLRPCHGDWVEDGDLSALNAYP | |
| | | EKRLLLRHPKGLCPACRLFGTGSYKGRVRFGFASLEND | |
| | | PEWLIPGKNPGDPFHGGPVMLSLLERPRPTWSIPGSDN | |
| | | KFKVPGRKFYVHHHAWKTIKDGNHPTTGKAIEQSPNNR | |
| | | TVEALAGGNSFSFEIAFENLKEWELGLLIHSLQLEKGL | |
| | | AHKLGMAKSMGFGSVEIDVESVRLRKDWKQWRNGNSEI | |
| | | PNWLGKGFAKLKEWFRDELDFIENLKKLLWFPEGDQAP | |
| | | RVCYPMLRKKDDPNGNSGYEELKDGEFKKEDRQKKLTT | |
| | | PWTPWAMAEYLASIFGTEKDKVNCSFYFKIGACRHGDR | |
| | | CSRLHNKPTFSQTIALLNIYRNPQNSSQSADGLRCAVS | |
| | | DVEMQEHYDEFFEEVFTEMEEKYGEVEEMNVCDNLGDH | |
| | | LVGNVYVKFRREEDAEKAVIDLNNRWFNGQPLHAELSP | |
| | | VTDFREACCRQYEMGECTRGGFCNFMHLKPISRELRRE | |
| | | LYGRRRKKHRSRSRSRERRSRSRDRGRGGGGGGGGGGG | |
| | | GRERDRRRSRDRERSGRFKRTADGSEFESPKKKRKV | |
| 518 | Nt- | MKRTADGSEFESPKKKRKVMAMQAAKRANIRLPPEVNR | 2002 |
| | SF3B6- | ILYIRNLPYKITAEEMYDIFGKYGPIRQIRVGNTPETR | |
| | Ct- | GTAYVVYEDIFDAKNACDHLSGFNVCNRYLVVLYYNAN | |
| | U2AF1 | RAFQKMDTKKKEEQLKLLKEKYGINTDPPKTTTMKISI | |
| | | EFLEPFRMTKWQESTRRNKNNKEFVRGQAFARWHRNKK | |
| | | DNTKGRPYITGTLLRSAVIRSAENLLTLSDGKISEKTC | |
| | | CPGKFDTEDKDRLLQLRQRSTLRWTDKNPCPDNAETYC | |
| | | PFCELLGRSGNDGKKAEKKDWRFRIHFGNLSLPGKPDF | |
| | | DGPKAIGSQRVLNRVDFKSGKAHDFFKAYEVDHTRFPR | |
| | | FEGEITIDNKVSAEARKLLCDSLKFTDRLCGALCVIRF | |
| | | DEYTPAADSGKQTENVQAEPNANLAEKTAEQIISILDD | |
| | | NKKTEYTRLLADAIRSLRRSSKLVAGLPKDHDGKDDHY | |
| | | LWDIGKKKKDENSVTIRQILTTSADTKELKNAGKWREF | |
| | | CEKLGEALYLKSKDMSGGLKITRRILGDAEFHGKPDRL | |
| | | EKSRSVSIGSVLKETVVCGELVAKTPFFFGAIDEDAKQ | |
| | | TDLQVLLTPDNKYRLPRSAVRGILRRDLQTYFDSPCNA | |
| | | ELGGRPCMCKTCRIMRGITVMDARSEYNAPPEIRHRTR | |
| | | INPFTGTVAEGALFNMEVAPEGIVFPFQLRYRGSEDGL | |
| | | PDALKTVLKWWAEGQAFMSGAASTGKGRFRMENAKYET | |
| | | LDLSDENQRNDYLKNWGWRDEKGLEELKKRLNSGLPEP | |
| | | GNYRDPKWHEINVSIEMASPFINGDPIRAAVDKRGTDV | |
| | | VTFVKYKAEGEEAKPVCAYKAESFRGVIRSAVARIHME | |
| | | DGVPLTELTHSDCECLLCQIFGSEYEAGKIRFEDLVFE | |
| | | SDPEPVTFDHVAIDRFTGGAADKKKEDDSPLPGSPARP | |
| | | LMLKGSFWIRRDVLEDEEYCKALGKALADVNNGLYPLG | |
| | | GKSAIGYGQVKSLGIKGDDKRISRLMNPAFDETDVAVP | |
| | | EKPKTDAEVRIEAEKVYYPHYFVEPHKKVEREEKPCGH | |
| | | QKFHEGRLTGKIRCKLITKTPLIVPDTSNDDFFRPADK | |
| | | EARKEKDEYHKSYAFFRLHKQIMIPGSELRGMVSSVYE | |
| | | TVTNSCFRIFDETKRLSWRMDADHQNVLQDFLPGRVTA | |
| | | DGKHIQKFSETARVPFYDKTQKHFDILDEQEIAGEKPV | |
| | | RMWVKRFIKRLSLVDPAKHPQKKQDNKWKRRKEGIATF | |
| | | IEQKNGSYYFNVVTNNGCTSFHLWHKPDNFDQEKLEGI | |
| | | QNGEKLDCWVRDSRYQKAFQEIPENDPDGWECKEGYLH | |
| | | VVGPSKVEFSDKKGDVINNFQGTLPSVPNDWKTIRTND | |
| | | FKNRKRKNEPVFCCEDDKGNYYTMAKYCETFFFDLKEN | |
| | | EEYEIPEKARIKYKELLRVYNNNPQAVPESVFQSRVAR | |
| | | ENVEKLKSGDLVYFKHNEKYVEDIVPVRISRTVDDRMI | |
| | | GKRMSADLRPCHGDWVEDGDLSALNAYPEKRLLLRHPK | |
| | | GLCPACRLFGTGSYKGRVRFGFASLENDPEWLIPGKNP | |
| | | GDPFHGGPVMLSLLERPRPTWSIPGSDNKFKVPGRKFY | |
| | | VHHHAWKTIKDGNHPTTGKAIEQSPNNRTVEALAGGNS | |
| | | FSFEIAFENLKEWELGLLIHSLQLEKGLAHKLGMAKSM | |
| | | GFGSVEIDVESVRLRKDWKQWRNGNSEIPNWLGKGFAK | |
| | | LKEWFRDELDFIENLKKLLWFPEGDQAPRVCYPMLRKK | |
| | | DDPNGNSGYEELKDGEFKKEDRQKKLTTPWTPWAMAEY | |
| | | LASIFGTEKDKVNCSFYFKIGACRHGDRCSRLHNKPTF | |
| | | SQTIALLNIYRNPQNSSQSADGLRCAVSDVEMQEHYDE | |
| | | FFEEVFTEMEEKYGEVEEMNVCDNLGDHLVGNVYVKFR | |
| | | REEDAEKAVIDLNNRWFNGQPLHAELSPVTDFREACCR | |
| | | QYEMGECTRGGFCNFMHLKPISRELRRELYGRRRKKHR | |
| | | SRSRSRERRSRSRDRGRGGGGGGGGGGGGRERDRRRSR | |
| | | DRERSGRFKRTADGSEFESPKKKRKV | |
| 519 | Nt- | MKRTADGSEFESPKKKRKVMAEYLASIFGTEKDKVNCS | 2002 |
| | U2AF1- | FYFKIGACRHGDRCSRLHNKPTFSQTIALLNIYRNPQN | |
| | Ct- | SSQSADGLRCAVSDVEMQEHYDEFFEEVFTEMEEKYGE | |
| | SF3B6 | VEEMNVCDNLGDHLVGNVYVKFRREEDAEKAVIDLNNR | |
| | | WFNGQPLHAELSPVTDFREACCRQYEMGECTRGGFCNF | |
| | | MHLKPISRELRRELYGRRRKKHRSRSRSRERRSRSRDR | |
| | | GRGGGGGGGGGGGGRERDRRRSRDRERSGRFTTTMKIS | |
| | | IEFLEPFRMTKWQESTRRNKNNKEFVRGQAFARWHRNK | |
| | | KDNTKGRPYITGTLLRSAVIRSAENLLTLSDGKISEKT | |
| | | CCPGKFDTEDKDRLLQLRQRSTLRWTDKNPCPDNAETY | |
| | | CPFCELLGRSGNDGKKAEKKDWRFRIHFGNLSLPGKPD | |
| | | FDGPKAIGSQRVLNRVDFKSGKAHDFFKAYEVDHTRFP | |
| | | RFEGEITIDNKVSAEARKLLCDSLKFTDRLCGALCVIR | |
| | | FDEYTPAADSGKQTENVQAEPNANLAEKTAEQIISILD | |
| | | DNKKTEYTRLLADAIRSLRRSSKLVAGLPKDHDGKDDH | |
| | | YLWDIGKKKKDENSVTIRQILTTSADTKELKNAGKWRE | |
| | | FCEKLGEALYLKSKDMSGGLKITRRILGDAEFHGKPDR | |
| | | LEKSRSVSIGSVLKETVVCGELVAKTPFFFGAIDEDAK | |
| | | QTDLQVLLTPDNKYRLPRSAVRGILRRDLQTYFDSPCN | |
| | | AELGGRPCMCKTCRIMRGITVMDARSEYNAPPEIRHRT | |
| | | RINPFTGTVAEGALFNMEVAPEGIVFPFQLRYRGSEDG | |
| | | LPDALKTVLKWWAEGQAFMSGAASTGKGRFRMENAKYE | |
| | | TLDLSDENQRNDYLKNWGWRDEKGLEELKKRLNSGLPE | |
| | | PGNYRDPKWHEINVSIEMASPFINGDPIRAAVDKRGTD | |
| | | VVTFVKYKAEGEEAKPVCAYKAESFRGVIRSAVARIHM | |
| | | EDGVPLTELTHSDCECLLCQIFGSEYEAGKIRFEDLVF | |
| | | ESDPEPVTFDHVAIDRFTGGAADKKKEDDSPLPGSPAR | |
| | | PLMLKGSFWIRRDVLEDEEYCKALGKALADVNNGLYPL | |
| | | GGKSAIGYGQVKSLGIKGDDKRISRLMNPAFDETDVAV | |
| | | PEKPKTDAEVRIEAEKVYYPHYFVEPHKKVEREEKPCG | |
| | | HQKFHEGRLTGKIRCKLITKTPLIVPDTSNDDFFRPAD | |
| | | KEARKEKDEYHKSYAFFRLHKQIMIPGSELRGMVSSVY | |
| | | ETVTNSCFRIFDETKRLSWRMDADHQNVLQDFLPGRVT | |
| | | ADGKHIQKFSETARVPFYDKTQKHFDILDEQEIAGEKP | |
| | | VRMWVKRFIKRLSLVDPAKHPQKKQDNKWKRRKEGIAT | |
| | | FIEQKNGSYYFNVVTNNGCTSFHLWHKPDNFDQEKLEG | |
| | | IQNGEKLDCWVRDSRYQKAFQEIPENDPDGWECKEGYL | |
| | | HVVGPSKVEFSDKKGDVINNFQGTLPSVPNDWKTIRTN | |
| | | DFKNRKRKNEPVFCCEDDKGNYYTMAKYCETFFFDLKE | |
| | | NEEYEIPEKARIKYKELLRVYNNNPQAVPESVFQSRVA | |
| | | RENVEKLKSGDLVYFKHNEKYVEDIVPVRISRTVDDRM | |
| | | IGKRMSADLRPCHGDWVEDGDLSALNAYPEKRLLLRHP | |
| | | KGLCPACRLFGTGSYKGRVRFGFASLENDPEWLIPGKN | |
| | | PGDPFHGGPVMLSLLERPRPTWSIPGSDNKFKVPGRKF | |
| | | YVHHHAWKTIKDGNHPTTGKAIEQSPNNRTVEALAGGN | |
| | | SFSFEIAFENLKEWELGLLIHSLQLEKGLAHKLGMAKS | |
| | | MGFGSVEIDVESVRLRKDWKQWRNGNSEIPNWLGKGFA | |
| | | KLKEWFRDELDFIENLKKLLWFPEGDQAPRVCYPMLRK | |
| | | KDDPNGNSGYEELKDGEFKKEDRQKKLTTPWTPWAMAM | |
| | | QAAKRANIRLPPEVNRILYIRNLPYKITAEEMYDIFGK | |
| | | YGPIRQIRVGNTPETRGTAYVVYEDIFDAKNACDHLSG | |
| | | FNVCNRYLVVLYYNANRAFQKMDTKKKEEQLKLLKEKY | |
| | | GINTDPPKKRTADGSEFESPKKKRKV | |
| 520 | Nt- | MKRTADGSEFESPKKKRKVMSDFDEFERQLNENKQERD | 2352 |
| | U2AF2- | KENRHRKRSHSRSRSRDRKRRSRSRDRRNRDQRSASRD | |
| | Ct- | RRRRSKPLTRGAKEEHGGLIRSPRHEKKKKVRKYWDVP | |
| | U2AF1 | PPGFEHITPMQYKAMQAAGQIPATALLPTMTPDGLAVT | |
| | | PTPVPVVGSQMTRQARRLYVGNIPFGITEEAMMDFFNA | |
| | | QMRLGGLTQAPGNPVLAVQINQDKNFAFLEFRSVDETT | |
| | | QAMAFDGIIFQGQSLKIRRPHDYQPLPGMSENPSVYVP | |
| | | GVVSTVVPDSAHKLFIGGLPNYLNDDQVKELLTSFGPL | |
| | | KAFNLVKDSATGLSKGYAFCEYVDINVTDQAIAGLNGM | |
| | | QLGDKKLLVQRASVGAKNATLVSPPSTINQTPVTLQVP | |
| | | GLMSSQVQMGGHPTEVLCLMNMVLPEELLDDEEYEEIV | |
| | | EDVRDECSKYGLVKSIEIPRPVDGVEVPGCGKIFVEFT | |
| | | SVFDCQKAMQGLTGRKFANRVVVTKYCDPDSYHRRDFW | |
| | | TTTMKISIEFLEPFRMTKWQESTRRNKNNKEFVRGQAF | |
| | | ARWHRNKKDNTKGRPYITGTLLRSAVIRSAENLLTLSD | |
| | | GKISEKTCCPGKFDTEDKDRLLQLRQRSTLRWTDKNPC | |
| | | PDNAETYCPFCELLGRSGNDGKKAEKKDWRFRIHFGNL | |
| | | SLPGKPDFDGPKAIGSQRVLNRVDFKSGKAHDFFKAYE | |
| | | VDHTRFPRFEGEITIDNKVSAEARKLLCDSLKFTDRLC | |
| | | GALCVIRFDEYTPAADSGKQTENVQAEPNANLAEKTAE | |
| | | QIISILDDNKKTEYTRLLADAIRSLRRSSKLVAGLPKD | |
| | | HDGKDDHYLWDIGKKKKDENSVTIRQILTTSADTKELK | |
| | | NAGKWREFCEKLGEALYLKSKDMSGGLKITRRILGDAE | |
| | | FHGKPDRLEKSRSVSIGSVLKETVVCGELVAKTPFFFG | |
| | | AIDEDAKQTDLQVLLTPDNKYRLPRSAVRGILRRDLQT | |
| | | YFDSPCNAELGGRPCMCKTCRIMRGITVMDARSEYNAP | |
| | | PEIRHRTRINPFTGTVAEGALFNMEVAPEGIVFPFQLR | |
| | | YRGSEDGLPDALKTVLKWWAEGQAFMSGAASTGKGRFR | |
| | | MENAKYETLDLSDENQRNDYLKNWGWRDEKGLEELKKR | |
| | | LNSGLPEPGNYRDPKWHEINVSIEMASPFINGDPIRAA | |
| | | VDKRGTDVVTFVKYKAEGEEAKPVCAYKAESFRGVIRS | |
| | | AVARIHMEDGVPLTELTHSDCECLLCQIFGSEYEAGKI | |
| | | RFEDLVFESDPEPVTFDHVAIDRFTGGAADKKKEDDSP | |
| | | LPGSPARPLMLKGSFWIRRDVLEDEEYCKALGKALADV | |
| | | NNGLYPLGGKSAIGYGQVKSLGIKGDDKRISRLMNPAF | |
| | | DETDVAVPEKPKTDAEVRIEAEKVYYPHYFVEPHKKVE | |
| | | REEKPCGHQKFHEGRLTGKIRCKLITKTPLIVPDTSND | |
| | | DFFRPADKEARKEKDEYHKSYAFFRLHKQIMIPGSELR | |
| | | GMVSSVYETVTNSCFRIFDETKRLSWRMDADHQNVLQD | |
| | | FLPGRVTADGKHIQKFSETARVPFYDKTQKHFDILDEQ | |
| | | EIAGEKPVRMWVKRFIKRLSLVDPAKHPQKKQDNKWKR | |
| | | RKEGIATFIEQKNGSYYFNVVTNNGCTSFHLWHKPDNF | |
| | | DQEKLEGIQNGEKLDCWVRDSRYQKAFQEIPENDPDGW | |
| | | ECKEGYLHVVGPSKVEFSDKKGDVINNFQGTLPSVPND | |
| | | WKTIRTNDFKNRKRKNEPVFCCEDDKGNYYTMAKYCET | |
| | | FFFDLKENEEYEIPEKARIKYKELLRVYNNNPQAVPES | |
| | | VFQSRVARENVEKLKSGDLVYFKHNEKYVEDIVPVRIS | |
| | | RTVDDRMIGKRMSADLRPCHGDWVEDGDLSALNAYPEK | |
| | | RLLLRHPKGLCPACRLFGTGSYKGRVRFGFASLENDPE | |
| | | WLIPGKNPGDPFHGGPVMLSLLERPRPTWSIPGSDNKF | |
| | | KVPGRKFYVHHHAWKTIKDGNHPTTGKAIEQSPNNRTV | |
| | | EALAGGNSFSFEIAFENLKEWELGLLIHSLQLEKGLAH | |
| | | KLGMAKSMGFGSVEIDVESVRLRKDWKQWRNGNSEIPN | |
| | | WLGKGFAKLKEWFRDELDFIENLKKLLWFPEGDQAPRV | |
| | | CYPMLRKKDDPNGNSGYEELKDGEFKKEDRQKKLTTPW | |
| | | TPWAMAEYLASIFGTEKDKVNCSFYFKIGACRHGDRCS | |
| | | RLHNKPTFSQTIALLNIYRNPQNSSQSADGLRCAVSDV | |
| | | EMQEHYDEFFEEVFTEMEEKYGEVEEMNVCDNLGDHLV | |
| | | GNVYVKFRREEDAEKAVIDLNNRWFNGQPLHAELSPVT | |
| | | DFREACCRQYEMGECTRGGFCNFMHLKPISRELRRELY | |
| | | GRRRKKHRSRSRSRERRSRSRDRGRGGGGGGGGGGGGR | |
| | | ERDRRRSRDRERSGRFKRTADGSEFESPKKKRKV | |
| 521 | PDF0954_ | MKRTADGSEFESPKKKRKVTTTMKISIEFLEPFRMTKW | 1326 |
| | Cas711S- | QESTRRNKNNKEFVRGQAFARWHRNKKDNTKGRPYITG | |
| | E279R- | TLLRSAVIRSAENLLTLSDGKISEKTCCPGKFDTEDKD | |
| | D1580R- | RLLQLRQRSTLRWTDKNPCPDNAETYCPFCELLGRSGN | |
| | point- | DGKKAEKKDWRFRIHFGNLSLPGKPDFDGPKAIGSQRV | |
| | mutationR174G | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVEPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSESFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAKRTADGSEFESPKKKRKV | |
| 522 | pDF0952_ | MKRTADGSEFESPKKKRKVTTTMKISIEFLEPFRMTKW | 1727 |
| | RBM17 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKDGEFK | |
| | | KEDRQKKLTTPWTPWAMSLYDDLGVETSDSKTEGWSKN | |
| | | FKLLQSQLQVKKAALTQAKSQRTKQSTVLAPVIDLKRG | |
| | | GSSDDRQIVDTPPHVAAGLKDPVPSGESAGEVLIPLAD | |
| | | EYDPMFPNDYEKVVKRQREERQRQRELERQKEIEEREK | |
| | | RRKDRHEASGFARRPDPDSDEDEDYERERRKRSMGGAA | |
| | | IAPPTSLVEKDKELPRDFPYEEDSRPRSQSSKAAIPPP | |
| | | VYEEQDRPRSPTGPSNSFLANMGGTVAHKIMQKYGFRE | |
| | | GQGLGKHEQGLSTALSVEKTSKRGGKIIVGDATEKDAS | |
| | | KKSDSNPLTEILKCPTKVVLLRNMVGAGEVDEDLEVET | |
| | | KEECEKYGKVGKCVIFEIPGAPDDEAVRIFLEFERVES | |
| | | AIKAVVDLNGRYFGGRVVKACFYNLDKFRVLDLAEQVK | |
| | | RTADGSEFESPKKKRKV | |
| 523 | pDF0952_ | MKRTADGSEFESPKKKRKVTTTMKISIEFLEPFRMTKW | 1451 |
| | SF3B6 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKDGEFK | |
| | | KEDRQKKLTTPWTPWAMAMQAAKRANIRLPPEVNRILY | |
| | | IRNLPYKITAEEMYDIFGKYGPIRQIRVGNTPETRGTA | |
| | | YVVYEDIFDAKNACDHLSGFNVCNRYLVVLYYNANRAF | |
| | | QKMDTKKKEEQLKLLKEKYGINTDPPKKRTADGSEFES | |
| | | PKKKRKV | |
| 524 | pDF0952_ | MKRTADGSEFESPKKKRKVTTTMKISIEFLEPFRMTKW | 1566 |
| | U2AF1 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKDGEFK | |
| | | KEDRQKKLTTPWTPWAMAEYLASIFGTEKDKVNCSFYF | |
| | | KIGACRHGDRCSRLHNKPTFSQTIALLNIYRNPQNSSQ | |
| | | SADGLRCAVSDVEMQEHYDEFFEEVFTEMEEKYGEVEE | |
| | | MNVCDNLGDHLVGNVYVKFRREEDAEKAVIDLNNRWFN | |
| | | GQPIHAELSPVTDFREACCRQYEMGECTRGGFCNFMHL | |
| | | KPISRELRRELYGRRRKKHRSRSRSRERRSRSRDRGRG | |
| | | GGGGGGGGGGGRERDRRRSRDRERSGRFKRTADGSEFE | |
| | | SPKKKRKV | |
| 525 | pDF0952_ | MKRTADGSEFESPKKKRKVTTTMKISIEFLEPFRMTKW | 1801 |
| | U2AF2 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWERDELDFIENLKKL | |
| | | LWFPEGDQAPRVCYPMLRKKDDPNGNSGYEELKDGEFK | |
| | | KEDRQKKLTTPWTPWAMSDFDEFERQLNENKQERDKEN | |
| | | RHRKRSHSRSRSRDRKRRSRSRDRRNRDQRSASRDRRR | |
| | | RSKPLTRGAKEEHGGLIRSPRHEKKKKVRKYWDVPPPG | |
| | | FEHITPMQYKAMQAAGQIPATALLPTMTPDGLAVTPTP | |
| | | VPVVGSQMTRQARRLYVGNIPFGITEEAMMDFFNAQMR | |
| | | LGGLTQAPGNPVLAVQINQDKNFAFLEFRSVDETTQAM | |
| | | AFDGIIFQGQSLKIRRPHDYQPLPGMSENPSVYVPGVV | |
| | | STVVPDSAHKLFIGGLPNYLNDDQVKELLTSFGPLKAF | |
| | | NLVKDSATGLSKGYAFCEYVDINVTDQAIAGINGMQLG | |
| | | DKKLLVQRASVGAKNATLVSPPSTINQTPVTLQVPGLM | |
| | | SSQVQMGGHPTEVLCLMNMVLPEELLDDEEYEEIVEDV | |
| | | RDECSKYGLVKSIEIPRPVDGVEVPGCGKIFVEFTSVF | |
| | | DCQKAMQGLTGRKFANRVVVTKYCDPDSYHRRDFWKRT | |
| | | ADGSEFESPKKKRKV | |
| 526 | pDF0953_ | MKRTADGSEFESPKKKRKVTTTMKISIEFLEPFRMTKW | 1727 |
| | RBM17 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMSLYDDLGVETSDSKTEGWSKN | |
| | | FKLLQSQLQVKKAALTQAKSQRTKQSTVLAPVIDLKRG | |
| | | GSSDDRQIVDTPPHVAAGLKDPVPSGFSAGEVLIPLAD | |
| | | EYDPMFPNDYEKVVKRQREERQRQRELERQKEIEEREK | |
| | | RRKDRHEASGFARRPDPDSDEDEDYERERRKRSMGGAA | |
| | | IAPPTSLVEKDKELPRDFPYEEDSRPRSQSSKAAIPPP | |
| | | VYEEQDRPRSPTGPSNSFLANMGGTVAHKIMQKYGFRE | |
| | | GQGLGKHEQGLSTALSVEKTSKRGGKIIVGDATEKDAS | |
| | | KKSDSNPLTEILKCPTKVVLLRNMVGAGEVDEDLEVET | |
| | | KEECEKYGKVGKCVIFEIPGAPDDEAVRIFLEFERVES | |
| | | AIKAVVDLNGRYFGGRVVKACFYNLDKFRVLDLAEQVK | |
| | | RTADGSEFESPKKKRKV | |
| 527 | pDF0953_ | MKRTADGSEFESPKKKRKVTTTMKISIEFLEPFRMTKW | 1451 |
| | SF3B6 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDEFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMAMQAAKRANIRLPPEVNRILY | |
| | | IRNLPYKITAEEMYDIFGKYGPIRQIRVGNTPETRGTA | |
| | | YVVYEDIFDAKNACDHLSGFNVCNRYLVVLYYNANRAF | |
| | | QKMDTKKKEEQLKLLKEKYGINTDPPKKRTADGSEFES | |
| | | PKKKRKV | |
| 528 | pDF0953_ | MKRTADGSEFESPKKKRKVTTTMKISIEFLEPFRMTKW | 1566 |
| | U2AF1 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVEPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMAEYLASIFGTEKDKVNCSFYF | |
| | | KIGACRHGDRCSRLHNKPTFSQTIALLNIYRNPQNSSQ | |
| | | SADGLRCAVSDVEMQEHYDEFFEEVFTEMEEKYGEVEE | |
| | | MNVCDNLGDHLVGNVYVKFRREEDAEKAVIDLNNRWFN | |
| | | GQPIHAELSPVTDFREACCRQYEMGECTRGGFCNFMHL | |
| | | KPISRELRRELYGRRRKKHRSRSRSRERRSRSRDRGRG | |
| | | GGGGGGGGGGGRERDRRRSRDRERSGRFKRTADGSEFE | |
| | | SPKKKRKV | |
| 529 | pDF0953_ | MKRTADGSEFESPKKKRKVTTTMKISIEFLEPFRMTKW | 1801 |
| | U2AF2 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMSDFDEFERQLNENKQERDKEN | |
| | | RHRKRSHSRSRSRDRKRRSRSRDRRNRDQRSASRDRRR | |
| | | RSKPLTRGAKEEHGGLIRSPRHEKKKKVRKYWDVPPPG | |
| | | FEHITPMQYKAMQAAGQIPATALLPTMTPDGLAVTPTP | |
| | | VPVVGSQMTRQARRLYVGNIPFGITEEAMMDFFNAQMR | |
| | | LGGLTQAPGNPVLAVQINQDKNFAFLEFRSVDETTQAM | |
| | | AFDGIIFQGQSLKIRRPHDYQPLPGMSENPSVYVPGVV | |
| | | STVVPDSAHKLFIGGLPNYLNDDQVKELLTSFGPLKAF | |
| | | NLVKDSATGLSKGYAFCEYVDINVTDQAIAGLNGMQLG | |
| | | DKKLLVQRASVGAKNATLVSPPSTINQTPVTLQVPGLM | |
| | | SSQVQMGGHPTEVLCLMNMVLPEELLDDEEYEEIVEDV | |
| | | RDECSKYGLVKSIEIPRPVDGVEVPGCGKIFVEFTSVF | |
| | | DCQKAMQGLTGRKFANRVVVTKYCDPDSYHRRDFWKRT | |
| | | ADGSEFESPKKKRKV | |
| 530 | pDF0954_ | MKRTADGSEFESPKKKRKVTTTMKISIEFLEPFRMTKW | 1727 |
| | RBM17 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMSLYDDLGVETSDSKTEGWSKN | |
| | | FKLLQSQLQVKKAALTQAKSQRTKQSTVLAPVIDLKRG | |
| | | GSSDDRQIVDTPPHVAAGLKDPVPSGFSAGEVLIPLAD | |
| | | EYDPMFPNDYEKVVKRQREERQRQRELERQKEIEEREK | |
| | | RRKDRHEASGFARRPDPDSDEDEDYERERRKRSMGGAA | |
| | | IAPPTSLVEKDKELPRDFPYEEDSRPRSQSSKAAIPPP | |
| | | VYEEQDRPRSPTGPSNSFLANMGGTVAHKIMQKYGFRE | |
| | | GQGLGKHEQGLSTALSVEKTSKRGGKIIVGDATEKDAS | |
| | | KKSDSNPLTEILKCPTKVVLLRNMVGAGEVDEDLEVET | |
| | | KEECEKYGKVGKCVIFEIPGAPDDEAVRIFLEFERVES | |
| | | AIKAVVDLNGRYFGGRVVKACFYNLDKFRVLDLAEQVK | |
| | | RTADGSEFESPKKKRKV | |
| 531 | pDF0954_ | MKRTADGSEFESPKKKRKVTTTMKISIEFLEPFRMTKW | 1451 |
| | SF3B6 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMAMQAAKRANIRLPPEVNRILY | |
| | | IRNLPYKITAEEMYDIFGKYGPIRQIRVGNTPETRGTA | |
| | | YVVYEDIFDAKNACDHLSGFNVCNRYLVVLYYNANRAF | |
| | | QKMDTKKKEEQLKLLKEKYGINTDPPKKRTADGSEFES | |
| | | PKKKRKV | |
| 532 | pDF0954_ | MKRTADGSEFESPKKKRKVTTTMKISIEFLEPFRMTKW | 1566 |
| | U2AF1 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMAEYLASIFGTEKDKVNCSFYF | |
| | | KIGACRHGDRCSRLHNKPTFSQTIALLNIYRNPQNSSQ | |
| | | SADGLRCAVSDVEMQEHYDEFFEEVFTEMEEKYGEVEE | |
| | | MNVCDNLGDHLVGNVYVKFRREEDAEKAVIDLNNRWFN | |
| | | GQPIHAELSPVTDFREACCRQYEMGECTRGGFCNFMHL | |
| | | KPISRELRRELYGRRRKKHRSRSRSRERRSRSRDRGRG | |
| | | GGGGGGGGGGGRERDRRRSRDRERSGRFKRTADGSEFE | |
| | | SPKKKRKV | |
| 533 | pDF0954_ | MKRTADGSEFESPKKKRKVTTTMKISIEFLEPFRMTKW | 1801 |
| | U2AF2 | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAMSDFDEFERQLNENKQERDKEN | |
| | | RHRKRSHSRSRSRDRKRRSRSRDRRNRDQRSASRDRRR | |
| | | RSKPLTRGAKEEHGGLIRSPRHEKKKKVRKYWDVPPPG | |
| | | FEHITPMQYKAMQAAGQIPATALLPTMTPDGLAVTPTP | |
| | | VPVVGSQMTRQARRLYVGNIPFGITEEAMMDFFNAQMR | |
| | | LGGLTQAPGNPVLAVQINQDKNFAFLEFRSVDETTQAM | |
| | | AFDGIIFQGQSLKIRRPHDYQPLPGMSENPSVYVPGVV | |
| | | STVVPDSAHKLFIGGLPNYLNDDQVKELLTSFGPLKAF | |
| | | NLVKDSATGLSKGYAFCEYVDINVTDQAIAGLNGMQLG | |
| | | DKKLLVQRASVGAKNATLVSPPSTINQTPVTLQVPGLM | |
| | | SSQVQMGGHPTEVLCLMNMVLPEELLDDEEYEEIVEDV | |
| | | RDECSKYGLVKSIEIPRPVDGVEVPGCGKIFVEFTSVF | |
| | | DCQKAMQGLTGRKFANRVVVTKYCDPDSYHRRDFWKRT | |
| | | ADGSEFESPKKKRKV | |
| 534 | Cas7-11 | MKRTADGSEFESPKKKRKVTTTMKISIEFLEPFRMTKW | 1792 |
| | lenti | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHKLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKVCTGSGEGRGSLLTCGDVEENPGPMAKPLSQEESTL | |
| | | IERATATINSIPISEDYSVASAALSSDGRIFTGVNVYH | |
| | | FTGGPCAELVVLGTAAAAAAGNLTCIVAIGNENRGILS | |
| | | PCGRCRQVLLDLHPGIKAIVKDSDGQPTAVGIRELLPS | |
| | | GYVWEG | |
| 467 | pDF0952 | MKRTADGSEFESPKKKRKVTTTMKISIEFLEPFRMTKW | 1326 |
| | | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | REGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWERDELDFIENLKKL | |
| | | LWFPEGDQAPRVCYPMLRKKDDPNGNSGYEELKDGEFK | |
| | | KEDRQKKLTTPWTPWAKRTADGSEFESPKKKRKV | |
| 535 | pDF0953 | MKRTADGSEFESPKKKRKVTTTMKISIEFLEPFRMTKW | 1326 |
| | | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAKRTADGSEFESPKKKRKV | |
| 521 | pDF0954 | MKRTADGSEFESPKKKRKVTTTMKISIEFLEPFRMTKW | 1326 |
| | | QESTRRNKNNKEFVRGQAFARWHRNKKDNTKGRPYITG | |
| | | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTRYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAKRTADGSEFESPKKKRKV | |
| 479 | pDF0951 | MKRTADGSEFESPKKKRKVTTTMKISIEFLEPFRMTKW | 1637 |
| | quadruple | QESTRRNKNNKEFVRGQAFARWHRNKKDNTKGRPYITG | |
| | mutant | TLLRSAVIRSAENLLTLSDGKISEKTCCPGKFDTEDKD | |
| | | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHKLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAKRTADGSEFESPKKK | |
| | | RKV | |
| 494 | pDF0964 | MKRTADGSEFESPKKKRKVTTTMKISIEFLEPFRMTKW | 1762 |
| | double | QESTRRNKNNKEFVRGQAFARWHRNKKDNTKGRPYITG | |
| | mutant | TLLRSAVIRSAENLLTLSDGKISEKTCCPGKFDTEDKD | |
| | SF3B6 | RLLQLRQRSTLRWTDKNPCPDNAETYCPFCELLGRSGN | |
| | fusion- | DGKKAEKKDWRFRIHFGNLSLPGKPDFDGPKAIGSQRV | |
| | aRY1589 | LNRVDFKSGKAHDFFKAYEVDHTRFPRFEGEITIDNKV | |
| | B1-C1 | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKKRTA | |
| | | DGSEFESPKKKRKV | |
| 495 | pDF0965 | MKRTADGSEFESPKKKRKVTTTMKISIEFLEPFRMTKW | 2112 |
| | double | QESTRRNKNNKEFVRGQAFARWHRNKKDNTKGRPYITG | |
| | mutant | TLLRSAVIRSAENLLTLSDGKISEKTCCPGKFDTEDKD | |
| | U2AF2 | RLLQLRQRSTLRWTDKNPCPDNAETYCPFCELLGRSGN | |
| | fusion- | DGKKAEKKDWRFRIHFGNLSLPGKPDFDGPKAIGSQRV | |
| | aRY1589 | LNRVDFKSGKAHDFFKAYEVDHTRFPRFEGEITIDNKV | |
| | E1 | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDADHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMSDFDEFERQLNENK | |
| | | QERDKENRHRKRSHSRSRSRDRKRRSRSRDRRNRDQRS | |
| | | ASRDRRRRSKPLTRGAKEEHGGLIRSPRHEKKKKVRKY | |
| | | WDVPPPGFEHITPMQYKAMQAAGQIPATALLPTMTPDG | |
| | | LAVTPTPVPVVGSQMTRQARRLYVGNIPFGITEEAMMD | |
| | | FFNAQMRLGGLTQAPGNPVLAVQINQDKNFAFLEFRSV | |
| | | DETTQAMAFDGIIFQGQSLKIRRPHDYQPLPGMSENPS | |
| | | VYVPGVVSTVVPDSAHKLFIGGLPNYLNDDQVKELLTS | |
| | | FGPLKAFNLVKDSATGLSKGYAFCEYVDINVTDQAIAG | |
| | | LNGMQLGDKKLLVQRASVGAKNATLVSPPSTINQTPVT | |
| | | LQVPGLMSSQVQMGGHPTEVLCLMNMVLPEELLDDEEY | |
| | | EEIVEDVRDECSKYGLVKSIEIPRPVDGVEVPGCGKIF | |
| | | VEFTSVFDCQKAMQGLTGRKFANRVVVTKYCDPDSYHR | |
| | | RDFWKRTADGSEFESPKKKRKV | |
| 496 | pDF0966 | MKRTADGSEFESPKKKRKVTTTMKISIEFLEPFRMTKW | 1762 |
| | triple | QESTRRNKNNKEFVRGQAFARWHRNKKDNTKGRPYITG | |
| | mutant | TLLRSAVIRSAENLLTLSDGKISEKTCCPGKFDTEDKD | |
| | SF3B6 | RLLQLRQRSTLRWTDKNPCPDNAETYCPFCELLGRSGN | |
| | fusion- | DGKKAEKKDWRFRIHFGNLSLPGKPDFDGPKAIGSQRV | |
| | aRY1589 | LNRVDFKSGKAHDFFKAYEVDHTRFPRFEGEITIDNKV | |
| | C2-D2 | SAEARKLLCDSLKFTDRLCGALCVIRFDEYTPAADSGK | |
| | | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMAMQAAKRANIRLPP | |
| | | EVNRILYIRNLPYKITAEEMYDIFGKYGPIRQIRVGNT | |
| | | PETRGTAYVVYEDIFDAKNACDHLSGFNVCNRYLVVLY | |
| | | YNANRAFQKMDTKKKEEQLKLLKEKYGINTDPPKKRTA | |
| | | DGSEFESPKKKRKV | |
| 497 | pDF0967 | MKRTADGSEFESPKKKRKVTTTMKISIEFLEPFRMTKW | 2112 |
| | triple | QESTRRNKNNKEFVRGQAFARWHRNKKDNTKGRPYITG | |
| | mutant | TLLRSAVIRSAENLLTLSDGKISEKTCCPGKFDTEDKD | |
| | U2AF2 | RLLQLRQRSTLRWTDKNPCPDNAETYCPFCELLGRSGN | |
| | fusion- | DGKKAEKKDWRFRIHFGNLSLPGKPDFDGPKAIGSQRV | |
| | aRY1589 | LNRVDFKSGKAHDFFKAYEVDHTRFPRFEGEITIDNKV | |
| | E2-F2- | SAEARKLLCDSLKFTDRLCGALCVIRFDEYTPAADSGK | |
| | G2-H2 | QTENVQAEPNANLAEKTAEQIISILDDNKKTEYTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRMDAKHQNVLQKFLPGRVTADGKHIQKFSET | |
| | | ARVPFYDKTQKHFDILDEQEIAGEKPVRMWVKRFIKRL | |
| | | SLVDPAKHPQKKQDNKWKRRKEGIATFIEQKNGSYYFN | |
| | | VVTNNGCTSFHLWHKPDNFDQEKLEGIQNGEKLDCWVR | |
| | | DSRYQKAFQEIPENDPDGWECKEGYLHVVGPSKVEFSD | |
| | | KKGDVINNFQGTLPSVPNDWKTIRTNDFKNRKRKNEPV | |
| | | FCCEDDKGNYYTMAKYCETFFFDLKENEEYEIPEKARI | |
| | | KYKELLRVYNNNPQAVPESVFQSRVARENVEKLKSGDL | |
| | | VYFKHNEKYVEDIVPVRISRTVDDRMIGKRMSADLRPC | |
| | | HGDWVEDGDLSALNAYPEKRLLLRHPKGLCPACRLFGT | |
| | | GSYKGRVRFGFASLENDPEWLIPGKNPGDPFHGGPVML | |
| | | SLLERPRPTWSIPGSDNKFKVPGRKFYVHHHAWKTIKD | |
| | | GNHPTTGKAIEQSPNNRTVEALAGGNSFSFEIAFENLK | |
| | | EWELGLLIHSLQLEKGLAHKLGMAKSMGFGSVEIDVES | |
| | | VRLRKDWKQWRNGNSEIPNWLGKGFAKLKEWFRDELDF | |
| | | IENLKKLLWFPEGDQAPRVCYPMLRKKDDPNGNSGYEE | |
| | | LKRGEFKKEDRQKKLTTPWTPWAMSDFDEFERQLNENK | |
| | | QERDKENRHRKRSHSRSRSRDRKRRSRSRDRRNRDQRS | |
| | | ASRDRRRRSKPLTRGAKEEHGGLIRSPRHEKKKKVRKY | |
| | | WDVPPPGFEHITPMQYKAMQAAGQIPATALLPTMTPDG | |
| | | LAVTPTPVPVVGSQMTRQARRLYVGNIPFGITEEAMMD | |
| | | FFNAQMRLGGLTQAPGNPVLAVQINQDKNFAFLEFRSV | |
| | | DETTQAMAFDGIIFQGQSLKIRRPHDYQPLPGMSENPS | |
| | | VYVPGVVSTVVPDSAHKLFIGGLPNYLNDDQVKELLTS | |
| | | FGPLKAFNLVKDSATGLSKGYAFCEYVDINVTDQAIAG | |
| | | LNGMQLGDKKLLVQRASVGAKNATLVSPPSTINQTPVT | |
| | | LQVPGLMSSQVQMGGHPTEVLCLMNMVLPEELLDDEEY | |
| | | EEIVEDVRDECSKYGLVKSIEIPRPVDGVEVPGCGKIF | |
| | | VEFTSVFDCQKAMQGLTGRKFANRVVVTKYCDPDSYHR | |
| | | RDFWKRTADGSEFESPKKKRKV | |
| 535 | pDF0953_ | MKRTADGSEFESPKKKRKVTTTMKISIEFLEPFRMTKW | 1326 |
| | Cas711S- | QESTRRNKNNKEFVRGQAFARWHRNKKDNTKGRPYITG | |
| | Y280K- | TLLRSAVIRSAENLLTLSDGKISEKTCCPGKFDTEDKD | |
| | D1580R | RLLQLRQRSTLRWTDKNPCPDNAETYCPFCELLGRSGN | |
| | | DGKKAEKKDWRFRIHFGNLSLPGKPDFDGPKAIGSQRV | |
| | | LNRVDFKSGKAHDFFKAYEVDHTRFPRFEGEITIDNKV | |
| | | SAEARKLLCDSLKFTDRLCGALCVIRFDEYTPAADSGK | |
| | | QTENVQAEPNANLAEKTAEQIISILDDNKKTEKTRLLA | |
| | | DAIRSLRRSSKLVAGLPKDHDGKDDHYLWDIGKKKKDE | |
| | | NSVTIRQILTTSADTKELKNAGKWREFCEKLGEALYLK | |
| | | SKDMSGGLKITRRILGDAEFHGKPDRLEKSRSVSIGSV | |
| | | LKETVVCGELVAKTPFFFGAIDEDAKQTDLQVLLTPDN | |
| | | KYRLPRSAVRGILRRDLQTYFDSPCNAELGGRPCMCKT | |
| | | CRIMRGITVMDARSEYNAPPEIRHRTRINPFTGTVAEG | |
| | | ALFNMEVAPEGIVFPFQLRYRGSEDGLPDALKTVLKWW | |
| | | AEGQAFMSGAASTGKGRFRMENAKYETLDLSDENQRND | |
| | | YLKNWGWRDEKGLEELKKRLNSGLPEPGNYRDPKWHEI | |
| | | NVSIEMASPFINGDPIRAAVDKRGTDVVTFVKYKAEGE | |
| | | EAKPVCAYKAESFRGVIRSAVARIHMEDGVPLTELTHS | |
| | | DCECLLCQIFGSEYEAGKIRFEDLVFESDPEPVTFDHV | |
| | | AIDRFTGGAADKKKFDDSPLPGSPARPLMLKGSFWIRR | |
| | | DVLEDEEYCKALGKALADVNNGLYPLGGKSAIGYGQVK | |
| | | SLGIKGDDKRISRLMNPAFDETDVAVPEKPKTDAEVRI | |
| | | EAEKVYYPHYFVEPHKKVEREEKPCGHQKFHEGRLTGK | |
| | | IRCKLITKTPLIVPDTSNDDFFRPADKEARKEKDEYHK | |
| | | SYAFFRLHKQIMIPGSELRGMVSSVYETVTNSCFRIFD | |
| | | ETKRLSWRGGGSRTVDDRMIGKRMSADLRPCHGDWVED | |
| | | GDLSALNAYPEKRLLLRHPKGLCPACRLFGTGSYKGRV | |
| | | RFGFASLENDPEWLIPGKNPGDPFHGGPVMLSLLERPR | |
| | | PTWSIPGSDNKFKVPGRKFYVHHHAWKTIKDGNHPTTG | |
| | | KAIEQSPNNRTVEALAGGNSFSFEIAFENLKEWELGLL | |
| | | IHSLQLEKGLAHKLGMAKSMGFGSVEIDVESVRLRKDW | |
| | | KQWRNGNSEIPNWLGKGFAKLKEWFRDELDFIENLKKL | |
| | | LWFPEGDQAPRVCYPMLRKKDDPNGNSGYEELKRGEFK | |
| | | KEDRQKKLTTPWTPWAKRTADGSEFESPKKKRKV | |
| | PDF1042 | | 0 |
| | USF1 OE | | |
| | target | | |

Table 16 shows the potential DNA sequences from Table 15 proteins for nuclease/reporter constructs.

Lengthy table referenced here
US20240100192A1-20240328-T00001
Please refer to the end of the specification for access instructions.

### EXAMPLES

While several experimental Examples are contemplated, these Examples are intended to be non-limiting.

### Example 1. RNA Writing with Cas7-11 Via 3′ Trans Splicing

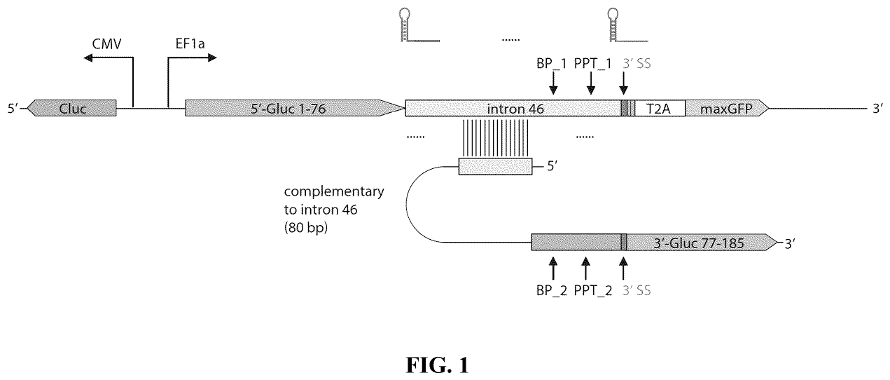

RNA writing with Cas7-11 via 3′ trans splicing and reconstituting full-length luciferase using same was demonstrated (FIG. 1). The targeted transcript only has the N-terminal Gluc fragment and is missing the rest of the protein. The trans-splicing template contains the C-terminal Gluc fragment, the 3′ splicing site signal, a branch point sequence, and poly pyrimidine tract (PPT). Splicing requires the presence of three main signals that directly participate in the reaction and that are present in every intron: the 5′ splice site (5SS); the 3′ splice site (3SS); and the branch point (BP). These signals, along with the polypyrimidine tract (PPT), are critical for correct spliceosome assembly. The trans-splicing template has a guide sequence that binds in the intron at the point we want RNA writing to begin (here it is intron 46). By using Cas7-11 to then cleave in the intron downstream of this trans-splicing template cargo guide, the normal downstream exons that would be competing for splicing with the trans-splicing template can be cleaved off. Using this approach, any single base edit, any sized insertion or any sized deletion can be made, providing new options for RNA based prevention and treatment of disease. These include, for example and without limitation, triplet repeat disorders, Rett syndrome, and Stargardt's disease.

Regarding the Luciferase analysis of trans-splicing efficiency, the medium containing the secreted luciferase was collected after 72 hours and its activity was measured using the Gaussia Luciferase Assay reagent (GAR-2B; Targeting Systems) and Cypridina (Vargula) luciferase assay reagent (VLAR-2; Targeting Systems) kits. Assays were performed in white 96-well plates on a plate reader (Biotek Synergy Neo 2) with an injection protocol. Luciferase measurements were normalized by dividing the Gluc values by the Cluc values, thus normalizing for any variation between wells.

### Example 2. RNA Writing with Cas7-11 Via 5′ Trans Splicing

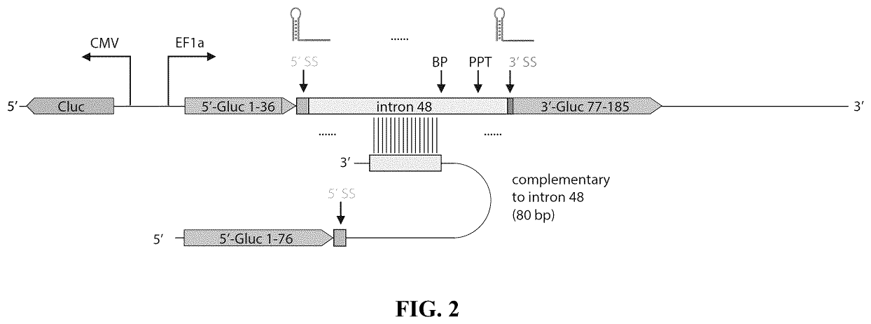

RNA writing with Cas7-11 via 5′ trans splicing and reconstituting full-length luciferase using same was demonstrated (FIG. 2). The targeted transcript only has the C-terminal Gluc fragment and is missing the rest of the protein. The trans-splicing template contains the N-terminal Gluc fragment and the 5′ splicing site signal. Splicing requires the presence of three main signals that directly participate in the reaction and that are present in every intron: the 5′ splice site (5SS); the 3′ splice site (3SS); and the branch point (BP). These signals, along with the polypyrimidine tract (PPT), are critical for correct spliceosome assembly. The trans-splicing template has a guide sequence that binds in the intron at the point we want RNA writing to begin (here it is intron 48). By using Cas7-11 to then cleave in the intron upstream of this trans-splicing template cargo guide, the normal upstream exons that would be competing for splicing can cleaved off with the trans splicing template.

### Example 3. RNA Writing with Cas7-11 Via Internal Trans Splicing

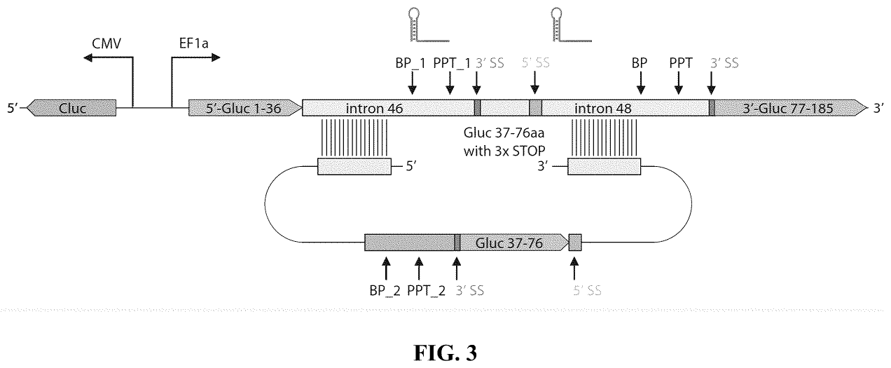

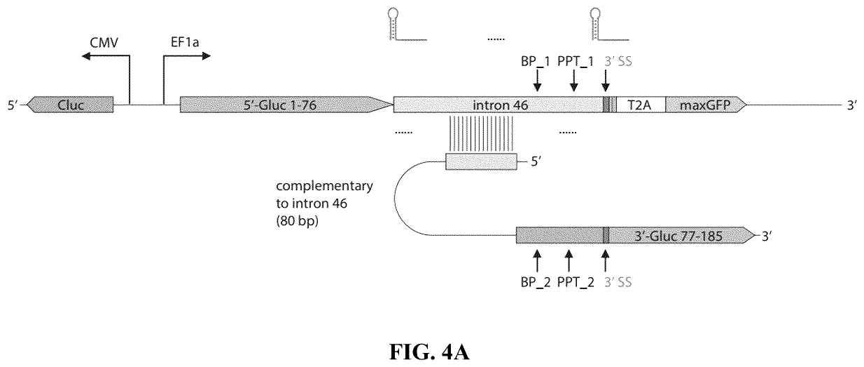

RNA writing with Cas7-11 via internal trans splicing and reconstituting full-length luciferase using same was demonstrated (FIG. 3). The targeted transcript only has the N and C terminal Gluc fragments and is missing the internal part of the protein. The trans-splicing template contains the internal Gluc fragment, the 3′ splicing site signal, 5′ splicing site signal, a branch point sequence, and poly pyrimidine tract (PPT). Splicing requires the presence of three main signals that directly participate in the reaction and that are present in every intron: the 5′ splice site (5SS); the 3′ splice site (3SS); and the branch point (BP). These signals, along with the polypyrimidine tract (PPT), are critical for correct spliceosome assembly. The trans-splicing template has a guide sequence that binds in the upstream (here intron 46) and downstream intron (here it is intron 48) at the point we want RNA writing to begin. By using Cas7-11 to then cleave in the intron in between the trans-splicing template guides, the normal internal exons that would be competing for splicing with can be cleaved off with the trans splicing template.

Internal trans splicing can be useful because it involves a small template replacing just the exon that needs to be targeted. 3′ and 5′ trans splicing can involve large sequence replacement on the order of thousands of base pairs. Exons, however, are generally only a few hundred bases meaning internal trans splicing can have a smaller trans splicing template, making cell delivery easier.

### Example 4. 3′ Trans-Splicing Activity on a 5′-Fragment of Gluc Pre-mRNA Target

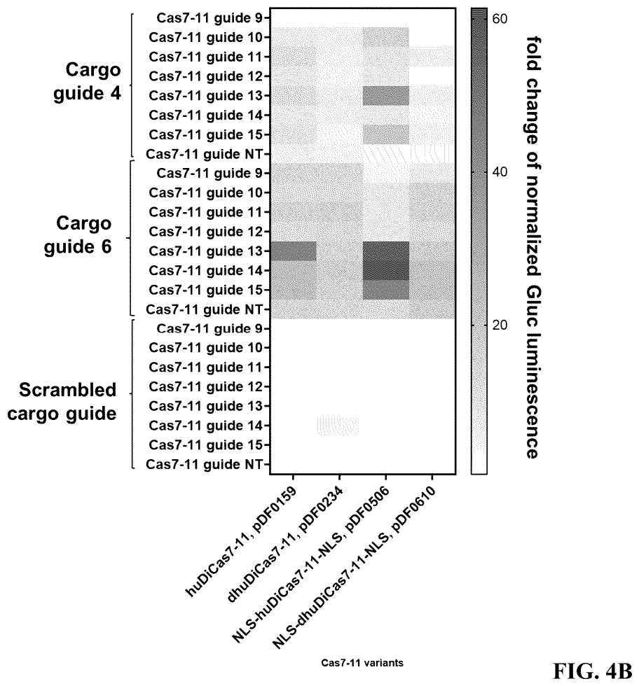

The 3′ trans-splicing activity on a 5′-fragment of Gluc pre-mRNA target (1-76 aa) was demonstrated (FIGS. 4A and 4B). A luciferase reporter to readout the efficiency of this process was developed. The luciferase reporter has the N-terminal fragment of Gluc and the missing C-terminal fragment can be supplied via Cas7-11 induced trans splicing. The trans splicing template contains the C-terminal Gluc fragment, the 3′ splicing site signal, a branch point sequence, and poly pyrimidine tract (PPT). Splicing requires the presence of three main signals that directly participate in the reaction and that are present in every intron: the 5′ splice site (5SS); the 3′ splice site (3SS); and the branch point (BP). These signals, along with the polypyrimidine tract (PPT), are critical for correct spliceosome assembly. The trans splicing template has a guide sequence that binds in the intron at the point we want RNA writing to begin (here it is intron 46). The cargo template was designed by fusing the following components (5′-3′): 80 bp binding domain to intron 46 of human COL7A1, 31 bp spacer-branch point-poly pyrimidine tract-3′ splicing site (AACGAGGAATTCTCTTCTTTTTTTTCTGCAG (SEQ ID NO: 610)), and Gluc 77-185aa coding sequence (with a single nucleotide difference from the target transcript). By using Cas7-11 to then cleave in the intron downstream of this trans template cargo guide, the normal downstream exons that would be competing for splicing with can be cleaved of with the trans splicing template. This, as shown in the heatmap, significantly boosts the rate of trans splicing.

The data shows that the choice of both Cargo guide and Cas7-11 guide are essential for effective trans-splicing, and that there are non-obvious rules for programming and design. In addition, the data show that localization of the Cas7-11 protein, via the NLS, can yield significant improvements to the efficiency of the trans splicing. Furthermore, the data show that editing is dependent on the cleavage activity of Cas7-11, as the “dhuDiCas7-11” variants had no improvement in activity versus the non-targeting guides.

FIG. 4A shows a schematic illustrating the DiCas7-11-assisted 3′ trans-splicing through target transcript cleavage. FIG. 4B shows a heat chart illustrating trans-splicing activity for Cargo template and Cas7-11 guides targets COL7A1 intron 46 sequence, which was placed upstream of the 5′-fragment of Gluc pre-mRNA target (intron 46 of human COL7A1 gene was inserted to the 3′ end of Glue 1-76 aa coding sequence). Successful trans-splicing resulted in an mRNA that encodes the full length Gluc protein. Trans-splicing efficiency was represented by Gluc chemiluminescence signal and normalized to Cluc signal, the latter of which was constantly expressed. Gluc/Cluc value for each condition was normalized to that with scrambled cargo template, non-targeting Cas7-11 guide, and NLS-dhuDiCas7-11-NLS (NT: scrambled non-targeting guide. BP: branch point. PPT: poly pyrimidine tract. SS: splice site).

### Example 5. 3′ Trans-Splicing Activity on 5′-Fragment of Gluc Pre-mRNA Target with Selected Cas7-11 Guides

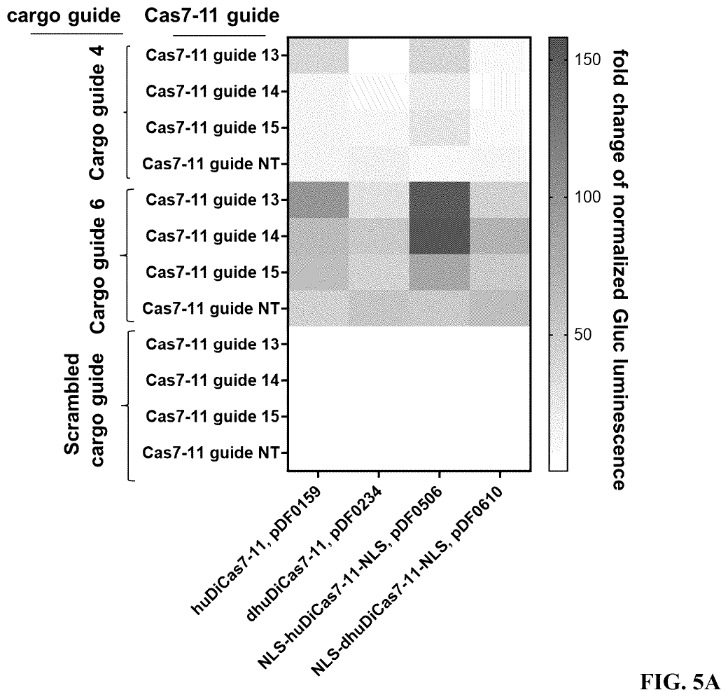

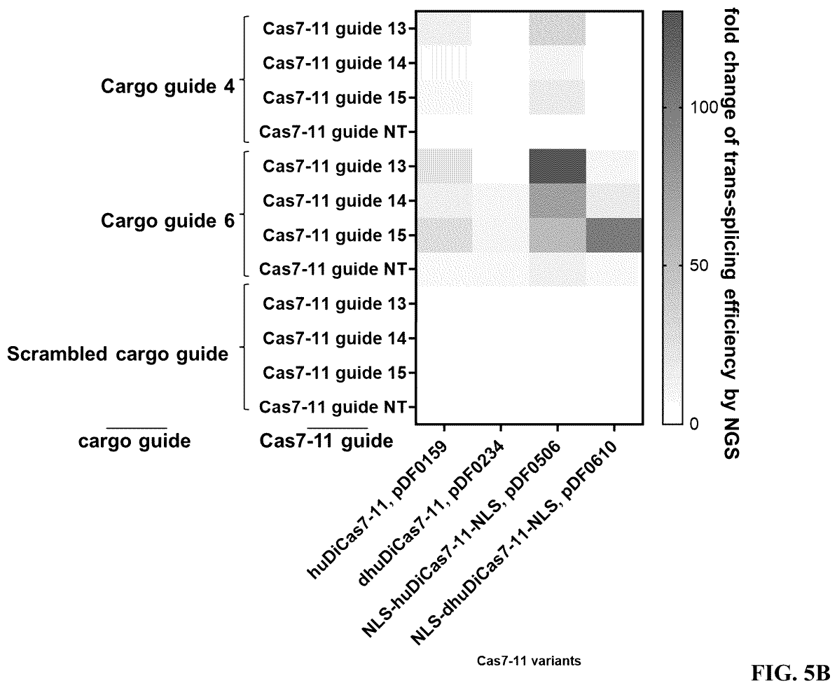

3′ trans-splicing activity on the 5′-fragment of Gluc pre-mRNA target (1-76 aa) with midi prepped plasmids and a smaller panel of Cas7-11 guides was assessed (FIGS. 5A and 5B). Cargo template and Cas7-11 guides targeted COL7A1 intron 48 sequence (intron 48 of human COL7A1 gene was inserted between Gluc 1-36aa and 77-185aa coding sequences). Successful trans-splicing resulted in an mRNA that encodes the full length Gluc protein.

FIG. 5A is a heat chart showing the trans-splicing efficiency represented by Gluc chemiluminescence signal and normalized to Cluc signal, the latter of which was constantly expressed. Gluc/Cluc value for each condition was normalized to that with scrambled cargo, non-targeting Cas7-11 guide, and NLS-dhuDiCas7-11-NLS. FIG. 5B is a heat chart showing the trans-splicing efficiency measured by NGS and probing for a single nucleotide change between the targeted pre-mRNA transcript and the cargo template. Trans-splicing efficiency was normalized to that with scrambled cargo template, non-targeting Cas7-11 guide, and NLS-dhuDiCas7-11-NLS. NT: scrambled non-targeting guide.

Regarding the NGS analysis of trans-splicing efficiency, cells were lysed after 72 hours by RNA lysis buffer (see, e.g., www.ncbi.nlm.nih.gov/pmc/articles/PMC5526071) for 8 min at room temperature and then stopped by 1/10 volume of RNA lysis stop buffer. Cell lysate was then used for first strand synthesis using the RevertAid First Strand cDNA Synthesis Kit (Thermo Fisher) with dT18 primer (SEQ ID NO: 611) or a gene specific primer. cDNA was then used for PCR amplification of the trans-splicing junction and sequenced with Illumina MiSeq. NGS data was analyzed by probing for the single nucleotide change between the targeted transcript and the cargo template.

Larger fold changes were obtained with some of the cargo guides and Cas7-11 guide combos with higher quality plasmid preps. The fold change by sequencing which shows that there are RNA level changes that match the protein level increases. It was observed that efficiency is dependent on four factors: cargo guide sequence and location, Cas7-11 guide sequence and location, localization of the Cas7-11 construct, and active RNA cleavage activity by the Cas7-11 construct.

### Example 6. Internal Trans-Splicing Activity on the Gluc Pre-mRNA Target

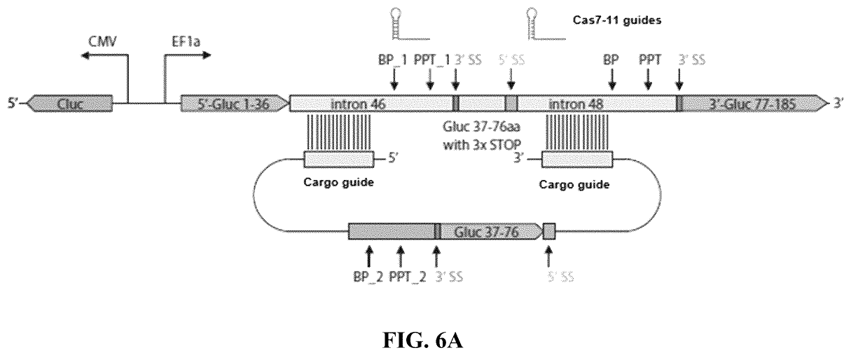

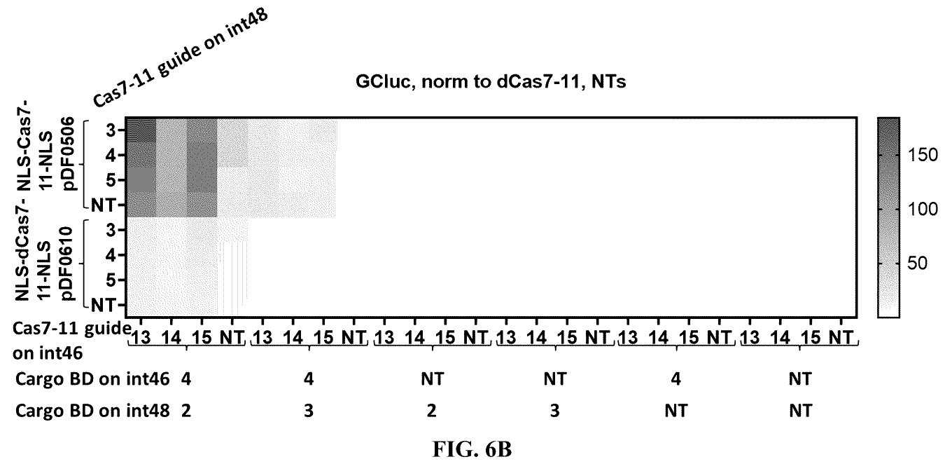

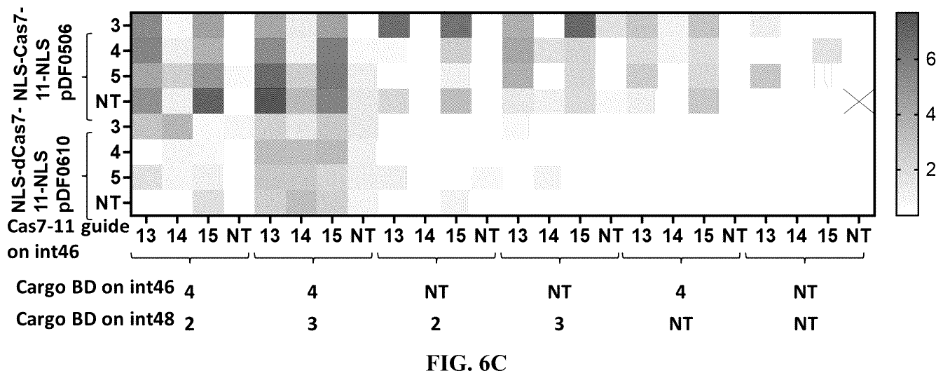

Internal trans-splicing activity on the Gluc pre-mRNA target, with 3×STOP codon inserted to the 37-76 aa coding sequenced flanked by intron 46 and intron 48 of COL7A1 in the targeted Gluc pre-mRNA was assessed (FIGS. 6A, 6B, and 6C). Intron 46 of human COL7A1 gene was inserted between Gluc 1-36aa and 37-76aa coding sequences, intron 48 of human COL7A1 gene was inserted between Gluc 37-76aa and 77-185aa coding sequences. In this reporter, three consecutive stop codons replaced 56-58aa to suppress Gluc expression in cis-splicing. The cargo template was designed by fusing the following components (5′-3′): 80 bp binding domain to intron 46 of human COL7A1, 31 bp spacer-branch point-poly pyrimidine tract-3′ splicing site (AACGAGGAATTCTCTTCTTTTTTTTCTGCAG (SEQ ID NO: 610)), Gluc 37-76aa coding sequence, 16 bp 5′ splice donor (GTAAGC)-spacer, and 80 bp binding domain to intron 48 of human COL7A1Successful trans-splicing resulted in an mRNA without cryptic stop codons for the expression of full length Gluc protein.

A schematic showing the DiCas7-11-assisted internal trans-splicing through target transcript cleavage is provided in FIG. 6A. A heat chart showing the trans-splicing efficiency represented by Gluc chemiluminescence signal and normalized to Cluc signal, the latter of which was constantly expressed, is presented in FIG. 6B. Gluc/Cluc value for each condition was normalized to that with scrambled cargo, non-targeting Cas7-11 guide, and NLS-dhuDiCas7-11-NLS. A heat chart showing the trans-splicing efficiency measured by NGS and probing for the replacement of 3×stop codon from the targeted pre-mRNA transcript to the cargo template is presented in FIG. 6C. Trans-splicing efficiency is represented by the percentage of reads carrying read-through codons (non-3×STOP codon) over all amplicons (NT: scrambled non-targeting guide; BP: branch point; PPT: poly pyrimidine tract; and SS: splice site).

It was observed that certain cargo guides work better with specific Cas7-11 guides to enable up to 185-fold protein activation. Internal trans splicing has for advantage to enable the replacement of a single exon, which can be on average a few hundred bases. 5′ and 3′ trans splicing can involve replacing thousands of base pairs of RNA transcript whereas internal trans splicing results in much smaller modifications, which are simpler and make delivery to cells easier.

It was observed that efficiency is dependent on multiple factors: cargo guide sequences and location, Cas7-11 guide sequences and location, and active RNA cleavage activity by the Cas7-11 construct. Since two cargo guides and two Cas7-11 guides are needed, there are additional parameters for optimization, and a successful construct can be generated by a combination of all these components. For instance, it was observed that the guide targeting intron 46 has a strong influence on the efficiency of the outcome.

It was observed that luciferase is not necessarily concordant with NGS readout efficiency, potentially due to the degradation of the wild-type transcript which increases the relative editing efficiencies in some NGS conditions. However, many similar trends with regards to guide selection hold.

### Example 7. 3′ Trans-Splicing Activity on Endogenous Pre-mRNA Targets in HEK293FT Cells

The 3′ trans-splicing activity on two endogenous pre-mRNA targets, MALAT1 and STAT3 transcripts, in HEK293FT cells were assessed. Trans-splicing efficiency was determined by NGS, probing for a single nucleotide change between the targeted pre-mRNA transcript and the cargo template.

Mammalian experiments were performed using the HEK293FT cell line, acquired from and authenticated by Thermo Fisher Scientific (R70007). HEK293FT cells were grown at 37° C. and 5% CO₂ in Dulbecco's modified Eagle medium with high glucose, sodium pyruvate and GlutaMAX (Thermo Fisher Scientific), supplemented with 1× penicillin-streptomycin (Thermo Fisher Scientific) and 10% fetal bovine serum (Thermo Fisher Scientific), and passaged using TrypLE Express (Thermo Fisher Scientific). For transfection, the HEK293FT cells were plated 16 h before transfection at seeding densities of 1.5×10⁴ cells per well, allowing cells to reach 90% confluency before the transfection. Cells were then transfected with Lipofectamine 3000 (Thermo Fisher Scientific), following the manufacturer's protocol with 10 ng of part or all of the following components (target Gluc-Clue plasmid, cargo template plasmid, Cas7-11 expression plasmid, and Cas7-11 guide expression plasmid) and pUC19 as a stuffer plasmid to make up a total of 100 ng plasmid per well.

The cargo template was designed by fusing the following components (5′-3′): 80 bp binding domain to intron 46 of human COL7A1, 31 bp spacer-branch point-poly pyrimidine tract-3′ splicing site (AACGAGGAATTCTCTTCTTTTTTTTCTGCAG (SEQ ID NO: 610)), and Gluc 77-185aa coding sequence (with a single nucleotide difference from the target transcript). For endogenous targets, the exon immediately following the targeted intron replaced the Gluc 77-185aa coding sequence (with a single nucleotide difference from the target transcript).

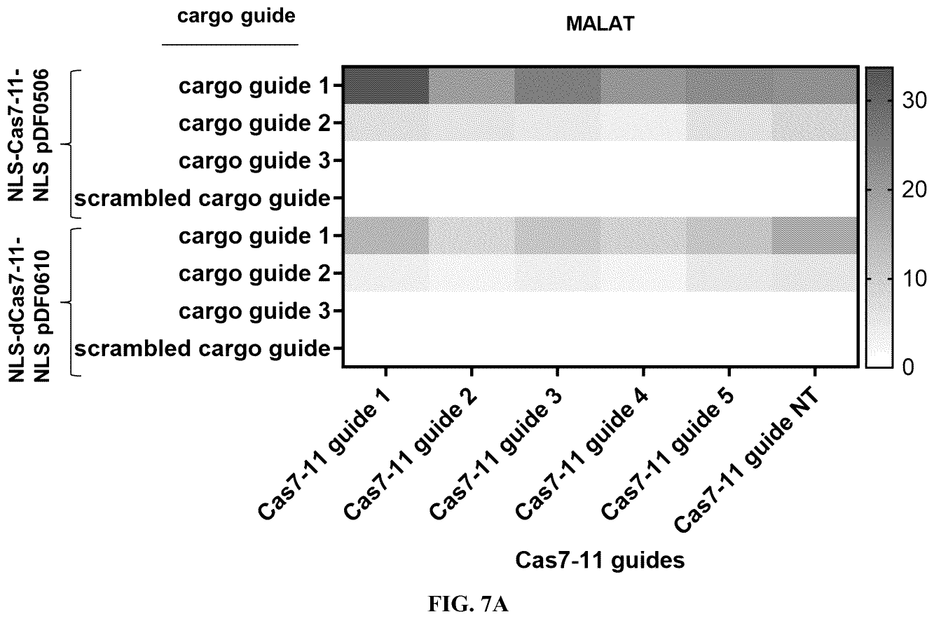

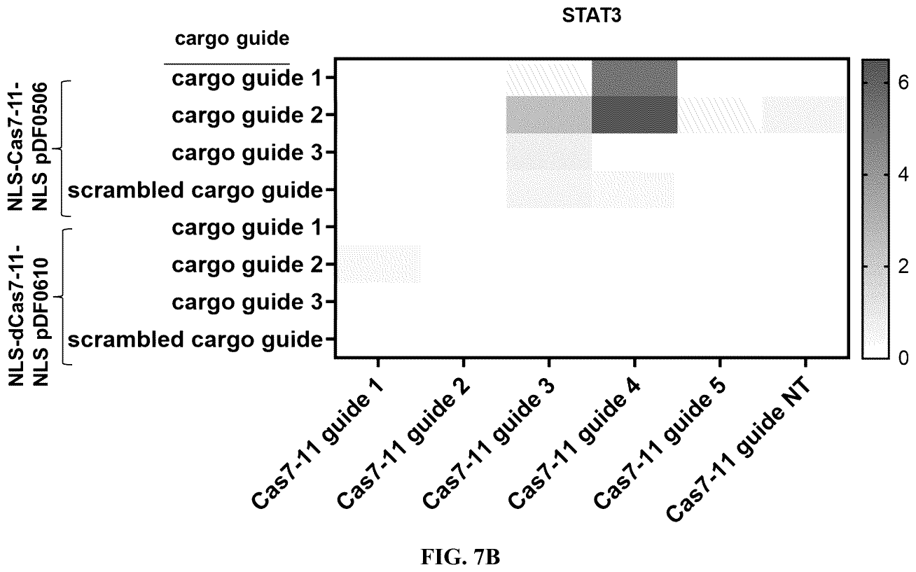

Results are shown in FIGS. 7A and 7B. FIG. 7A is a heat chart showing the transfection of three different cargo templates in combination with Cas7-11 and guides for targeting of intron 2 of MALAT pre-mRNA (the heatmap is percent editing as measured by NGS). FIG. 7B is a heat chart showing the transfection of three different cargo templates in combination with Cas7-11 and guides for targeting of intron 5 of STAT3 pre-mRNA (the heatmap is percent editing as measured by NGS).

It was observed by RNA editing (NGS readout) that high editing can be achieved with Cas7-11 induced trans splicing. Without cas7-11 (non-targeting guides or NT) the trans splicing efficiency was found to be lower. It was also observed that the selection of the Cas7-11 and cargo guide is key, with synergistic effects, and Cas7-11 cleavage is required.

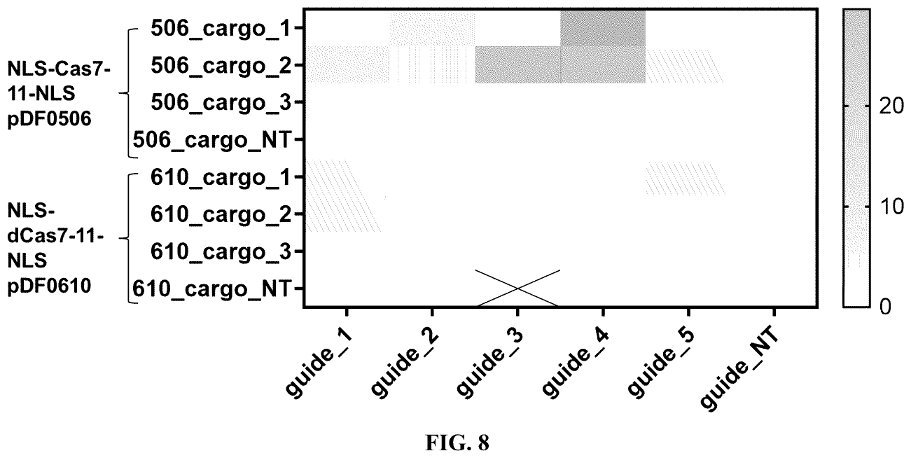

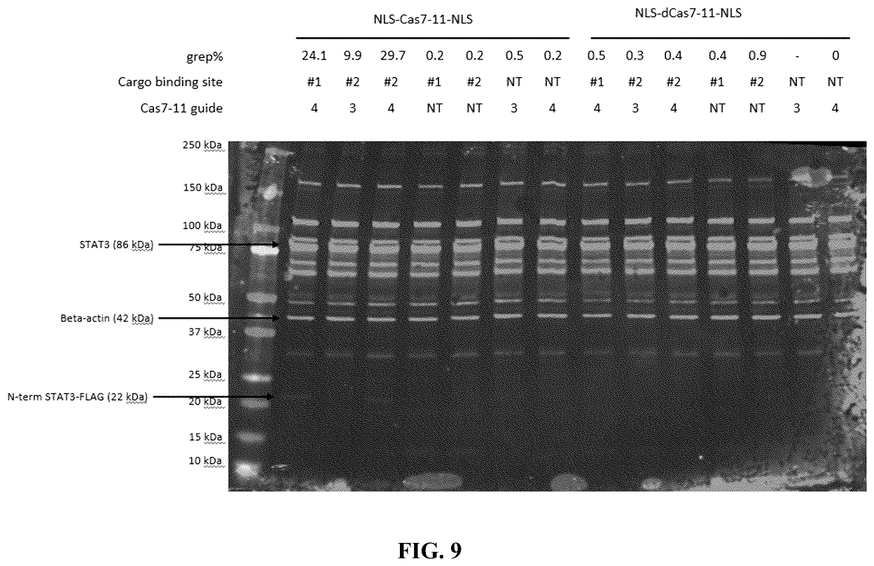

Additional results are shown in FIGS. 8 and 9. FIG. 8 is a heat chart showing that the STAT3 is similar to the last slide except with even better editing due to better transfection conditions showing that we can improve 3′ TS on STAT3 by up to 60-fold and up to about 29.7% editing. FIG. 9 is a gel stained via western blot that was prepared to probe if protein level effects can be seen from the 3′ TS of STAT3 presented in FIG. 8. Since the trans splicing with Cas7-11 truncates the STAT3 protein, a shift in protein size from 86 kDA to 22 kDA was expected. Smaller STAT3 protein showing up in lanes 1 and 3 were observed, which corresponds to some of the conditions with the highest trans splicing efficiency in FIG. 8. This indicates that proteins in cells can be changed via RNA writing.

### Example 8. 3′ Trans-Splicing Activity on the 5′-Fragment of Gluc Pre-mRNA Target with a Fusion of Cargo Guide and Cas7-11 Guide

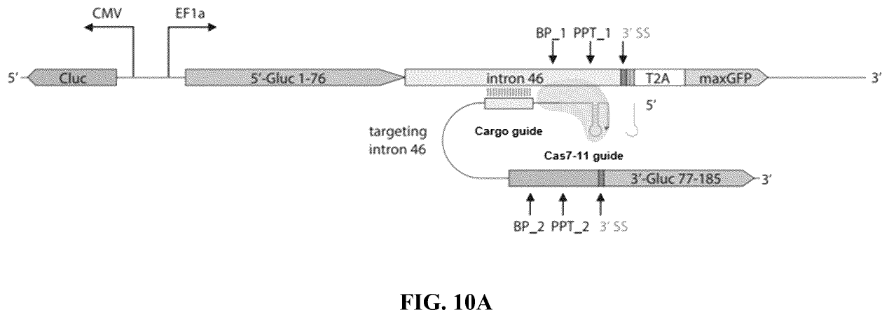

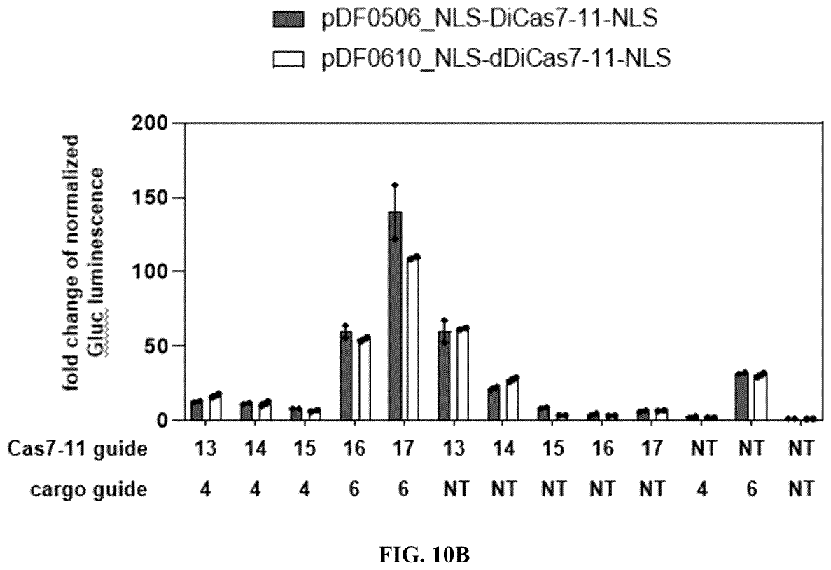

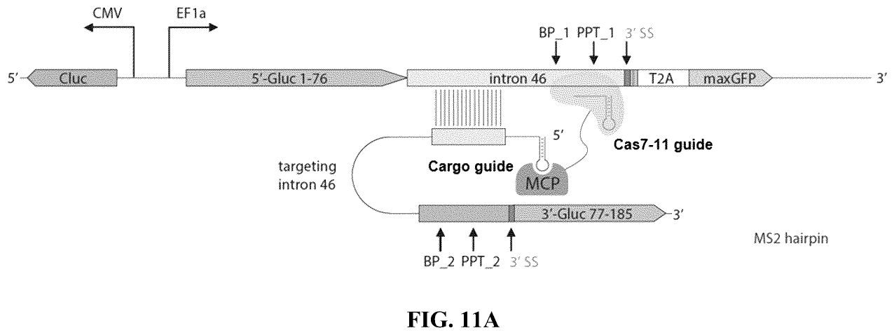

3′ trans-splicing activity on a 5′-fragment of Gluc pre-mRNA target with a pre-crRNA-cargo binding domain-Gluc 77-185aa cargo template was assessed (FIGS. 10A and 10B; NT: scrambled non-targeting guide). Pre-mature crRNA targeting intron 46 and cargo guide targeting intron 46 were fused from 5′-3′ direction on the cargo template, followed by 31 bp spacer-branch point-poly pyrimidine tract-3′ splicing site (AACGAGGAATTCTCTTCTTTTTTTTCTGCAG (SEQ ID NO: 610)), and Gluc 77-185aa coding sequence (with a single nucleotide difference from the target transcript).

It was observed that this approach enables up to 150-fold trans splicing protein activation.

### Example 9. 3′ Trans-Splicing Activity on a 5′-Fragment of Gluc Pre-mRNA Target with a Fusion of Cargo Guide and MS2 Hairpin as Well as Cas7-11-MCP Fusion Proteins

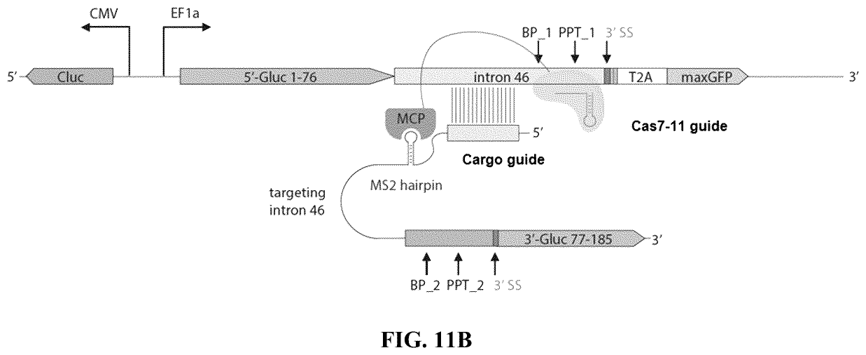

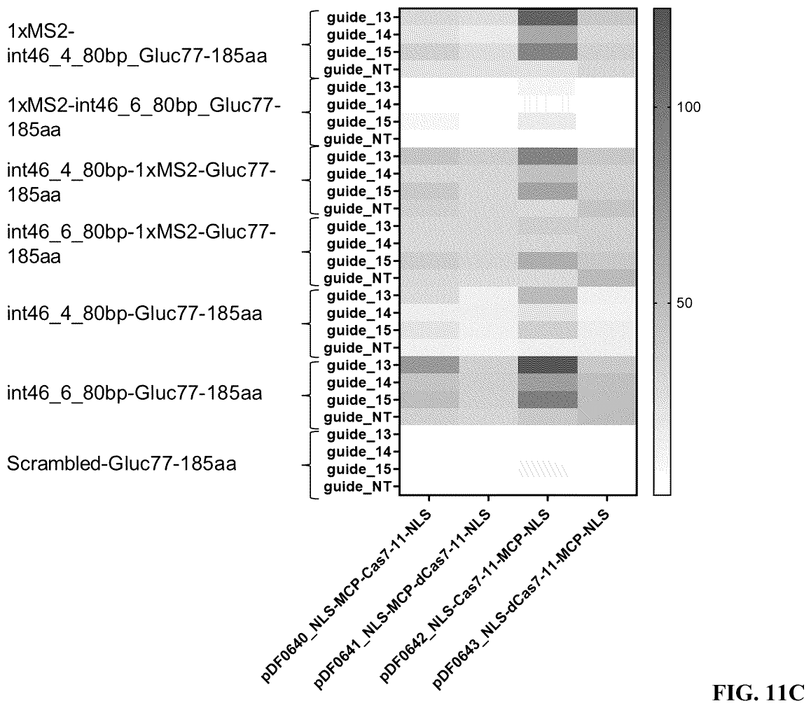

3′ trans-splicing activity on a 5′-fragment of Gluc pre-mRNA target using a combination of Cas7-11-MCP fusion protein variants and MS2-cargo binding domain-Gluc 77-185aa cargo template variants were assessed (FIGS. 11A and 11B). MS2 hairpin and cargo guide 4 or 6 were fused from 5′-3′ direction on the cargo template, followed by 31 bp spacer-branch point-poly pyrimidine tract-3′ splicing site (AACGAGGAATTCTCTTCTTTTTTTTCTGCAG (SEQ ID NO: 610)), and Gluc 77-185aa coding sequence (with a single nucleotide difference from the target transcript). In a different design, the positions of MS2 hairpin and cargo guide were reversed in the fusion cargo template, followed by the other elements as mentioned above.

Trans-splicing efficiency was represented by Gluc chemiluminescence signal and normalized to Cluc signal, the latter of which was constantly expressed (FIG. 11C). Gluc/Cluc value for each condition was normalized to that with scrambled cargo template, non-targeting Cas7-11 guide, and NLS-dhuDiCas7-11-MCP-NLS (NT: scrambled non-targeting guide). Additional increases in trans splicing Gluc activation were observed. For example, Cargo 4 went from 42-fold activation to 110-fold activation with the MS2 recruitment. The selection of components was found to be important.

### Example 10. Internal Trans-Splicing Activity on the Gluc Pre-mRNA Target

Internal trans-splicing activity on the Gluc pre-mRNA target, with 3×STOP codon inserted to the 37-76 aa coding sequenced flanked by intron 46 and intron 48 of COL7A1 in the targeted Glue pre-mRNA were assessed. Successful trans-splicing resulted in an mRNA without cryptic stop codons for the expression of full length Gluc protein.

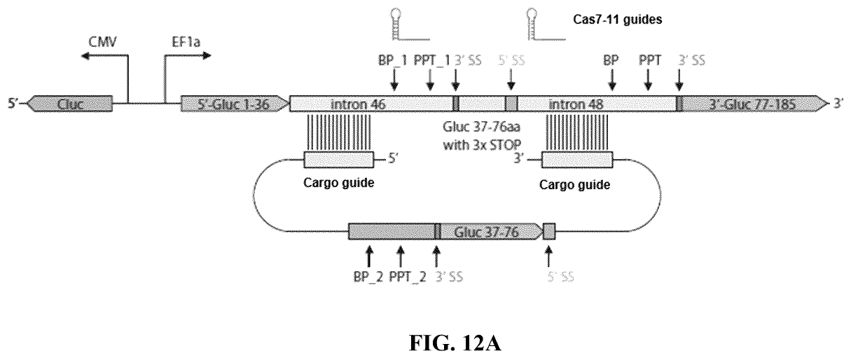

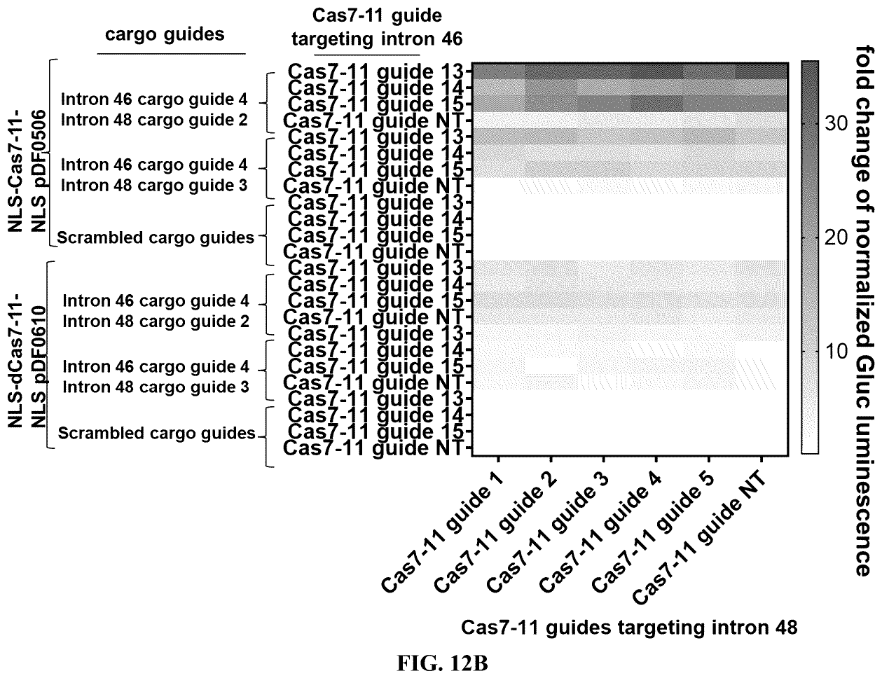

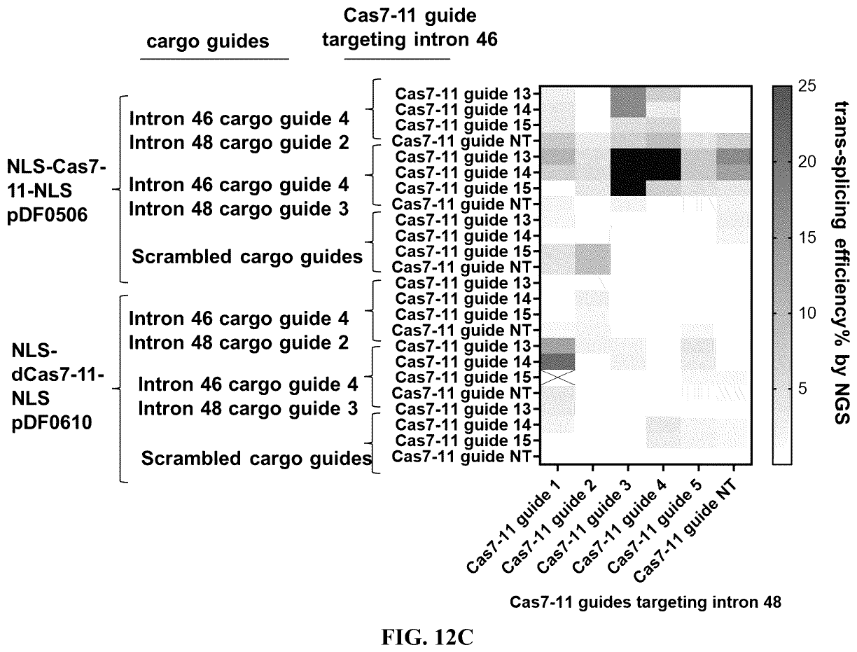

The DiCas7-11-assisted internal trans-splicing through target transcript cleavage is shown in FIG. 12A. Trans-splicing efficiency was represented by Gluc chemiluminescence signal and normalized to Cluc signal, the latter of which was constantly expressed, as shown in FIG. 12B. Gluc/Cluc value for each condition was normalized to that with scrambled cargo, non-targeting Cas7-11 guide, and NLS-dhuDiCas7-11-NLS. Trans-splicing efficiency was measured by NGS, probing for the replacement of 3×stop codon from the targeted pre-mRNA transcript to the cargo template as shown in FIG. 12C. Trans-splicing efficiency was normalized to that with scrambled cargo template, non-targeting Cas7-11 guide, and NLS-dhuDiCas7-11-NLS (NT: scrambled non-targeting guide. BP: branch point. PPT: poly pyrimidine tract. SS: splice site).

### Example 11. 3′ Endogenous Trans-Splicing Rate for STAT3 Gene

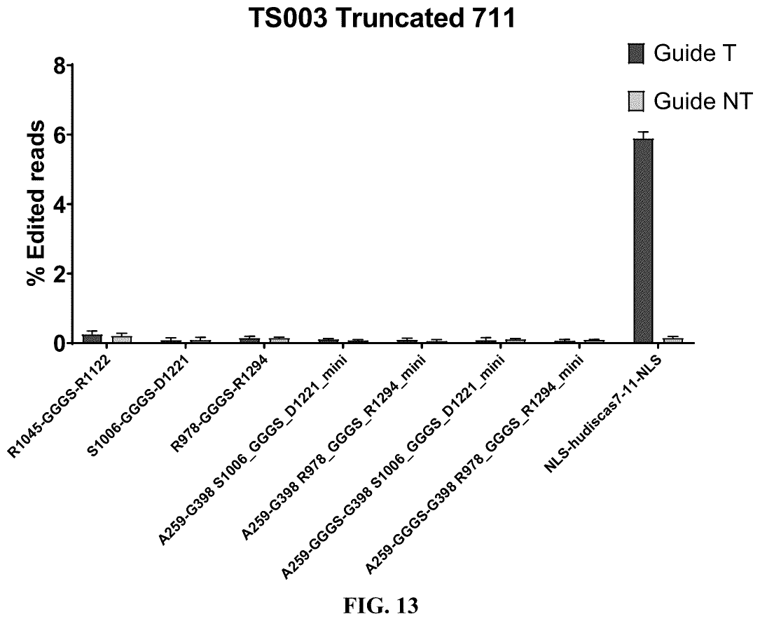

The 3′ endogenous trans-splicing rates (%) for the gene STAT3 were assessed using one common cargo replacing STAT3 exon 6 and either a Cas7-11 guide RNA targeting the STAT3 intron 5 or a guide scrambled sequence (FIG. 13). Different truncated versions of the disCas7-11 (plotted on x axis, see FIG. 13) were compared for editing efficiency.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Smaller Cas7-11 nucleases are of interest as a lower overall size allows for more options for delivery (e.g., the use of AAV vectors or combinations of constructs). This example demonstrates that smaller, truncated Cas7-11 variants cause a major drop-off in efficiency of trans-splicing, likely due to a loss of catalytic activity.

### Example 12. 3′ Endogenous Trans-Splicing Rate for STAT3 Gene

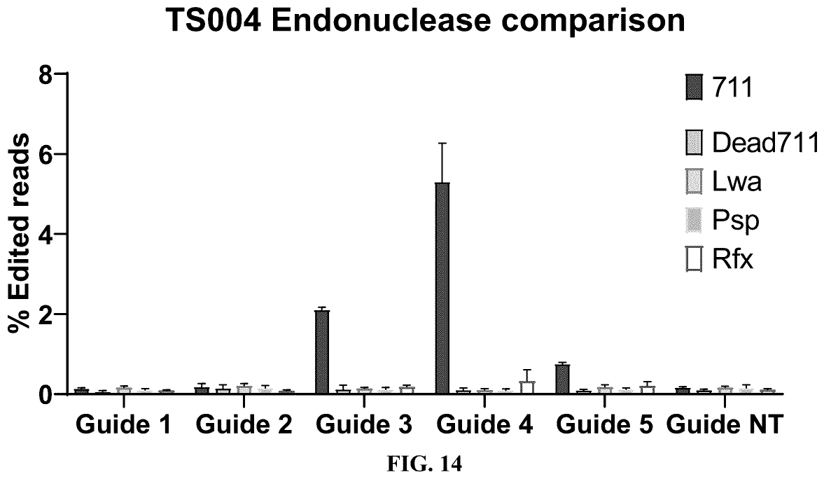

The 3′ endogenous trans-splicing rates (%) for the gene STAT3 were assessed using one common cargo replacing STAT3 exon 6 and a set of Cas7-11 or Cas13 guides targeting intron 5 or a scrambled sequence, arrayed around a position that induced efficient splicing for Cas7-11 constructs (FIG. 14). Five different nucleases were compared: the disCas7-11, a catalytically inactive disCas7-11, and 3 major orthologs of Cas13 (Lwa, Psp, and Rfx Cas13).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Smaller nucleases are of interest as a lower overall size allows for more options for delivery (e.g., the use of AAV vectors or combinations of constructs). Similarly, orthologous enzymes such as the Cas13s can be useful for the trans-splicing mechanism if they shower higher enzymatic activity. This example shows that Cas13s are inefficient at inducing trans-splicing relative to the Cas7-11 compared here. One potential justification for this is that the Cas13 constructs lack the N- and C-terminal SV40 NLS sequences in the Cas7-11 version, potentially limiting their transit to the nucleus and therefor their ability to bind or target the precursor mRNA.

### Example 13. 3′ Endogenous Trans-Splicing Rate for STAT3 Gene

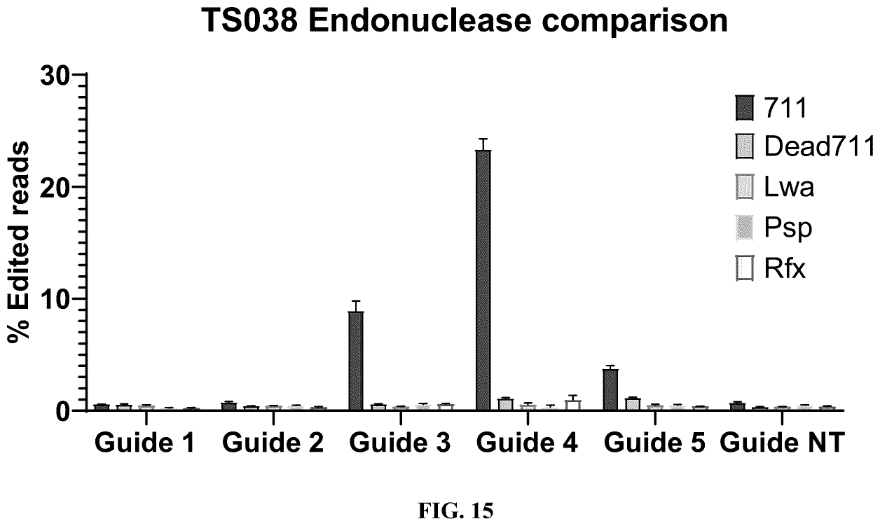

The 3′ endogenous trans-splicing rates (%) for the gene STAT3 were assessed using one common cargo replacing STAT3 exon 6 and a set of Cas7-11 or Cas13 guides targeting intron 5 or a scrambled sequence, arrayed around a position that induced efficient splicing for Cas7-11 constructs (FIG. 15). Five different nucleases were compared: the disCas7-11, a catalytically inactive disCas7-11, and 3 major orthologs of Cas13 (Lwa, Psp, and Rfx Cas13).

The constructs were transfected at a 96-well scale on HEK293FT cells in DMEM 10% FBS.

### Materials & Methods

For endogenous splicing experiments, transfections were carried out using:
10 ng of guide
10 ng of cargo plasmid
40 ng of Cas7-11 plasmid
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Smaller nucleases are of interest as a lower overall size allows for more options for delivery (e.g., the use of AAV vectors or combinations of constructs). Similarly, orthologous enzymes such as the Cas13s can be useful for the trans-splicing mechanism. This example shows that Cas13s are inefficient at inducing trans-splicing relative to the Cas7-11. Cas13s, in this example, are expressed with N- and C-terminal SV40 NLS sequences.

### Example 14. 3′ Endogenous Trans-Splicing Rate for PABPC1 Gene

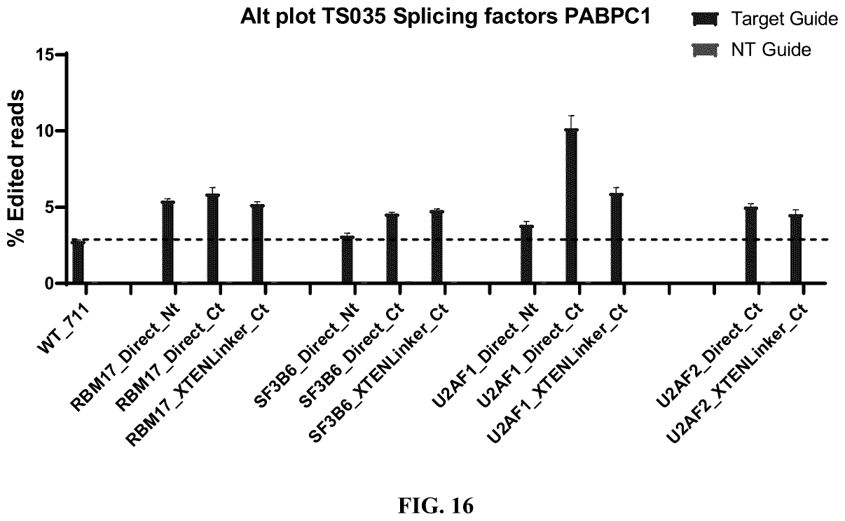

3′ endogenous trans-splicing rates (%) for the gene PABPC1 were assessed using one common cargo replacing the PABPC1 terminal exon 14 and either a PABPC1 intron 13 or scrambled guide (FIG. 16). The wildtype disCas7-11 nuclease was compared against constructs with a direct (no intervening linker) or indirect (XTEN linker intervening) fusion to various splicing proteins (RAMB17, SF3B6, U2AF1, or U2AF2) at their N- or C-terminals.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Nearly all fusions of spliceosome proteins tested show an increased splicing rate relative to the Cas7-11 only construct. In combination with the other genes tested using the same set of constructs, certain genes structures or intron sizes can benefit to greater degrees from recruitment of different splicing proteins, potentially indicating a different spliceosome composition or mechanism.

### Example 15. 3′ Endogenous Trans-Splicing Rate for STAT3 Gene

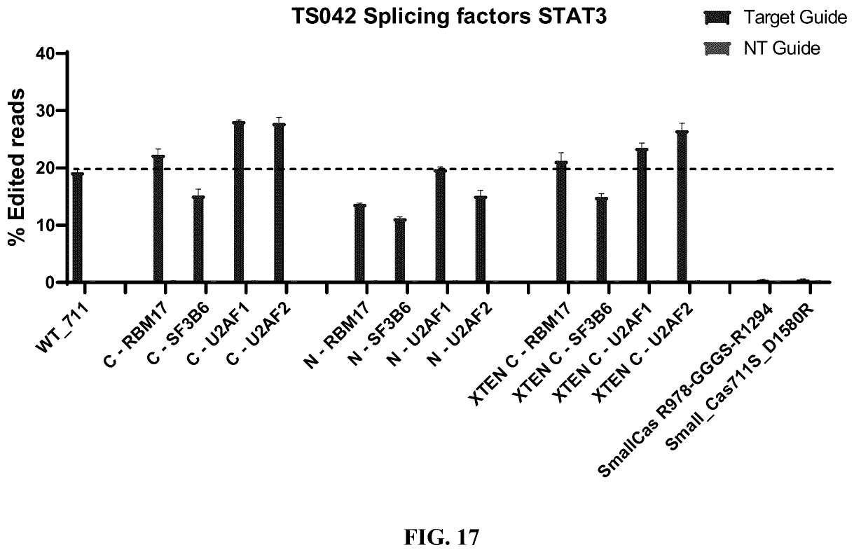

The 3′ endogenous trans-splicing rates (%) for the gene STAT3 were assessed using one common cargo replacing the STAT3 exon 6 and either a STAT3 intron 5 or scrambled guide (FIG. 17). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested show an increased splicing rate relative to the Cas7-11 only construct. In combination with the other genes tested using the same set of constructs, certain genes structures or intron sizes can benefit to greater degrees from recruitment of different splicing proteins, potentially indicating a different spliceosome composition or mechanism.

### Example 16. 3′ Endogenous Trans-Splicing Rate for PPIB Gene

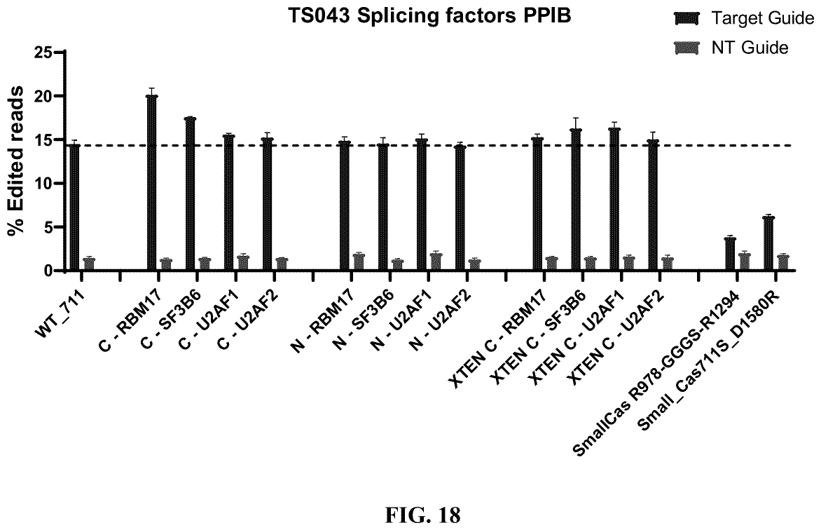

The 3′ endogenous trans-splicing rates (%) for the gene PPIB were assessed using one common cargo replacing the PPIB terminal exon and either a PPIB intron 4 or scrambled guide (FIG. 18). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested increase trans-splicing rates relative to the wildtype Cas7-11. In combination with the other genes tested using the same set of constructs, certain genes structures or intron sizes can benefit to greater degrees from recruitment of different splicing proteins, potentially indicating a different spliceosome composition or mechanism.

### Example 17. 3′ Endogenous Trans-Splicing Rate for TOP2A Gene

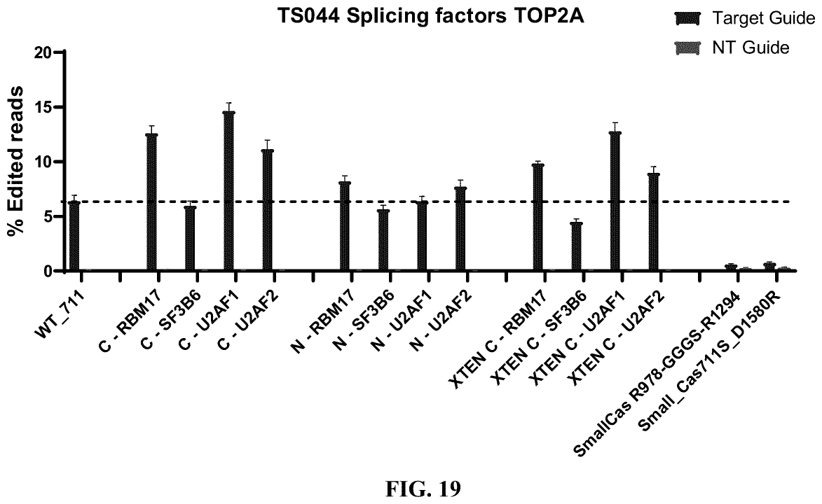

The 3′ endogenous trans-splicing rates (%) for the gene TOP2A were assessed using one common cargo replacing the TOP2A exon 21 and either a TOP2A intron 20 or scrambled guide (FIG. 19). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested increase trans-splicing rates relative to the wildtype Cas7-11, while others perform similarly or worse. In combination with the other genes tested using the same set of constructs, certain genes structures or intron sizes can benefit to greater degrees from recruitment of different splicing proteins, potentially indicating a different spliceosome composition or mechanism.

### Example 18. 3′ Endogenous Trans-Splicing Rate for PPIB Gene

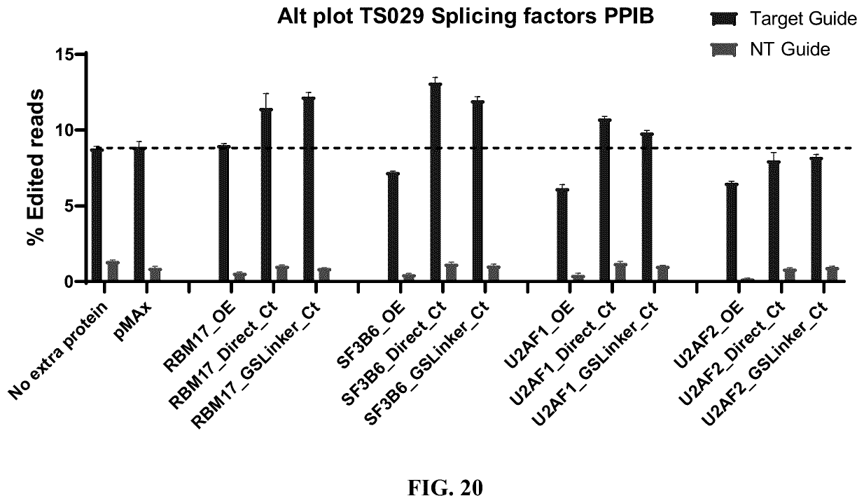

The 3′ endogenous trans-splicing rates (%) for the gene PPIB were assessed using one common cargo replacing the PPIB terminal exon and either a PPIB intron 4 or scrambled guide (FIG. 20). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals. Constructs with GS linkers replacing the XTENs were also considered.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested increase trans-splicing rates relative to the wildtype Cas7-11. In combination with the other genes tested using the same set of constructs, certain genes structures or intron sizes can benefit to greater degrees from recruitment of different splicing proteins, potentially indicating a different spliceosome composition or mechanism.

### Example 19. 3′ Endogenous Trans-Splicing Rate for STAT3 Gene

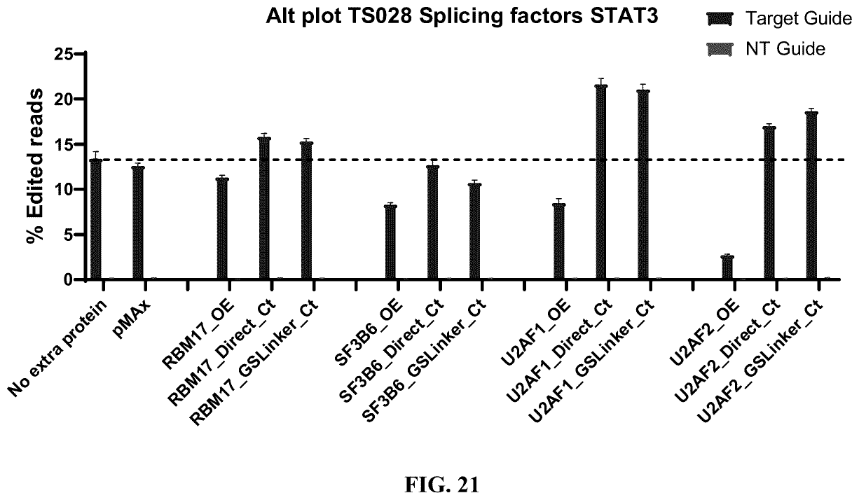

The 3′ endogenous trans-splicing rates (%) for the gene STAT3 were assessed using one common cargo replacing the STAT3 exon 6 and either a STAT3 intron 5 or scrambled guide (FIG. 21). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals. Constructs with GS linkers replacing the XTENs were also considered.

### Materials & Methods

The constructs were transfected at a 96-well scale on HEK293FT cells in DMEM 10% FBS. For endogenous splicing experiments, transfections were carried out using:
10 ng of guide
10 ng of cargo plasmid
40 ng of Cas7-11 plasmid
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested show an increased splicing rate relative to the Cas7-11 only construct. In combination with the other genes tested using the same set of constructs, certain genes structures or intron sizes can benefit to greater degrees from recruitment of different splicing proteins, potentially indicating a different spliceosome composition or mechanism.

### Example 20. 3′ Endogenous Trans-Splicing Rate for TOP2A Gene

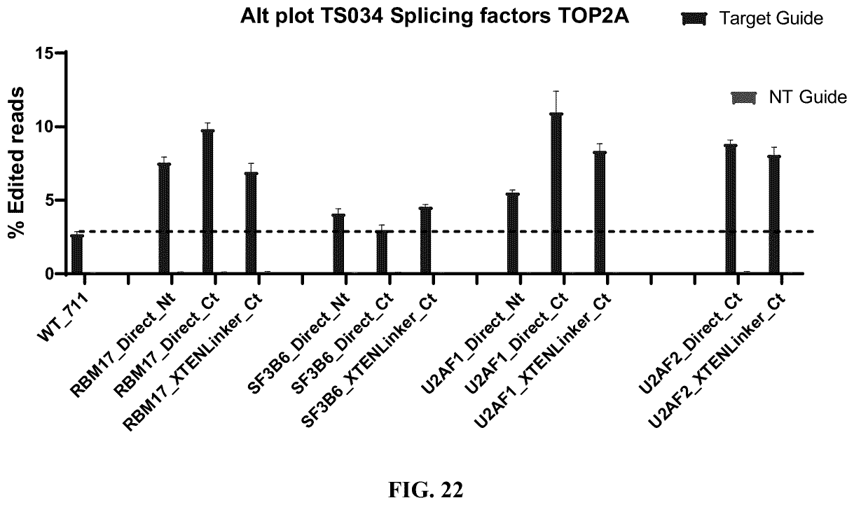

The 3′ endogenous trans-splicing rates (%) for the gene TOP2A were assessed using one common cargo replacing the TOP2A exon 21 and either a TOP2A intron 20 or scrambled guide (FIG. 22). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals. Constructs with GS linkers replacing the XTENs were also considered.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested increase trans-splicing rates relative to the wildtype Cas7-11, while others perform similarly or worse. In combination with the other genes tested using the same set of constructs, certain genes structures or intron sizes can benefit to greater degrees from recruitment of different splicing proteins, potentially indicating a different spliceosome composition or mechanism.

### Example 21. 3′ Endogenous Trans-Splicing Rate %) for SHANK3 Gene

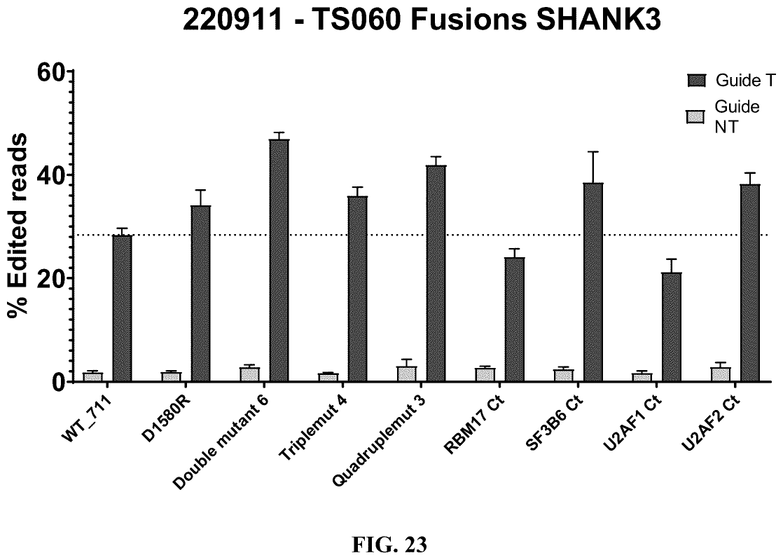

The 3′ endogenous trans-splicing rates (%) for the gene SHANK3 were assessed using one common cargo replacing the SHANK3 exon 21 and either a SHANK3 intron 20 or scrambled guide (FIG. 23). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing.

### Example 22. 3′ Endogenous Trans-Splicing Rate for SHANK3 Gene

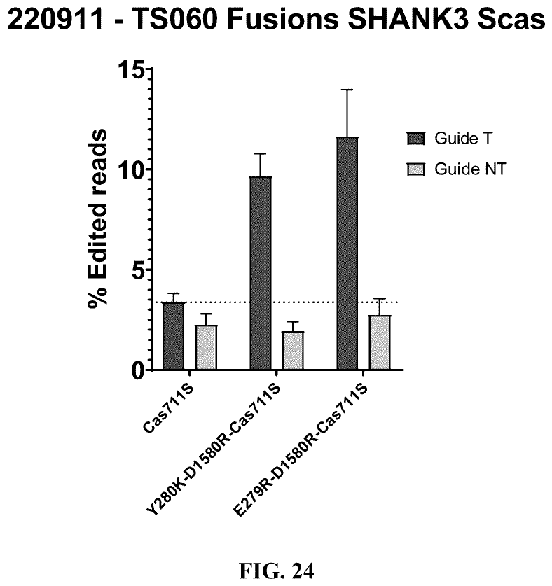

The 3′ endogenous trans-splicing rates (%) for the gene SHANK3 were assessed using one common cargo replacing the SHANK3 exon 21 and either a SHANK3 intron 20 or scrambled guide (FIG. 24). Different truncated versions of the disCas7-11 (plotted on x axis, see FIG. 24) were compared for editing efficiency. Mutations that confer higher catalytic activity for the full-length disCas7-11 were onto the best-performing truncated Cas7-11 (1006-GGGS-1221).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Smaller Cas7-11 nucleases are of interest as a lower overall size allows for more options for delivery (e.g., the use of AAV vectors or combinations of constructs). While wildtype truncated Cas7-11S performs less relative to the full-length construct, mutagenesis of the small Cas7-11 recovers some of the catalytic efficiency, potentially allowing for a smaller overall effector for trans-splicing applications requiring it.

### Example 23. 5′ Endogenous Trans-Splicing Rate for HTT Gene

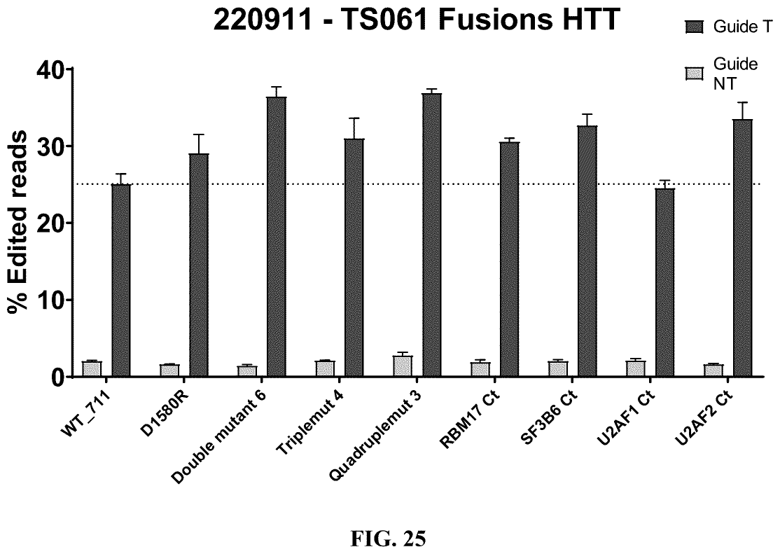

The 5′ endogenous trans-splicing rates (%) for the gene HTT were assessed using one common cargo replacing HTT exon 1 and either a HTT intron 1 or scrambled guide (FIG. 25). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

Several fusions of spliceosome proteins tested increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing.

### Example 24. 3′ Endogenous Trans-Splicing Rate for STAT3 and PPIB Genes

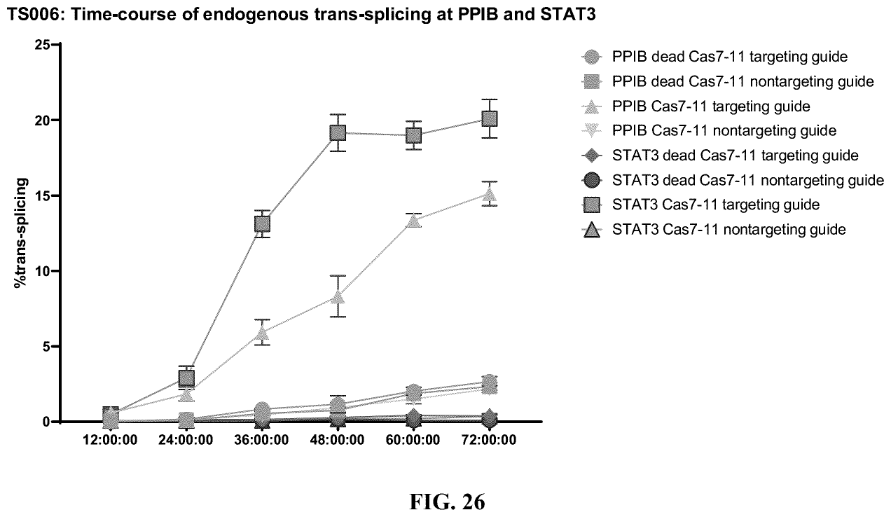

The 3′ endogenous trans-splicing rates (%) for the gene STAT3 were assessed using one common cargo replacing STAT3 exon 6 and a targeting or nontargeting guide for STAT3 intron 5 (FIG. 26). The 3′ endogenous trans-splicing rate (%) for PPIB terminal exon 14 was also assessed similarly with a guide targeting intron 4 or a scrambled sequence. As shown on FIG. 26, trans-splicing efficiency was plotted as a timeline, with timepoints every 12 hours across 3 days.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Trans-splicing kinetics were measured by assaying replacement rate over time, starting with 12 hours post-transfection. This example indicates that rates increase rapidly within the first 48 hours of introduction of the Cas7-11, guide, and cargo, and then likely plateau after −3 days post transfection. This example further suggests that there is a delay before trans-splicing can occur efficiency, likely corresponding to the timing of translation of the nuclease, and that the majority of the rate is attained within 72 hours of transfection, which can be relevant for certain dosing applications.

### Example 25. 3′ Endogenous Trans-Splicing Rate for STAT3 Gene

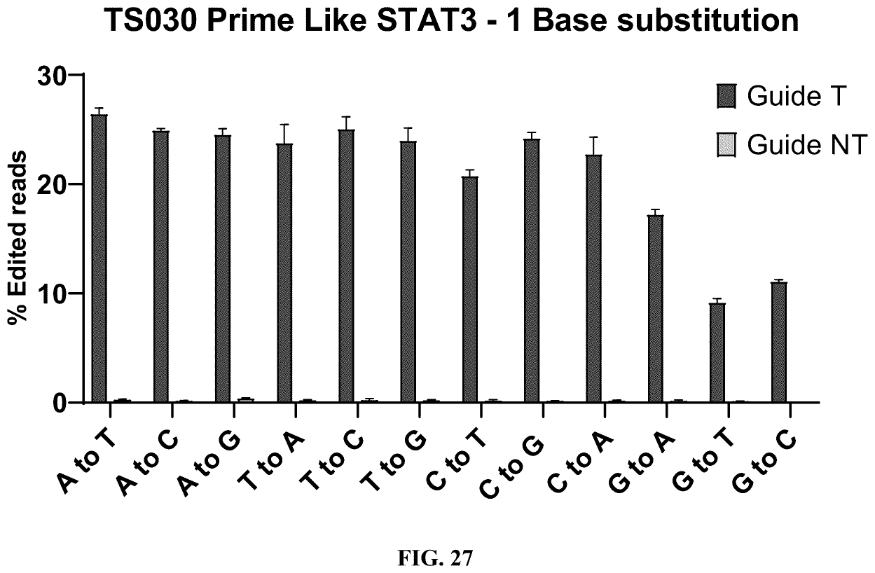

The 3′ endogenous trans-splicing rates (%) for the gene STAT3 were assessed using one common cargo structure replacing STAT3 exon 6 and a targeting or nontargeting guide for STAT3 intron 5 (FIG. 27). Variations of the inserted sequence were compared by inducing mutations to generate each possible single base conversion (e.g., A→G, C, or T).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure. Sequence variation within the exon inserted in trans has only a minimal effect on splicing rates, with the exception of the conversions of G→A, C, or T. This difference is likely due to changing the first nucleotide of the inserted exon, which is known to be a part of the -NNGTNNN- splice acceptor motif found at the start of nearly all mammalian exons. This example shows that a variety of cargos can be inserted (allowing for applications such as the correction of mutations), but that the initial splice acceptor “GT” should be conserved as it participates in the splicing reaction.

### Example 26. 3′ Endogenous Trans-Splicing Rate for STAT3 Gene

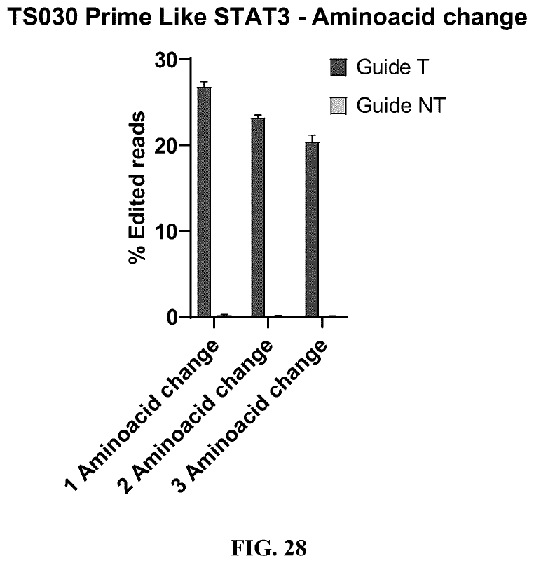

The 3′ endogenous trans-splicing rates (%) for the gene STAT3 were assessed using one common cargo structure replacing STAT3 exon 6 and a targeting or nontargeting guide for STAT3 intron 5 (FIG. 28). Variations of the inserted sequence were compared by inducing mutations to change bases of the inserted exon either 1, 2, or 3 residues at a time.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure. Sequence variation within the exon inserted in trans has only a minimal effect on splicing rates, indicating that trans-splicing can generate single or multiple residue changes to the inserted cargos (relevant to applications for correcting disease mutations or inducing a desired minor change). The apparent small reduction in splicing rate with 2 or 3 residue changes may be due to a marginal increase in the amplicon length for these cargos, as primers read across the inserted region.

### Example 27. 3′ Endogenous Trans-Splicing Rate for STAT3 Gene

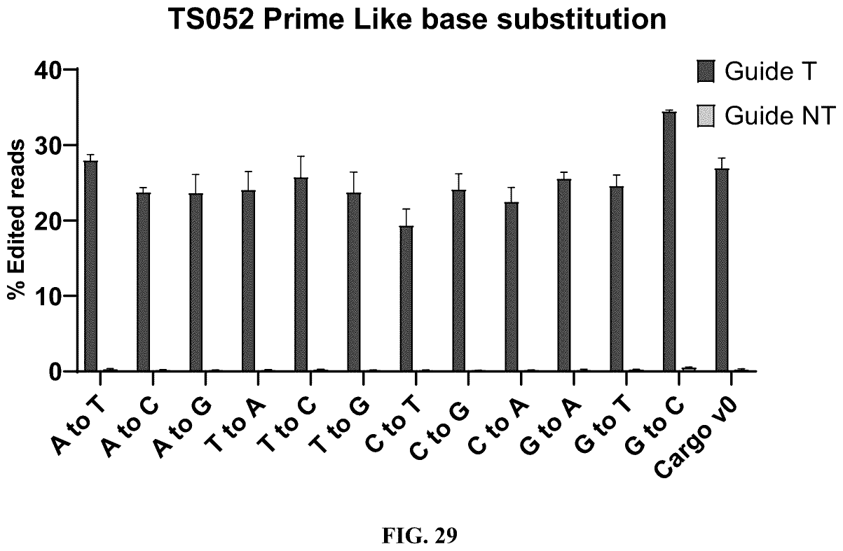

The 3′ endogenous trans-splicing rates (%) for the gene STAT3 were assessed using one common cargo structure replacing STAT3 exon 6 and a targeting or nontargeting guide for STAT3 intron 5 (FIG. 29). Variations of the inserted sequence were compared by inducing mutations to generate each possible single base conversion (e.g., A→G, C, or T).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure. Sequence variation within the exon inserted in trans has only a minimal effect on splicing rates. This experiment constructs with the initial comparison in that the G-base conversions are not on the first G within the exon, which is known to be a part of the -NNGTNNN-splice acceptor motif found at the start of nearly all mammalian exons, confirming that non-first Gs can be replaced without negatively affecting splicing rates. This example shows that a variety of cargos can be inserted (allowing for applications such as the correction of mutations).

### Example 28. 3′ Endogenous Trans-Splicing Rate for PPIB Gene

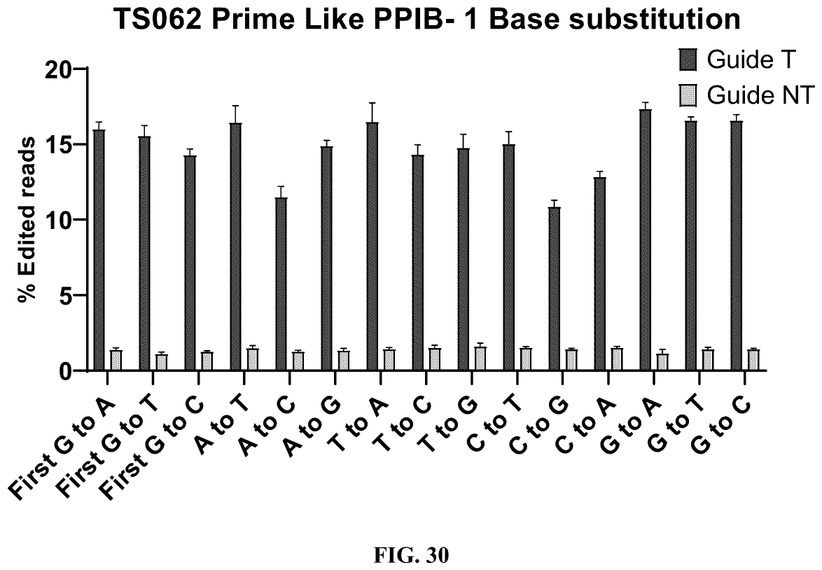

The 3′ endogenous trans-splicing rates (%) for the gene PPIB were assessed using one common cargo replacing the PPIB terminal exon and either a PPIB intron 4 or scrambled guide (FIG. 30). Variations of the inserted sequence were compared by inducing mutations to generate each possible single base conversion (e.g., A→G, C, or T).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure. Sequence variation within the exon inserted in trans has only a minimal effect on splicing rates. This experiment shows that a variety of cargos can be inserted (allowing for applications such as the correction of mutations) for a second target.

Unlike the STAT3 target, PPIB splicing rates are less affected by replacement of the first G (from the -NNNAGNNN-) splice acceptor motif.

This data supports the STAT3 insertion data, suggesting that the observed behaviour is generalizable to multiple endogenous targets.

### Example 29. 3′ Endogenous Trans-Splicing Rate for PPIB Gene

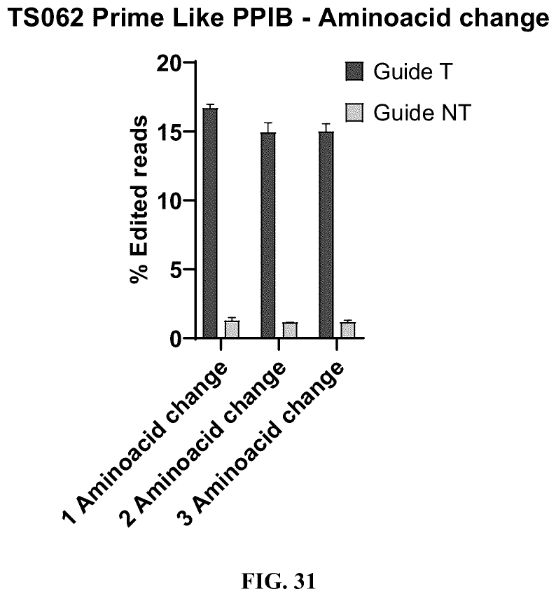

The 3′ endogenous trans-splicing rates (%) for the gene PPIB were assessed using one common cargo replacing the PPIB terminal exon and either a PPIB intron 4 or scrambled guide (FIG. 31). Variations of the inserted sequence were compared by inducing mutations to change bases of the inserted exon either 1, 2, or 3 residues at a time.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure. Sequence variation within the exon inserted in trans has only a minimal effect on splicing rates, indicating that trans-splicing can generate single or multiple residue changes to the inserted cargos (relevant to applications for correcting disease mutations or inducing a desired minor change).

This data supports the STAT3 insertion data, suggesting that the observed behaviour is generalizable to multiple endogenous targets.

### Example 30. 3′ Endogenous Trans-Splicing Rate for PPIB Gene

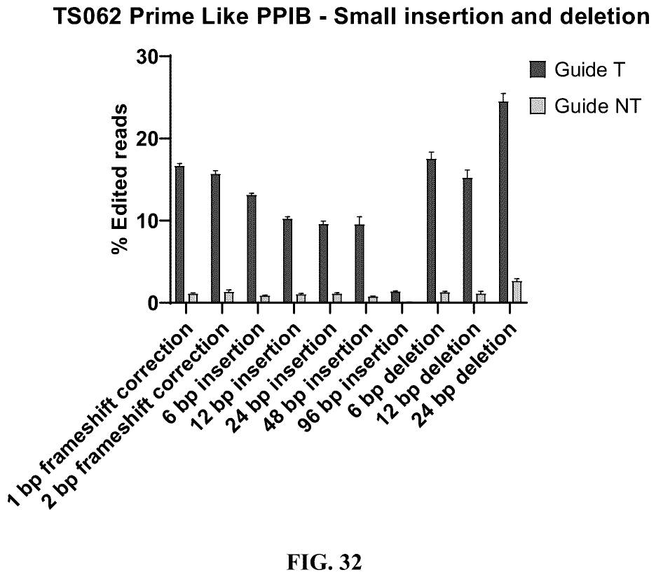

The 3′ endogenous trans-splicing rates (%) for the gene PPIB were assessed using one common cargo replacing the PPIB terminal exon and either a PPIB intron 4 or scrambled guide (FIG. 32). Variations of the inserted sequence were generated with insertions of the sizes reported across the x axis (from 1-96 bp, see FIG. 32) as well as deletions from 6-24 bp.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure. Sequence variation within the exon inserted in trans has only a minimal effect on splicing rates, indicating that trans-splicing can generate single or multiple residue changes to the inserted cargos (relevant to applications for correcting disease mutations or inducing a desired minor change).

The apparent small reduction in splicing rate with increasingly large insertions, and conversely the increase with deletions, can be due to an increase in the amplicon length for these cargos, as primers read across the inserted region and therefore can be biased against in the readout. However, this example shows that large structural changed can be made to the cargos without impairing the overall ability to splice.

### Example 31. 3′ Endogenous Trans-Splicing Rate for PPIB (PP), USF1 (U), STAT3 (S), PABPC1 (PA), and TOP2A(T) Genes

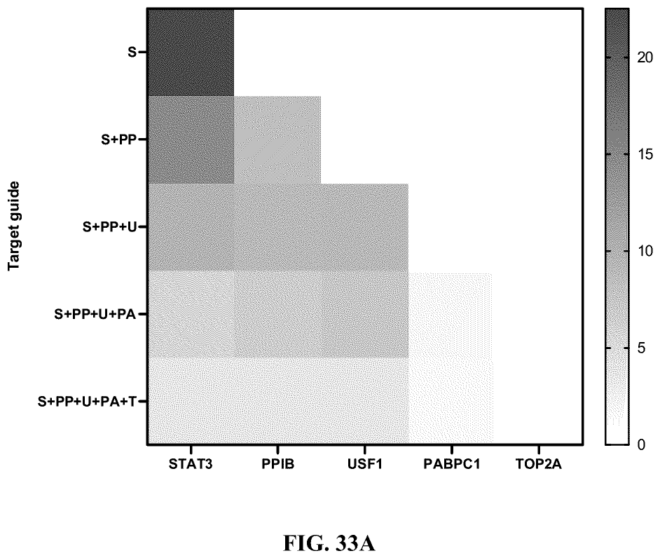

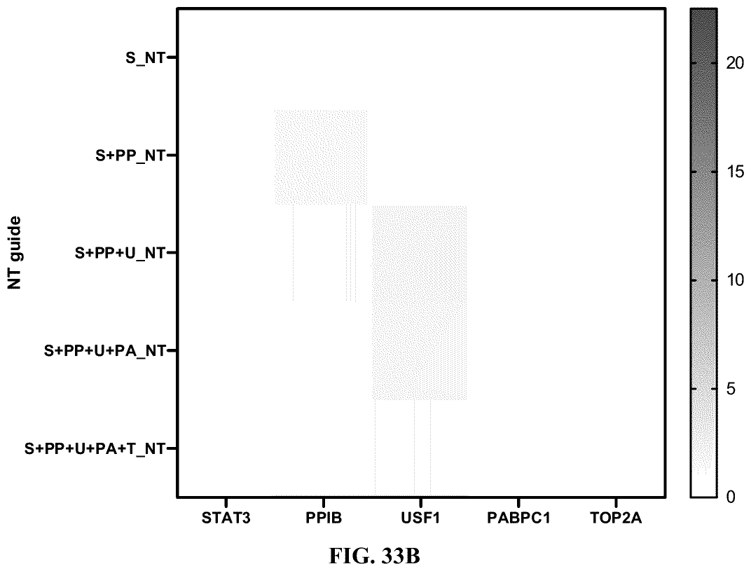

The 3′ endogenous trans-splicing rates (%) for the genes PPIB (PP), USF1 (U), STAT3 (S), PABPC1 (PA), and TOP2A(T) edited simultaneously within the same conditions were assessed (FIG. 33A and FIG. 33B). The heat maps from FIG. 33A and FIG. 33B show splicing rate (0-25%) per target (across the X axis) for each of the combinations (shown on the y axis).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

For this example, 10 ng of guide and cargo were used per gene assayed in each condition—therefore, total DNA amounts vary relative to the constant amount of nuclease transfected.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

These results demonstrate that trans-splicing is multiplex-able—in the sense that multiple endogenous transcripts can be edited with relatively stable efficiency concurrently. Applications for this type of multiplexing (several cargos into several targets, vs several cargos into a single target) can be the tagging of genes concurrently, or replacement of multiple therapeutically relevant genes, or barcoding of specific transcripts for visualization of sequencing purposes.

### Example 32. 3′ Endogenous Trans-Splicing Rate for STAT3 Gene

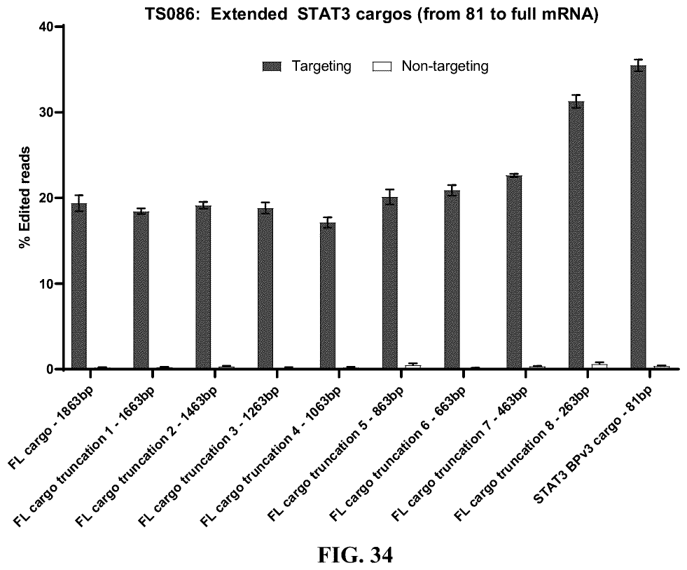

The 3′ endogenous trans-splicing rates (%) for the gene STAT3 were assessed using one common cargo structure replacing STAT3 exon 6 and a targeting or nontargeting guide for STAT3 intron 5 (FIG. 34). In FIG. 34, the original cargo is shown furthest to the right, with cargos to the left of it having increasingly large sizes (up to insertion of the entire STAT3 transcript).

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to demonstrate that cargo insertion rates are largely independent of the actual cargo structure or size. It shows that cargos ranging from 80 bp to almost 2 kb can be inserted at the STAT3 locus with comparable efficiency (especially for cargos between 463 bp and 1863 bp, for which there is limited, if any, reduction in rates). This suggests that splicing efficiency likely has little to do with the structure of the cargo and indicates that it can be possible to insert large sequences using this trans-splicing strategy.

### Example 33. 3′ Endogenous Trans-Splicing Rate for PPIB Gene

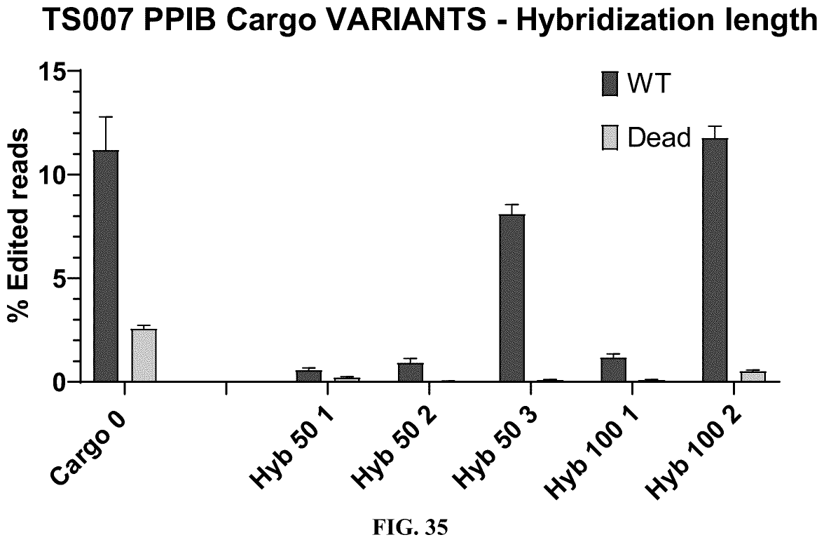

The 3′ endogenous trans-splicing rates (%) for the gene PPIB were assessed using one common cargo replacing the PPIB terminal exon and either a PPIB intron 4 or scrambled guide (FIG. 35). Truncations of the hybridization region were tested for impact on overall splicing rate at PPIB. Truncations tested include 50 bp reductions of the original cargo from the 5′ and 3′ ends (totaling 100 bp), or 50 bp from each side.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Shorter hybridization regions, and smaller overall cargo sizes, are important for applications requiring a compact editing system.

This example assessed the minimal binding region for the trans-splicing cargo. Shorter hybridization regions have a relatively significant impact on splicing rates, with cargos that remove the 3′ 50 bp having the largest effect. This suggests that this particular region can be essential for the function of the cargo. These data suggest that relatively efficient splicing can occur even with cargos with shorter hybridizations than the ones used in this example—provided that they span or bind to critical regions of the intron.

### Example 34. 3′ Endogenous Trans-Splicing Rate for PPIB Gene

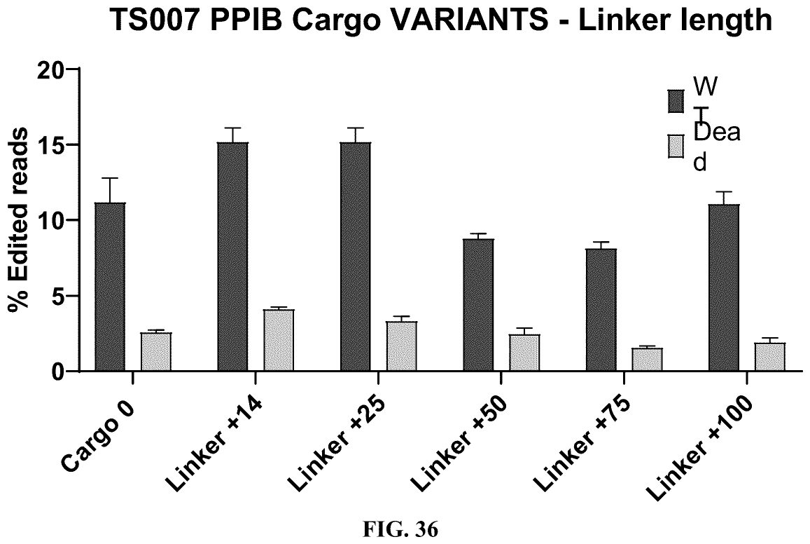

The 3′ endogenous trans-splicing rates (%) for the gene PPIB were assessed using one common cargo replacing the PPIB terminal exon and either a PPIB intron 4 or scrambled guide (FIG. 36). Cargos with insertions in linker region between hybridization and replacement exon were tested, wherein the linkers range from 14 bp to 100 bp at the largest.

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to explore how different structural arrangements of the cargo elements affect splicing rates. As different exons and introns range in size and position, varying the length or flexibility of the cargo can lead to an improvement of splicing rates due to sterics or accessibility of the various splicing components.

This example suggests that for PPIB, longer cargos can improve rates, with 14 bp and 25 bp longer linkers showing a modest improvement over the baseline cargo structure. Therefore, linker length can be an additional angle for tuning the efficiency of trans-splicing.

### Example 35. 3′ Endogenous Trans-Splicing Rate for PPIB Gene

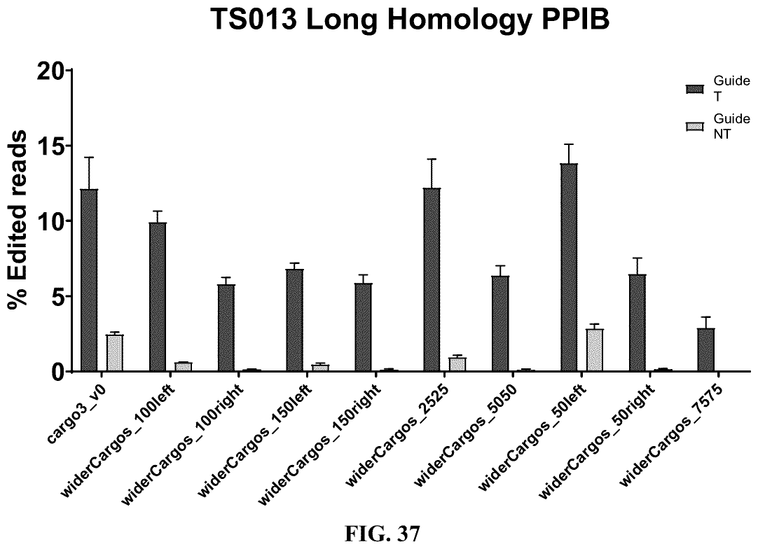

The 3′ endogenous trans-splicing rates (%) for the gene PPIB were assessed using one common structure cargo replacing the PPIB terminal exon and either a PPIB intron 4 or scrambled guide (FIG. 37). As shown on FIG. 37, cargos based on the original cargo structure, but with larger hybridization regions were compared across the x axis, and the following bases were added (from left to right):
100 bp 5′;
100 bp 3′;
150 bp 5′;
150 bp 3′;
25 bp 5′ and 3′;
50 bp 5′ and 3′;
50 bp 5′;
50 bp 3′; and
75 bp 5′ and 3′.

### Materials & Methods

The constructs were transfected at a 96-well scale on HEK293FT cells in DMEM 10% FBS. For endogenous splicing experiments, transfections were carried out using:
10 ng of guide
10 ng of cargo plasmid
40 ng of Cas7-11 plasmid
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example aims to explore how different sizes of homology region greater than the original cargo could affect splicing rates. A longer homology region could theoretically bind more favourably or interact with a region of the intron that biases splicing further towards the splicing product. However, these results indicate that larger cargos are likely less efficient, potentially due to factors such as secondary structure or covering of necessary intron elements. Interestingly, the size and position of the cargo can also change its behavior in the NT guide situation, potentially indicating that certain cargo designs would have more or less “background” splicing.

### Example 36. 3′ Endogenous Trans-Splicing Rate for STAT3 Gene

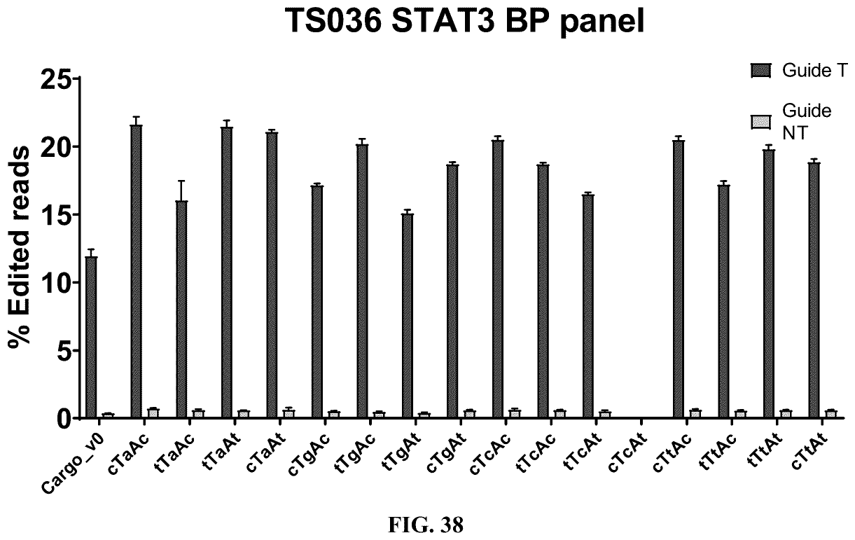

The 3′ endogenous trans-splicing rates (%) for the gene STAT3 were assessed using one common cargo structure replacing STAT3 exon 6 and a targeting or nontargeting guide for STAT3 intron 5 (FIG. 38). Variations of the original cargo with different branchpoint motifs were compared for effect on splicing rates. The motif sequences tested are shown across the x-axis of FIG. 38.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

One of the major motifs relevant to mammalian splicing is the branch point, generally found upstream of the 3′ exon within the intron. For mammalian splicing, the consensus motif is yUnAy. In this example, every variation of this motif was tested for its impact on splicing efficiency. Relative to the original, non-consensus-motif cargo, nearly all variations tested perform significantly better, with motifs such as cTtAc or cTaAc delivering ˜2× higher splicing rates for STAT3.

Therefore, the inclusion and engineering of different branchpoints can be highly relevant for improving trans-splicing rates in this system, orthogonal to other improvements to nuclease efficiency or cargo structure.

### Example 37. 3′ Endogenous Trans-Splicing Rate for PPIB Gene

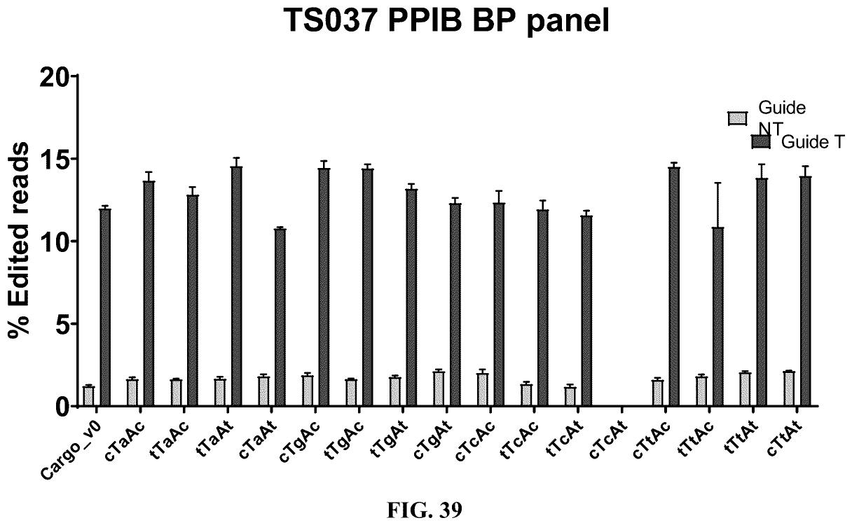

The 3′ endogenous trans-splicing rates (%) for the gene PPIB were assessed using one common structure cargo replacing the PPIB terminal exon and either a PPIB intron 4 or scrambled guide (FIG. 39). Variations of the original cargo with different branchpoint motifs were compared for effect on splicing rates. The motif sequences tested are shown across the x-axis in FIG. 39.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

One of the major motifs relevant to mammalian splicing is the branch point, generally found upstream of the 3′ exon within the intron. For mammalian splicing, the consensus motif is yUnAy. In this example, variations of this motif were tested for its impact on splicing efficiency. Relative to the original, non-consensus-motif cargo, most of the variations tested performed significantly better, with motifs such as cTtAc or cTaAc delivering ˜2× higher splicing rates for PPIB.

Therefore, the inclusion and engineering of different branchpoints can be highly relevant for improving trans-splicing rates in this system, orthogonal to other improvements to nuclease efficiency or cargo structure.

### Example 38. 3′ Endogenous Trans-Splicing Rate for SHANK3 Gene, Exon 21

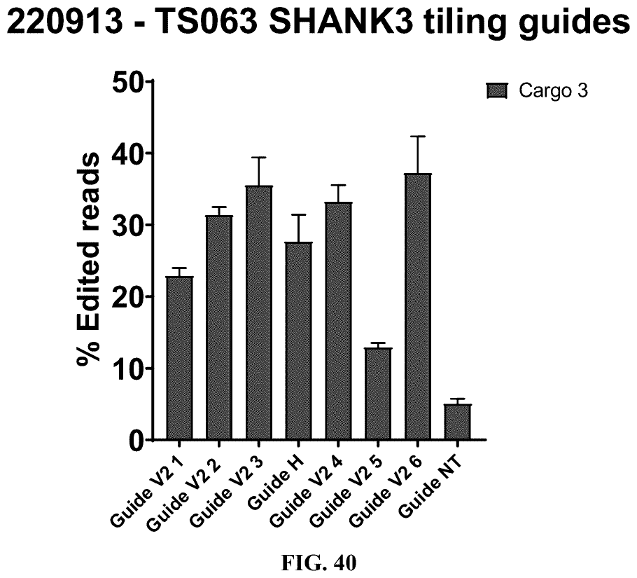

The 3′ endogenous trans-splicing rates (%) for the gene SHANK3, exon 21 were assessed (FIG. 40). Cargos were tested in combination with a set of arranged around a previous best guide identified in an initial screen, with guides 1-3 binding upstream of the previous best “guide H” and guides 4-6 binding downstream.

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Cleavage activity in the trans-splicing reaction is due both to nuclease activity and guide binding+accessibility. By tiling guides around positions that perform well, fine tuning of cleavage activity can be accomplished. This example shows that tiling (testing guides in close proximity to other working guides) can further increase splicing efficiency for a given locus of interest.

### Example 39. 3′ Endogenous Trans-Splicing Rate for SHANK3 Gene

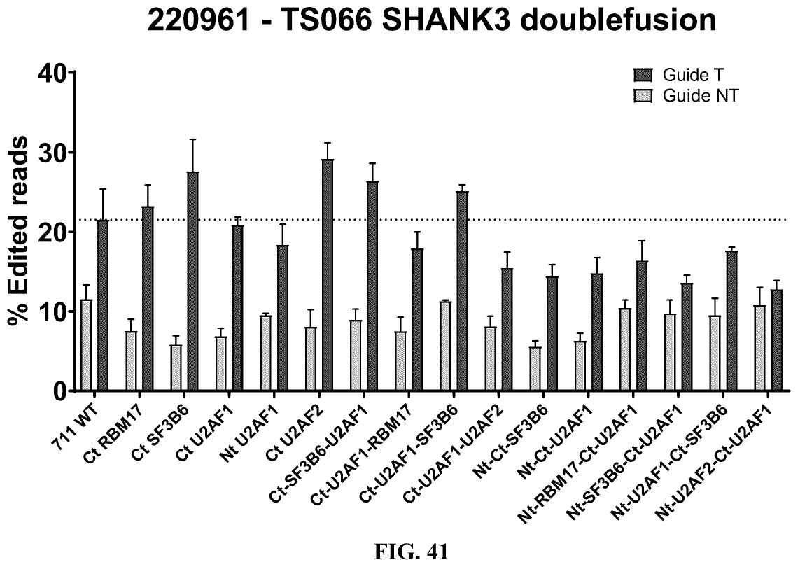

The 3′ endogenous trans-splicing rates (%) for the gene SHANK3 were assessed using one common cargo replacing the SHANK3 exon 21 and either a SHANK3 intron 20 or scrambled guide (FIG. 41). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity. Constructs that combine working N- and C-terminal fusion constructs into editors with multiple fusions (both N- and C-, or tandem N- and C-) were also tested.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins increase trans-splicing rates relative to the wildtype Cas7-11. Additionally, tandem or both N- and C-terminal constructs performed better than wildtype Cas7-11 in this comparison.

### Example 40. 3′ Endogenous Trans-Splicing Rate for STAT3 Gene

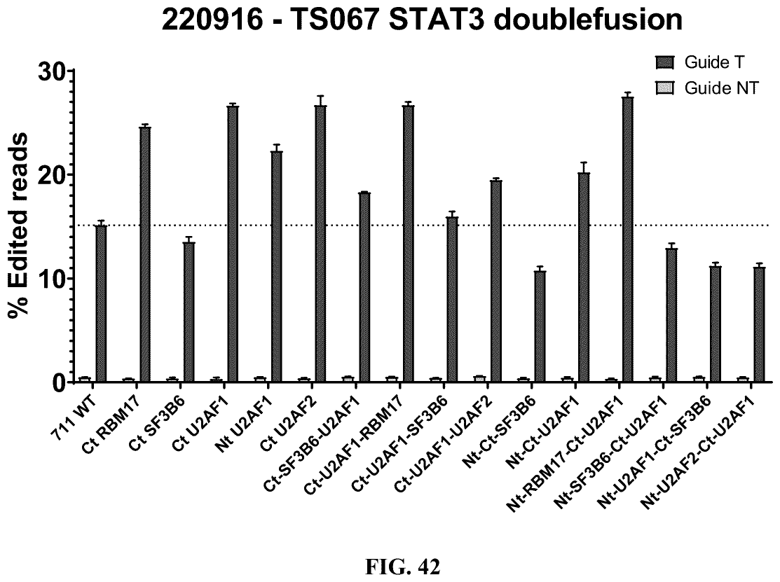

The 3′ endogenous trans-splicing rates (%) for the gene STAT3 were assessed using one common cargo structure replacing STAT3 exon 6 and a targeting or nontargeting guide for STAT3 intron 5 (FIG. 42). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity. Constructs that combine working N- and C-terminal fusion constructs into editors with multiple fusions (both N- and C-, or tandem N- and C-) were also tested.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins were found to increase trans-splicing rates relative to the wildtype Cas7-11. Additional, tandem or both N- and C-terminal constructs were found to perform much better than wildtype Cas7-11 in this comparison.

Together with the other genes tested with these constructs, it is observed that there can be a gene or exon specific effect from fusions—potentially due to different spliceosome components or behaviour dependent on the situation.

### Example 41. 3′ Endogenous Trans-Splicing Rate for PPIB and STAT3 Genes Either Alone or Edited Simultaneously

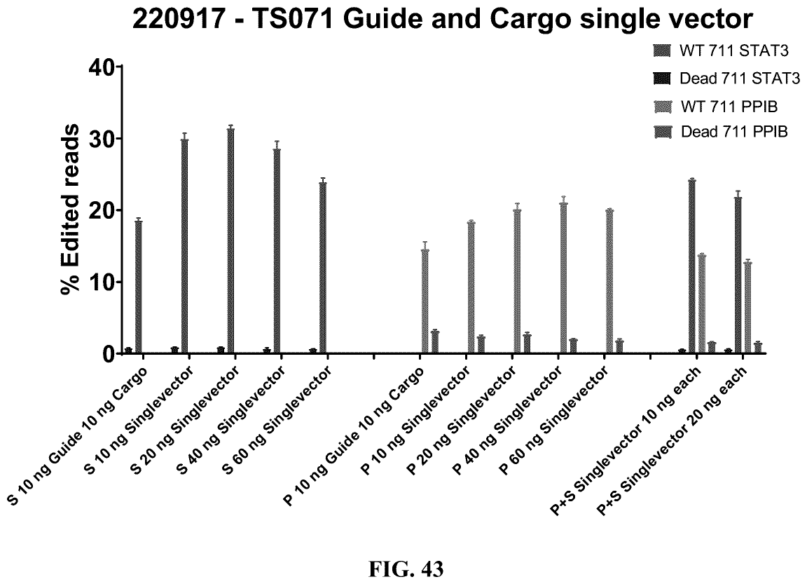

The 3′ endogenous trans-splicing rates (%) for the genes PPIB and STAT3 either alone or edited simultaneously were assessed (FIG. 43). In FIG. 43, shown on the x-axis are conditions with either the conventional guide+cargo combination, or a single guide and cargo carrying plasmid substituting for the two. Also shown are conditions where single-vector guide cargo plasmids for multiple genes are co-transfected.

The constructs were transfected at a 96-well scale on HEK293FT cells in DMEM 10% FBS. For endogenous splicing experiments, transfections were carried out using:
10 ng of guide;
10 ng of cargo plasmid, or 20+ng of single vector constructs; and
40 ng of Cas7-11 plasmid,
   co-transfected using Lipofectamine 3000.

Multiple DNA amounts of guide and cargo were used per gene assayed in each condition. Single vector constructs were tested at 10, 20, 40, or 60 ng per target. RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This result shows that relative to the triple transfection strategy (furthest left for each gene), a double transfection where guide and cargo are combined onto a single plasmid boosts efficiency. This is likely due to improved delivery of the constructs to the same cell.

This result is encouraging for future applications leveraging AAV or other viral delivery mechanisms, where keeping the number of parts involved to the minimum is essential for efficient delivery.

### Example 42. 3′ Endogenous Trans-Splicing Rate for STAT3 Gene

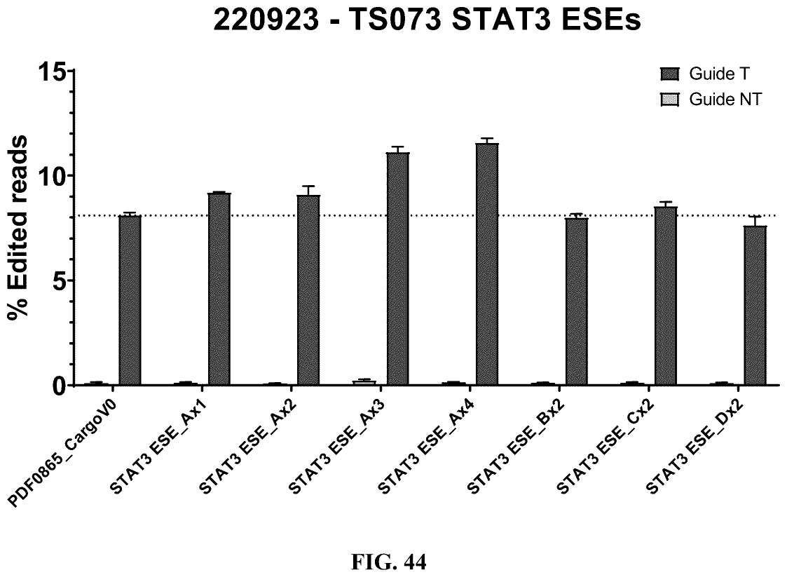

The 3′ endogenous trans-splicing rates (%) for the gene STAT3 were assessed using one common cargo structure replacing STAT3 exon 6 and a targeting or nontargeting guide for STAT3 intron 5 (FIG. 44). The original cargo (furthest left in FIG. 44) was compared against variants with ESE sequence motifs inserted 3′ to the inserted region.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Exonic splicing enhancers (ESEs) are DNA sequence motifs suggested to have a role in biasing the inclusion of one exon over another. In this example, short ESE motifs are included downstream of the cargo to see whether they boost trans-splicing rates in this context.

Several of the ESEs tested can improve trans-splicing for this STAT3 exon, while others perform similarly or worse than the original cargo. Therefore, ESEs can be used to further optimize specific trans-splicing cargos.

### Example 43. 3′ Endogenous Trans-Splicing Rate for SHANK3 Gene

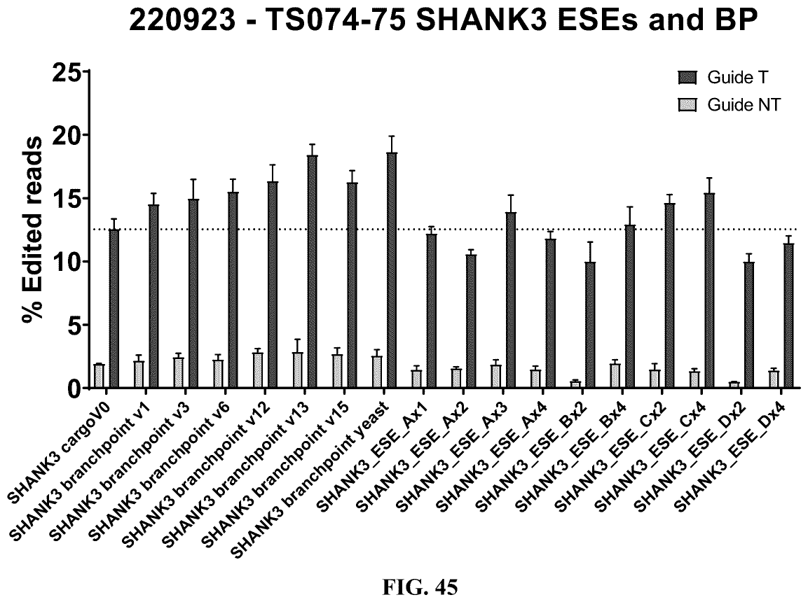

The 3′ endogenous trans-splicing rates (%) for the gene SHANK3 were assessed using one common cargo structure replacing SHANK3 exon 21 and a targeting or nontargeting guide for SHANK3 intron 20 (FIG. 45). The original cargo (furthest left in FIG. 45) was compared against variants with ESE sequence motifs inserted 3′ to the inserted region. Variations of the original cargo with different branchpoint motifs, compared for effect on splicing rates, were also assessed. These sequences were found to be the best performing branchpoint motifs from a comprehensive screen on PPIB and STAT3.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Exonic splicing enhancers (ESEs) are DNA sequence motifs suggested to have a role in biasing the inclusion of one exon over another. In this example, short ESE motifs are included downstream of the cargo to see whether they boost trans-splicing rates in this context. Several of the ESEs tested can improve trans-splicing for this STAT3 exon, while others perform similarly or worse than the original cargo. Therefore, ESEs can provide an addition way to further optimize trans-splicing cargos.

Similarly, the branch point sequences confer a boost to trans-splicing rates, in alignment with results from other genes tested that show that the inclusion of specific splice motifs can have a large improvement on rates.

### Example 44. 3′ Endogenous Trans-Splicing Rate for STAT3 Gene

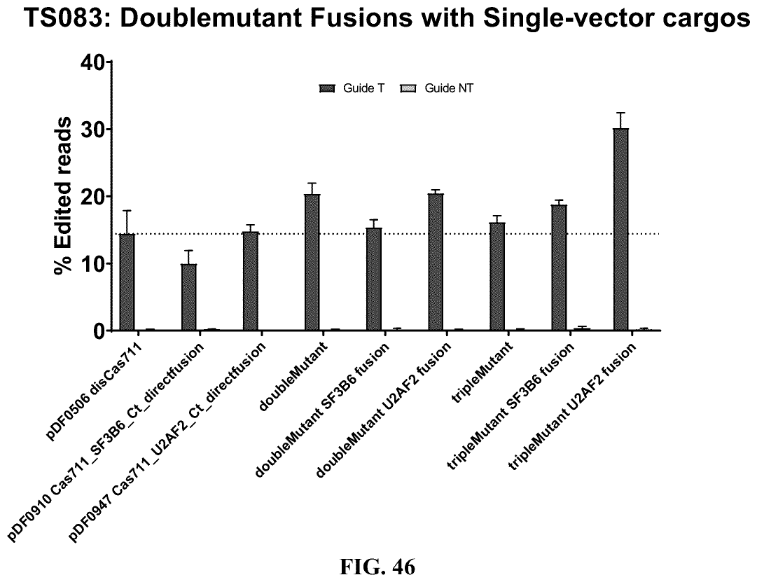

The 3′ endogenous trans-splicing rates (%) for the gene STAT3 were assessed using one common cargo structure replacing STAT3 exon 6 and a targeting or nontargeting guide for STAT3 intron 5 combined on a single plasmid (FIG. 46). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing. These improvements further scale with the single-vector versions of the STAT3 constructs, where guides and cargos are cloned into a single plasmid. This shows that multiple orthogonal methods for improving efficiency are compatible with each other and can be developed in parallel for a given target.

### Example 45. 3′ Endogenous Trans-Splicing Rate for SHANK3 Gene

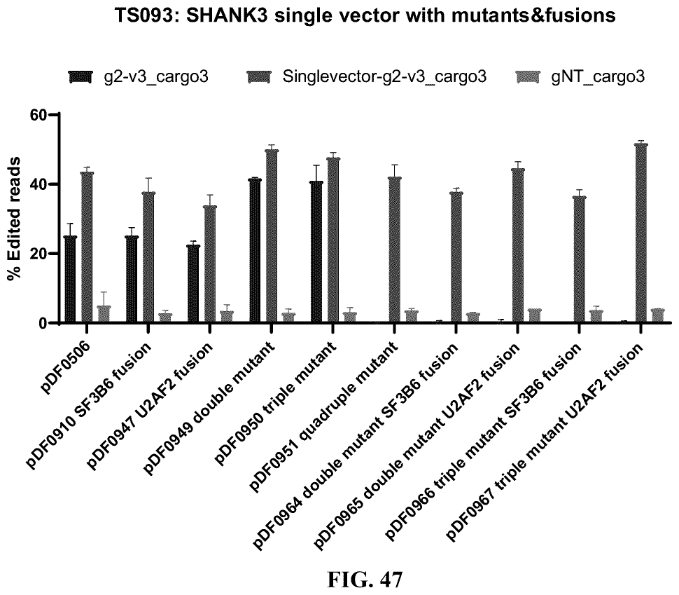

The 3′ endogenous trans-splicing rates (%) for the gene SHANK3 were assessed using one common cargo structure replacing SHANK3 exon 21 and a targeting or nontargeting guide for SHANK3 intron 20 combined on a single plasmid (FIG. 47). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins were found to increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing. These improvements further scale with the single vector versions of the SHANK3 constructs, where guides and cargos are cloned into a single plasmid. This shows that multiple orthogonal methods for improving efficiency are compatible with each other and can be developed in parallel for a given target, and that the improvements seen for other genes (e.g., STAT3) are generalizable.

### Example 46. 3′ Endogenous Trans-Splicing Rate for STAT3 and PPIB Genes

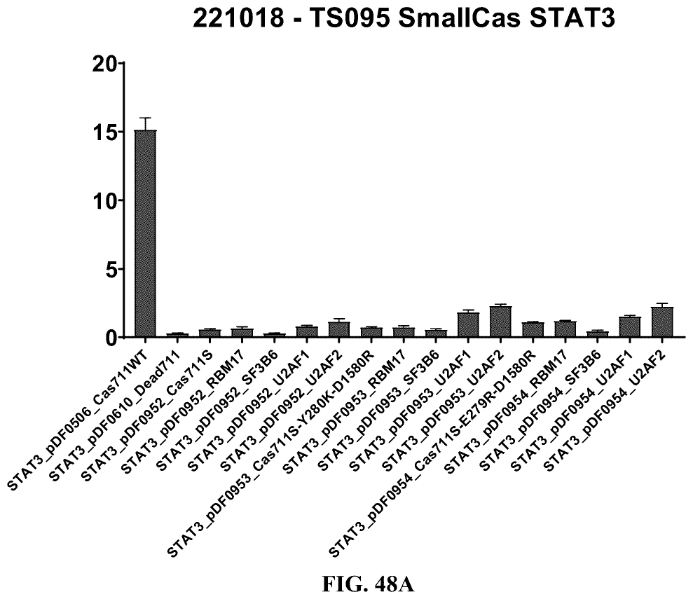

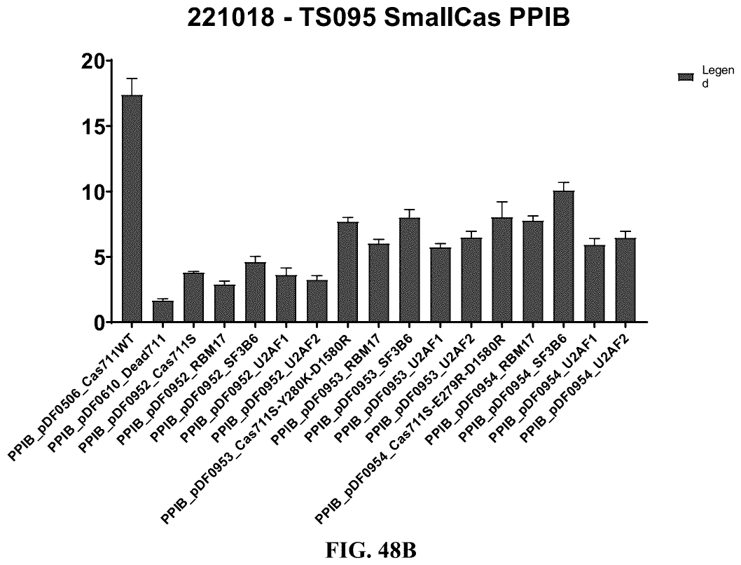

The 3′ endogenous trans-splicing rates (%) for the genes STAT3 and PPIB were assessed (FIG. 48A and FIG. 48B). The 3′ endogenous trans-splicing rate (%) for the gene STAT3 was probed using one common cargo replacing STAT3 exon 6 and either a Cas7-11 guide RNA targeting the STAT3 intron 5 or a guide scrambled sequence. The 3′ endogenous trans-splicing rate (%) for the gene PPIB was probed using one common structure cargo replacing the PPIB terminal exon and either a PPIB intron 4 or scrambled guide with the same set of truncated spliceosome fusions. Different truncated versions of the disCas7-11 (plotted on x axis of FIG. 48A and FIG. 48B) were compared for editing efficiency and tested combined with a direct (no intervening linker) or indirect (XTEN linker intervening) fusion to various splicing proteins (RMB17, SF3B6, U2AF1, or U2AF2) at their N- or C-terminals.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Smaller Cas7-11 nucleases are of interest as a lower overall size allows for more options for delivery (e.g., the use of AAV vectors or combinations of constructs). This example demonstrates that smaller, truncated Cas7-11 variants cause a major drop-off in efficiency of trans-splicing, likely due to a loss of catalytic activity. However, fusions of Cas7-1 is with splicing proteins (as previously done for the full-length disCas7-11) can rescue the overall splicing rate, in particular for PPIB.

### Example 47. 3′ Endogenous Trans-Splicing Rate for SHANK3 Gene

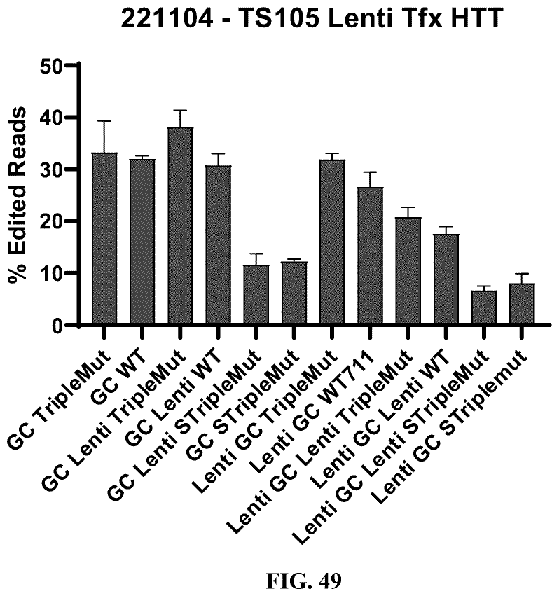

The 3′ endogenous trans-splicing rates (%) for the gene SHANK3 were assessed using conventional or lentiviral vectors (FIG. 49). Wild type, triple mutant and small Cas7-11 either in conventional or lentiviral vectors, were combined with either a conventional or lentiviral single vector (GC) expressing a guide RNA targeting the SHANK3 intron 20 and a cargo replacing SHANK3 exon 21. Different combinations (on the x-axis from FIG. 49) were compared for editing efficiency. The constructs cloned into lentiviral vectors were compared with the conventional vectors to see if there is any functional loss caused by the lentiviral backbone.

### Materials & Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Lentiviral packaging of Cas7-11, guide and cargo vectors is of interest as it enables to generate cell lines stably expressing these constructs. This approach allows for the editing efficiency to be not limited by the transfection efficiency and enables editing in primary cells that are difficult to transfect.

### Example 48. 3′ Endogenous Trans-Splicing Rate for SHANK3 Gene

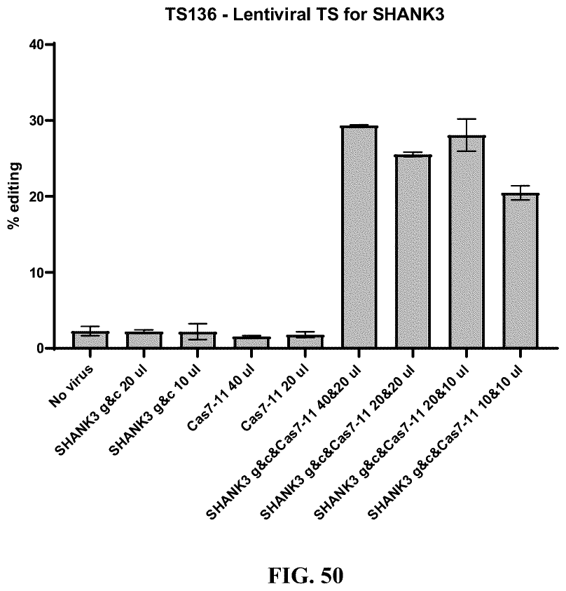

The 3′ endogenous trans-splicing rates (%) for the gene SHANK3 were assessed using different volumes of 2 lentiviruses either alone or in combination (FIG. 50). The first virus was designed to package a vector expressing a guide RNA targeting the SHANK3 intron 20 and a cargo replacing SHANK3 exon 21. The second virus was designed to package a vector expressing Cas7-11. On the bar graph of FIG. 50, each condition is noted on the x-axis, and editing efficiency for each condition is shown on the y-axis.

### Materials & Methods

Lentiviruses were produced in HEK293FT cells cultured in T225 flasks, by transfection of 30 g of packaging plasmid (psPAX2), 30 g of envelope plasmid (VSV-G), and 30 g of transfer plasmid (lenti Cas7-11 or lenti guide&cargo) using 270 μL of polyethylene imine (PEI). Media containing lentiviruses were harvested after 48 h of transfection, ultracentrifuged for 2 h at 120,000 g, and concentrated 100× by resuspending in PBS. HEK293FT cells were infected with lentiviruses at a 96-well scale in DMEM 10% FBS, by following virus volumes:
0, 10 or 20 μl of guide and cargo viruses; and
0, 20 or 40 μl of Cas7-11 viruses.

RNA was harvested 7 days post-transduction and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Lentiviral packaging of Cas7-11, guide and cargo vectors are of interest as it enables to generate cell lines stably expressing these constructs. This approach allows for the editing efficiency to be not limited by the transfection efficiency and enables editing in primary cells that are difficult to transfect.

In this example, lentiviruses packaging Cas7-11 or single guide and cargo vectors were used to infect HEK293FT cells. About 30% editing was observed in the cells co-infected with both lentiviruses.

### Example 49. 3′ Endogenous Trans-Splicing of PPIB Gene

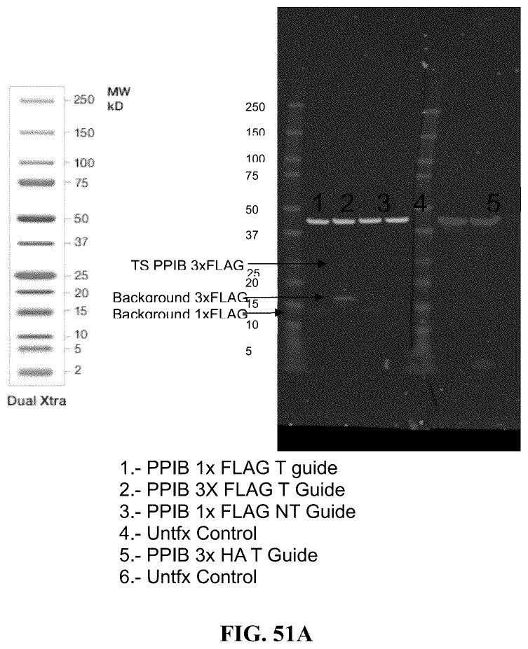

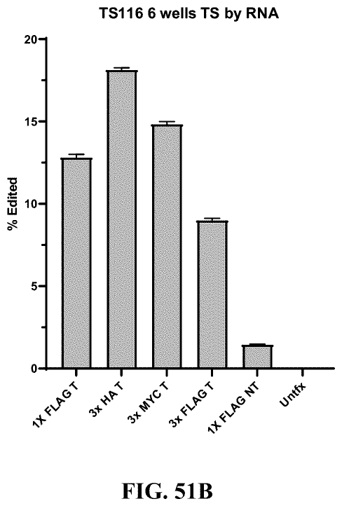

The 3′ endogenous trans-splicing of the gene PPIB was assessed using a cargo replacing the PPIB terminal exon and containing 1× or 3×Flag or 1×HA tags, and either a PPIB intron 4 targeting or scrambled guide RNA (FIG. 51A and FIG. 51B). The bar graph of FIG. 51B reports the 3′ endogenous trans-splicing rate (%) for the gene PPIB as a confirmation of the Western blot of FIG. 51A.

The constructs were transfected at a 6-well scale on HEK293FT cells in DMEM 10% FBS. For endogenous splicing experiments, transfections were carried out using:
400 ng of guide;
400 ng of cargo plasmid; and
1600 ng of Cas7-11 plasmid,
   co-transfected using Lipofectamine 3000.

Each condition was transfected on 2 wells. 3 days post-transfection, RNA was harvested from 1 well, and protein was harvested from the other well, by specific lysis buffers.

Harvested RNA reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

Protein concentration was determined by BCA assay, and equal amounts from each sample were run on Bio-Rad 4-20% Mini-PROTEAN gel. They were transferred on nitrocellulose membrane via Thermo iBlot-2 transfer device, blocked for 1 h at RT, and incubated overnight with primary antibody at 4° C. Next day, membrane was washed before and after incubation with secondary antibody for 1 h at RT and imaged by LI-COR Odyssey Scanner.

### Discussion

Even though trans-splicing occurs in the RNA-level, a goal of using this tool is replacing disease-related mutant proteins with the wild type versions. Therefore, to validate editing results in RNA-level, and show the translation of trans-spliced product, Western blot is one of the most important techniques.

### Example 50. 3′ Trans-Splicing Rate for USF1 Gene

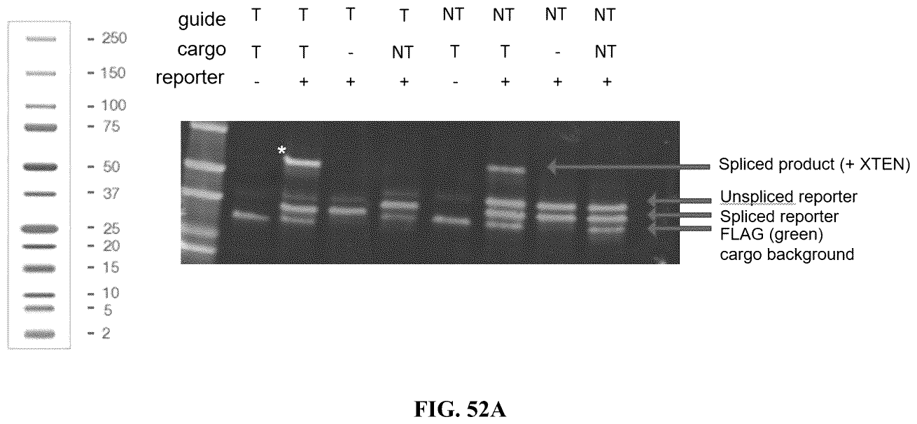

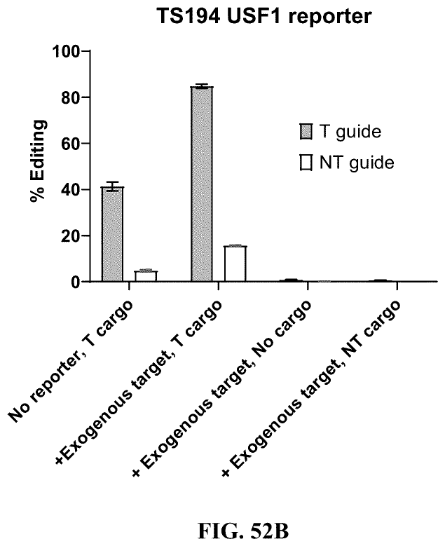

The 3′ trans-splicing of the gene USF1 was assessed using 4 different components: first, either a non-targeting or a targeting cargo replacing the USF1 terminal exon and containing an XTEN linker and 3×Flag tag; second, a USF1 intron 10 targeting or scrambled guide RNA; third, a reporter plasmid containing USF1 cDNA with intron 10 in between the all the upstream and downstream exons; and fourth, Cas7-11 was included in all conditions (FIG. 52A and FIG. 52B). The bar graph of FIG. 52B reports the 3′ trans-splicing rate (%) for the gene USF1 as a confirmation of the Western blot from FIG. 52A. With Cas7-11 and targeting guide, about 40% and 80% of editing were obtained for endogenous or reporter trans-splicing, respectively.

### Materials and Methods

The constructs were transfected at a 6-well scale on HEK293FT cells in DMEM 10% FBS. Transfections were carried out using:
400 ng of guide;
400 ng of cargo plasmid; and
1600 ng of Cas7-11 plasmid,
   co-transfected using Lipofectamine 3000.

Each condition was transfected on 2 wells. 3 days post-transfection, RNA was harvested from 1 well, and protein was harvested from the other well, by specific lysis buffers.

Harvested RNA reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

Protein concentration was determined by BCA assay, and equal amounts from each sample were run on Bio-Rad 4-20% Mini-PROTEAN gel. They were transferred on nitrocellulose membrane via Thermo iBlot-2 transfer device, blocked for 1 h at RT, and incubated overnight with primary antibody at 4° C. Next day, membrane was washed before and after incubation with secondary antibody for 1 h at RT, and imaged by LI-COR Odyssey Scanner.

### Discussion

Even though trans-splicing occurs in the RNA-level, a goal of using this tool is replacing disease-related mutant proteins with the wild type versions. Therefore, to validate editing results in RNA-level, and show the translation of trans-spliced product, Western blot is one of the most important techniques.

### Example 51. 3′ Trans-Splicing Rate for gLuc Gene

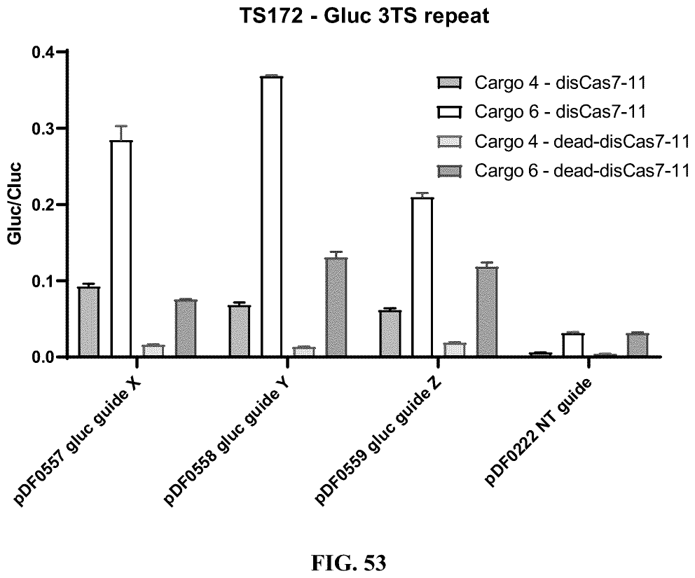

The 3′ trans-splicing rates (%) for the gene gLuc were assessed in a reporter plasmid (FIG. 53). Effects of 2 different cargo, together with 3 targeting and 1 non-targeting guide, and either functional or dead Cas7-11 on 3′ trans-splicing were measured. Positive effects of a functional Cas7-11 and targeting guides over non-targeting is clear. Cargo 6 and guide Y were selected for the further experiments.

### Materials and Methods

To represent the actual trans-splicing, cDNA expressing gLuc were split with an intron between them, and a coding region was truncated to eliminate any background. Truncated region, together with the downstream part of the gene were included in the cargo. This way, only after a targeted trans-splicing, a functional gLuc will be expressed. In the same plasmid, a full length cLuc gene was used as a transfection control.

The constructs were transfected at a 96-well scale on HEK293FT cells in DMEM 10% FBS. Transfections were carried out using:
10 ng of guide;
10 ng of cargo plasmid;
10 ng of reporter plasmid; and
40 ng of Cas7-11 plasmid,
   co-transfected using Lipofectamine 3000.

Culture media containing secreted luciferase was collected after 2 days of transfection. 201 from each well, together with the Gaussia Luciferase Assay reagent (GAR-2B; Targeting Systems) or the Cypridina Luciferase Assay reagent (VLAR-2; Targeting Systems) were used to perform gLuc and cLuc assays, according to the manufacturer's instructions. Luminescence were measured on a Biotek Synergy Neo 2 reader. gLuc/cLuc values were used to represent trans-splicing ratio to normalize the transfection efficiency between wells.

### Discussion

A reporter system for 3′ trans-splicing provides a fast and easy way to test effects of different constructs on trans-splicing rate. It can be used as a first step for screening new constructs to find the ones improving trans-splicing, before moving on to the endogenous 3′ trans-splicing.

### Example 52. 5′ Trans-Splicing Rate for HTT Gene

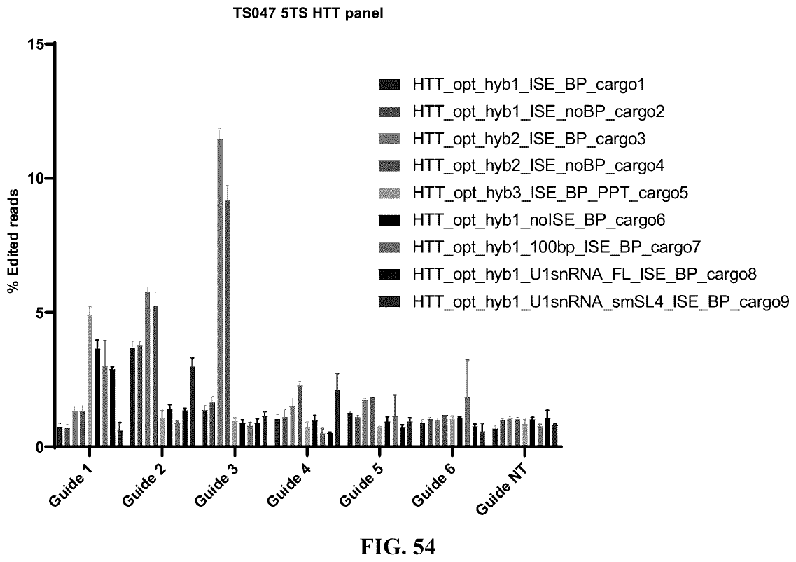

The 5′ trans-splicing rate (%) for HTT exon 1 were assessed using cargos that bind to intron 1 and a guide targeting the terminal end of the cargo hybridization or a scrambled NT sequence (FIG. 54). Variations of the cargo that incorporate different sequence motifs, including 5′ splicing consensus motifs, branch points, snRNA recognition sequences, predicted splicing enhancer sequences, or combinations of the above were compared for effect on splicing rates. Different guide sequences tiled around best performing guides from an initial screen were transfected with each cargo to determine whether guide placement further improves rates.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example compares a wide range of different 5′ splicing architectures for cargos targeting the first exon of HTT. Several of these components have effects on the splicing rate, with the original cargo listed 2^{nd} from right performing nearly as well as the best hit. The largest improvements to splicing rates come from inclusion of the branch point and GURAGU motif (known to participate in the splicing reaction).

These results suggest that a relatively basic structure for a cargo accomplishes most of the splicing rate for 5′ trans-splicing and confirms that the ISE and GURAGU/GUUAGU are essential for highly efficient splicing. Different cargo structures also show different preferences for guides.

### Example 53. 5′ Trans-Splicing Rate for HTT Gene

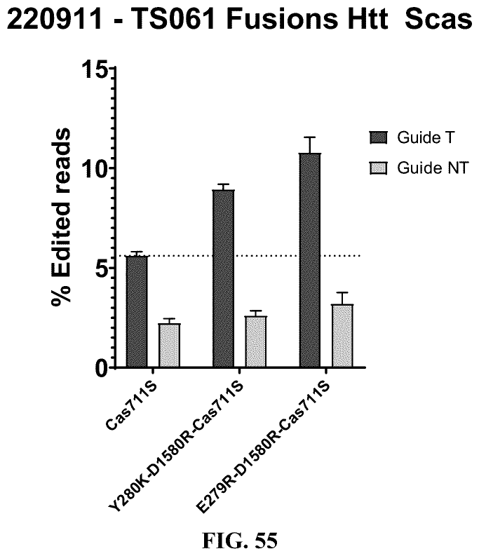

The 5′ trans-splicing rates (%) for HTT exon 1 were assessed using cargo constructs with hybridization regions that bind intron 1 of the HTT premRNA and either a scrambled guide or a guide that binds and cleaves the end of the hybridization (FIG. 55). Splicing rate was reported for both truncated Cas7-11 and truncated Cas7-11 with top-performing mutations from a screen on full-length Cas7-11.

### Materials and Method

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example tests constructs to be used for an AAV packaging system for trans-splicing for 5′ splicing of HTT. The truncated Cas7-11 necessary for AAV packaging (to fit within the size constraints of an AAV backbone) performs less than the full length cas7-11 wt, which reflect equivalent results from other comparisons of full length and truncated nucleases. It is likely that the smaller constructs needed for AAV delivery of trans-splicing components can reduce splicing efficiency. However, the degree to which can be variable and related to specific genes.

### Example 54. 5′ Trans-Splicing Rate for HTT Gene

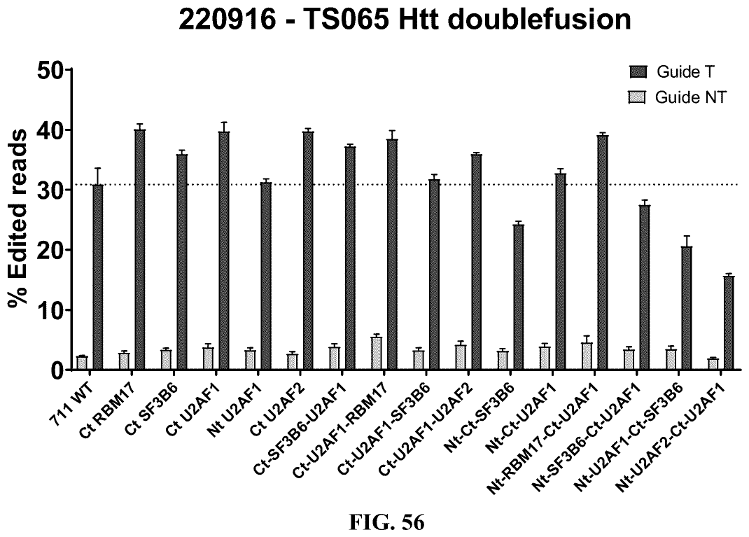

The 5′ trans-splicing rates (%) for HTT exon 1 were assessed using cargos that bind to intron 1 and a guide targeting the terminal end of the cargo hybridization, or a scrambled NT sequence combined on a single plasmid (FIG. 56). The wildtype disCas7-11 nuclease as compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing. These improvements further scale with the single vector versions of the HTT constructs, where guides and cargos are cloned into a single plasmid. This shows that multiple orthogonal methods for improving efficiency are compatible with each other and can be developed in parallel for a given target, and that the improvements seen for other genes (e.g., STAT3) are generalizable.

### Example 55. 5′ Trans-Splicing Rate for HTT Gene

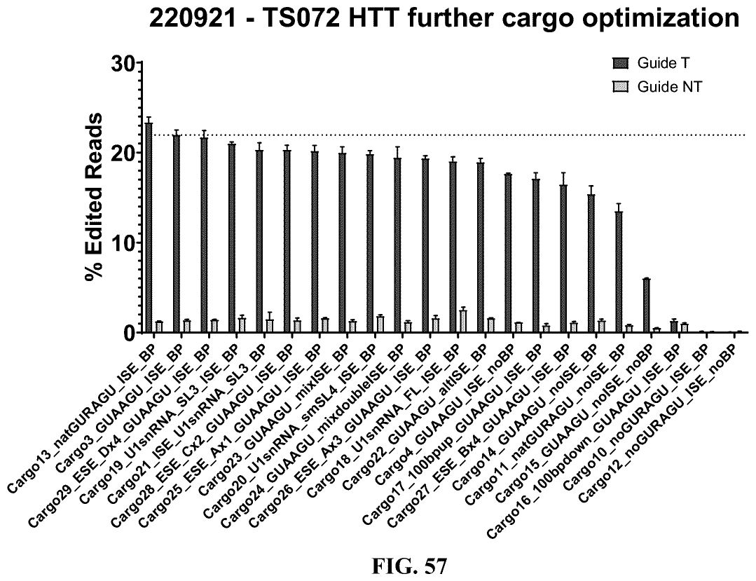

The 5′ trans-splicing rate (%) for HTT exon 1 were assessed using cargos that bind to intron 1 and a guide targeting the terminal end of the cargo hybridization or a scrambled NT sequence (FIG. 57). Variations of the cargo that incorporate different sequence motifs, including 5′ splicing consensus motifs, branch points, snRNA recognition sequences, predicted splicing enhancer sequences, or combinations of the above were compared for effect on splicing rates.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example compares a wide range of different 5′ splicing architectures for cargos targeting the first exon of HTT. Several of these components have effects on the splicing rate, with the original cargo listed 2^{nd} from right performing nearly as well as the best hit. The largest improvements to splicing rates come from inclusion of the branch point and GURAGU motif (known to participate in the splicing reaction).

These results suggest that a relatively basic structure for a cargo accomplishes most of the splicing rate for 5′ trans-splicing and confirms that the ISE and GURAGU/GUUAGU are essential for highly efficient splicing.

### Example 56. 5′ Trans-Splicing Rate for HTT Gene

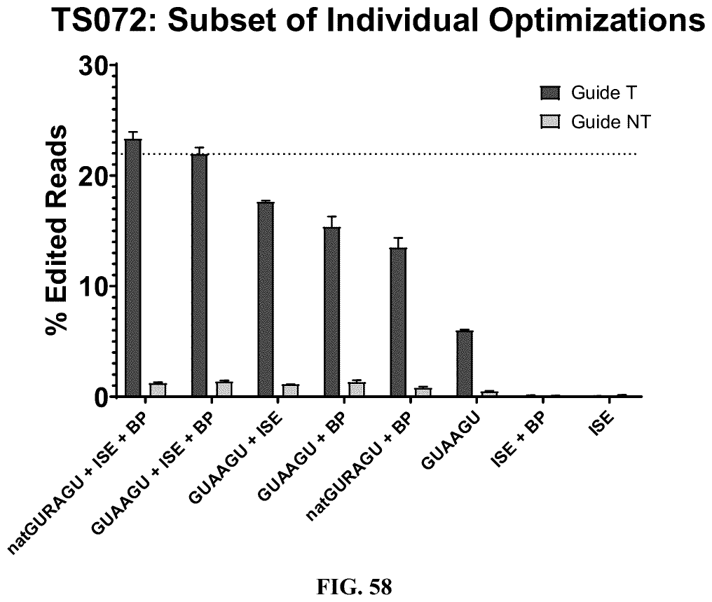

The 5′ trans-splicing rate (%) for HTT exon 1 were assessed using cargos that bind to intron 1 and a guide targeting the terminal end of the cargo hybridization or a scrambled NT sequence (FIG. 58). Variations of the cargo that incorporate different sequence motifs, including 5′ splicing consensus motifs, branch points, snRNA recognition sequences, predicted splicing enhancer sequences, or combinations of the above were compared for effect on splicing rates.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

FIG. 58 is a subset of a previous larger panel which compares a wide range of different 5′ splicing architectures for cargos targeting the first exon of HTT. Several of these components have effects on the splicing rate, with the original cargo listed 2nd from right performing nearly as well as the best hit. The largest improvements to splicing rates come from inclusion of the branch point and GURAGU motif (known to participate in the splicing reaction).

These results suggest that a relatively basic structure for a cargo accomplishes most of the splicing rate for 5′ trans-splicing and confirms that the ISE and GURAGU/GUUAGU are essential for highly efficient splicing. In particular, cargos without a GURAGU motif can be incapable of splicing (3rd from right includes, compared to 2 furthest right in FIG. 58).

### Example 57. 5′ Trans-Splicing Rate for HTT Gene

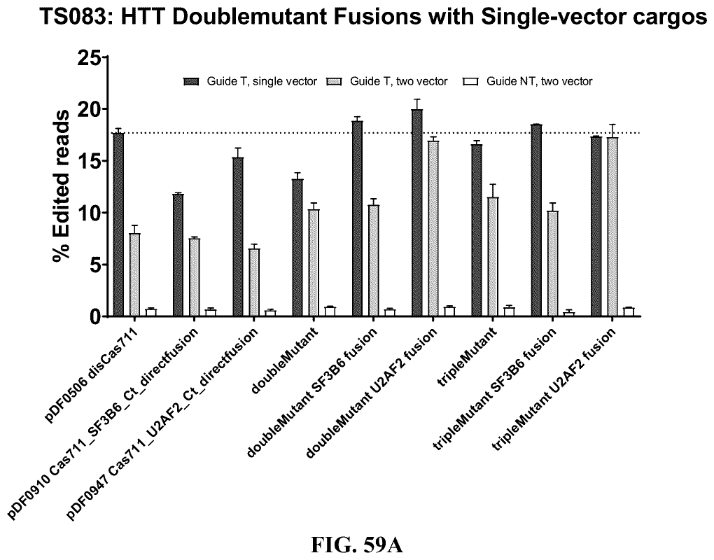

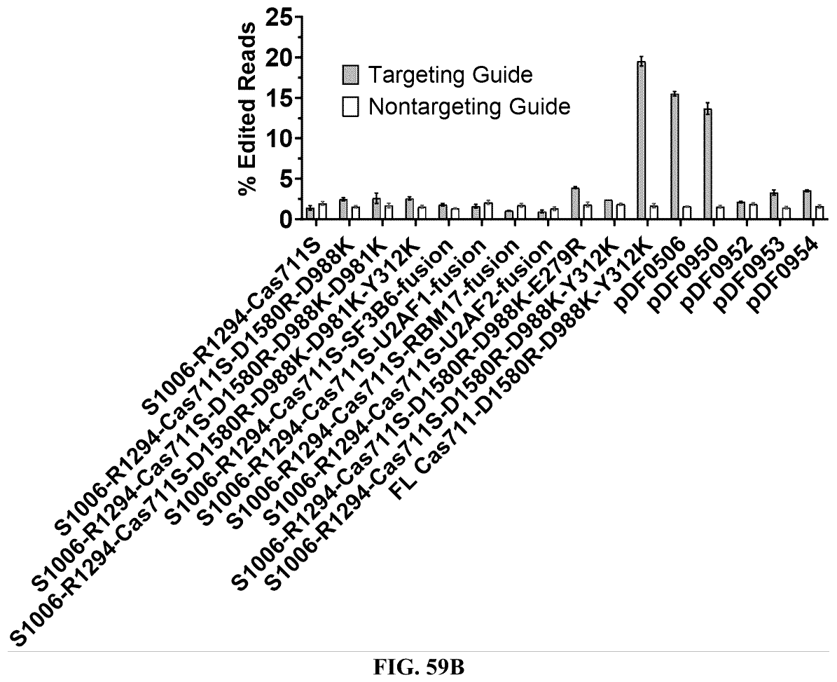

The 5′ trans-splicing rate (%) for HTT exon 1 were assessed using cargos that bind to intron 1 and a guide targeting the terminal end of the cargo hybridization, or a scrambled NT sequence combined on a single plasmid (FIG. 59A). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

### Materials and Method

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing. These improvements further scale with the single vector versions of the HTT constructs, where guides and cargos are cloned into a single plasmid. However, the improvement seen with single vector constructs from nuclease engineering is less pronounced than with the two-vector equivalent, potentially indicating a saturation or rate-limiting step being resolved by the single vectors.

### Example 57. 5′ Trans-Splicing Rate for HTT Gene

The 5′ trans-splicing rates (%) for HTT gene were assessed (FIG. 59B). Trans-splicing editing rates are plotted for a set of constructs that include Cas7-11 mutants, mutants fused to splicing proteins, and “small” cas7-11 constructs with internal truncations with mutations or fusions. This figure reports a 5′ splicing rate for HTT exon 1, using cargos that bind to intron 1 and a guide targeting the terminal end of the cargo hybridization or a scrambled NT sequence. The wildtype disCas7-11 nuclease is compared here against constructs with a direct fusion to various splicing proteins (RMB17, SF3B6, U2AF1, or U2AF2) at their N- or C-terminals, as well as constructs generated through rounds of mutagenesis of disCas7-11 that show higher catalytic activity.

These results show modest improvements to splicing rates from each of the orthogonal and combined engineering strategies on top of the “small” Cas7-11 chassis. In some aspects, the overall performance of the small Cas7-11 is lower than the full-length Cas7-11 constructs compared here.

All constructs were transfected at a 96-well scale on HEK293FT cells in DMEM 10% FBS. For endogenous splicing experiments, transfections were carried out using
10 ng of guide
10 ng of cargo plasmid
40 ng of Cas7-11 plasmid
   co-transfected using Lipofectamine 3000.

RNA was harvested 3 days post-transfection and reverse transcribed using the Thermo RevertAid cDNA prep kid with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Example 58. 5′ Trans-Splicing Rate for USF1 Gene

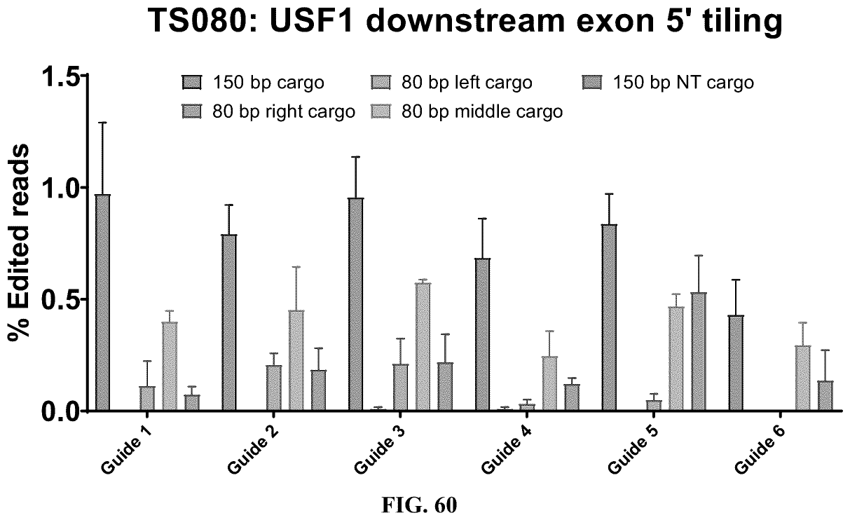

The 5′ trans-splicing rates (%) for USF1 exon 9 were assessed using cargo constructs with hybridization regions that bind intron 9 of the USF1 premRNA and either a scrambled guide or a guide that binds and cleaves upstream of the hybridization region (FIG. 60). Cargos with different hybridization lengths were tiled across the intron in question and crossed with an array of guides cleaving at different positions within the intron.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

The results from this example suggest a present but inefficient trans-splicing rate in a guide-and-cargo dependent fashion for a terminal intron of USF1. Specific cargos show a larger overall rate and ratio of background activity (where guide 6 represents a nontargeting guide, or no Cas7-11 condition).

These results suggest that cas7-11 is also able to enhance 5′ splicing rates by removing upstream cis exons, or through binding the transcript, although the overall rates are lower relative to those accomplished through 3′ trans-splicing.

### Example 59. 5′ and 3′ Trans-Splicing Rates for HTT and SHANK3 Genes Respectively

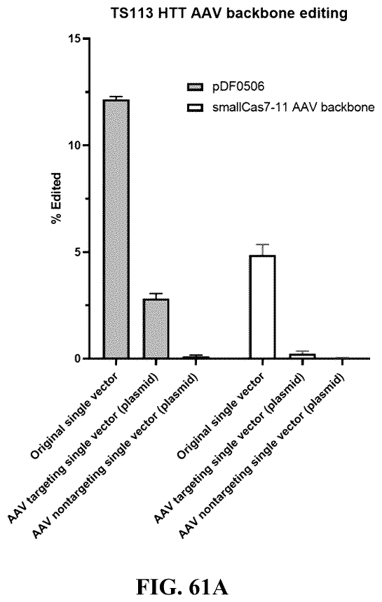

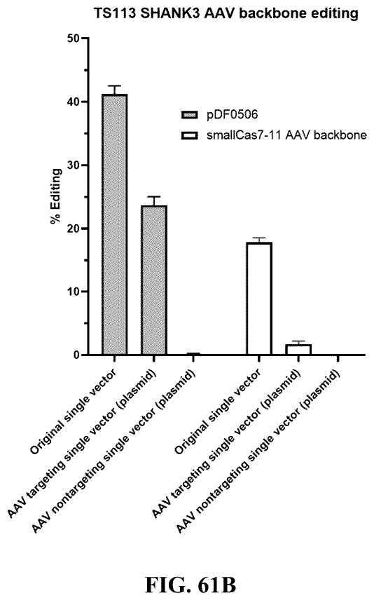

The 5′ trans-splicing rates (%) for the gene HTT (FIG. 61A) and 3′ trans-splicing rate (%) for the gene SHANK3 (FIG. 61B) were assessed. The rate for the HTT gene was probed using cargo constructs with hybridization regions that bind intron 1 of the HTT premRNA and either a scrambled guide or a guide that binds and cleaves the end of the hybridization. The rate for the SHANK3 exon 21 was probed with a cargo and guide binding within intron 20. Cargo and guide constructs were expressed either from a normal plasmid (single vector) or a subcloned AAV backbone construct. Splicing rate was reported for both Cas7-11 and a truncated Cas7-11 subcloned into an AAV expression backbone.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example tests constructs to be used for an AAV packaging system for trans-splicing, either for 5′ trans-splicing of HTT or 3′ trans-splicing of SHANK3. The truncated Cas7-11 necessary for AAV packaging performs less than the full length cas7-11 wt, which reflect equivalent results from other comparisons of full length and truncated nucleases. Furthermore, the AAV single vector with guide and cargo performs less for HTT editing, but still retains ˜60% of the original editing rate for SHANK3.

It is likely that the smaller constructs needed for AAV delivery of trans-splicing components can reduce splicing efficiency. However, the degree to which can be variable and related to specific genes.

### Example 60. 5′ Trans-Splicing Rate for PABPC1 Gene

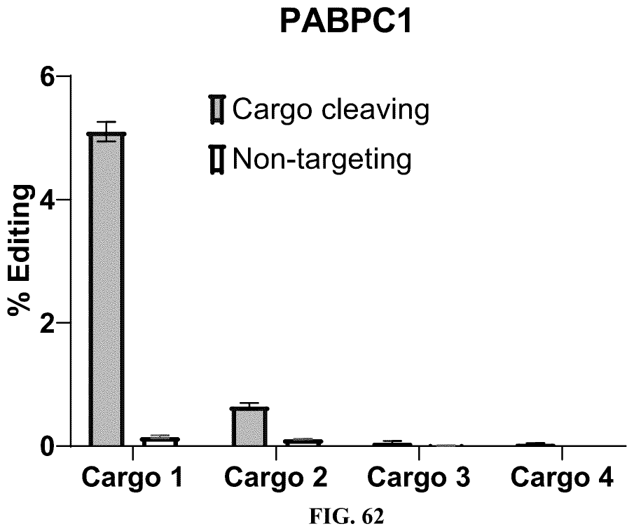

The 5′ trans-splicing rates (%) for PABPC1 exon 1 were assessed using cargo constructs with hybridization regions that bind intron 1 of the PABPC1 premRNA and either a scrambled guide or a guide that binds and cleaves the end of the hybridization (FIG. 62).

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

In this example, the Cas7-11 serves to cleave the 3′ end of the 5′ trans-splicing cargo, effectively removing any trailing sequences from the plasmid, specifically the polyA tail. This has a beneficial effect on splicing rates, potentially due to a decrease in nuclear export and translation of the un-spliced cargo.

Together with results from other targets, these results show that polyA removal is important for 5′ trans-splicing.

### Example 61. 5′ Trans-Splicing Rate for RPL41 Gene

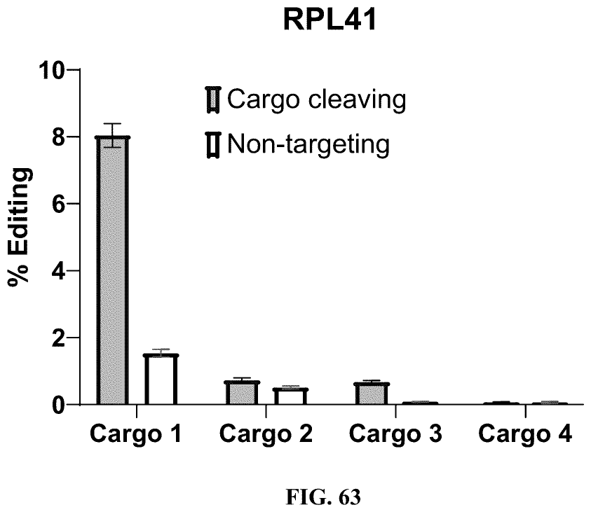

The 5′ trans-splicing rates (%) for RPL41 exon 1 were assessed using cargo constructs with hybridization regions that bind intron 1 of the RPL41 premRNA and either a scrambled guide or a guide that binds and cleaves the end of the hybridization (FIG. 63).

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

In this example, the Cas7-11 serves to cleave the 3′ end of the 5′ trans-splicing cargo, effectively removing any trailing sequences from the plasmid, in particular the polyA tail. This has a beneficial effect on splicing rates, potentially due to a decrease in nuclear export and translation of the un-spliced cargo.

Together with results from other targets, these results show that polyA removal is important for 5′ trans-splicing.

### Example 62. 5′ Trans-Splicing Rate for HTT Gene

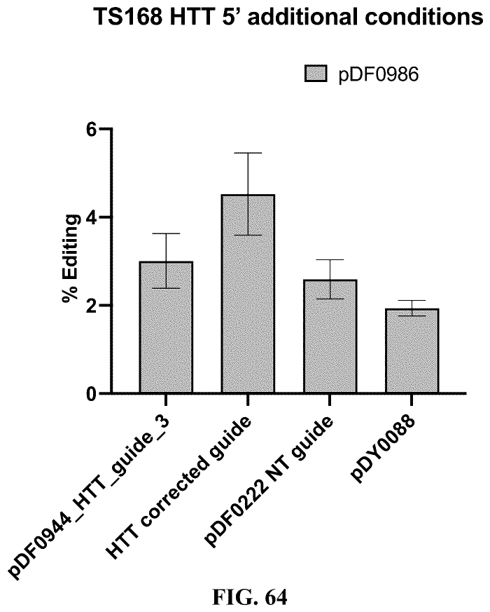

The 5′ trans-splicing rates (%) for HTT exon 1 were assessed using the original cargo construct (FIG. 64). Cargo construct was transfected either with a cargo targeting guide, a guide targeting the HTT intron 1, a scrambled (nontargeting) guide, or an RFP plasmid in place of the guide.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example serves to test how different methods of removing the polyA tail and trailing sequences from a 5′ trans-splicing cargo affect overall rates for HTT. Cargos show good efficiency with cargo cleaving and intron targeting guides, but also moderate activity without a guide present, presently a possibility for reasonable rates without the Cas7-11 constructs.

### Example 63. 5′ Trans-Splicing Rate for HTT Gene

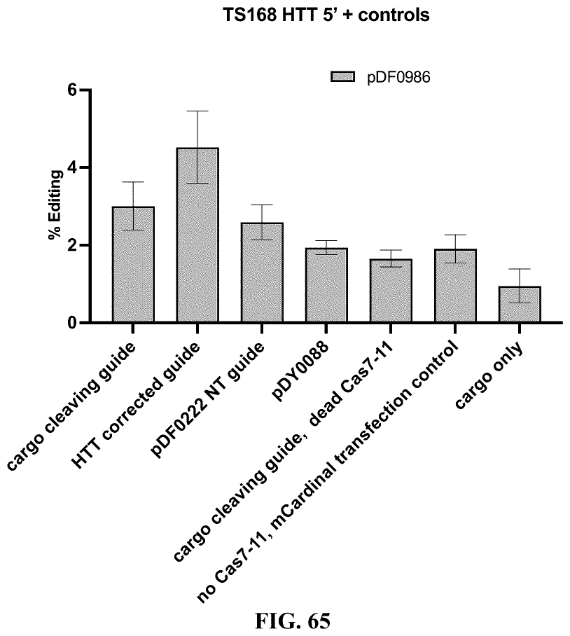

The 5′ trans-splicing rates (%) for HTT exon 1 were assessed using the original cargo construct (FIG. 65). Cargo construct was transfected either with a cargo targeting guide, a guide targeting the HTT intron 1, a scrambled (nontargeting) guide, or an RFP plasmid in place of the guide. Additional controls where cargos are transfected with no Cas7-11 or catalytically inactive Cas7-11s were also probed.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example serves to test how different methods of removing the polyA tail and trailing sequences from a 5′ trans-splicing cargo affect overall rates for HTT. Cargos show good efficiency with cargo cleaving and intron targeting guides, but also moderate activity without a guide present, presently a possibility for reasonable rates without the Cas7-11 constructs.

### Example 64. 5′ Trans-Splicing Rate for HTT Gene

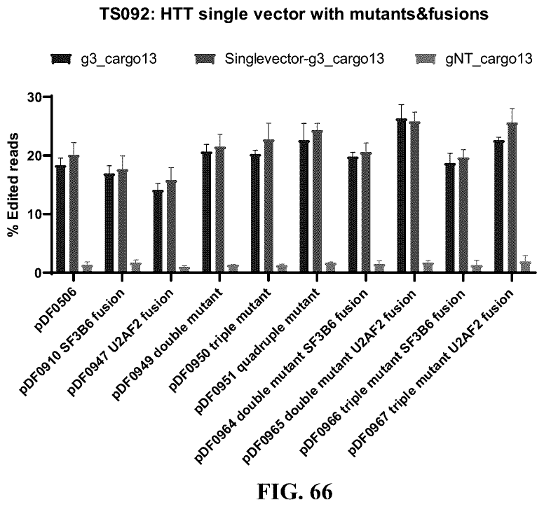

The 5′ trans-splicing rates (%) for HTT exon 1 were assessed using cargos that bind to intron 1 and a guide targeting the terminal end of the cargo hybridization, or a scrambled NT sequence combined on a single plasmid (FIG. 66). The wildtype disCas7-11 nuclease was compared against constructs with a direct (no intervening linker) or indirect (XTEN linker intervening) fusion to various splicing proteins (RMB17, SF3B6, U2AF1, or U2AF2) at their N- or C-terminals, as well as constructs generated through rounds of mutagenesis of disCas7-11 that show higher catalytic activity.

### Materials and Methods

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within the inserted or wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

Fusions of splicing related proteins, such as the various members of the spliceosome, to the Cas7-11 effector can provide an orthogonal (not cleavage dependent) mechanism for increasing trans-splicing rates. This fusion approach is analogous to how CRISPR-based technologies such as Prime editing or CRISPR activation gain efficiency through recruitment or delivery of relevant factors with the major effector.

In this example, several fusions of spliceosome proteins increase trans-splicing rates relative to the wildtype Cas7-11. Similarly, improvements to catalytic activity of the disCas7-11 improve overall trans-splicing rates, suggesting that intron cleavage is a rate-limiting factor for trans-splicing. These improvements further scale with the single vector versions of the HTT constructs, where guides and cargos are cloned into a single plasmid. This shows that multiple orthogonal methods for improving efficiency are compatible with each other and can be developed in parallel for a given target, and that the improvements seen for other genes (e.g., STAT3) are generalizable.

### Example 65. 5′ Trans-Splicing Rate for HTT Gene

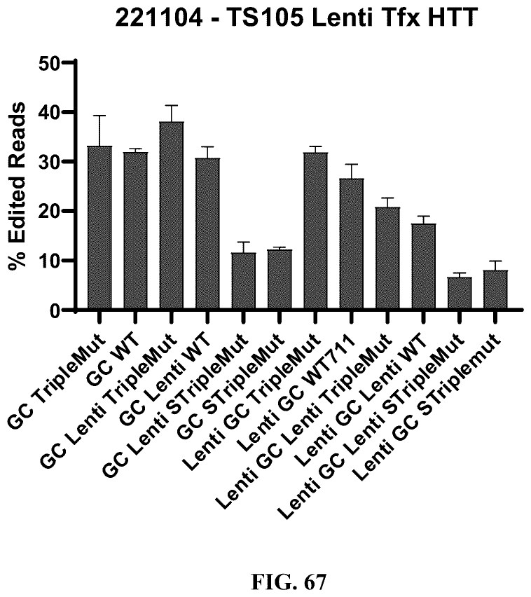

The 5′ trans-splicing rates (%) for HTT exon 1 were assessed using a cargo construct with a hybridization region that binds intron 1 of the HTT premRNA (FIG. 67). Cargo and guide constructs were expressed from a normal plasmid (single vector), while nuclease constructs were expressed from normal plasmid or subcloned lentiviral backbone. Shown are splicing rates for triple mutant Cas7-11 and wtCas7-11.

### Materials and Method

The constructs were transfected at a 96-well scale on HEK293FT cells in DMEM 10% FBS. For endogenous splicing experiments, transfections were carried out using:
10 ng of guide;
10 ng of cargo plasmid; and
40 ng of Cas7-11 plasmid,
   co-transfected using Lipofectamine 3000.

RNA was harvested 3 days post-transfection and reverse transcribed using the Thermo RevertAid cDNA prep kit with gene specific reverse transcription primers binding within downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

### Discussion

This example tests several lentiviral expression backbones before lentiviral packaging. Efficient splicing with transient transfection of lentiviral backbone should indicate the potential for efficient splicing post lentiviral production. It was observed that several lenti constructs perform similarly or better than the conventional plasmid Cas7-11, with full length lentiviral constructs still performing more efficiency than the truncated equivalents.

### LIST OF REFERENCES

All publications and references cited herein are expressly incorporated herein by reference in their entirety.
U.S. Patent Application Publication No. US2004/0018622.
International Patent Application Publication No. WO2005/070023A2.
European Patent Application Publication No. EP2151248A1.
Anzalone A V, Koblan L W, Liu D R. Genome editing with CRISPR-Cas nucleases, base editors, transposases and prime editors. Nature Biotechnology 2020:824-44. https://doi.org/10.1038/s41587-020-0561-9.
Soppe J A, Lebbink R J. Antiviral Goes Viral: Harnessing CRISPR/Cas9 to Combat Viruses in Humans. Trends Microbiol 2017; 25:833-50.
Abudayyeh O O, Gootenberg J S, Essletzbichler P. RNA targeting with CRISPR-Cas13. Nature 2017.
Smargon A A, Cox D B T, Pyzocha N K, Zheng K, Slaymaker I M, Gootenberg J S, et al. Cas13b Is a Type VI-B CRISPR-Associated RNA-Guided RNase Differentially Regulated by Accessory Proteins Csx27 and Csx28. Mol Cell 2017; 65:618-630.e7.
Konermann S, Lotfy P, Brideau N J, Oki J, Shokhirev M N, Hsu P D. Transcriptome Engineering with RNA-Targeting Type VI-D CRISPR Effectors. Cell 2018; 0: https://doi.org/10.1016/j.cell.2018.02.033.
Cox D B T, Gootenberg J S, Abudayyeh O O, Franklin B, Kellner M J, Joung J, et al. RNA editing with CRISPR-Cas13. Science 2017; 358:1019-27.
Wilson C, Chen P J, Miao Z, Liu D R. Programmable m6A modification of cellular RNAs with a Cas13-directed methyltransferase. Nat Biotechnol 2020. https://doi.org/10.1038/s41587-020-0572-6.
Abudayyeh O O, Gootenberg J S, Konermann S, Joung J, Slaymaker I M, Cox D B T, et al. C2c2 is a single-component programmable RNA-guided RNA-targeting CRISPR effector. Science 2016; 353:aaf5573.
Meeske A J, Nakandakari-Higa S, Marraffini L A. Cas13-induced cellular dormancy prevents the rise of CRISPR-resistant bacteriophage. Nature 2019. https://doi.org/10.1038/s41586-019-1257-5.
Wang Q, Liu X, Zhou J, Yang C, Wang G, Tan Y, et al. The CRISPR-Cas13a Gene-Editing System Induces Collateral Cleavage of RNA in Glioma Cells. Adv Sci 2019; 1:1901299.
Wang L, Zhou J, Wang Q, Wang Y, Kang C. Rapid design and development of CRISPR-Cas13a targeting SARS-CoV-2 spike protein. Theranostics 2021; 11:649-64.
Engreitz J, Abudayyeh O, Gootenberg J, Zhang F. CRISPR Tools for Systematic Studies of RNA Regulation. Cold Spring Harb Perspect Biol 2019; 11: https://doi.org/10.1101/cshperspect.a035386.
Shmakov S, Abudayyeh O O, Makarova K S, Wolf Y I, Gootenberg J S, Semenova E, et al. Discovery and Functional Characterization of Diverse Class 2 CRISPR-Cas Systems. Mol Cell 2015; 60:385-97.
Shmakov S, Smargon A, Scott D, Cox D, Pyzocha N, Yan W, et al. Diversity and evolution of class 2 CRISPR-Cas systems. Nat Rev Microbiol 2017; 15:169-82.
Shmakov S A, Faure G, Makarova K S, Wolf Y I, Severinov K V, Koonin E V. Systematic prediction of functionally linked genes in bacterial and archaeal genomes. Nat Protoc 2019. https://doi.org/10.1038/s41596-019-0211-1.
Pourcel C, Touchon M, Villeriot N, Vernadet J-P, Couvin D, Toffano-Nioche C, et al. CRISPRCasdb a successor of CRISPRdb containing CRISPR arrays and cas genes from complete genome sequences, and tools to download and query lists of repeats and spacers. Nucleic Acids Research 2019. https://doi.org/10.1093/nar/gkz915.
Edgar R C. PILER-CR: fast and accurate identification of CRISPR repeats. BMC Bioinformatics 2007; 8:18.
Anantharaman V, Makarova K S, Burroughs A M, Koonin E V, Aravind L. Comprehensive analysis of the HEPN superfamily: identification of novel roles in intra-genomic conflicts, defense, pathogenesis and RNA processing. Biol Direct 2013; 8:15.
Wang R, Li H. The mysterious RAMP proteins and their roles in small RNA-based immunity. Protein Sci 2012; 21:463-70.
Makarova K S, Wolf Y I, Iranzo J, Shmakov S A, Alkhnbashi O S, Brouns S J J, et al. Evolutionary classification of CRISPR-Cas systems: a burst of class 2 and derived variants. Nat Rev Microbiol 2019; 18:67-83.
Harrington L B, Burstein D, Chen J S, Paez-Espino D, Ma E, Witte I P, et al. Programmed DNA destruction by miniature CRISPR-Cas14 enzymes. Science 2018; 362:839-42.
Ye Y, Zhang Q. Characterization of CRISPR RNA transcription by exploiting stranded metatranscriptomic data. RNA 2016; 22:945-56.
Zetsche B, Heidenreich M, Mohanraju P, Fedorova I, Kneppers J, DeGennaro E M, et al. Multiplex gene editing by CRISPR-Cpf1 using a single crRNA array. Nat Biotechnol 2017; 35:31-4.
Ozcan A, Krajeski R, Ioannidi E, Lee B, Gardner A, Makarova K S, et al. Programmable RNA targeting with the single-protein CRISPR effector Cas7-11. Nature 2021. https://doi.org/10.1038/s41586-021-03886-5.
Marshall R, Maxwell C S, Collins S P, Jacobsen T, Luo M L, Begemann M B, et al. Rapid and Scalable Characterization of CRISPR Technologies Using an E. coli Cell-Free Transcription-Translation System. Mol Cell 2018; 69:146-157.e3.
Teng F, Li J, Cui T, Xu K, Guo L, Gao Q, et al. Enhanced mammalian genome editing by new Cas12a orthologs with optimized crRNA scaffolds. Genome Biol 2019; 20:15.
Oakes B L, Fellmann C, Rishi H, Taylor K L, Ren S M, Nadler D C, et al. CRISPR-Cas9 Circular Permutants as Programmable Scaffolds for Genome Modification. Cell 2019; 176:254-267.e16.
Palazzo A F, Lee E S. Sequence determinants for nuclear retention and cytoplasmic export of mRNAs and lncRNAs. Front Genet 2018; 9:440.

LENGTHY TABLES
The patent application contains a lengthy table section. A copy of the table is available in electronic form from the USPTO web site (https://seqdata.uspto.gov/?pageRequest=docDetail&DocID=US20240100192A1). An electronic copy of the table will also be available from the USPTO upon request and payment of the fee set forth in 37 CFR 1.19(b)(3).

What is claimed:

1. A composition for nucleic acid editing comprising:
a) a trans-splicing template polynucleotide comprising a cargo guide sequence complementary to a portion of an intron or exon sequence of a target RNA sequence, optionally wherein the trans-splicing template polynucleotide further comprises an integration sequence, a 3′ splicing site sequence, a branch point sequence, and a polypyrimidine tract sequence, and wherein each sequence is operably connected in any order; and
b) a Cas7-11 enzyme sequence coupled to a guide RNA sequence that is complementary to a portion of the intron or exon sequence of the target RNA sequence that is upstream, downstream, or overlapping of the portion of the intron or exon sequence that is complementary to the cargo guide sequence.

2. The composition of claim 1, wherein the trans-splicing template polynucleotide comprises a nucleic acid sequence about 80% identical, about 90% identical, about 95% identical, about 99% identical, or identical to any one of the nucleic acid sequences of SEQ ID NOS: 106-171 and 179-184.

3. The composition of claim 1, wherein the Cas7-11 enzyme sequence comprises a nucleic acid sequence about 80% identical, about 90% identical, about 95% identical, about 99% identical, or identical to any one of the nucleic acid sequences of SEQ ID NOS: 1-18.

4. The composition of claim 1, wherein the guide RNA sequence comprises a nucleic acid sequence about 80% identical, about 90% identical, about 95% identical, about 99% identical, or identical to any one of the nucleic acid sequences of SEQ ID NOS: 19-105 and 205-261.

5. A method of editing the target RNA sequence of claim 1 in a cell, the method comprising:
a) providing to the cell the trans-splicing template polynucleotide of claim 1, a vector comprising the trans-splicing template polynucleotide of claim 1, or a particle comprising the trans-splicing template polynucleotide of claim 1;
b) providing to the cell a polynucleotide translating the Cas7-11 enzyme sequence of claim 1, a vector comprising a polynucleotide translating the Cas7-11 enzyme sequence of claim 1, or a particle comprising a polynucleotide translating the Cas7-11 enzyme sequence of claim 1;
c) providing to the cell a polynucleotide expressing the guide RNA sequence of claim 1, a vector comprising a polynucleotide expressing the guide RNA sequence of claim 1, or a particle comprising a polynucleotide expressing the guide RNA sequence of claim 1; and
d) editing the target RNA sequence via 3′ trans-splicing.

6. A method of treating or preventing a genetically inherited disease in a subject in need thereof, the method comprising administering to the subject an effective amount of:
a) the trans-splicing template polynucleotide of claim 1, a vector comprising the trans-splicing template polynucleotide of claim 1, or a particle comprising the trans-splicing template polynucleotide of claim 1;
b) a polynucleotide translating the Cas7-11 enzyme sequence of claim 1, a vector comprising a polynucleotide translating the Cas7-11 enzyme sequence of claim 1, or a particle comprising a polynucleotide translating the Cas7-11 enzyme sequence of claim 1; and
c) a polynucleotide expressing the guide RNA sequence of claim 1, a vector comprising a polynucleotide expressing the guide RNA sequence of claim 1, or a particle comprising a polynucleotide expressing the guide RNA sequence of claim 1.

7. The method of claim 6, wherein the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

8. A composition for nucleic acid editing comprising:
a) a trans-splicing template polynucleotide comprising a cargo guide sequence complementary to a portion of an intron or exon sequence of a target RNA sequence, optionally wherein the trans-splicing template polynucleotide further comprises an integration sequence, a 5′ splicing site sequence, an intronic signal enhancer (ISE) sequence, and/or an exonic signal enhancer (ESE) sequence, wherein each sequence is operably connected in any order; and
b) a Cas7-11 enzyme sequence coupled to a guide RNA sequence that is complementary to a portion of the intron or exon sequence of the target RNA sequence that is upstream, downstream, or overlapping of the portion of the intron or exon sequence that is complementary to the cargo guide sequence.

9. The composition of claim 8, wherein the trans-splicing template comprises a nucleic acid sequence about 80% identical, about 90% identical, about 95% identical, about 99% identical, or identical to any one of the nucleic acid sequences of SEQ ID NOS: 106-171 and 179-184.

10. The composition of claim 8, wherein the Cas7-11 enzyme sequence comprises a nucleic acid sequence about 80% identical, about 90% identical, about 95% identical, about 99% identical, or identical to any one of the nucleic acid sequences of SEQ ID NOS: 1-18.

11. The composition of claim 8, wherein the guide RNA comprises a nucleic acid sequence about 80% identical, about 90% identical, about 95% identical, about 99% identical, or identical to any one of the nucleic acid sequences of SEQ ID NOS: 19-105 and 205-261.

12. A method of editing the target RNA sequence of claim 8 in a cell, the method comprising:
a) providing to the cell the trans-splicing template polynucleotide of claim 8, a vector comprising the trans-splicing template polynucleotide of claim 8, or a particle comprising the trans-splicing template polynucleotide of claim 8;
b) providing to the cell a polynucleotide translating the Cas7-11 enzyme sequence of claim 8, a vector comprising a polynucleotide translating the Cas7-11 enzyme sequence of claim 8, or a particle comprising a polynucleotide translating the Cas7-11 enzyme sequence of claim 8;
c) providing to the cell a polynucleotide expressing the guide RNA sequence of claim 8, a vector comprising a polynucleotide expressing the guide RNA sequence of claim 8, or a particle comprising a polynucleotide expressing the guide RNA sequence of claim 8; and
d) editing the target RNA sequence via 5′ trans-splicing.

13. A method of treating or preventing a genetically inherited disease in a subject in need thereof, the method comprising administering to the subject an effective amount of:
a) the trans-splicing template polynucleotide of claim 8, a vector comprising the trans-splicing template polynucleotide of claim 8, or a particle comprising the trans-splicing template polynucleotide of claim 8;
b) a polynucleotide translating the Cas7-11 enzyme sequence of claim 8, a vector comprising a polynucleotide translating the Cas7-11 enzyme sequence of claim 8, or a particle comprising a polynucleotide translating the Cas7-11 enzyme sequence of claim 8; and
c) a polynucleotide expressing the guide RNA sequence of claim 8, a vector comprising a polynucleotide expressing the guide RNA sequence of claim 8, or a particle comprising a polynucleotide expressing the guide RNA sequence of claim 8.

14. The method of claim 13, wherein the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

15. A system for nucleic acid editing comprising:
a) a trans-splicing template polynucleotide comprising a first cargo guide sequence complementary to a portion of the first intron or exon sequence of a target RNA sequence and a second cargo guide sequence complementary to a portion of the second intron or exon sequence of the target RNA sequence, optionally wherein the trans-splicing template polynucleotide further comprises an integration sequence, a 3′ splicing site sequence, a 5′ splicing site sequence, a branch point sequence, and/or a polypyrimidine tract sequence, wherein each sequence is operably connected in any order; and
b) a first Cas7-11 enzyme sequence coupled to a first guide RNA sequence that is complementary to a portion of the first intron or exon sequence of the target RNA sequence that is upstream, downstream, or overlapping of the portion of the intron sequence that is complementary to the first cargo guide sequence; and
c) optionally a second Cas7-11 enzyme sequence coupled to a second guide RNA sequence that is complementary to a portion of the second intron or exon sequence of the target RNA sequence that is upstream, downstream, or overlapping of the portion of the intron sequence that is complementary to the second cargo guide sequence.

16. The composition of claim 15, wherein the trans-splicing template comprises a nucleic acid sequence about 80% identical, about 90% identical, about 95% identical, about 99% identical, or identical to any one of the nucleic acid sequences of SEQ ID NOS: 106-171 and 179-184.

17. The composition of claim 15, wherein the Cas7-11 enzyme sequence comprises a nucleic acid sequence about 80% identical, about 90% identical, about 95% identical, about 99% identical, or identical to any one of the nucleic acid sequences of SEQ ID NOS: 1-18.

18. The composition of claim 15, wherein the guide RNA comprises a nucleic acid sequence about 80% identical, about 90% identical, about 95% identical, about 99% identical, or identical to any one of the nucleic acid sequences of SEQ ID NOS: 19-105 and 205-261.

19. A method of editing the target RNA sequence of claim 15 in a cell, the method comprising:
a) providing to the cell the trans-splicing template polynucleotide of claim 15, a vector comprising the trans-splicing template polynucleotide of claim 15, or a particle comprising the trans-splicing template polynucleotide of claim 15;
b) providing to the cell a polynucleotide translating the first Cas7-11 enzyme sequence of claim 15, a vector comprising a polynucleotide translating the first Cas7-11 enzyme sequence of claim 15, or a particle comprising a polynucleotide translating the first Cas7-11 enzyme sequence of claim 15;
c) providing to the cell a polynucleotide translating the second Cas7-11 enzyme sequence of claim 15, a vector comprising a polynucleotide translating the second Cas7-11 enzyme sequence of claim 15, or a particle comprising a polynucleotide translating the second Cas7-11 enzyme sequence of claim 15;
d) providing to the cell a polynucleotide expressing the guide RNA sequence of claim 15, a vector comprising a polynucleotide expressing the guide RNA sequence of claim 15, or a particle comprising a polynucleotide expressing the guide RNA sequence of claim 15; and
e) editing the target RNA sequence via internal trans-splicing.

20. A method of treating or preventing a genetically inherited disease in a subject in need thereof, the method comprising administering to the subject an effective amount of:
a) the trans-splicing template polynucleotide of claim 15, a vector comprising the trans-splicing template polynucleotide of claim 15, or a particle comprising the trans-splicing template polynucleotide of claim 15;
b) a polynucleotide translating the first Cas7-11 enzyme sequence of claim 15, a vector comprising a polynucleotide translating the first Cas7-11 enzyme sequence of claim 15, or a particle comprising a polynucleotide translating the first Cas7-11 enzyme sequence of claim 15;
c) a polynucleotide translating the second Cas7-11 enzyme sequence of claim 15, a vector comprising a polynucleotide translating the second Cas7-11 enzyme sequence of claim 15, or a particle comprising a polynucleotide translating the second Cas7-11 enzyme sequence of claim 15; and
d) a polynucleotide expressing the guide RNA sequence of claim 15, a vector comprising a polynucleotide expressing the guide RNA sequence of claim 15, or a particle comprising a polynucleotide expressing the guide RNA sequence of claim 15.

21. The method of claim 20, wherein the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.
